(19) United States
(12) Patent Application Publication
(10) Pub. No.: US 2024/0199648 A1
(43) Pub. Date: Jun. 20, 2024

(54) MAP4K1 INHIBITORS

(71) Applicant: Blueprint Medicines Corporation, Cambridge, MA (US)

(72) Inventors: Jason D. Brubaker, Cambridge, MA (US); Michael J. Burke, Cambridge, MA (US); Joshua T. Close, Cambridge, MA (US); Thomas A. Dineen, Cambridge, MA (US); Chandrasekhar V. Miduturu, Cambridge, MA (US); Emanuele Perola, Cambridge, MA (US)

(21) Appl. No.: 18/280,900

(22) PCT Filed: Mar. 7, 2022
(86) PCT No.: PCT/US2022/019168

§ 371 Date: Sep. 7, 2023

Related U.S. Application Data
(60) Provisional application No. 63/221,710, filed on Jul. 14, 2021.
(60) Provisional application No. 63/157,919, filed on Mar. 8, 2021.

(51) Int. Cl. C07D 519/00; A61K 31/4725; C07D 491/052

(52) U.S. Cl. CPC C07D 519/00; A61K 31/4725; C07D 491/052

(57) ABSTRACT

One embodiment of the disclosure is a compound represented by Formula I or a pharmaceutically acceptable salt thereof. The variables in Formula I are defined herein. Compounds of Formula I are MAP4K1 inhibitors, which can be used to treat a diseases or disorders in a subject that benefits from control of MAP4K1 activity.

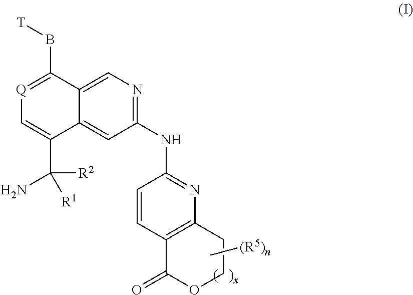

### CROSS-REFERENCE TO RELATED PATENT APPLICATIONS

This application claims the benefit of priority to U.S. Provisional Application No. 63/157,919, filed on Mar. 8, 2021, and to U.S. Provisional Application No. 63/221,710, filed on Jul. 14, 2021, the entire contents of both of which are incorporated herein by reference.

### FIELD

This application is directed to MAP4K1 inhibitors and methods for their use, such as to control the activity of MAP4K1 in a subject.

### BACKGROUND

MAP4K1, also known as hematopoietic progenitor kinase 1 (HPK1), was originally cloned from hematopoietic progenitor cells (Hu, M. C., et al., Genes Dev, 1996. 10(18): p. 2251-64). MAP4K1 is of particular interest as a target, because it is predominantly expressed in hematopoietic cells such as T cells, B cells, macrophages, dendritic cells, neutrophils, and mast cells (Hu, M. C., et al, Genes Dev, 1996. 10(18): p. 2251-64; Kiefer, F., et al, EMBO J, 1996. 15(24): p. 7013-25). MAP4K1 kinase activity has been shown to be induced upon activation of T cell receptors (TCR) (Liou, J., et al., Immunity, 2000. 12(4): p. 399-408), B cell receptors (BCR) (Liou, J., et al., Immunity, 2000. 12(4): p. 399-408), transforming growth factor receptor (TGF-R) (Wang, W., et al, J Biol Chem, 1997. 272(36): p. 22771-5; Zhou, G., et al, J Biol Chem, 1999. 274(19): p. 13133-8), or Gₛ-coupled PGE₂ receptors (EP2 and EP4) (Ikegami, R, et al, J Immunol, 2001. 166(7): p. 4689-96). As such, MAP4K1 regulates diverse functions of various immune cells.

MAP4K1 is important in regulating the functions of various immune cells and it has been implicated in autoimmune diseases and anti-tumor immunity (Shui, J. W., et al, Nat Immunol, 2007. 8(1): p. 84-91; Wang, X., et al, J Biol Chem, 2012. 287(14): p. 11037-48). Those observations suggested that attenuation of MAP4K1 activity may contribute to autoimmunity in patients. Furthermore, MAP4K1 may also control anti-tumor immunity via T cell-dependent mechanisms. In the PGE2-producing Lewis lung carcinoma tumor model, the tumors developed more slowly in MAP4K1 knockout mice as compared to wild-type mice (see US 2007/0087988). In addition, it was shown that adoptive transfer of MAP4K1 deficient T cells was more effective in controlling tumor growth and metastasis than wild-type T cells (Alzabin, S., et al., Cancer Immunol Immunother, 2010. 59(3): p. 419-29). Similarly, bone marrow derived dendritic cells (BMDCs) from MAP4K1 knockout mice were more efficient to mount a T cell response to eradicate Lewis lung carcinoma as compared to wild-type BMDCs (Alzabin, S., et al., J Immunol, 2009. 182(10): p. 6187-94). Data obtained from MAP4K1 kinase dead mice demonstrated that MAP4K1 kinase activity is critical in conferring suppressive functions of MAP4K1 in a wide range of immune cells including CD4+, CD8+, DC, NK to T regulatory cells (Tregs) and inactivation of kinase domain was sufficient to elict robust anti-tumor immune responses. Liu et al., PLoS ONE 14(3):e0212670 https://doi.org/10.1371/journal.pone.0212670. Moreover, loss of MAP4K1 kinase function suppresses tumor growth in preclinical tumor models and therapeutic co-blockade of MAP4K1 kinase and PD-L1 enhances anti-tumor responses. Hernandez S. et al., Cell Reports 2018 25: p. 80-94. Recently presented results show tumor growth inhibition in a CT-26 syngeneic mouse model using a small molecule (Seungmook, L., Cancer research.AACR Journal, 2019, Abstract 4150). These data have validated MAP4K1 as a novel drug target for enhancing antitumor immunity. Accordingly, there is a need for new compounds that modulate MAP4K1 activity for the treatment of MAP4K1-dependent diseases or disorders such as cancer, viral infection, and other diseases and disorders.

### SUMMARY

Provided herein are compounds, or pharmaceutically acceptable salts thereof, and compositions which inhibit MAP4K1, thereby enhancing an immune response in a subject. For example, the IC₅₀ values for inhibition of MAP4K1 provided in Table 3 demonstrate that these compounds are potent inhibitors of MAP4K1. Also disclosed are methods of using the compounds and compositions described herein for treating cancer and viral infection.

One embodiment of the disclosure is a compound represented by Formula I:

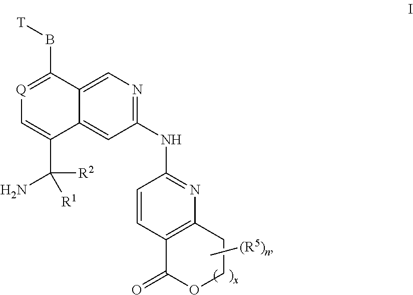

or a pharmaceutically acceptable salt thereof,
wherein:
T is selected from

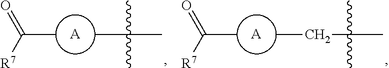

L¹-C(O)—NR¹¹R¹², -L¹-O—C(O)—NR¹¹R¹² and -L²-NR¹³—C(O)—R¹⁴;
Ring A is C₄₋₆ cycloalkyl or 4-6 membered heterocycle containing nitrogen, wherein said cycloalkyl or heterocycle is optionally substituted with 1-2 R⁶; each R⁶ is independently selected from CH₃, methoxy, CF₃, CH₂F, and CHF₂;
L¹ and L² are each independently selected from C₁-C₃ alkylene optionally substituted with 1-2 R¹⁵;
each s is independently selected from 1, 2, and 3;
B is O or NH;
Q is N or CH;
x is 0, 1, or 2;
R¹ and R² are each independently selected from hydrogen, C₁₋₆ alkyl, C₃₋₆ cycloalkyl, and 4 to 6-membered heterocycle, wherein said alkyl is optionally substituted with 1-2 R³; each R³ is independently selected from halogen, hydroxyl and OR⁴; or
each R⁴ is independently selected from C₁₋₃ alkyl, CF₃, CH₂F, and CHF₂;
each R⁵ is independently selected from C₁₋₂ alkyl, CF₃, CH₂F, and CHF₂, or
two R⁵ attached to the same carbon atom taken together with the carbon atom to which they attach form C₃₋₅ cycloalkyl; or two R⁵ attached to two adjacent carbon atoms taken together with the two adjacent carbon atoms to which they attach form C₄₋₆ cycloalkyl;
n is 0, 1, 2, 3, or 4;
R⁷ is selected from C₁₋₃ alkyl, C₃₋₆ cycloalkyl, OC₁₋₄ alkyl, NR⁹R¹⁰, and 3-5 membered heterocycle containing nitrogen or oxygen, wherein said alkyl, cycloalkyl, and heterocycle is optionally substituted with 1-3 R⁸;
each R⁸ is independently selected from halogen, C₁₋₃ alkyl, CF₃, CH₂F, CHF₂, hydroxyl, OC₁₋₃ alkyl, OCF₃, OCH₂F, and OCHF₂;
R⁹ is selected from C₁₋₂ alkyl;
R¹⁰ is selected from C₁₋₂ alkyl;
R¹¹ is selected from C₁₋₃alkyl;
R¹² is selected from C₁₋₃ alkyl; or R¹¹ and R¹² taken together with the nitrogen atom to which they attach form 4 to 6-membered heterocycle containing nitrogen;
R¹³ is H or C₁₋₂alkyl;
R¹⁴ is C₁₋₃alkyl, or R¹³ and R¹⁴ taken together with the atoms to which they attach form 4 to 6-membered heterocycle containing nitrogen; and
each R¹⁵ is independently selected from halogen, methoxy, and C₁₋₂alkyl, or two R¹⁵ taken together with the two adjacent carbon atoms to which they attach form cyclopropyl.

Another embodiment of the disclosure is a pharmaceutical composition comprising a pharmaceutically acceptable carrier or excipient and a compound disclosed herein or a pharmaceutically acceptable salt thereof.

Another embodiment of the disclosure is a method of treating a MAP4K1-dependent disorder or disease (e.g., treating a cancer) in a subject in need thereof, comprising administering to the subject an effective amount of a compound disclosed herein or a pharmaceutically acceptable salt thereof or a pharmaceutical composition comprising the compound(s).

Another embodiment of the disclosure is the use of a compound disclosed herein or a pharmaceutically acceptable salt thereof or a pharmaceutical composition comprising the compound(s), for the preparation of a medicament for treating a MAP4K1-dependent disorder or disease (e.g., treating a cancer) in a subject in need thereof.

Another embodiment of the disclosure is a compound disclosed herein or a pharmaceutically acceptable salt thereof or a pharmaceutical composition comprising the compound(s), for use in treating a MAP4K1-dependent disorder or disease (e.g., treating a cancer) in a subject in need thereof.

### DETAILED DESCRIPTION

The disclosed compounds or pharmaceutically acceptable salts thereof are MAP4K1 inhibitors, which can be used for treating a MAP4K1-dependent disorder or disease. Such diseases or disorders include cancer and viral infection.

### Compound Embodiments

Example embodiments include:

First embodiment: a compound represented by Formula I, or a pharmaceutically acceptable salt thereof. The variables in Formula I are described in the summary above.

Second embodiment: a compound represented by Formula I, or a pharmaceutically acceptable salt thereof, wherein T is selected from

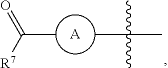

-L¹-C(O)NR¹¹R¹², and -L²-NR¹³C(O)R¹⁴.

Third embodiment: a compound represented by Formula I, or a pharmaceutically acceptable salt thereof, wherein L¹ is selected from —(CH₂)₃— and:

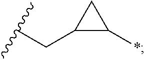

L² is —(CH₂)₂—; R¹ and R² are each independently selected from hydrogen, C₁₋₄ alkyl, C₃₋₄ cycloalkyl, and 4 to 5-membered heterocycle, wherein said alkyl is optionally substituted with one R³; each R³ is independently selected from F, Cl, hydroxyl and OR⁴; each R⁴ is independently selected from CH₃, CH₂CH₃, and CF₃; each R⁵ is independently selected from CH₃ and CF₃, or two R⁵ attached to the same carbon atom taken together with the carbon atom to which they attach form C₃₋₄ cycloalkyl; or two R⁵ attached to two adjacent carbon atoms taken together with the two adjacent carbon atoms to which they attach form C₄₋₅ cycloalkyl; n is 1, 2, 3, or 4; R⁷ is selected from C₁₋₂ alkyl, C₃₋₅ cycloalkyl, OC₁₋₃ alkyl, NR⁹R¹⁰, and 3-5 membered heterocycle containing nitrogen or oxygen, wherein said alkyl, cycloalkyl, or heterocycle is optionally substituted with 1-3 R⁸; each R⁸ is independently selected from halogen, C₁₋₂ alkyl, CF₃, CHF₂, hydroxyl, OC₁₋₂ alkyl, OCF₃, and OCHF₂; R⁹ is CH₃; R¹⁰ is CH₃; R¹¹ is CH₃; R¹² is CH₃; R¹³ is CH₃; and R¹⁴ is CH₃; or R¹³ and R¹⁴ taken together with the atoms to which they attach form 5-membered heterocycle containing nitrogen, wherein:

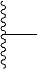

represents the point of attachment to B; and [symbol] represents the point of attachment to —C(O)NR¹¹R¹; and the remainder of the variables in Formula I are described above in the first and/or second embodiments.

Fourth embodiment: a compound represented by Formula II:

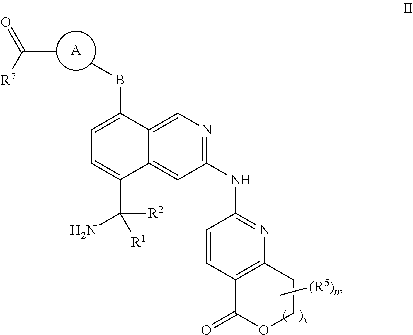

or a pharmaceutically acceptable salt thereof. The variables in Formula II are described above in the first, second and/or third embodiments.

Fifth embodiment: a compound represented by by Formula III:

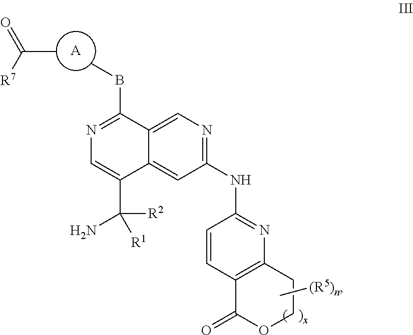

or a pharmaceutically acceptable salt thereof. The variables in Formula III are described above in the first, second and/or third embodiments.

Sixth embodiment: a compound represented by Formula IV(A) or IV(B):

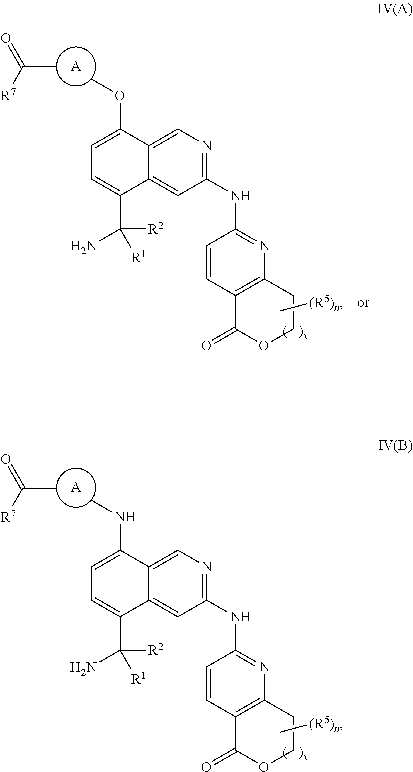

or a pharmaceutically acceptable salt thereof. The variables in Formulae IV(A) and IV(B) are described above in the first, second and/or third embodiments.

Seventh embodiment: a compound represented by Formula V(A) or V(B):

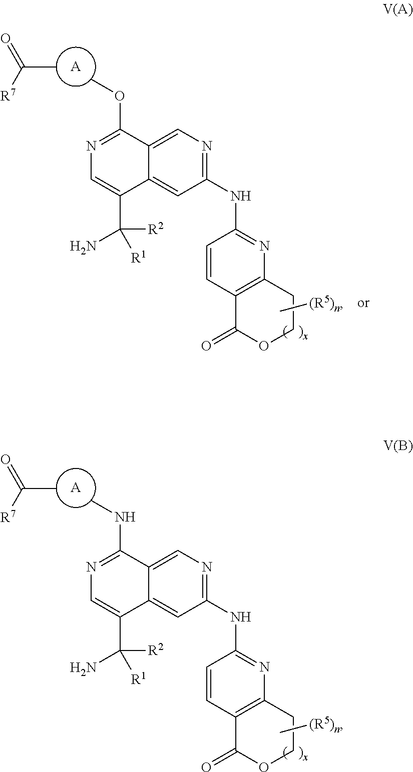

or a pharmaceutically acceptable salt thereof. The variables in Formulae V(A) and V(B) are described above in the first, second and/or third embodiments.

Eighth embodiment: a compound represented by Formula VI(A), VI(B), or VI(C):

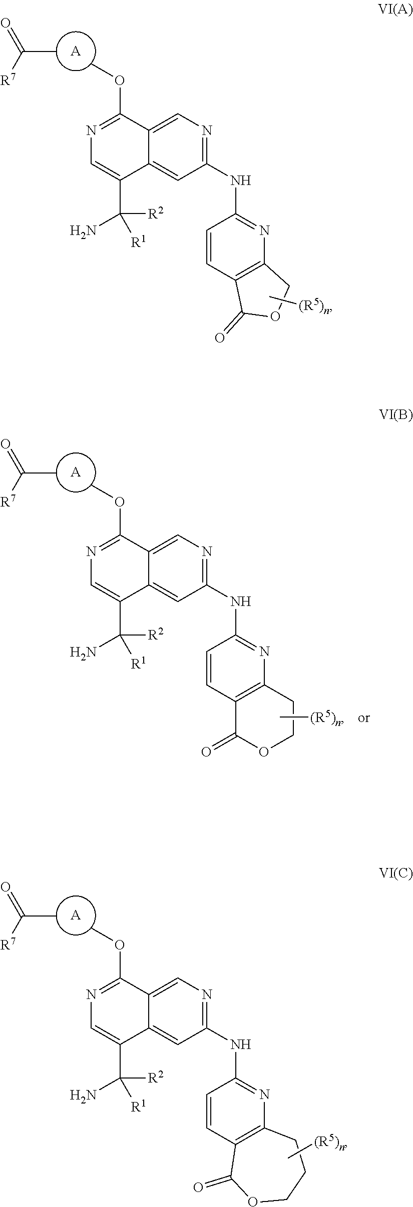

or a pharmaceutically acceptable salt thereof. The variables in Formulae VI(A), VI(B), and VI(C) are described above in the first, second and/or third embodiments.

Ninth embodiment: a compound represented by Formulae I, II, III, IV(A), IV(B), V(A), V(B), VI(A), VI(B), or VI(C), or a pharmaceutically acceptable salt thereof, wherein ring A is selected from cyclobutylene, azetidinylene, and pyrrolidinylene, wherein said cyclobutylene, azetidinylene, or pyrrolidinylene is optionally substituted with one R⁶. The remainder of the variables in Formulae I, II, III, IV(A), IV(B), V(A), V(B), VI(A), VI(B), and VI(C) are described above in the first, second and/or third embodiments.

Tenth embodiment: a compound represented by Formulae I, II, III, IV(A), IV(B), V(A), V(B), VI(A), VI(B), or VI(C), or a pharmaceutically acceptable salt thereof, wherein Ring A is C₄₋₆ cycloalkyl optionally substituted with 1-2 R⁶, and R⁷ is NR⁹R¹⁰ or 3-5 membered heterocycle containing nitrogen, wherein a ring nitrogen of the 3-5 membered heterocycle is bonded to R⁷—C(O [symbol] , and the heterocycle is optionally substituted with 1-3 R⁸; or Ring A is 4-6 membered heterocycle containing nitrogen, wherein a ring nitrogen of the 4-6 membered heterocycle is bonded to R⁷—C(O)—, and the heterocycle is optionally substituted with 1-2 R⁶. The remainder of the variables in Formulae I, II, III, IV(A), IV(B), V(A), V(B), VI(A), VI(B), and VI(C) are described above in the first, second and/or third embodiments.

Eleventh embodiment: a compound represented by Formula VII:

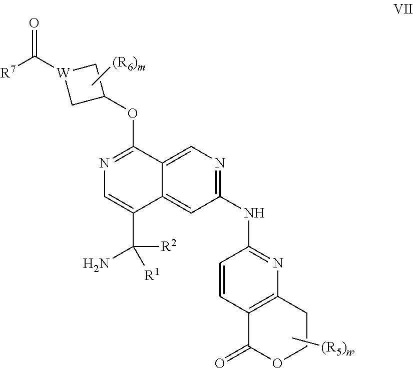

or a pharmaceutically acceptable salt thereof, wherein W is N or CH; and m is 0 or 1. The remainder of the variables in Formula VII are described above in the first, second and/or third embodiments.

Twelfth embodiment: a compound represented by Formula VII(A) or VII(B):

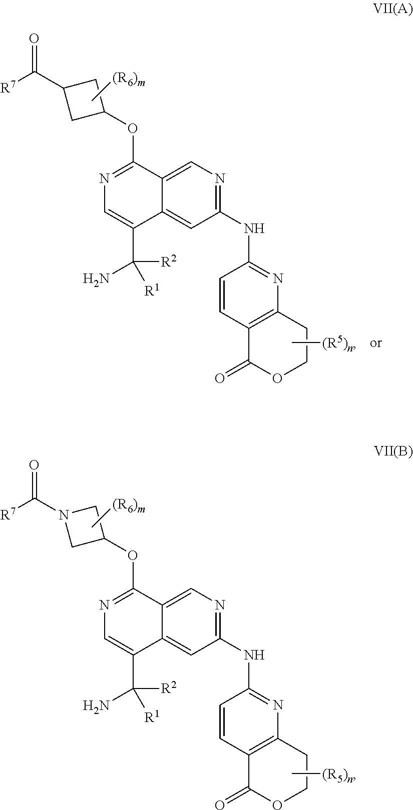

or a pharmaceutically acceptable salt thereof, wherein m is 0 or 1. The variables in Formulae VII(A) and VII(B) are described above in the first, second and/or third embodiments.

Thirteenth embodiment: a compound represented by Formula VII(C):

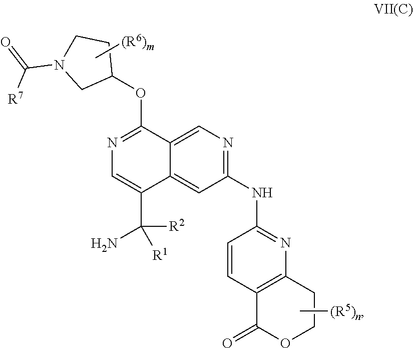

or a pharmaceutically acceptable salt thereof, wherein m is 0 or 1. The remainder of the variables in Formula VII(C) are described above in the first, second and/or third embodiments.

Fourteenth embodiment: a compound represented by Formula VIII(A), VIII(B), or VIII(C):

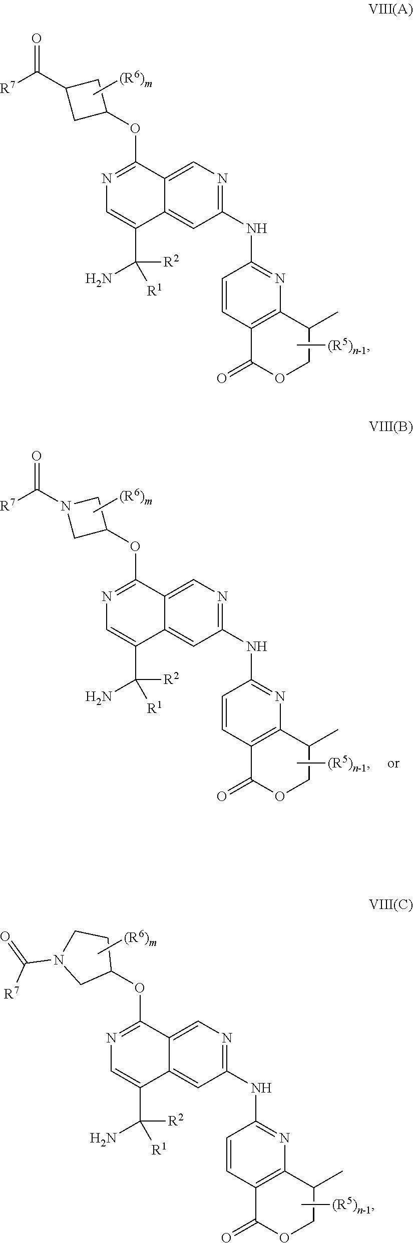

or a pharmaceutically acceptable salt thereof, wherein m is 0 or 1. The remainder of the variables in Formulae VIII(A), VIII(B), and VIII(C) are described above in the first, second and/or third embodiments.

Fifteenth embodiment: a compound represented by Formula IX(A), IX(B), or IX(C):

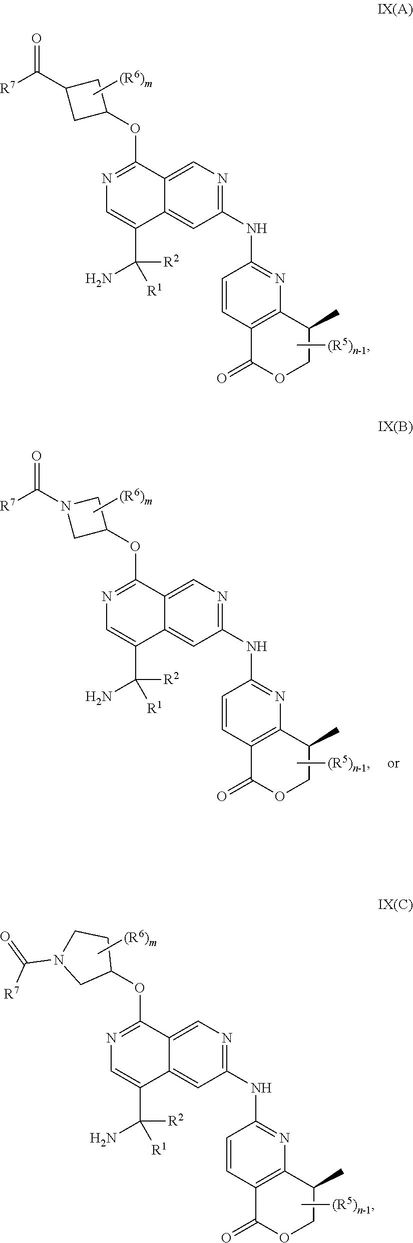

or a pharmaceutically acceptable salt thereof. The variables in Formulae IX(A), IX(B), and IX(C) are as described above in the first, second, third and/or fourteenth embodiments.

Sixteenth embodiment: a compound represented by Formula X(A), X(B), or X(C):

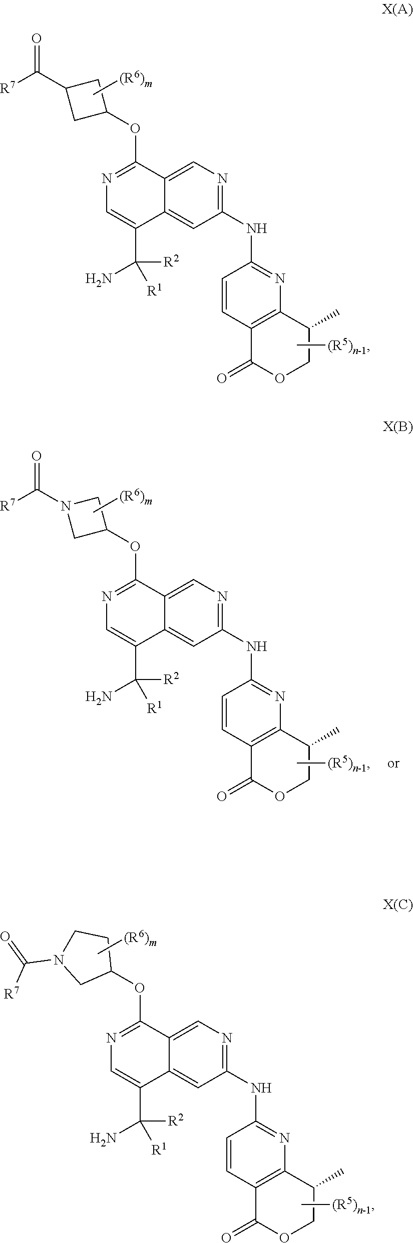

or a pharmaceutically acceptable salt thereof. The variables in Formulae X(A), X(B), and X(C) are as described above in the first, second, third and/or fourteenth embodiments.

Seventeenth embodiment: a compound represented by any one of Formulae I, II, III, IV(A), IV(B), V(A), V(B), VI(A), VI(B), VI(C), VII, VII(A), VII(B), VII(C), VIII(A), VIII(B), VIII(C), IX(A), IX(B), IX(C), X(A), X(B) and X(C), or a pharmaceutically acceptable salt thereof, wherein R⁶ is CH₃. The remainder of the variables in Formulae I, II, III, IV(A), IV(B), V(A), V(B), VI(A), VI(B), VI(C), VII, VII(A), VII(B), VII(C), VIII(A), VIII(B), VIII(C), IX(A), IX(B), IX(C), X(A), X(B) and X(C) are described above in the first, second, third, ninth, tenth, eleventh, thirteenth and/or fourteenth embodiments.

Eighteenth embodiment: a compound represented by any one of Formulae I, II, III, IV(A), IV(B), V(A), V(B), VI(A), VI(B), VI(C), VII, VII(A), VII(B), VII(C), VIII(A), VIII(B), VIII(C), IX(A), IX(B), IV(C), X(A), X(B) and X(C), or a pharmaceutically acceptable salt thereof, wherein R¹ and R² are each independently selected from hydrogen, CH₃, CH₂CH₃, and CH₂OCH₃. The remainder of the variables in Formulae I, II, III, IV(A), IV(B), V(A), V(B), VI(A), VI(B), VI(C), VII, VII(A), VII(B), VII(C), VIII(A), VIII(B), VIII(C), IX(A), IX(B), IV(C), X(A), X(B) and X(C) are described above in the first, second, third, ninth, tenth, eleventh, thirteenth, fourteenth and/or seventeenth embodiments.

Nineteenth embodiment: a compound represented by any one of Formulae I, II, III, IV(A), IV(B), V(A), V(B), VI(A), VI(B), VI(C), VII, VII(A), VII(B), VII(C), VIII(A), VIII(B), VIII(C), IX(A), IX(B), IV(C), X(A), X(B) and X(C), or a pharmaceutically acceptable salt thereof, wherein each R⁵ is CH₃, or two R⁵ attached to the same carbon atom taken together with the carbon atom to which they attach form cyclopropyl; and n is 1, 2, 3, and 4. The remainder of the variables in Formulae I, II, III, IV(A), IV(B), V(A), V(B), VI(A), VI(B), VI(C), VII, VII(A), VII(B), VII(C), VIII(A), VIII(B), VIII(C), IX(A), IX(B), IV(C), X(A), X(B) and X(C) are described above in the first, second, third, ninth, tenth, eleventh, thirteenth, fourteenth, seventeenth and/or eighteenth embodiments.

Twentieth embodiment: a compound represented by any one of Formulae I, II, III, IV(A), IV(B), V(A), V(B), VI(A), VI(B), VI(C), VII, VII(A), VII(B), VII(C), VIII(A), VIII(B), VIII(C), IX(A), IX(B), IV(C), X(A), X(B) and X(C), or a pharmaceutically acceptable salt thereof, wherein R⁷ is selected from CH₃, CH₂CH₃, N(CH₃)₂, OC₁₋₂alkyl, cyclopropyl, azetidinyl, oxetanyl, and tetrahydrofuranyl, wherein R⁷ is optionally substituted with 1-2 R⁸; and each R⁸ is independently selected from F, CH₃, CH₂CH₃, OH, OCH₃, and CF₃. The remainder of the variables in Formulae I, II, III, IV(A), IV(B), V(A), V(B), VI(A), VI(B), VI(C), VII, VII(A), VII(B), VII(C), VIII(A), VIII(B), VIII(C), IX(A), IX(B), IV(C), X(A), X(B) and X(C) are described above in the first, second, third, ninth, tenth, eleventh, thirteenth, fourteenth, seventeenth, eighteenth and/or nineteenth embodiments.

Twenty first embodiment: a compound represented by any one of Formulae I, II, III, IV(A), IV(B), V(A), V(B), VI(A), VI(B), VI(C), VII, VII(A), VII(B), VII(C), VIII(A), VIII(B), VIII(C), IX(A), IX(B), IV(C), X(A), X(B) and X(C), or a pharmaceutically acceptable salt thereof, wherein when R⁷—C(O [symbol] is bonded to a ring carbon, R⁷ is N(CH₃)₂ or azetidinyl, wherein said azetidinyl is optionally substituted with 1-2 R⁸; when R⁷—C(O [symbol] is bonded to a ring nitrogen, R⁷ is selected from CH₃, CH₂CH₃, N(CH₃)₂, OC₁₋₂alkyl, cyclopropyl, azetidinyl, oxetanyl, and tetrahydrofuranyl, wherein said CH₃, CH₂CH₃, OC₁₋₂alkyl, cyclopropyl, azetidinyl, oxetanyl or tetrahydrofuranyl is optionally substituted with 1-2 R⁸; and each R⁸ is independently selected from F, CH₃, CH₂CH₃, OH, OCH₃, and CF₃. The remainder of the variables in Formulae I, II, III, IV(A), IV(B), V(A), V(B), VI(A), VI(B), VI(C), VII, VII(A), VII(B), VII(C), VIII(A), VIII(B), VIII(C), IX(A), IX(B), IV(C), X(A), X(B) and X(C) are described above in the first, second, third, ninth, tenth, eleventh, thirteenth, fourteenth, seventeenth, eighteenth and/or nineteenth embodiments.

Twenty second embodiment: a compound represented by any one of Formulae I, II, III, IV(A), IV(B), V(A), V(B), VI(A), VI(B), VI(C), VII, VII(A), VII(B), VII(C), VIII(A), VIII(B), VIII(C), IX(A), IX(B), IV(C), X(A), X(B) and X(C), or a pharmaceutically acceptable salt thereof, wherein R⁷ is independently selected from CH₃, CH₂CH₃, N(CH₃)₂, OCH₃, OCH₂CH₃, CH₂OCH₃,

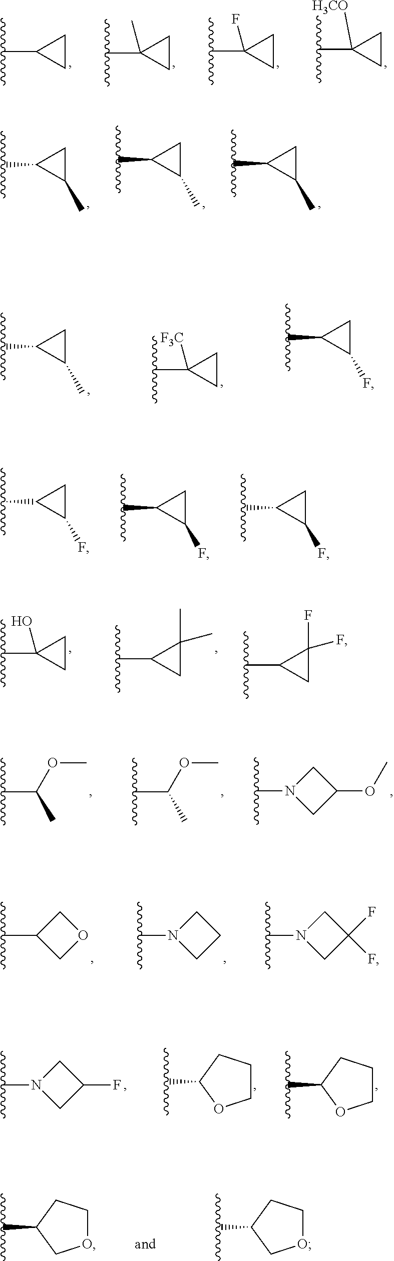

wherein

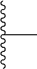

represents the point of attachment to —C(O). The remainder of the variables in Formulae I, II, III, IV(A), IV(B), V(A), V(B), VI(A), VI(B), VI(C), VII, VII(A), VII(B), VII(C), VIII(A), VIII(B), VIII(C), IX(A), IX(B), IV(C), X(A), X(B) and X(C) are described above in the first, second, third, ninth, tenth, eleventh, thirteenth, fourteenth, seventeenth, eighteenth and/or nineteenth embodiments.

Twenty third embodiment: a compound represented by any one of Formulae I, II, III, IV(A), IV(B), V(A), V(B), VI(A), VI(B), VI(C), VII, VII(A), VII(B), VII(C), VIII(A), VIII(B), VIII(C), IX(A), IX(B), IV(C), X(A), X(B) and X(C), or a pharmaceutically acceptable salt thereof, wherein when R⁷—C(O [symbol] is bonded to a ring carbon, R⁷ is selected from N(CH₃)₂,

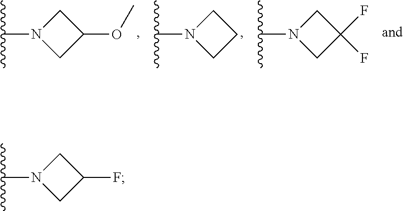

when R⁷—C(O [symbol] is bonded to a ring nitrogen, R⁷ is selected from CH₃, CH₂CH₃, N(CH₃)₂, OCH₃, OCH₂CH₃, CH₂OCH₃,

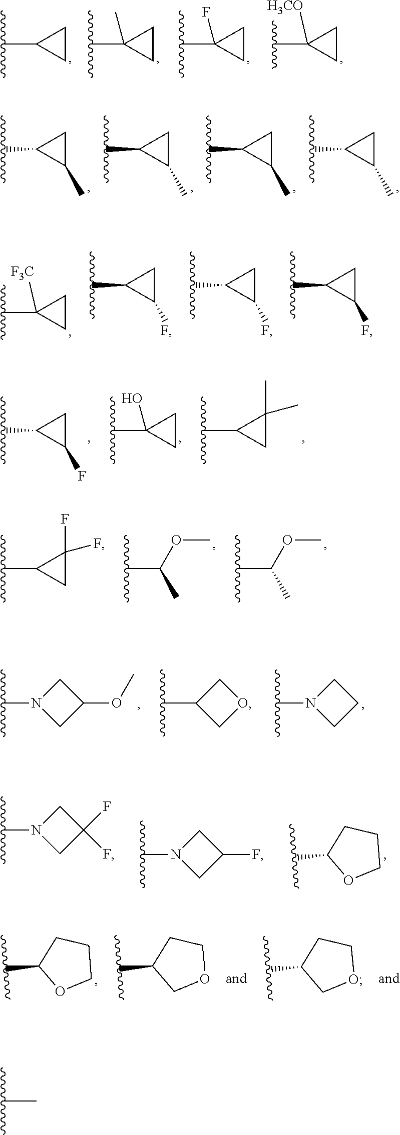

and represents the point of attachment to —C(O). The remainder of the variables in Formulae I, II, III, IV(A), IV(B), V(A), V(B), VI(A), VI(B), VI(C), VII, VII(A), VII(B), VII(C), VIII(A), VIII(B), VIII(C), IX(A), IX(B), IV(C), X(A), X(B) and X(C) are described above in the first, second, third, ninth, tenth, eleventh, thirteenth, fourteenth, seventeenth, eighteenth and/or nineteenth embodiments.

Twenty fourth embodiment: a compound represented by Formula XI:

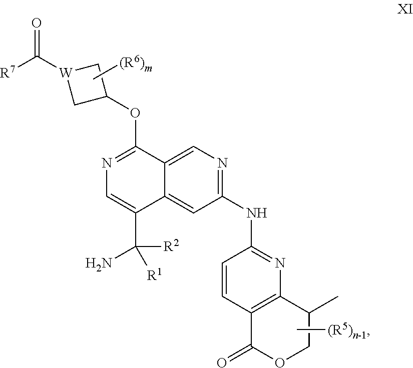

or a pharmaceutically acceptable salt thereof, wherein W is N or CH; R¹ and R² are each independently selected from hydrogen, CH₃, and CH₂CH₃; each R⁵ is CH₃; n is 2 or 3; R⁶ is CH₃; m is 0 or 1; R⁷ is selected from CH₃, CH₂CH₃, cyclopropyl,

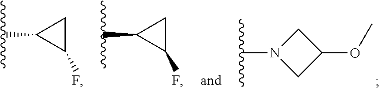

and wherein

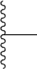

represents the point of attachment to —C(O).

Twenty fifth embodiment: a compound represented by Formula XI, or a pharmaceutically acceptable salt thereof, wherein when R⁷—C(O [symbol] is bonded to a ring nitrogen, R⁷ is selected from CH₃, CH₂CH₃, cyclopropyl,

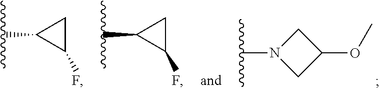

and when R⁷—C(O [symbol] is bonded to a ring carbon, R⁷ is

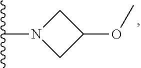 [SMILES: *N1CC(OC)C1]

wherein

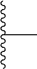

represents the point of attachment —C(O). The remainder of the variables in Formula XI are described above in the twenty fourth embodiment.

Twenty sixth embodiment: a compound represented by Formula XII(A) or XII(B):

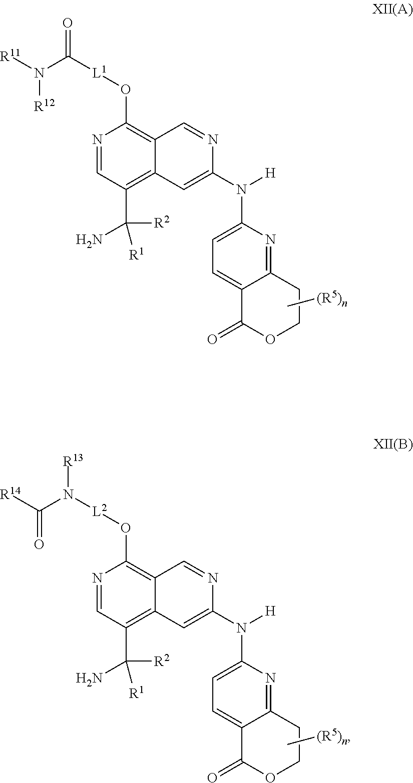

or a pharmaceutically acceptable salt thereof. The remainder of the variables in Formulae XII(A) and XII(B) are described above in the first and/or third embodiments.

Twenty seventh embodiment: a compound represented by any one of Formulae I, XII(A) and XII(B), or a pharmaceutically acceptable salt thereof, wherein: L¹ is selected from —(CH₂)₃— and

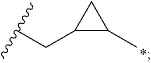 [SMILES: *CC1CC1C]

and L² is —(CH₂)₂—, wherein:

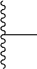

represents the point of attachment to B; and [symbol] represents the point of attachment to —C(O)NR¹¹R¹². The remainder of the variables in Formulae I, XII(A) and XII(B) are described above in the first, second and/or third embodiments.

Twenty eighth embodiment: a compound represented by any one of Formulae I, XII(A) and XII(B), or a pharmaceutically acceptable salt thereof, wherein: R¹ is CH₃; R² is CH₃; each R⁵ is CH₃; n is 0, 1, 2 or 3; R¹¹ is CH₃; R¹² is CH₃; R¹³ is CH₃; and R¹⁴ is CH₃; or R¹³ and R¹⁴ taken together with the nitrogen atom to which they attach form pyrrolidinone ring. The remainder of the variables in Formulae I, XII(A) and XII(B) are described above in the first, second, third and/or twenty seventh embodiments.

The disclosure also includes the compounds depicted in Table 1 and prepared in the Exemplification, both the neutral form and pharmaceutically acceptable salts thereof. The synthetic protocol used to prepare compounds in Table 1 is listed in the last column of Table 1 and full details for each synthetic protocol are described in Schemes 1-5 in the General Synthetic Methods and Intermediates section.

TABLE 1

| | | | | Synthetic |
|---|---|---|---|---|
| Cpd. | | LCMS | | Protocol and |
| No. | Structure | (M + 1) | NMR | Intermediates |
| 1 | 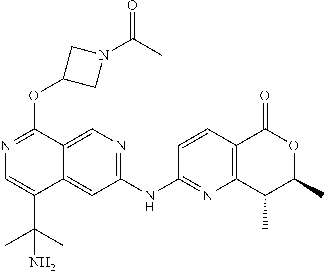 [SMILES: CC(=O)N1CC(Oc2ncc(C(C)(C)N)c3cc(Nc4ccc5c(n4)[C@@H](C)[C@H](C)OC5=O)ncc23)C1] | 491 | ¹H-NMR (400 MHz, CD₃OD): δ ppm 9.51 (s, 1H), 9.05 (s, 1H), 8.52 (s, 1H), 8.20 (d, J = 8.7 Hz, 1H), 8.04 (s, 1H), 7.40 (d, J = 8.7 Hz, 1H), 5.82-5.56 (m, 1H), 4.78- 4.65 (m, 2H), 4.51 (dd, J = 6.7, 11.2 Hz, 1H), 4.43 (dd, J = 3.9, 10.0 Hz, 1H), 4.14 (dd, J = 4.0, 11.4 Hz, 1H), 3.08- 2.92 (m, 1H), 2.00 (d, J = 6.6 Hz, 6H), 1.96 (s, 3H), 1.53 (d, J = 7.2 Hz, 3H), 1.48 (d, J = 6.5 Hz, 3H). | Scheme 1, Intermediate 7 and Intermediate 1 |
| 2 | 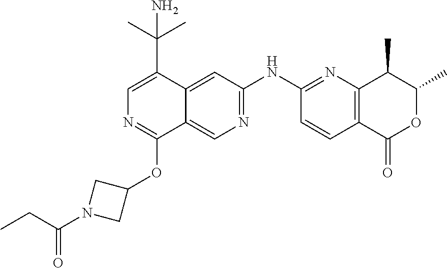 [SMILES: CCC(=O)N1CC(Oc2ncc(C(C)(C)N)c3cc(Nc4ccc5c(n4)[C@@H](C)[C@H](C)OC5=O)ncc23)C1] | 505 | ¹H-NMR (400 MHz, CD₃OD): δ ppm 9.47 (s, 1 H), 8.96 (s, 1 H), 8.61-8.45 (m, 1 H), 8.17-8.12 (m, 1 H), 8.00 (s, 1 H), 7.38 (d, J = 8.8 Hz, 1 H), 5.71-5.57 (m, 1 H), 4.75- 4.62 (m, 2 H), 4.52-4.45 (m, 1 H), 4.42-4.36 (m, 1 H), 4.15- 4.09 (m, 1 H), 3.03-2.96 (m, 1 H), 2.27-2.18 (m, 2 H), 2.01 (d, J = 7.2 Hz, 6 H), 1.53-1.43 (m, 6 H), 1.15-1.10 (m, 3 H). | Scheme 1, Intermediate 9 and Intermediate 1 |
| 3 | 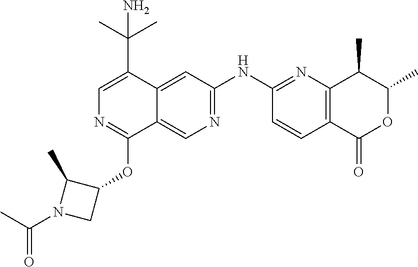 [SMILES: CC(=O)N1C[C@@H](Oc2ncc(C(C)(C)N)c3cc(Nc4ccc5c(n4)[C@@H](C)[C@H](C)OC5=O)ncc23)[C@@H]1C] | 505 | ¹H-NMR (400 MHz, CDCl₃): δ ppm 9.39 (s, 1H), 9.29 (s, 1H), 8.25 (d, J = 8.8 Hz, 1H), 8.12 (d, J = 2.4 Hz, 1H), 8.02 (s, 1H), 7.25 (d, J = 8.8 Hz, 1H), 5.15-5.12 (m, 1H), 4.61- 4.40 (m, 3H), 4.15-4.10 (m, 1H), 3.03-2.96 (m, 1H), 1.95 (d, J = 15.6 Hz, 3H), 1.76 (s, 6H), 1.73-1.66 (m, 7H), 1.55- 1.51 (m, 6H). | Scheme 1, Intermediate 10, and Intermediate 1 |
| 4 | 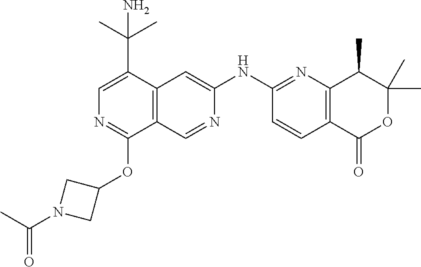 [SMILES: CC(=O)N1CC(Oc2ncc(C(C)(C)N)c3cc(Nc4ccc5c(n4)[C@@H](C)C(C)(C)OC5=O)ncc23)C1] | 505 | ¹H-NMR (400 MHz, CD₃OD): δ ppm 9.43 (s, 1H), 9.39 (s, 1H), 8.17 (s, 1H), 8.14 (d, J = 8.4 Hz, 1H), 7.23 (d, J = 8.8 Hz, 1H), 5.65-5.57 (m, 1H), 4.77-4.68 (m, 1H), 4.55-4.45 (m, 1H), 4.44-4.35 (m, 1H), 4.16-4.09 (m, 1H), 3.09-3.00 (m, 1H), 1.93 (s, 3H), 1.81 (d, J = 8.0 Hz, 6H), 1.54 (s, 3H), 1.45 (s, 3H), 1.40 (d, J = 7.2 Hz, 3H). | Scheme 1, Intermediate 3 and Intermediate 7 |
| 5 | 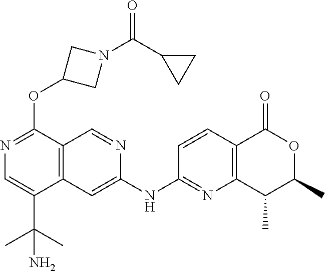 [SMILES: C[C@@H]1OC(=O)c2ccc(Nc3cc4c(C(C)(C)N)cnc(OC5CN(C(=O)C6CC6)C5)c4cn3)nc2[C@H]1C] | 517 | ¹H-NMR (400 MHz, CD₃OD): δ ppm 9.50 (s, 1H), 9.05 (s, 1H), 8.50 (s, 1H), 8.18 (d, J = 8.4 Hz, 1H), 8.03 (s, 1H), 7.38 (d, J = 8.4 Hz, 1H), 5.73-5.61 (m, 1H), 4.56- 4.46 (m, 2H), 4.15-4.07 (m, 1H), 3.06-2.95 (m, 1H), 1.97 (d, J = 6.0 Hz, 6H), 1.70-1.58 (m, 1H), 1.54-1.41 (m, 6H), 0.94-0.80 (m, 4H). | Scheme 1, Intermediate 8 and Intermediate 1 |
| 6 | 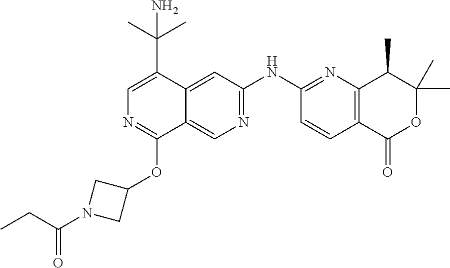 [SMILES: CCC(=O)N1CC(Oc2ncc(C(C)(C)N)c3cc(Nc4ccc5c(n4)[C@@H](C)C(C)(C)OC5=O)ncc23)C1] | 519 | ¹H-NMR (400 MHz, CD₃OD): δ ppm 9.42 (s, 1 H), 9.30 (s, 1 H), 8.54 (s, 1 H), 8.17-8.07 (m, 2 H), 7.23 (d, J = 8.4 Hz, 1 H), 5.70-5.50 (m, 1 H), 4.74-4.67 (m, 1 H), 4.52-4.44 (m, 1 H), 4.40-4.33 (m, 1 H), 4.15-4.08 (m, 1 H), 3.06-2.97 (m, 1 H), 2.27-2.18 (m, 2 H), 1.86 (d, J = 8.8 Hz, 6 H), 1.53 (s, 3 H), 1.36-1.48 (m, 6 H), 1.16-1.08 (m, 3 H). | Scheme 1, Intermediate 9 and Intermediate 3 |
| 7 | 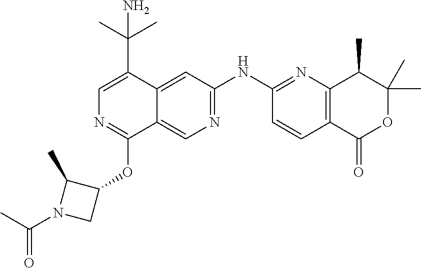 [SMILES: CC(=O)N1C[C@@H](Oc2ncc(C(C)(C)N)c3cc(Nc4ccc5c(n4)[C@@H](C)C(C)(C)OC5=O)ncc23)[C@@H]1C] | 519 | ¹H-NMR (400 MHz, CD₃OD): δ ppm 9.42 (d, J = 3.6 Hz, 2H), 8.22 (d, J = 2.4 Hz, 1H), 8.15 (d, J = 8.8 Hz, 1H), 7.24 (d, J = 8.4 Hz, 1H), 5.25-5.14 (m, 1H), 4.75-4.61 (m, 1H), 4.57-4.39 (m, 1H), 4.32-4.02 (m, 1H), 3.13-2.98 (m, 1H), 1.96 (d, J = 15.6 Hz, 3H), 1.82 (d, J = 7.6 Hz, 6H), 1.77-1.65 (m, 3H), 1.56 (s, 3H), 1.47 (s, 3H), 1.42 (d, J = 7.2 Hz, 3H) | Scheme 1, Intermediate 10 and Intermediate 3 |
| 8 | 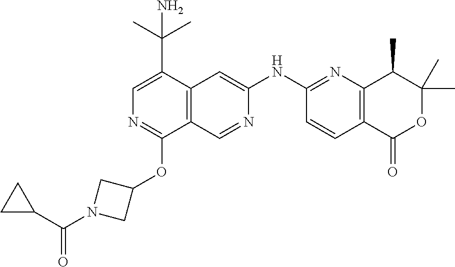 [SMILES: C[C@@H]1c2nc(Nc3cc4c(C(C)(C)N)cnc(OC5CN(C(=O)C6CC6)C5)c4cn3)ccc2C(=O)OC1(C)C] | 531 | ¹H-NMR (400 MHz, CD₃OD): δ ppm 9.50 (s, 1 H), 9.17 (s, 1 H), 8.48 (s, 1 H), 8.16 (d, J = 8.8 Hz, 1 H), 8.01 (s, 1 H), 7.30 (d, J = 8.8 Hz, 1 H), 5.71-5.65 (m, 1 H), 4.84 (s, 1 H), 4.63-4.40(m, 2 H), 4.15-4.08 (m, 1 H), 3.04-2.95 (m, 1 H), 2.01 (d, J = 12.4 Hz, 6 H), 1.69-1.59 (m, 1 H), 1.54 (s, 3 H), 1.45 (s, 3 H), 1.41 (d, J = 7.2 Hz, 3 H), 0.94-0.81 (m, 4 H). | Scheme 1, Intermediate 8 and Intermediate 3 |
| 9 | 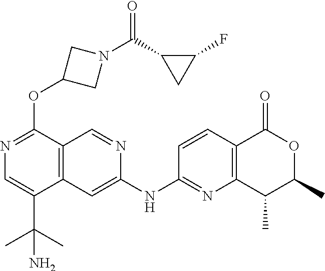 [SMILES: C[C@@H]1OC(=O)c2ccc(Nc3cc4c(C(C)(C)N)cnc(OC5CN(C(=O)[C@H]6C[C@H]6F)C5)c4cn3)nc2[C@H]1C] | 535 | ¹H-NMR (400 MHz, CD₃OD): δ ppm 9.45 (d, J = 4.0 Hz, 1H), 9.20 (s, 1H), 8.21-8.09 (m, 2H), 7.33 (d, J = 8.4 Hz, 1H), 5.70-5.68 (m, 1H), 4.73-4.43 (m, 4H), 4.20- 4.15 (m, 1H), 3.08-2.98 (m, 1H), 1.90-1.83 (m, 7H), 1.75- 1.58 (m, 2H), 1.55-1.45 (m, 6H), 1.13-1.05 (m, 1H). | Scheme 2, Intermediate 1 and Enantiomer of Intermediate 11 |
| 10 | 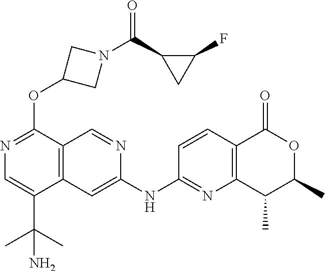 [SMILES: C[C@@H]1OC(=O)c2ccc(Nc3cc4c(C(C)(C)N)cnc(OC5CN(C(=O)[C@@H]6C[C@@H]6F)C5)c4cn3)nc2[C@H]1C] | 535 | ¹H-NMR (400 MHz, CD₃OD): δ ppm 9.46 (d, J = 3.6 Hz, 1H), 9.25 (s, 1H), 8.21 (s, 1H), 8.17 (d, J = 8.8 Hz, 1H), 7.34 (d, J = 8.4 Hz, 1H), 5.78-5.55 (m, 1H), 5.04-4.91 (m, 1H), 4.84-4.74 (m, 1H), 4.73-4.63 (m, 1H), 4.63-4.42 (m, 2H), 4.23-4.09 (m, 1H), 3.10-3.03 (m, 1H), 1.94-1.85 (m, 1H), 1.82 (d, J = 4.8 Hz, 6H), 1.76-1.64 (m, 1H), 1.53 (d, J = 7.6 Hz, 3H), 1.48 (d, J = 6.8 Hz, 3H), 1.20-1.05 (m, 1H). | Scheme 1, Intermediate 11 and Intermediate 1 |
| 11 | 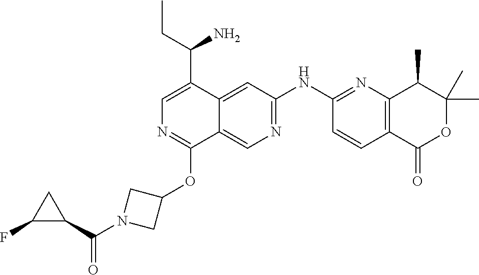 [SMILES: CC[C@@H](N)c1cnc(OC2CN(C(=O)[C@@H]3C[C@@H]3F)C2)c2cnc(Nc3ccc4c(n3)[C@@H](C)C(C)(C)OC4=O)cc12] | 549 | ¹H-NMR (400 MHz, CD₃OD): δ ppm 9.45 (s, 1H), 8.86 (s, 1H), 8.55 (s, 1H), 8.18-8.14 (m, 2H), 7.31 (d, J = 8.8 Hz, 1H), 5.70 (s, 1H), 4.81-4.76 (m, 1H), 4.61-4.52 (m, 4H), 4.21-4.18 (m, 1H), 3.23-3.17 (m, 1H), 2.12-2.03 (m, 2H), 1.90 (m, 1H), 1.55- 1.46 (m, 1H), 1.46 (s, 3H), 1.12-1.10 (m, 1H), 1.07-1.02 (m, 3H). | Scheme 3, Intermediate 3 and Intermediate 11 |
| | or | | | |
| | 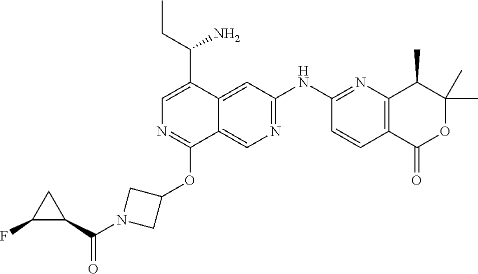 [SMILES: CC[C@H](N)c1cnc(OC2CN(C(=O)[C@@H]3C[C@@H]3F)C2)c2cnc(Nc3ccc4c(n3)[C@@H](C)C(C)(C)OC4=O)cc12] | | | |
| 12 | 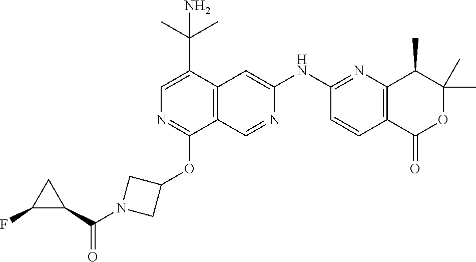 [SMILES: C[C@@H]1c2nc(Nc3cc4c(C(C)(C)N)cnc(OC5CN(C(=O)[C@@H]6C[C@@H]6F)C5)c4cn3)ccc2C(=O)OC1(C)C] | 549 | ¹H-NMR (400 MHz, CD₃OD): δ ppm 9.44 (d, J = 3.2 Hz, 1H), 9.41 (s, 1H), 8.20 (s, 1H), 8.14 (d, J = 8.8 Hz, 1H), 7.23 (d, J = 8.8 Hz, 1H), 5.73-5.59 (m, 1H), 4.97-4.89 (m, 1H), 4.82-4.72 (m, 1H), 4.59-4.43 (m, 2H), 4.21-4.15 (m, 1H), 3.09-3.01 (m, 1H), 1.93-1.84 (m, 1H), 1.80 (d, J = 7.2 Hz, 6H), 1.74-1.64 (m, 1H), 1.55 (s, 3H), 1.46 (s, 3H), 1.41 (d, J = 6.8 Hz, 3H), 1.19-1.05 (m, 1H). | Scheme 2, Intermediate 3 and Intermediate 11 |
| 13 | 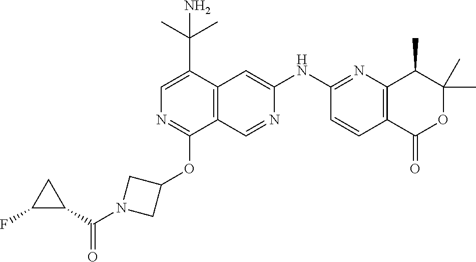 [SMILES: C[C@@H]1c2nc(Nc3cc4c(C(C)(C)N)cnc(OC5CN(C(=O)[C@H]6C[C@H]6F)C5)c4cn3)ccc2C(=O)OC1(C)C] | 549 | ¹H-NMR (400 MHz, CD₃OD): δ ppm 9.44 (d, J = 3.6 Hz, 1H), 9.40 (s, 1H), 8.19 (s, 1H), 8.14 (d, J = 8.8 Hz, 1H), 7.23 (d, J = 8.8 Hz, 1H), 5.71-5.62 (m, 1H), 4.96-4.88 (m, 1H), 4.82-4.71 (m, 1H), 4.60-4.42 (m, 2H), 4.22-4.12 (m, 1H), 3.09-3.01 (m, 1H), 1.92-1.84 (m, 1H), 1.81 (d, J = 7.2 Hz, 6H), 1.75-1.63 (m, 1H), 1.54 (s, 3H), 1.45 (s, 3H), 1.40 (d, J = 7.2 Hz, 3H), 1.18-1.06 (m, 1H). | Scheme 2, Intermediate 3 and Enantiomer of Intermediate 11 |
| 14 | 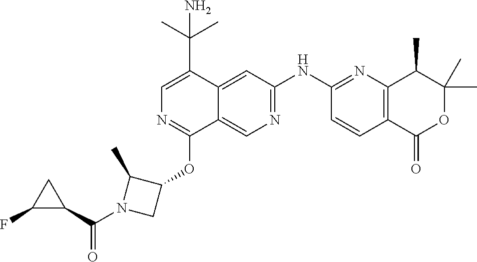 [SMILES: C[C@@H]1c2nc(Nc3cc4c(C(C)(C)N)cnc(O[C@@H]5CN(C(=O)[C@@H]6C[C@@H]6F)[C@H]5C)c4cn3)ccc2C(=O)OC1(C)C] | 563 | ¹H-NMR (400 MHz, CD₃OD): δ ppm 9.50-9.37 (m, 2H), 8.23 (s, 1H), 8.15 (d, J = 8.8 Hz, 1H), 7.24 (d, J = 8.4 Hz, 1H), 5.29-5.20 (m, 1H), 4.96-4.91 (m, 1H), 4.81- 4.74 (m, 1H), 4.60-4.44 (m, 1H), 4.43-4.06 (m, 1H), 3.13- 2.98 (m, 1H), 1.93-1.83 (m, 2H), 1.82 (d, J = 7.6 Hz, 6H), 1.69 (d, J = 6.8 Hz, 3H), 1.56 (s, 3H), 1.47 (s, 3H), 1.42 (d, J = 7.2 Hz, 3H), 1.19-1.01 (m, 1H). | Scheme 1, Intermediate 12 and Intermediate 3 |
| 15 | 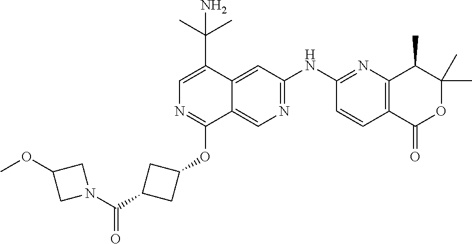 | 575 | ¹H-NMR (400 MHz, CD₃OD): δ ppm 9.41 (s, 1H), 9.18 (s, 1H), 8.52 (s, 1H), 8.16 (d, J = 8.8 Hz, 1H), 8.02 (s, 1H), 7.27 (d, J = 8.8 Hz, 1H), 5.40-5.30 (m, 1H), 4.45- 4.35 (m, 1H), 4.40-4.32 (m, 1H), 4.20-4.17 (m, 1H), 4.15- 4.05 (m, 1H), 3.85-3.78 (m, 1H), 3.05-2.90 (m, 2H), 2.85- 2.75 (m, 2H), 2.51-2.41 (m, 2H), 1.96 (d, J = 11.4 Hz, 6H), 1.54 (s, 3H), 1.45 (s, 3H), 1.41 (d, J = 7.2 Hz, 3H). | Scheme 4, Intermediate 3 |
| 16 | 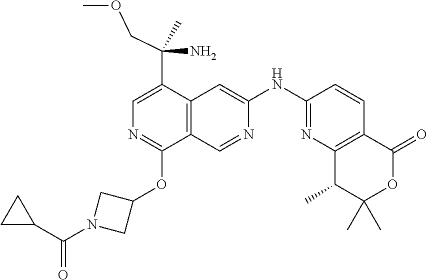 [SMILES: COC[C@@](C)(N)c1cnc(OC2CN(C(=O)C3CC3)C2)c2cnc(Nc3ccc4c(n3)[C@@H](C)C(C)(C)OC4=O)cc12] | 561 | ¹H NMR (400 MHz, CD₃OD) δ ppm 9.43 (s, 2H), 8.2 (s, 1H), 8.15 (d, J = 8.8 Hz, 1H), 7.23 (d, J = 8.8 Hz, 1H), 5.70-5.65 (m, 1H), 4.85 (s, 1H), 4.54- 4.49 (m, 2H), 4.17 (d, J = 4.0 Hz, 1H), 3.97-3.87 (m, 2H), 3.37 (s, 3H), 3.09- 3.06 (m, 1H), 1.76 (s, 3H), 1.64-1.48 (m, 3H), 1.42 (d, J = 8.8 Hz, 3H), 0.92-0.86 (m, 1H). | Scheme 1, Intermediate 3 and Intermediate 16 |
| 17 | 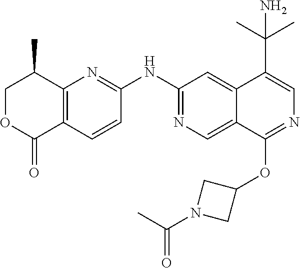 [SMILES: CC(=O)N1CC(Oc2ncc(C(C)(C)N)c3cc(Nc4ccc5c(n4)[C@H](C)COC5=O)ncc23)C1] | 477 | ¹H NMR (400 MHz, CD₃OD): δ ppm 9.42 (s, 1H), 9.37 (s, 1H), 8.18 (s, 1H), 8.15 (d, J = 8.8 Hz, 1H), 7.23 (d, J = 8.8 Hz, 1H), 5.65-5.58 (m, 1H), 4.74-4.68 (m, 2H), 4.53- 4.35 (m, 3H), 4.16-4.09 (m, 1H), 3.23-3.15 (m, 1H), 1.94 (s, 3H), 1.81 (d, J = 6.8 Hz, 6H), 1.49 (d, J = 7.2 Hz, 3H). | Scheme 1, Intermediate 20, and Intermediate 7, second eluting isomer by chiral SFC Column: Daicel Chiralpak AD (250 mm × 30 mm, 10 um); Mobile Phase: [0.1% NH3H2O IPA]; B%: 40%-40%) |
| | or | | | |
| | 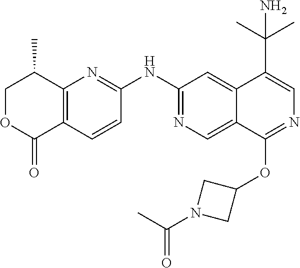 [SMILES: CC(=O)N1CC(Oc2ncc(C(C)(C)N)c3cc(Nc4ccc5c(n4)[C@@H](C)COC5=O)ncc23)C1] | | | |
| 18 | 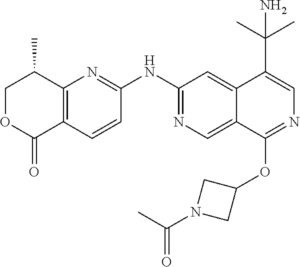 [SMILES: CC(=O)N1CC(Oc2ncc(C(C)(C)N)c3cc(Nc4ccc5c(n4)[C@@H](C)COC5=O)ncc23)C1] | 477 | ¹H NMR (400 MHz, CD₃OD): δ ppm 9.43 (s, 1H), 9.35 (s, 1H), 8.19- 8.12 (m, 2H), 7.23 (d, J = 8.8 Hz, 1H), 5.65-5.58 (m, 1H), 4.74-4.68 (m, 2H), 4.53-4.36 (m, 3H), 4.17- 4.09 (m, 1H), 3.23-3.15 (m, 1H), 1.94 (s, 3H), 1.84 (d, J = 6.8 Hz, 6H), 1.49 (d, J = 7.2 Hz, 3H). | Scheme 1, Intermediate 20, and Intermediate 7, first eluting isomer by chiral SFC Column: Daicel Chiralpak AD (250 mm × 30 mm, 10 um); Mobile Phase: [0.1% NH3H2O IPA]; B%: 40%-40%) |
| | or | | | |
| | 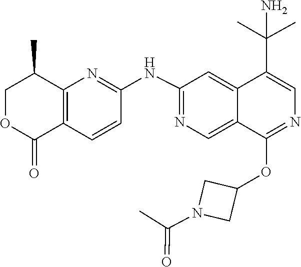 [SMILES: CC(=O)N1CC(Oc2ncc(C(C)(C)N)c3cc(Nc4ccc5c(n4)[C@H](C)COC5=O)ncc23)C1] | | | |
| 19 | 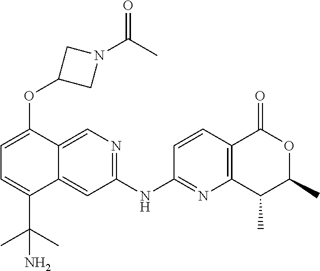 [SMILES: CC(=O)N1CC(Oc2ccc(C(C)(C)N)c3cc(Nc4ccc5c(n4)[C@@H](C)[C@H](C)OC5=O)ncc23)C1] | 490 | ¹H NMR (400 MHz, CD₃OD): δ ppm 9.49 (s, 1H), 9.11 (s, 1H), 8.53 (s, 1H), 8.12 (d, J = 8.8 Hz, 1H), 7.53 (d, J = 8.4 Hz, 1H), 7.28 (d, J = 8.8 Hz, 1H), 6.62 (d, J = 8.0 Hz, 1H), 5.30-5.27 (m, 1H), 4.75-4.68 (m, 1H), 4.67- 4.65 (m, 1H), 4.60-4.50 (m, 1H), 4.50-4.35 (m, 1H), 4.15-4.05 (m, 1H), 3.05- 2.09 (m, 1H), 1.99 (s, 3H), 1.97 (s, 3H), 1.51 (s, 3H), 1.49 (s, 3H), 1.45 (s, 3H), 1.44 (s, 3H). | Scheme 2, Intermediate 1, Intermediate 38, and acetyl chloride |
| 20 | 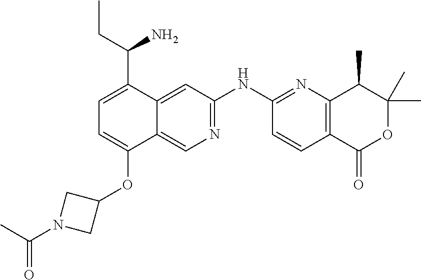 [SMILES: CC[C@@H](N)c1ccc(OC2CN(C(C)=O)C2)c2cnc(Nc3ccc4c(n3)[C@@H](C)C(C)(C)OC4=O)cc12] | 504 | ¹H NMR (400 M Hz, CD₃OD): δ ppm 9.42 (s, 1H), 8.91 (s, 1H), 8.09 (d, J = 8.8 Hz, 1H), 7.70-7.64 (m, 1H), 7.20 (d, J = 8.4 Hz, 1H), 6.63 (d, J = 8.00 Hz, 1H), 5.31- 5.22 (m, 1H), 4.76-4.70 (m, 1H), 4.59-4.49 (m, 2H), 4.43- 4.37 (m, 1H), 4.15-4.08 (m, 1H), 3.17-3.09 (m, 1H), 2.01- 1.84 (m, 5H), 1.53-1.47 (m, 6H), 1.45 (s, 3H) 0.97 (t, J = 7.4 Hz, 3H). | Scheme 5, Intermediate 3, Intermediate 39, and acetyl chloride |
| | or | | | |
| | 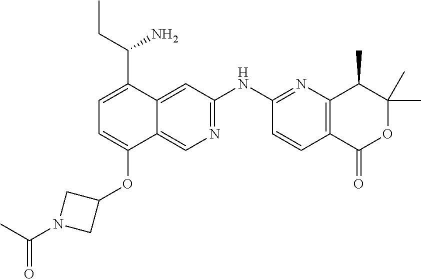 [SMILES: CC[C@H](N)c1ccc(OC2CN(C(C)=O)C2)c2cnc(Nc3ccc4c(n3)[C@@H](C)C(C)(C)OC4=O)cc12] | | | |
| 21 | 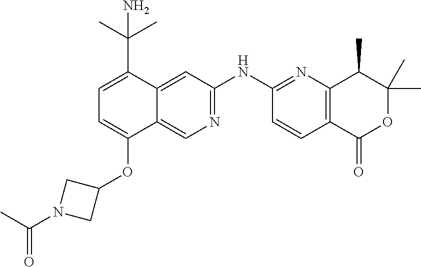 [SMILES: CC(=O)N1CC(Oc2ccc(C(C)(C)N)c3cc(Nc4ccc5c(n4)[C@@H](C)C(C)(C)OC5=O)ncc23)C1] | 504 | ¹H-NMR (400 MHz, CD₃OD): δ ppm 9.50 (s, 1H), 9.25 (s, 1H), 8.51 (s, 1H), 8.11 (d, J = 8.8 Hz, 1H), 7.49 (d, J = 8.4 Hz, 1H), 7.21 (d, J = 8.8 Hz, 1H), 6.64 (d, J = 8.4 Hz, 1H), 5.30 (s, 1H), 4.78- 4.74 (m, 1H), 4.53-4.51(m, 1H), 4.44-4.41 (m, 1H), 4.15- 4.10 (m, 1H), 3.00-2.90 (m, 1H), 2.05 (s, 3H), 2.02 (s, 3H), 1.95 (s, 3H), 1.54 (s, 3H), 1.45 (s, 3H), 1.41 (d, J = 7.6 Hz, 3H). | Scheme 2, Intermediate 3, Intermediate 38, and acetyl chloride |
| 22 | 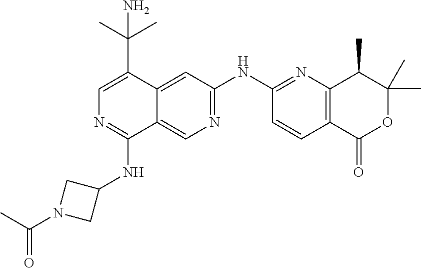 [SMILES: CC(=O)N1CC(Nc2ncc(C(C)(C)N)c3cc(Nc4ccc5c(n4)[C@@H](C)C(C)(C)OC5=O)ncc23)C1] | 504 | ¹H NMR (400 MHz, CD₃OD): δ ppm 9.35 (s, 1H), 9.29 (s, 1H), 8.16-8.09 (m, 2H), 7.24 (d, J = 8.8 Hz, 1H), 4.78-4.56 (m, 2H), 4.48-4.39 (m, 1H), 4.24-4.17 (m, 1H), 4.09-4.00 (m, 1H), 3.11-3.01 (m, 1H), 1.94 (s, 3H), 1.79 (d, J = 7.2 Hz, 6H), 1.55 (s, 3H), 1.46 (s, 3H), 1.41 (d, J = 7.2 Hz, 3H). | Scheme 1, Intermediate 3 and Intermediate 43 |
| 23 | 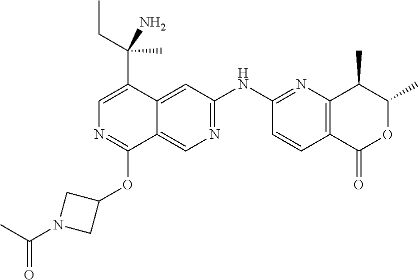 [SMILES: CC[C@@](C)(N)c1cnc(OC2CN(C(C)=O)C2)c2cnc(Nc3ccc4c(n3)[C@@H](C)[C@H](C)OC4=O)cc12] | 505 | ¹H-NMR (400 MHz, CD₃OD): δ ppm 9.42 (s, 1H), 9.20 (s, 1H), 8.18-8.11 (m, 2H), 7.30 (d, J = 8.8 Hz, 1H), 5.64-5.60 (m, 1H), 4.73-4.66 (m, 2H), 4.51-4.50 (m, 1H), 4.41-4.43 (m, 1H), 4.17-4.15 (m, 1H), 3.05-3.02 (m, 1H), 2.41-2.35 (m, 1H), 2.17-2.14 (m, 1H), 1.95 (s, 3H), 1.74 (s, 3H), 1.52-4.46 (m, 6H), 0.74 (t, J = 7.6 Hz, 1H). | Scheme 1, Intermediate 1 and Intermediate 45 |
| 24 | 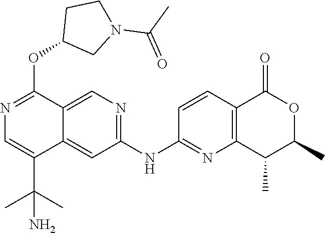 [SMILES: CC(=O)N1CC[C@@H](Oc2ncc(C(C)(C)N)c3cc(Nc4ccc5c(n4)[C@@H](C)[C@H](C)OC5=O)ncc23)C1] | 505 | ¹H NMR (400 MHz, CD₃OD): δ ppm 9.35 (s, 1H), 9.21 (d, J = 2.0 Hz, 1H), 8.23 (s, 1H), 8.15 (d, J = 8.8 Hz, 1H), 7.31 (d, J = 8.4 Hz, 1H), 5.98-5.70 (m, 1H), 4.76-4.61 (m, 1H), 4.12-3.61 (m, 4H), 3.11-2.98 (m, 1H), 2.53-2.30 (m, 2H), 2.22-2.04 (m, 3H), 1.81 (d, J = 4.8 Hz, 6H), 1.57-1.43 (m, 6H). | Scheme 2, Intermediate 1, Intermediate 47, and Acetyl chloride |
| 25 | 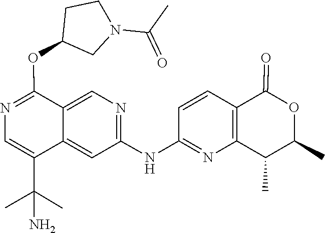 [SMILES: CC(=O)N1CC[C@H](Oc2ncc(C(C)(C)N)c3cc(Nc4ccc5c(n4)[C@@H](C)[C@H](C)OC5=O)ncc23)C1] | 505 | ¹H-NMR (400 MHz, DMSO- d₆) δ ppm 10.67 (d, 1H, J = 6.8 Hz), 9.41 (d, 1H, J = 6.0 Hz), 9.31 (d, 1H, J = 5.2 Hz), 8.20 (s, 1H), 8.09 (d, 1H, J = 8.8 Hz), 7.42 (dd, 1H, J = 8.4, 6.8 Hz), 5.83-5.68 (m, 1H), 4.64-4.61 (m, 1H), 3.93 (dd, 0.5H, J = 11.6, 4.8 Hz,), 3.78-3.58 (m, 3H), 3.55-3.44 (m, 0.5H), 3.01-2.98 (m, 1H), 2.39-2.18 (m, 2H), 2.01 (s, 3H), 1.66 (s, 6H), 1.48-1.36 (m, 6H). | Scheme 2, Intermediate 1, Intermediate 28, and Acetyl chloride |
| 26 | 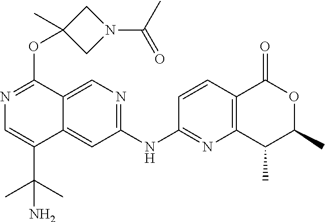 [SMILES: CC(=O)N1CC(C)(Oc2ncc(C(C)(C)N)c3cc(Nc4ccc5c(n4)[C@@H](C)[C@H](C)OC5=O)ncc23)C1] | 505 | ¹H NMR (400 MHz, CD₃OD): δ ppm 9.38 (s, 1H), 9.22 (s, 1H), 8.22 (s, 1H), 8.16 (d, J = 8.4 Hz, 1H), 7.32 (d, J = 8.4 Hz, 1H), 4.69-4.64 (m, 2H), 4.44-4.35 (m, 2H), 4.19 (d, J = 10.8 Hz, 1H), 3.08-3.02 (m, 1H), 1.95-1.92 (m, 6H), 1.84-1.80 (m, 6H), 1.53 (d, J = 7.2 Hz, 3H), 1.47 (d, J = 6.4 Hz, 3H). | Scheme 1, Intermediate 1 and Intermediate 48 |
| 27 | 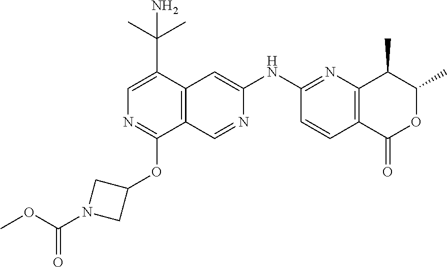 | 507 | ¹H NMR (500 MHz, DMSO) δ 10.70 (s, 1H), 9.45 (s, 1H), 9.41 (s, 1H), 8.16 (s, 1H), 8.10 (d, J = 8.7 Hz, 1H), 7.40 (d, J = 8.7 Hz, 1H), 5.54 (tt, J = 6.8, 4.0 Hz, 1H), 4.61 (p, J = 5.4 Hz, 2H), 4.33 (dd, J = 10.8, 6.8 Hz, 1H), 4.26 (dd, J = 9.9, 3.9 Hz, 1H), 4.01 (dd, J = 10.9, 3.9 Hz, 1H), 3.01 (p, J = 6.9 Hz, 1H), 1.82 (s, 3H), 1.66 (d, J = 3.0 Hz, 6H), 1.45 (d, J = 7.1 Hz, 3H), 1.39 (d, J = 6.5 Hz, 3H). | Scheme 1, Intermediate 1 and Intermediate 35 |
| 28 | 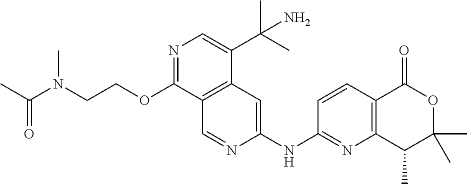 [SMILES: CC(=O)N(C)CCOc1ncc(C(C)(C)N)c2cc(Nc3ccc4c(n3)[C@@H](C)C(C)(C)OC4=O)ncc12] | 507 | ¹H NMR (400 MHz, CD₃OD): δ ppm 9.44-9.30 (m, 2H), 8.22 (d, J = 7.6 Hz, 1H), 8.15 (d, J = 8.8 Hz, 1H), 7.24 (dd, J = 2.0, 8.8 Hz, 1H), 4.80- 4.75 (m, 1H), 4.72-4.68 (m, 1H), 3.99-3.87 (m, 2H), 3.25- 3.20 (m, 1H), 3.26-3.03 (m, 3H), 2.28-2.07 (m, 3H), 1.81 (d, J = 7.6 Hz, 6H), 1.58-1.45 (m, 6H), 1.42 (d, J = 7.6 Hz, 3H) | Scheme 1, Intermediate 3 and Intermediate 49 |
| 29 | 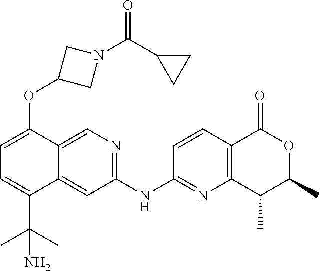 [SMILES: C[C@@H]1OC(=O)c2ccc(Nc3cc4c(C(C)(C)N)ccc(OC5CN(C(=O)C6CC6)C5)c4cn3)nc2[C@H]1C] | 516 | ¹H-NMR (400 MHz, CD₃OD): δ ppm 9.50 (s, 1H), 9.12 (s, 1H), 8.53 (s, 1H), 8.12 (d, J = 8.8 Hz, 1H), 7.55 (d, J = 8.4 Hz, 1H), 7.28 (d, J = 8.8 Hz, 1H), 6.64 (d, J = 8.4 Hz, 1H), 5.34-5.32 (m, 1H), 4.85-4.75 (m, 1H), 4.55-4.40 (m, 2H), 4.15-4.05 (m, 1H), 3.05-2.95 (m, 1 H), 1.98 (s, 3H), 1.97 (s, 3H), 1.60-1.50 (m, 1H), 1.47 (d, J = 12.0 Hz, 3H), 1.47 (d, J = 7.6 Hz, 3H), 1.45 (d, J = 6.4 Hz, 3H), 0.91-0.85 (m, 4H). | Scheme 2, Intermediate 1. Intermediate 38, and cyclopropane carbonyl chloride |
| 30 | 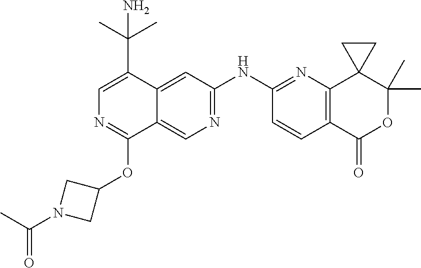 [SMILES: CC(=O)N1CC(Oc2ncc(C(C)(C)N)c3cc(Nc4ccc5c(n4)C4(CC4)C(C)(C)OC5=O)ncc23)C1] | 517 | ¹H NMR (400 M Hz, CDC13): δ ppm 9.47 (s, 1 H), 8.38 (s, 1 H), 8.16 (d, J = 8.8 Hz, 1 H), 8.08 (s, 1 H), 7.60 (d, J = 8.8 Hz, 1 H), 5.66-5.57 (m, 1 H), 4.76-4.69 (m, 1 H), 4.53- 4.47(m, 1 H), 4.45-4.35 (m, 1 H), 4.16-4.08(m,1 H), 1.94 (s, 3 H), 1.85 (s, 6 H), 1.59-1.54 (m, 2 H), 1.42 (s, 6 H), 1.33- 1.27 (m, 2 H). | Scheme 1, Intermediate 27, and Intermediate 7 |
| 31 | 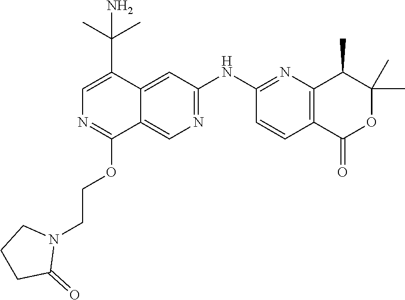 [SMILES: C[C@@H]1c2nc(Nc3cc4c(C(C)(C)N)cnc(OCCN5CCCC5=O)c4cn3)ccc2C(=O)OC1(C)C] | 519 | ¹H NMR (400 MHz, CD₃OD): δ ppm 9.38 (s, 1H), 9.29 (s, 1H), 8.16 (d, J = 8.8 Hz, 1H), 8.13 (s, 1H), 7.27 (d, J = 8.8 Hz, 1H), 4.72-4.69 (m, 2H), 3.85-3.82 (m, 2H), 3.69-3.65 (m, 2H), 3.05-3.02 (m, 1H), 2.44-2.33 (m, 2H), 2.07 (m, 2H), 1.92 (s, 3H), 1.89 (s, 3H), 1.56 (s, 3H), 1.47 (s, 3H), 1.42 (d, J = 7.2 Hz, 3H). | Scheme 1, Intermediate 3 and Intermediate 50 |
| 32 | 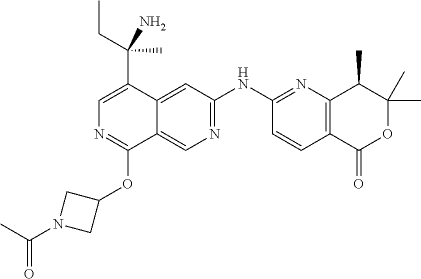 [SMILES: CC[C@@](C)(N)c1cnc(OC2CN(C(C)=O)C2)c2cnc(Nc3ccc4c(n3)[C@@H](C)C(C)(C)OC4=O)cc12] | 519 | ¹H-NMR (400 MHz, CD₃OD): δ ppm 9.38 (s, 1H), 9.37 (d, J = 9.2 Hz, 1H), 8.16 (s, 1H), 8.10 (d, J = 8.8 Hz, 1H), 7.18 (d, J = 8.8 Hz, 1H), 5.61-5.57 (m, 1H), 4.73-4.36 (m, 2H), 4.14-4.13 (m, 1H), 3.05-3.00 (m, 1H), 2.36-2.31 (m, 1H), 2.18-2.16 (m, 1H), 1.93 (s, 3H), 1.73 (s, 3H), 1.53 (s, 3H), 1.43 (s, 3H), 0.72 (t, J = 7.2 Hz, 3H). | Scheme 1, Intermediate 3 and Intermediate 45 |
| 33 | 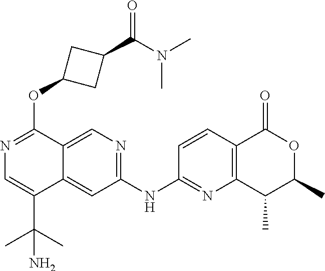 [SMILES: C[C@@H]1OC(=O)c2ccc(Nc3cc4c(C(C)(C)N)cnc(O[C@H]5C[C@@H](C(=O)N(C)C)C5)c4cn3)nc2[C@H]1C] | 519 | ¹H NMR (400 MHz, CD₃OD): δ ppm 9.35 (s, 1H), 9.18 (s, 1H), 8.18 (s, 1H), 8.15 (d, J = 8.4 Hz, 1H), 7.30 (d, J = 8.4 Hz, 1H), 5.43-5.36 (m, 1H), 4.68-4.65 (m, 1H), 3.23-3.19 (m, 1H), 3.08 (s, 3H), 3.06-3.02 (m, 1H), 2.97 (s, 3H), 2.87-2.84 (m, 2H), 2.48-2.43 (m, 2H), 1.80 (d, J = 4.4 Hz, 6H), 1.52 (d, J = 7.2 Hz, 3H), 1.47 (d, J = 6.8 Hz, 3H). | Scheme 1, Intermediate 1 and Intermediate 51 |
| 34 | 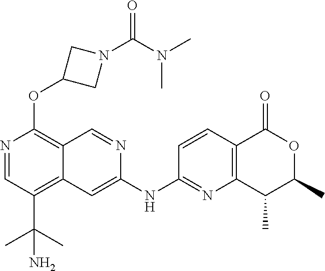 [SMILES: C[C@@H]1OC(=O)c2ccc(Nc3cc4c(C(C)(C)N)cnc(OC5CN(C(=O)N(C)C)C5)c4cn3)nc2[C@H]1C] | 520 | | Scheme 1, Intermediate 1 and Intermediate 37 |
| 35 | 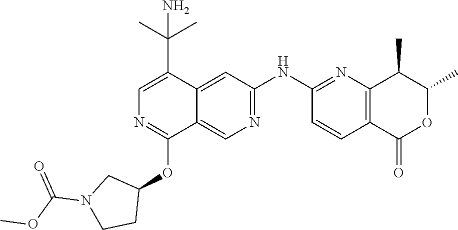 | 521 | ¹H-NMR (400 MHz, DMSO- d₆) δ ppm 10.67 (s, 1H), 9.46- 9.36 (m, 1H), 9.29 (s, 1H), 8.18 (s, 1H), 8.08 (d, 1H, J = 8.8 Hz), 7.40 (d, 1H, J = 8.4 Hz), 5.71 (s, 1H), 4.70-4.55 (m, 1H), 3.80-3.67 (m, 1H), 3.64-3.53 (m, 6H), 3.04-2.94 (m, 1H), 2.30-2.19 (m, 2H), 2.19-1.90 (m, 2H), 1.66 (d, 6H, J = 1.2 Hz), 1.47-1.41 (m, 3H), 1.38 (d, 3H, J = 6.4 Hz). | Scheme 1, Intermediate 1, Intermediate 28, and methyl carbono chloridate |
| 36 | 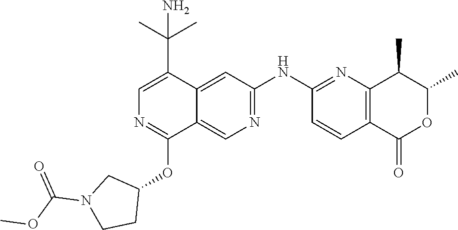 | 521 | ¹H NMR (400 MHz, CD₃OD): δ ppm 9.34 (s, 1H), 9.22 (s, 1H), 8.23 (s, 1H), 8.16 (d, J = 8.8 Hz, 1H), 7.32 (d, J = 8.8 Hz, 1H), 5.82 (s, 1H), 4.73- 4.62 (m, 1H), 3.89-3.62 (m, 7H), 3.10-2.99 (m, 1H), 2.38- 2.37 (m, 2H), 1.81 (d, J = 5.4 Hz, 6H), 1.53 (d, J = 7.2 Hz, 3H), 1.48 (d, J = 6.5 Hz, 3H). | Scheme 1, Intermediate 1, Intermediate 47, and methyl carbono chloridate |
| 37 | 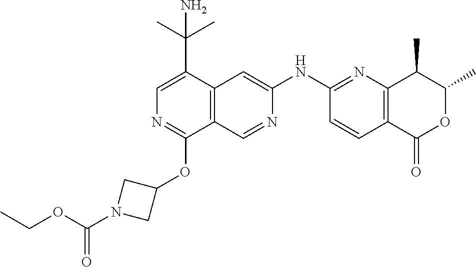 [SMILES: CCOC(=O)N1CC(Oc2ncc(C(C)(C)N)c3cc(Nc4ccc5c(n4)[C@@H](C)[C@H](C)OC5=O)ncc23)C1] | 521 | ¹H NMR (500 MHz, DMSO) δ 10.70 (s, 1H), 9.41 (s, 2H), 8.14 (s, 1H), 8.10 (d, J = 8.6 Hz, 1H), 7.40 (d, J = 8.7 Hz, 1H), 5.58 - 5.52 (m, 1H), 4.61 (p, J = 6.7 Hz, 1H), 4.40 (s, 2H), 4.05 (p, J = 7.8, 7.1 Hz, 4H), 3.01 (p, J = 6.8 Hz, 1H), 1.67 (d, J = 3.1 Hz, 6H), 1.45 (d, J = 7.1 Hz, 3H), 1.39 (d, J = 6.4 Hz, 3H), 1.19 (t, J = 7.2 Hz, 3H). | Scheme 1, Intermediate 1 and Intermediate 36 |
| 38 | 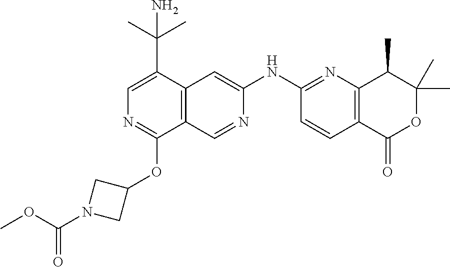 | 521 | ¹H NMR (500 MHz, DMSO) δ 10.73 (s, 1H), 9.58 (s, 1H), 9.40 (s, 1H), 8.16 (s, 1H), 8.08 (d, J = 8.6 Hz, 1H), 7.33 (d, J = 8.7 Hz, 1H), 5.55 (tt, J = 6.8, 4.1 Hz, 1H), 4.42 (d, J = 8.8 Hz, 2H), 4.09 (s, 2H), 3.60 (s, 3H), 3.01 (q, J = 7.1 Hz, 1H), 1.67 (d, J = 3.5 Hz, 6H), 1.45 (s, 3H), 1.39 (d, J = 14.6 Hz, 3H), 1.33 (d, J = 7.2 Hz, 3H). | Scheme 1, Intermediate 3 and Intermediate 35 |
| 39 | 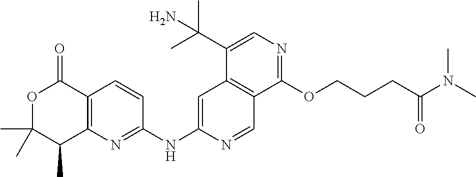 | 521 | ¹H NMR (400 MHz, CD3OD): δ ppm 9.40 (s, 1 H), 9.22 (s, 1 H), 8.15 (d, J = 8.4 Hz, 1 H), 8.06 (s, 1 H), 7.26 (d, J = 8.8 Hz, 1 H), 4.60-4.57 (m, 2 H), 3.09 (s, 3 H), 3.05- 2.99 (m, 1 H), 2.94 (s, 3 H), 2.68-2.62 (m, 2 H), 2.27-2.18 (m, 2 H), 1.92 (d, J = 10.4 Hz, 6 H), 1.54 (s, 3 H), 1.47-1.38 (m, 6 H). | Scheme 1, Intermediate 3 and Intermediate 52 |
| 40 | 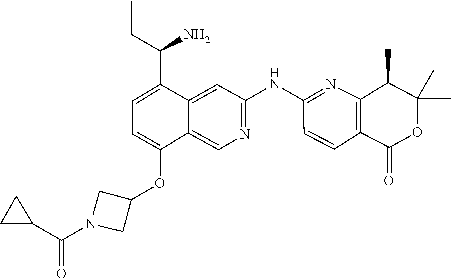 [SMILES: CC[C@@H](N)c1ccc(OC2CN(C(=O)C3CC3)C2)c2cnc(Nc3ccc4c(n3)[C@@H](C)C(C)(C)OC4=O)cc12] | 530 | ¹H NMR (400 M Hz, CD₃OD): δ ppm 9.43 (s, 1H), 8.91 (s, 1H), 8.09 (d, J = 8.8 Hz, 1H), 7.67 (d, J = 8.0 Hz, 1H), 7.20 (d, J = 8.8 Hz, 1H), 6.65 (d, J = 8.0 Hz, 1H), 5.39- 5.27 (m, 1H), 4.90 (s, 1H), 4.60-4.46 (m, 3H), 4.16-4.07 (m, 1H), 3.20-3.09 (m, 1 H), 2.03-1.82 (m, 2H), 1.71-1.60 (m, 1H), 1.54-1.48 (m, 6H), 1.45 (s, 3H), 0.97 (t, J = 7.4 Hz, 3H), 0.94-0.82 (m, 4H). | Scheme 5, Intermediate 3, Intermediate 39, and cyclopropane carbonyl chloride |
| | or | | | |
| | 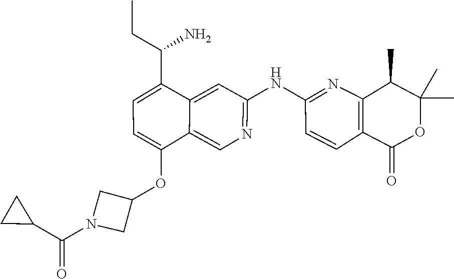 [SMILES: CC[C@H](N)c1ccc(OC2CN(C(=O)C3CC3)C2)c2cnc(Nc3ccc4c(n3)[C@@H](C)C(C)(C)OC4=O)cc12] | | | |
| 41 | 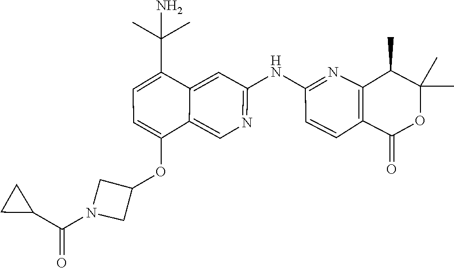 [SMILES: C[C@@H]1c2nc(Nc3cc4c(C(C)(C)N)ccc(OC5CN(C(=O)C6CC6)C5)c4cn3)ccc2C(=O)OC1(C)C] | 530 | ¹H-NMR (400 MHz, CD₃OD): δ ppm 9.52 (s, 1H), 9.33 (s, 1H), 8.55 (s, 1H), 8.13 (d, J = 8.8 Hz, 1H), 7.58 (d, J = 8.4 Hz, 1H), 7.22 (d, J = 8.8 Hz, 1H), 6.66 (d, J = 8.4 Hz, 1H), 5.35 (s, 1H), 4.93- 4.90 (m, 1H), 4.60-4.53 (m, 1H), 3.10-2.90 (m, 1H), 2.01(s, 3H), 1.99 (s, 3H), 1.70-1.65 (m, 1H), 1.56 (s, 3H), 1.47 (s, 3H), 1.42 (d, J = 7.6 Hz, 3H), 1.0-0.80 (m, 4H). | Scheme 2, Intermediate 3, Intermediate 38, and cyclopropane carbonyl chloride |
| 42 | 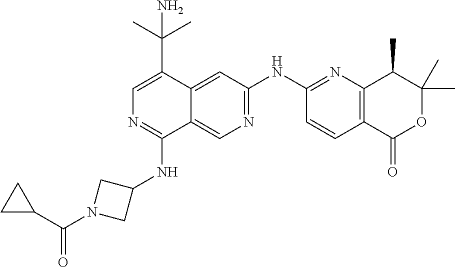 [SMILES: C[C@@H]1c2nc(Nc3cc4c(C(C)(C)N)cnc(NC5CN(C(=O)C6CC6)C5)c4cn3)ccc2C(=O)OC1(C)C] | 530 | ¹H NMR (400 MHz, CD₃OD): δ ppm 9.37 (s, 1H), 9.29 (s, 1H), 8.17-8.09 (m, 2H), 7.24 (d, J = 8.8 Hz, 1H), 4.82-4.77 (m, 2H), 4.49-4.38 (m, 1H), 4.36-4.25 (m, 1H), 4.09-4.00 (m, 1H), 3.06 (q, J = 7.2 Hz, 1H), 1.79 (d, J = 7.2 Hz, 6H), 1.69-1.60 (m, 1H), 1.59-1.44 (m, 6H), 1.41 (d, J = 7.2 Hz, 3H), 0.99-0.81 (m, 4H). | Scheme 1, Intermediate 3 and Intermediate 42 |
| 43 | 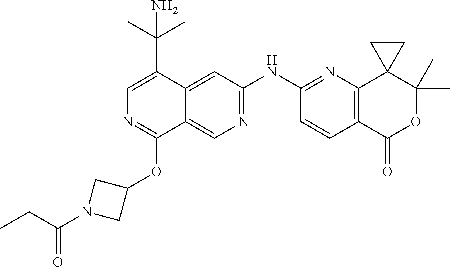 [SMILES: CCC(=O)N1CC(Oc2ncc(C(C)(C)N)c3cc(Nc4ccc5c(n4)C4(CC4)C(C)(C)OC5=O)ncc23)C1] | 531 | ¹H NMR (400 MHz, CDCl3): δ ppm 9.40 (s, 1 H), 8.54 (s, 1 H), 8.41 (s, 1 H), 8.18 -8.03 (m, 2 H), 7.53 (d, J = 8.8 Hz, 1 H), 5.59-5.58 (m, 1 H), 4.74-4.64 (m, 1 H), 4.52-4.43 (m, 1 H), 4.40-4.32 (m, 1 H), 2.27-2.16 (m, 2 H), 1.80 (s, 6 H), 1.55 (s, 2 H), 1.40 (s, 6 H), 1.28 (s, 2 H), 1.15-1.07 (m, 3 H). | Scheme 1, Intermediate 27, and Intermediate 9 |
| 44 | 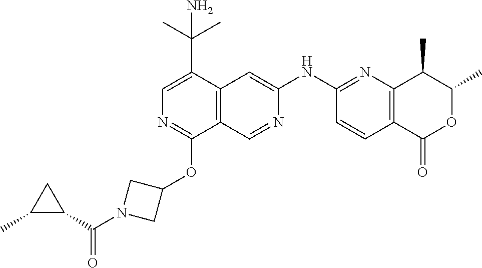 | 531 | ¹H NMR (400 MHz, CD₃OD): δ ppm 9.47 (d, J = 4.4 Hz, 1H), 9.13 (s, 1H), 8.17 (d, J = 8.4 Hz, 1H), 8.10 (s, 1H), 7.35 (d, J = 8.8 Hz, 1H), 5.71-5.62 (m, 1H), 4.82-4.74 (m, 1H), 4.70-4.65 (m, 1H), 4.55-4.40 (m, 2H), 4.18-4.10 (m, 1H), 3.08-2.99 (m, 1H), 1.90 (d, J = 5.2 Hz, 6H), 1.72- 1.62 (m, 1H), 1.52 (d, J = 7.2 Hz, 3H), 1.47 (d, J = 6.8 Hz, 3H), 1.38-1.31 (m, 1H), 1.17-1.11 (m, 3H), 1.00-0.93 (m, 1H), 0.91-0.85 (m, 1H). | Scheme 1, Intermediate 1 and Intermediate 53 |
| 45 | 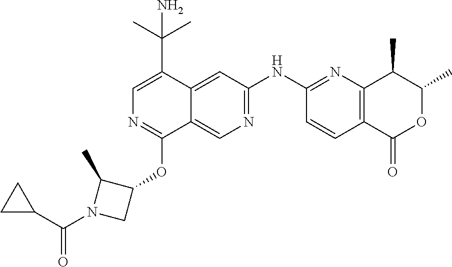 [SMILES: C[C@@H]1OC(=O)c2ccc(Nc3cc4c(C(C)(C)N)cnc(O[C@@H]5CN(C(=O)C6CC6)[C@H]5C)c4cn3)nc2[C@H]1C] | 531 | ¹H NMR (400 MHz, CD₃OD): δ ppm 9.44 (s, 1H), 9.24 (s, 1H), 8.23 (s, 1H), 8.17 (d, J = 8.8 Hz, 1H), 7.33 (d, J = 8.8 Hz, 1H), 5.30-5.20 (m, 1H), 4.83-4.75 (m, 1H), 4.72-4.64 (m, 1H), 4.54-4.42 (m, 1H), 4.42-4.03 (m, 1H), 3.11-2.96 (m, 1H), 1.85-1.79 (m, 7H), 1.70-1.57 (m, 3H), 1.56-1.44 (m, 6H), 1.06-0.79 (m, 4H). | Scheme 1, Intermediate 1 and Intermediate 63 |
| 46 | 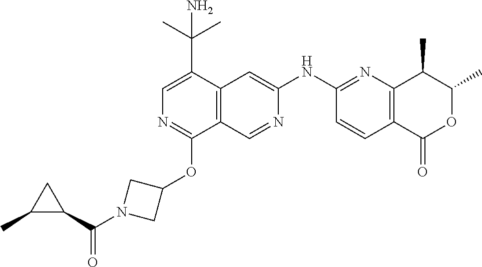 | 531 | ¹H NMR (400 MHz, CD₃OD): δ ppm 9.43 (d, J = 4.4 Hz, 1H), 9.22 (s, 1H), 8.19 (s, 1H), 8.15 (d, J = 8.4 Hz, 1H), 7.31 (d, J = 8.4 Hz, 1H), 5.69-5.62 (m, 1H), 4.82-4.74 (m, 1H), 4.70-4.63 (m, 1H), 4.54-4.36 (m, 2H), 4.18-4.10 (m, 1H), 3.09-3.00 (m, 1H), 1.79 (d, J = 4.8 Hz, 6H), 1.72- 1.63 (m, 1H), 1.51 (d, J = 7.2 Hz, 3H), 1.47 (d, J = 6.8 Hz, 3H), 1.38-1.29 (m, 1H), 1.17- 1.10 (m, 3H), 0.99-0.92 (m, 1H), 0.91-0.83 (m, 1H). | Scheme 2, Intermediate 1, Intermediate 9, Step 1 and (1R,2S)-2- methylcyclo propane-1- carboxylic acid |
| 47 | 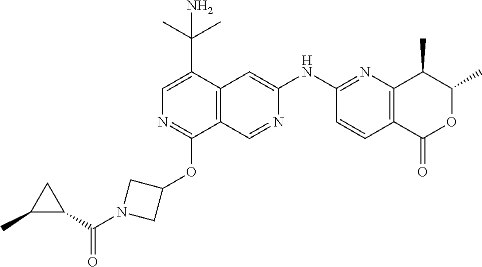 | 531 | ¹H NMR (400 MHz, CD₃OD): δ ppm 9.44 (s, 1H), 9.21 (s, 1H), 8.18 (s, 1H), 8.15 (d, J = 8.8 Hz, 1H), 7.31 (d, J = 8.4 Hz, 1H), 5.68-5.62 (m, 1H), 4.69-4.63 (m, 1H), 4.60-4.58 (m, 1H), 4.52-4.43 (m, 2H), 4.15-4.08 (m, 1H), 3.07-3.00 (m, 1H), 1.79 (d, J = 4.8 Hz, 6H), 1.51 (d, J = 7.2 Hz, 3H), 1.45 (d, J = 6.4 Hz, 3H), 1.36-1.27 (m, 2H), 1.15- 1.06 (m, 4H), 0.73-0.65 (m, 1H). | Scheme 2, Intermediate 1, Intermediate 9, Step 1, and (1S,2S)-2- methylcyclo propane-1 - carboxylic acid |
| 48 | 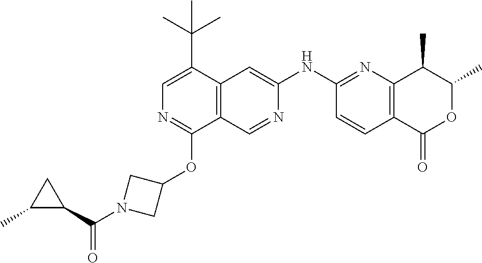 | 531 | ¹H NMR (400 MHz, CD₃OD): δ ppm 9.44 (s, 1H), 9.23 (s, 1H), 8.19 (s, 1H), 8.16 (d, J = 8.8 Hz, 1H), 7.32 (d, J = 8.8 Hz, 1H), 5.68-5.62 (m, 1H), 4.70-4.64 (m, 1H), 4.61-4.56 (m, 1H), 4.52-4.43 (m, 2H), 4.15-4.08 (m, 1H), 3.08-3.00 (m, 1H), 1.79 (d, J = 4.8 Hz, 6H), 1.51 (d, J = 7.2 Hz, 3H), 1.46 (d, J = 6.4 Hz, 3H), 1.37-1.29 (m, 2H), 1.17- 1.07 (m, 4H), 0.73-0.65 (m, 1H). | Scheme 2, Intermediate 1, Intermediate 9, Step 1, and (1R,2R)-2- methylcyclo propane-1- carboxylic acid |
| 49 | 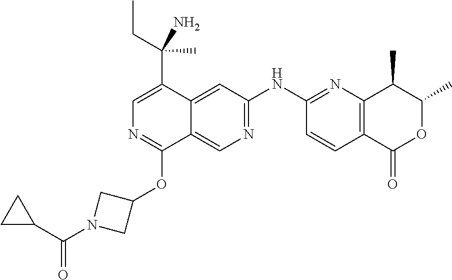 [SMILES: CC[C@@](C)(N)c1cnc(OC2CN(C(=O)C3CC3)C2)c2cnc(Nc3ccc4c(n3)[C@@H](C)[C@H](C)OC4=O)cc12] | 531 | ¹H NMR (400 MHz, CD₃OD): δ ppm 9.55 (s, 1H), 8.94 (s, 1H), 8.20 (d, J = 8.8 Hz, 1H), 7.96 (s, 1H), 7.42 (d, J = 8.4 Hz, 1H), 5.74-5.69 (m, 1H), 4.81-4.78 (m, 1H), 4.72- 4.68 (m, 1H), 4.57-4.52 (m, 2H), 4.17-4.14 (m, 1H), 3.03 (m, 1H), 2.74-2.72 (m, 1H), 2.38-2.36 (m, 1H), 2.03 (s, 3H), 1.67-1.66 (m, 1H), 1.53- 1.48 (m, 6H), 0.92-0.85 (m, 7H). | Scheme 1, Intermediate 1 and Intermediate 46 |
| 50 | 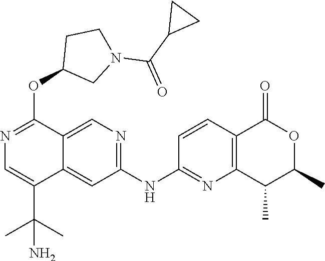 [SMILES: C[C@@H]1OC(=O)c2ccc(Nc3cc4c(C(C)(C)N)cnc(O[C@H]5CCN(C(=O)C6CC6)C5)c4cn3)nc2[C@H]1C] | 531 | ¹H-NMR (400 MHz, DMSO- d₆) δ ppm 10.67 (d, 1H, J = 6.8 Hz), 9.42 (d, 1H, J = 7.6 Hz), 9.31 (d, 1H, J = 2.4 Hz), 8.20 (d, 1H, J = 2.4 Hz), 8.09 (d, 1H, J = 8.8 Hz), 7.46-7.37 (m, 1H), 5.89-5.68 (m, 1H), 4.61 (t, 1H, J = 6.0 Hz), 4.15- 4.04 (m, 1H), 3.98-3.84 (m, 2H), 3.74-3.62 (m, 1H), 3.61- 3.47 (m, 1H), 3.00-3.29 (m, 1H), 2.42-2.35 (m, 1H), 2.29- 2.21 (m, 1H), 1.89-1.72 (m, 1H), 1.67 (s, 6H), 1.48-1.35 (m, 6H), 0.80-0.66 (m, 4H). | Scheme 2, Intermediate 1, Intermediate 28, and cyclopropanec arbonyl chloride |
| 51 | 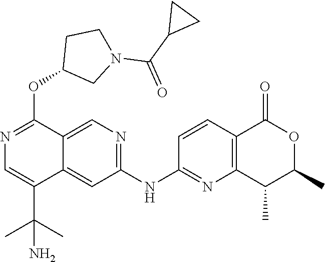 [SMILES: C[C@@H]1OC(=O)c2ccc(Nc3cc4c(C(C)(C)N)cnc(O[C@@H]5CCN(C(=O)C6CC6)C5)c4cn3)nc2[C@H]1C] | 531 | ¹H NMR (400 MHz, CD₃OD): δ ppm 9.35 (s, 1H), 9.22 (d, J = 4.8 Hz, 1H), 8.23 (d, J = 2.8 Hz, 1H), 8.16 (d, J = 8.8 Hz, 1H), 7.32 (d, J = 8.8 Hz, 1H), 6.03-5.79 (m, 1H), 4.77-4.58 (m, 1H), 4.25-3.96 (m, 2H), 3.93-3.64 (m, 2H), 3.11-2.98 (m, 1H), 2.61-2.24 (m, 2H), 1.97-1.83 (m, 1H), 1.81 (d, J = 4.4 Hz, 6H), 1.53 (d, J = 7.2 Hz, 3H), 1.48 (d, J = 7.2 Hz, 3H), 1.01-0.75 (m, 4H). | Scheme 2, Intermediate 1, Intermediate 47, and cyclopropane carbonyl chloride |
| 52 | 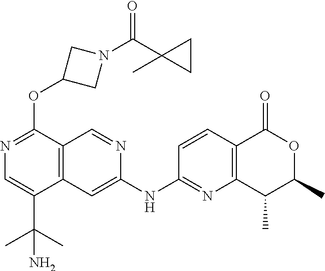 [SMILES: C[C@@H]1OC(=O)c2ccc(Nc3cc4c(C(C)(C)N)cnc(OC5CN(C(=O)C6(C)CC6)C5)c4cn3)nc2[C@H]1C] | 531 | ¹H NMR (400 M Hz, CD₃OD): δ ppm 9.48 (s, 1H), 9.21 (s, 1H), 8.21 (s, 1H), 8.15 (d, J = 8.4 Hz, 1H), 7.31 (d, J = 8.8 Hz, 1H), 5.64-5.58 (m, 1H), 4.98 (m, 1H), 4.72- 4.58 (m, 3H), 4.20 (m, 1H), 3.08-2.98 (m, 1H), 1.81 (d, J = 4.4 Hz, 6H), 1.53 (d, J = 7.2 Hz, 3H) 1.48 (d, J = 6.4 Hz, 3H), 1.35 (s, 3H), 1.06-1.04 (m, 2H), 0.60-0.57(m, 2H). | Scheme 1, Intermediate 1 and Intermediate 57 |
| 53 | 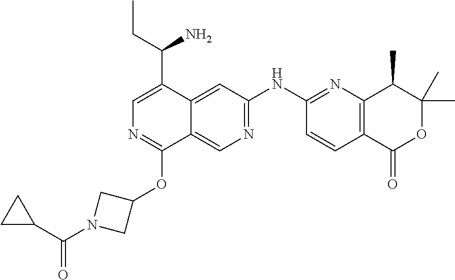 [SMILES: CC[C@@H](N)c1cnc(OC2CN(C(=O)C3CC3)C2)c2cnc(Nc3ccc4c(n3)[C@@H](C)C(C)(C)OC4=O)cc12] | 531 | ¹H-NMR (400 MHz, CD₃OD): δ ppm 9.46 (d, J = 3.6 Hz, 1H), 8.81 (s, 1H), 8.21-8.12 (m, 2H), 7.31 (d, J = 8.8 Hz, 1H), 5.68 (s, 1H), 4.67-4.59 (m, 1H), 4.51 (d, J = 6.0, 10.4 Hz, 2H), 4.18-4.09 (m, 1H), 3.19 (d, J = 7.2 Hz, 1H), 2.17-2.05 (m, 2H), 1.70- 1.60 (m, 1H), 1.55-1.50 (m, 6H), 1.45 (s, 3H), 1.34-1.24 (m, 1H), 1.04 (d, J = 7.6 Hz, 3H), 0.92-0.85 (m, 4H) | Scheme 3, Intermediate 3 and Intermediate 65 |
| | or | | | |
| | 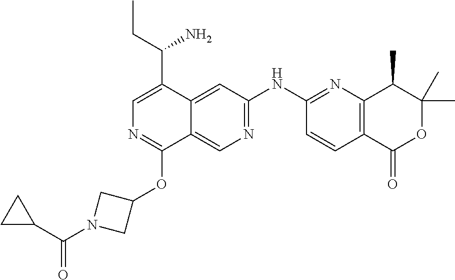 [SMILES: CC[C@H](N)c1cnc(OC2CN(C(=O)C3CC3)C2)c2cnc(Nc3ccc4c(n3)[C@@H](C)C(C)(C)OC4=O)cc12] | | | |
| 54 | 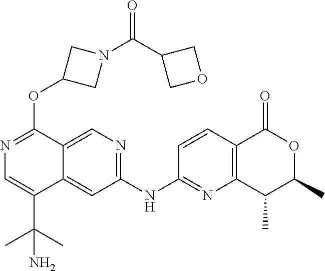 [SMILES: C[C@@H]1OC(=O)c2ccc(Nc3cc4c(C(C)(C)N)cnc(OC5CN(C(=O)C6COC6)C5)c4cn3)nc2[C@H]1C] | 533 | ¹H NMR (400 MHz, CD₃OD): δ ppm 9.42 (s, 1H), 9.22 (s, 1H), 8.17 (s, 1H), 8.15 (d, J = 8.8 Hz, 1H), 7.31 (d, J = 8.4 Hz, 1H), 5.67-5.58 (m, 1H), 4.81-4.77 (m, 1H), 4.68-4.62 (m, 2H), 4.61-4.56 (m, 3H), 4.55-4.50 (m, 1H), 4.32-4.25 (m, 1H), 4.20-4.14 (m, 1H), 4.04-3.95 (m, 1H), 3.06-3.00 (m, 1H), 1.79 (d, J = 4.4 Hz, 6H), 1.51 (d, J = 7.2 Hz, 3H), 1.46 (d, J = 6.4 Hz, 3H). | Scheme 2, Intermediate 1. Intermediate 9, Step 1, and oxetane-3- carboxylic acid |
| 55 | 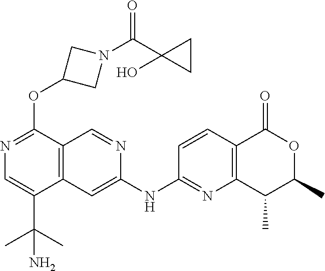 [SMILES: C[C@@H]1OC(=O)c2ccc(Nc3cc4c(C(C)(C)N)cnc(OC5CN(C(=O)C6(O)CC6)C5)c4cn3)nc2[C@H]1C] | 533 | ¹H NMR (400 MHz, CD3OD): δ ppm 9.49 (s, 1H), 9.06 (s, 1H), 8.52 (s, 1H), 8.17 (d, J = 8.4 Hz, 1H), 8.04 (s, 1H), 7.36 (d, J = 8.4 Hz, 1H), 5.65-5.60 (m, 1H), 5.05- 4.65 (m, 4H), 4.17-4.10 (m, 1H), 3.05-3.01 (m, 1H), 2.00- 1.96 (m, 6H), 1.55-1.45 (m, 6H), 1.25-1.20 (m, 2H), 0.96- 0.90 (m, 2H). | Scheme 2, Intermediate 1, Intermediate 9, Step 1, and 1-hydroxy cyclopropane- 1-carboxylic acid |
| 56 | 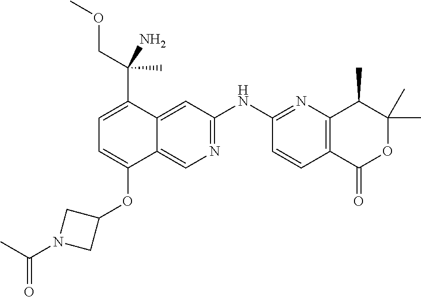 [SMILES: COC[C@@](C)(N)c1ccc(OC2CN(C(C)=O)C2)c2cnc(Nc3ccc4c(n3)[C@@H](C)C(C)(C)OC4=O)cc12] | 534 | ¹H NMR (400 MHz, CD3OD): δ ppm 9.50 (s, 1 H), 9.35 (s, 1 H), 8.53 (s, 1 H), 8.10 (d, J = 8.8 Hz, 1 H), 7.58 (d, J = 8.4 Hz, 1 H), 7.18 (d, J = 8.8 Hz, 1 H), 6.64 (d, J = 8.4 Hz, 1 H), 5.34-5.23 (m, 1 H), 4.79-4.72 (m, 1 H), 4.56-4.49 (m, 1 H), 4.44-4.38 (m, 1 H), 4.20-4.15 (m, 1 H), 4.13-4.07 (m, 1 H), 3.99-3.93 (m, 1 H), 3.39 (s, 3 H), 3.05-2.97 (m, 1 H), 1.95 (d, J = 4.4 Hz, 6 H), 1.55 (s, 3 H), 1.47 (s, 3 H), 1.39 (d, J = 7.2 Hz, 3 H). | Scheme 2, Intermediate 3, Intermediate 67, and acetyl chloride |
| 57 | 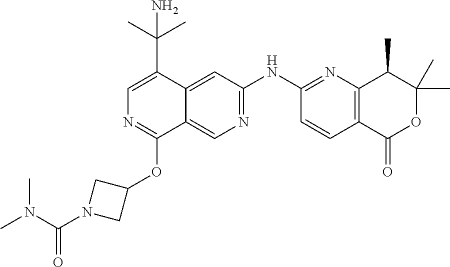 | 534 | ¹H-NMR (400 MHz, CDCl₃): δ ppm 9.42-9.37 (m, 2H), 8.28-8.14 (m, 2H), 8.11 (s, 1H), 7.16 (d, J = 8.8 Hz, 1H), 5.59-5.50 (m, 1H), 4.49-4.45 (m, 2H), 4.20-4.17 (m, 2H), 2.99 (m, 1H), 2.90 (s, 6H), 1.75 (s, 8H), 1.49 (d, J = 12.0 Hz, 6H), 1.43 (d, J = 7.2 Hz, 3H). | Scheme 1, Intermediate 3 and Intermediate 37 |
| 58 | 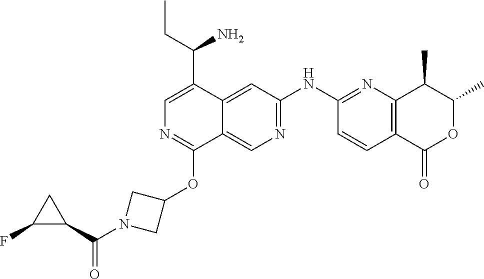 [SMILES: CC[C@@H](N)c1cnc(OC2CN(C(=O)[C@@H]3C[C@@H]3F)C2)c2cnc(Nc3ccc4c(n3)[C@@H](C)[C@H](C)OC4=O)cc12] | 535 | ¹H NMR (400 MHz, CD₃OD): δ ppm 9.70 (d, J = 2.0 Hz, 1H), 8.75 (s, 1H), 8.51 (s, 1H), 8.23 (d, J = 8.0 Hz, 2H), 8.17 (d, J = 8.8 Hz, 1H), 7.35-7.28 (m, 1H), 5.743-5.67 (m, 1H), 4.80-4.77 (m, 2H), 4.58-4.53 (m, 3H), 4.19-4.18 (s, 1H), 3.15-3.07 (m, 1H), 2.20-2.16 (m, 2H), 1.91-1.89 (m, 1H), 1.73-1.67 (m, 1H), 1.62-1.61 (m, 3H), 1.57-1.55 (m, 3H), 1.23-1.20 (m, 1H), 1.07-1.04 (m, 3H). | Scheme 3, Intermediate 1 and Intermediate 13 |
| | or | | | |
| | 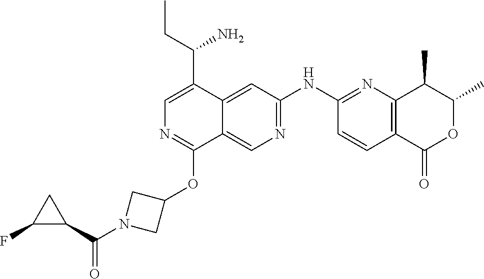 [SMILES: CC[C@H](N)c1cnc(OC2CN(C(=O)[C@@H]3C[C@@H]3F)C2)c2cnc(Nc3ccc4c(n3)[C@@H](C)[C@H](C)OC4=O)cc12] | | | |
| 59 | 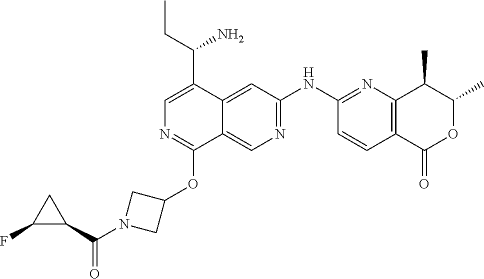 [SMILES: CC[C@H](N)c1cnc(OC2CN(C(=O)[C@@H]3C[C@@H]3F)C2)c2cnc(Nc3ccc4c(n3)[C@@H](C)[C@H](C)OC4=O)cc12] | 535 | | Scheme 2, Intermediate 1, Intermediate 9, Step 1, and (1S,2R)-2- fluorocyclo propane-1- carboxylic acid |
| 60 | 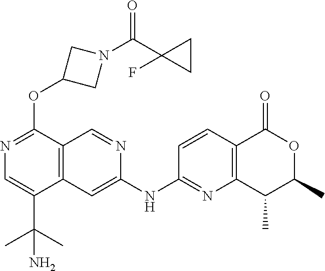 [SMILES: C[C@@H]1OC(=O)c2ccc(Nc3cc4c(C(C)(C)N)cnc(OC5CN(C(=O)C6(F)CC6)C5)c4cn3)nc2[C@H]1C] | 535 | ¹H NMR (400 MHz, CD₃OD): δ ppm 9.47 (s, 1H), 9.25 (s, 1H), 8.21 (s, 1H), 8.17 (d, J = 8.8 Hz, 1H), 7.34 (d, J = 8.8 Hz, 1H), 5.69-5.68 (m, 1H), 5.10-5.00 (m, 1H), 4.80- 4.75 (m, 1H), 4.61 (s, 4H), 4.30 - 4.20 (m, 1H), 3.07 - 3.04 (m, 1H), 1.82 (d, J = 4.8 Hz, 6H), 1.53 (d, J = 7.2 Hz, 6H), 1.35 (s, 2H), 1.32- 1.28 (m, 2H). | Scheme 2, Intermediate 1, Intermediate 9, Step 1, and 1-fluorocyclo propane-1 - carboxylic acid |
| 61 | 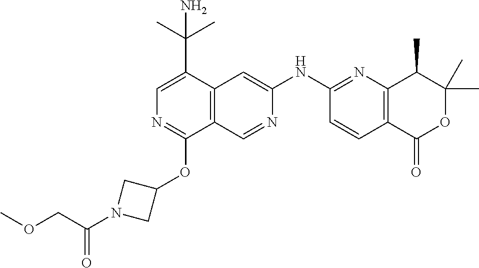 | 535 | ¹H NMR (400 MHz, CD₃OD): δ ppm 9.50 (s, 1H), 9.21 (s, 1H), 8.19 (s, 1H), 8.03 (s, 1H), 7.31 (d, J = 8.8 Hz, 1H), 5.71- 5.48 (m, 1H), 4.85-4.80 (m, 1H), 4.60 (s, 1H), 4.59- 4.50 (m, 1H), 4.20 (m, 1H), 4.07 (s, 2H), 3.42 (s, 3H), 3.03 (m, 1H) , 2.02 (m, 6H), 1.56 (s, 3H), 1.47 (s, 3H), 1.43 (d, J = 7.2 Hz, 3H). | Scheme 1, Intermediate 3 and Intermediate 54 |
| 62 | 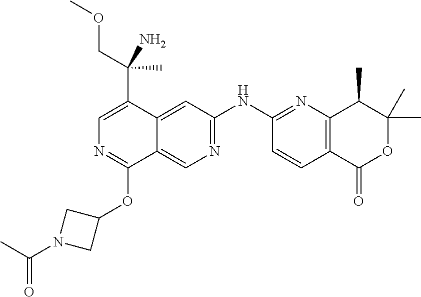 [SMILES: COC[C@@](C)(N)c1cnc(OC2CN(C(C)=O)C2)c2cnc(Nc3ccc4c(n3)[C@@H](C)C(C)(C)OC4=O)cc12] | 535 | ¹H NMR (CH₃OH-d₄, 400 MHz): δ 9.42 (2H, s), 8.16 (1H, s), 8.13 (1H, d, J = 8.7 Hz), 5.60 (1H, s), 4.71 (1H, t, J = 8.4 Hz), 4.61 (1H, s), 4.48 (1H, t, J = 8.8 Hz), 4.38 (1H, d, J = 10.0 Hz), 4.12 (1H, d, J = 11.4 Hz), 3.90-3.93 (1H, m), 3.86 (1H, d, J = 9.1 Hz), 3.34 (3H, s), 3.04 (1H, d, J = 7.3 Hz), 1.93 (3H, s), 1.74 (3H, s), 1.54 (3H, s), 1.45 (3H, s), 1.39 (3H, d, J = 7.1 Hz). | Scheme 1, Intermediate 3 and Intermediate 29 |
| 63 | 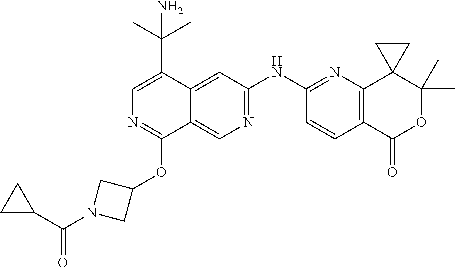 [SMILES: CC(C)(N)c1cnc(OC2CN(C(=O)C3CC3)C2)c2cnc(Nc3ccc4c(n3)C3(CC3)C(C)(C)OC4=O)cc12] | 543 | ¹H NMR (400 MHz, CD₃OD): δ ppm 9.51 (s, 1H), 8.50 (s, 1H), 8.28 (s, 1H), 8.18 (d, J = 8.8 Hz, 1H), 8.02 (s, 1H), 7.67 (d, J = 8.4 Hz, 1H), 5.71-5.61 (m, 1H), 4.81 (s, 1H), 4.55-4.45 (m, 2H), 4.19-4.07 (m, 1H), 1.91 (s, 6H), 1.69-1.59 (m, 1H), 1.58- 1.5 (m, 2H), 1.42 (s, 6H), 1.34-1.27 (m, 2H), 0.94-0.81 (m, 4H). | Scheme 1, Intermediate 27 and Intermediate 8 |
| 64 | 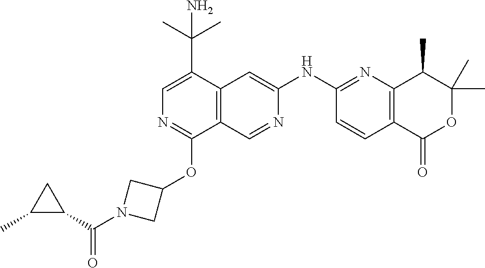 | 545 | ¹H NMR (400 MHz, CD₃OD): δ ppm 9.47 (d, J = 4.4 Hz, 1H), 9.29 (s, 1H), 8.15 (d, J = 8.4 Hz, 1H), 8.10 (s, 1H), 7.27 (d, J = 8.4 Hz, 1H), 5.71-5.61 (m, 1H), 4.84-4.74 (m, 1H), 4.63-4.39 (m, 2H), 4.20-4.02 (m, 1H), 3.07-2.99 (m, 1H), 1.91 (d, J = 10.0 Hz, 6H), 1.73-1.63 (m, 1H), 1.54 (s, 3H), 1.46 (s, 3H), 1.41 (d, J = 7.2 Hz, 3H), 1.38-1.33 (m, 1H), 1.18-1.10 (m, 3H), 1.01- 0.93 (m, 1H), 0.90-0.85 (m, 1H). | Scheme 1, Intermediate 3, Intermediate 9, Step 1, and (1S,2R)-2- methylcyclo propane-1- carboxylic acid |
| 65 | 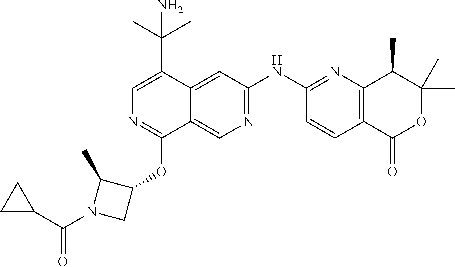 [SMILES: C[C@@H]1c2nc(Nc3cc4c(C(C)(C)N)cnc(O[C@@H]5CN(C(=O)C6CC6)[C@H]5C)c4cn3)ccc2C(=O)OC1(C)C] | 545 | ¹H NMR (400 MHz, CD₃OD): δ ppm 9.43 (d, J = 8.4 Hz, 2H), 8.23 (s, 1H), 8.15 (d, J = 8.8 Hz, 1H), 7.24 (d, J = 8.8 Hz, 1H), 5.31-5.19 (m, 1H), 4.84-4.74 (m, 1H), 4.56-4.43 (m, 1H), 4.41-4.01 (m, 1H), 3.11-3.01 (m, 1H), 1.82 (d, J = 6.8 Hz, 7H), 1.73-1.58 (m, 3H), 1.57-1.45 (m, 6H), 1.42 (d, J = 7.2 Hz, 3H), 1.02-0.80 (m, 4H). | Scheme 1, Intermediate 3 and Intermediate 63 |
| 66 | 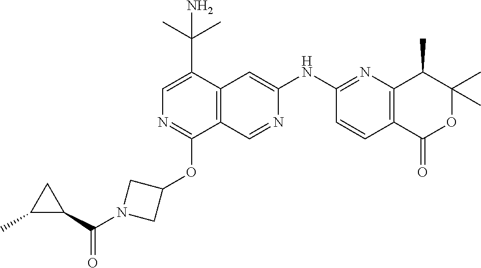 | 545 | ¹H NMR (400 MHz, CD₃OD): δ ppm 9.48 (s, 1H), 9.26 (s, 1H), 8.16 (d, J = 8.8 Hz, 1H), 8.08 (s, 1H), 7.28 (d, J = 8.8 Hz, 1H), 5.73-5.62 (m, 1H), 4.82 (s, 1H), 4.54-4.45 (m, 2H), 4.18-4.05 (m, 1H), 3.07-2.98 (m, 1H), 1.94 (d, J = 10.8 Hz, 6H), 1.55 (s, 3H), 1.46 (s, 3H), 1.42 (d, J = 7.2 Hz, 3H), 1.38-1.29 (m, 2H), 1.17-1.12 (m, 3H), 1.12-1.07 (m, 1H), 0.74-0.66 (m, 1H). | Scheme 2, Intermediate 3, Intermediate 9, Step 1, and (1R,2R)-2- methylcyclo propane-1- carboxylic acid |
| 67 | 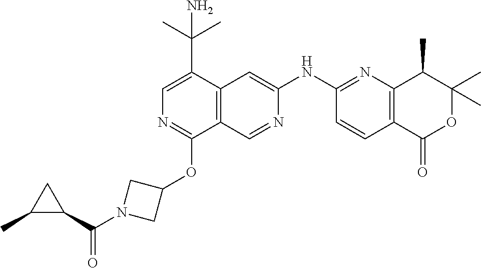 | 545 | ¹H NMR (400 MHz, CD₃OD): δ ppm 9.44 (d, J = 2.0 Hz, 1H), 9.40 (s, 1H), 8.20 (s, 1H), 8.14 (d, J = 8.8 Hz, 1H), 7.23 (d, J = 8.8 Hz, 1H), 5.71-5.61 (m, 1H), 4.82 (s, 1H), 4.54-4.41 (m, 2H), 4.18- 4.07 (m, 1H), 3.11-2.99 (m, 1H), 1.81 (d, J = 7.2 Hz, 6H), 1.55 (s, 3H), 1.46 (s, 3H), 1.44-1.39 (m, 3H), 1.38-1.28 (m, 2H), 1.18-1.12 (m, 3H), 1.12-1.06 (m, 1H), 0.73-0.66 (m, 1H). | Scheme 2, Intermediate 3, Intermediate 9, Step 1, and (1R,2S)-2- methylcyclo propane-1- carboxylic acid |
| 68 | 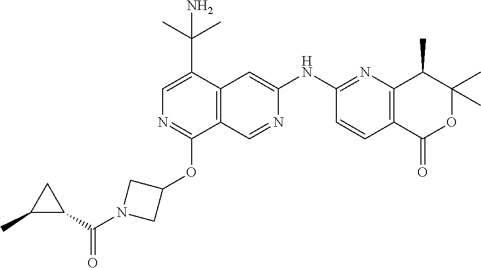 | 545 | ¹H NMR (400 MHz, CD₃OD): δ ppm 9.44 (d, J = 2.0 Hz, 1H), 9.40 (s, 1H), 8.20 (s, 1H), 8.14 (d, J = 8.8 Hz, 1H), 7.23 (d, J = 8.8 Hz, 1H), 5.71-5.61 (m, 1H), 4.82 (s, 1H), 4.54-4.41 (m, 2H), 4.18- 4.07 (m, 1H), 3.11-2.99 (m, 1H), 1.81 (d, J = 7.2 Hz, 6H), 1.55 (s, 3H), 1.46 (s, 3H), 1.44-1.39 (m, 3H), 1.38-1.28 (m, 2H), 1.18-1.12 (m, 3H), 1.12-1.06 (m, 1H), 0.73-0.66 (m, 1H). | Scheme 2, Intermediate 3, Intermediate 9, Step 1, and (15,2S)-2- methylcyclo propane-1- carboxylic acid |
| 69 | 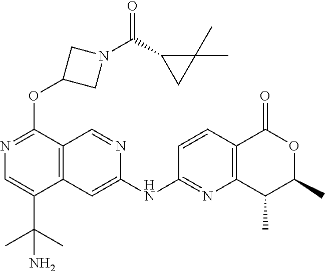 [SMILES: C[C@@H]1OC(=O)c2ccc(Nc3cc4c(C(C)(C)N)cnc(OC5CN(C(=O)[C@H]6CC6(C)C)C5)c4cn3)nc2[C@H]1C] | 545 | ¹H NMR (400 MHz, CD₃OD): δ ppm 9.46 (d, J = 3.2 Hz, 1H), 9.25 (s, 1H), 8.21 (s, 1H), 8.17 (d, J = 8.8 Hz, 1H), 7.34 (d, J = 8.8 Hz, 1H), 5.69-5.62 (m, 1H), 4.76-4.68 (m, 1H), 4.64-4.61 (m, 1H), 4.58-4.33 (m, 2H), 4.18-4.12 (m, 1H), 3.09-3.00 (m, 1H), 1.82 (d, J = 4.4 Hz, 6H), 1.56- 1.46 (m, 7H), 1.25-1.16 (m, 7H), 1.12-1.08 (m, 1H), 0.86- 0.80 (m, 1H). | Scheme 2, Intermediate 1, Intermediate 9, Step 1, and (S)-2,2- dimethylcyclo propane-1- carboxylic acid |
| 70 | 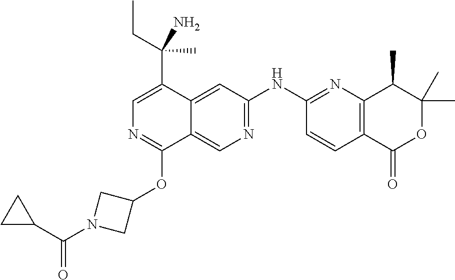 [SMILES: CC[C@@](C)(N)c1cnc(OC2CN(C(=O)C3CC3)C2)c2cnc(Nc3ccc4c(n3)[C@@H](C)C(C)(C)OC4=O)cc12] | 545 | ¹H NMR (400 MHz, CD₃OD): δ ppm 9.54 (s, 1H), 9.13 (s, 1H), 8.19 (d, J = 8.4 Hz, 1H), 7.96 (d, J = 2.0 Hz, 1H), 7.33 (d, J = 8.4 Hz, 1H), 5.74-5.69 (m, 1H), 4.70 (s, 1H), 4.57-4.51 (m, 2H), 4.17- 4.14 (m, 1H), 3.01-2.97 (m, 1H), 2.74-2.71 (m, 1H), 2.41- 2.40 (m, 1H), 2.03 (s, 3H), 1.68- 1.67(m, 1H), 1.56 (s, 3H), 1.48 (s, 3H), 1.42 (d, J = 6.8 Hz, 3H), 0.93-0.86 (m, 7H). | Scheme 1, Intermediate 3 and Intermediate 46 |
| 71 | 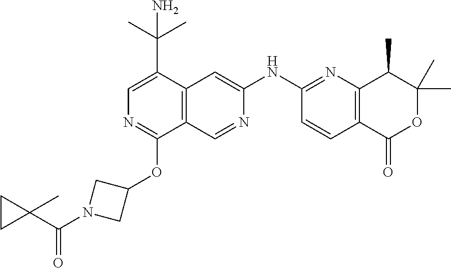 [SMILES: C[C@@H]1c2nc(Nc3cc4c(C(C)(C)N)cnc(OC5CN(C(=O)C6(C)CC6)C5)c4cn3)ccc2C(=O)OC1(C)C] | 545 | ¹H NMR (400 M Hz, CD₃OD): δ ppm 9.49 (s, 1H), 9.23 (s, 1H), 8.16 (d, J = 8.8 Hz, 1H), 8.05 (s, 1H), 7.28 (d, J = 8.8 Hz, 1H), 5.69-5.58 (m, 1H), 5.00-4.89 (m, 1H), 4.66- 4.43 (m, 2H), 4.30-4.00 (m, 1H), 3.07-2.95 (m, 1H), 1.96 (d, J = 11.2 Hz, 6H), 1.54 (s, 3H), 1.45 (s, 3H), 1.41 (d, J = 7.2 Hz, 3H), 1.35 (s, 3H), 1.08-1.03 (m, 2H), 0.62-0.57 (m, 2H). | Scheme 1, Intermediate 3 and Intermediate 57 |
| 72 | 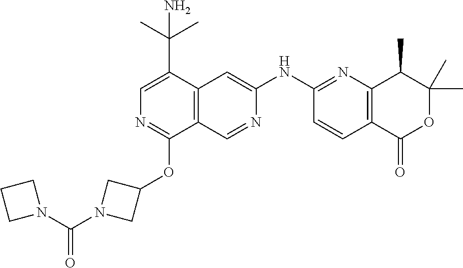 [SMILES: C[C@@H]1c2nc(Nc3cc4c(C(C)(C)N)cnc(OC5CN(C(=O)N6CCC6)C5)c4cn3)ccc2C(=O)OC1(C)C] | 546 | ¹H-NMR (400 MHz, CD₃OD): δ ppm 9.40 (s, 1H), 9.32 (s, 1H), 8.14 (d, J = 8.8 Hz, 1H), 8.12 (s, 1H), 7.24 (d, J = 8.8 Hz, 1H), 5.62-5.54 (m, 1H), 4.49-4.45 (m, 2H), 4.14- 4.11 (m, 2H), 4.04 (d, J = 7.6 Hz, 4H), 3.04 (d, J = 7.2 Hz, 1H), 2.30 (d, J = 7.6 Hz, 2H), 1.86 (d, J = 8.8 Hz, 6H), 1.55 (s, 3H), 1.46 (s, 3H), 1.41 (d, J = 7.2 Hz, 3H) | Scheme 1, Intermediate 3 and Intermediate 69 |
| 73 | 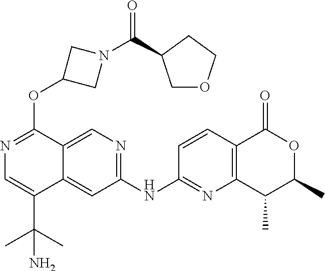 [SMILES: C[C@@H]1OC(=O)c2ccc(Nc3cc4c(C(C)(C)N)cnc(OC5CN(C(=O)[C@H]6CCOC6)C5)c4cn3)nc2[C@H]1C] | 547 | ¹H NMR (400 MHz, CD₃OD): δ ppm 9.43 (s, 1H), 9.16 (s, 1H), 8.18-8.09 (m, 2H), 7.36-7.27 (m, 1H), 5.67- 5.58 (m, 1H), 4.80-4.72 (m, 1H), 4.70-4.62 (m, 1H), 4.54- 4.39 (m, 2H), 4.19-4.10 (m, 1H), 4.02-3.97 (m, 1H), 3.93- 3.86 (m, 1H), 3.85-3.75 (m, 2H), 3.22-3.10 (m, 1H), 3.06- 2.96 (m, 1H), 2.24-2.03 (m, 2H), 1.84 (d, J = 5.2 Hz, 6H), 1.51 (d, J = 7.2 Hz, 3H), 1.46 (d, J = 6.8 Hz, 3H). | Scheme 1, Intermediate 1 and Intermediate 61 |
| 74 | 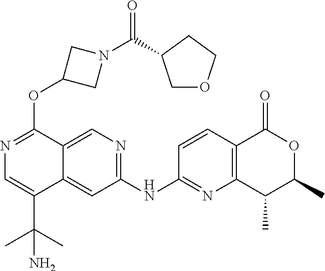 [SMILES: C[C@@H]1OC(=O)c2ccc(Nc3cc4c(C(C)(C)N)cnc(OC5CN(C(=O)[C@@H]6CCOC6)C5)c4cn3)nc2[C@H]1C] | 547 | ¹H NMR (400 M Hz, CD₃OD): δ ppm 9.44 (s, 1H), 9.17 (s, 1H), 8.15 (d, J = 8.8 Hz, 1H), 8.14 (s, 1H), 7.32 (d, J = 8.8 Hz, 1H), 5.67-5.56 (m, 1H), 4.80-4.73 (m, 1H), 4.71- 4.63 (m, 1H), 4.54-4.39 (m, 2H), 4.18-4.10 (m, 1H), 4.02- 3.93 (m, 1H), 3.93-3.86 (m, 1H), 3.85-3.75 (m, 2H), 3.21- 3.11 (m, 1H), 3.07-2.98 (m, 1H), 2.23-2.03 (m, 2H), 1.84 (d, J = 5.2 Hz, 6H), 1.51 (d, J = 7.2 Hz, 3H), 1.46 (d, J = 6.4 Hz, 3H). | Scheme 1, Intermediate 1 and Intermediate 60 |
| 75 | 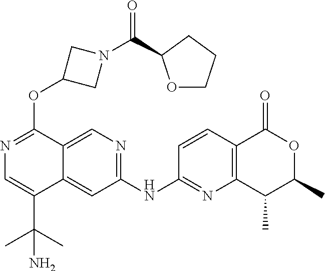 [SMILES: C[C@@H]1OC(=O)c2ccc(Nc3cc4c(C(C)(C)N)cnc(OC5CN(C(=O)[C@H]6CCCO6)C5)c4cn3)nc2[C@H]1C] | 547 | ¹H NMR (400 MHz, CD₃OD): δ ppm 9.50 (d, J = 2.0 Hz, 1H), 8.99 (s, 1H), 8.18 (d, J = 8.8 Hz, 1H), 7.99 (s, 1H), 7.40 (d, J = 8.8 Hz, 1H), 5.73-5.59 (m, 1H), 4.98-4.91 (m, 1H), 4.76-4.63 (m, 1H), 4.56-4.42 (m, 3H), 4.20-4.13 (m, 1H), 4.00-3.90 (m, 1H), 3.89-3.81 (m, 1H), 3.07-2.95 (m, 1H), 2.32-2.17 (m, 1H), 2.11-2.06 (m, 1H), 2.04 (d, J = 6.8 Hz, 6H), 2.00-1.90 (m, 2H), 1.52 (d, J = 7.2 Hz, 3H), 1.47 (d, J = 6.4 Hz, 3H). | Scheme 1, Intermediate 1 and Intermediate 58 |
| 76 | 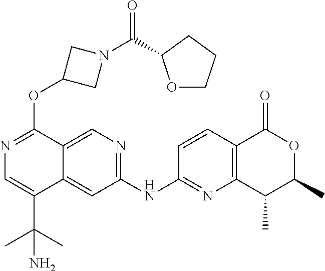 [SMILES: C[C@@H]1OC(=O)c2ccc(Nc3cc4c(C(C)(C)N)cnc(OC5CN(C(=O)[C@@H]6CCCO6)C5)c4cn3)nc2[C@H]1C] | 547 | ¹H NMR (400 MHz, CD₃OD): δ ppm 9.41 (d, J = 1.2 Hz, 1H), 9.22 (s, 1H), 8.18 (s, 1H), 8.14 (d, J = 8.4 Hz, 1H), 7.30 (d, J 8.8 Hz, 1H), 5.66-5.57 (m, 1H), 5.10-4.97 (m, 1H), 4.70-4.62 (m, 1H), 4.53-4.41 (m, 3H), 4.20-4.12 (m, 1H), 3.99-3.89 (m, 1H), 3.89-3.81 (m, 1H), 3.08-2.98 (m, 1H), 2.29-2.18 (m, 1H), 2.10-2.00 (m, 1H), 1.98-1.87 (m, 2H), 1.78 (d, J = 4.8 Hz, 6H), 1.51 (d, J = 7.2 Hz, 3H), 1.46 (d, J = 6.4 Hz, 3H). | Scheme 1, Intermediate 1 and Intermediate 59 |
| 77 | 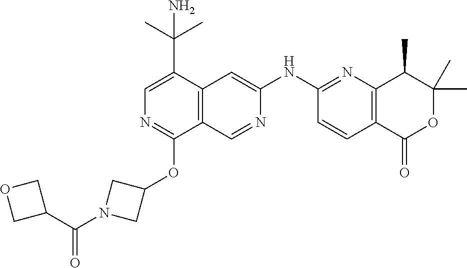 [SMILES: C[C@@H]1c2nc(Nc3cc4c(C(C)(C)N)cnc(OC5CN(C(=O)C6COC6)C5)c4cn3)ccc2C(=O)OC1(C)C] | 547 | ¹H NMR (400 MHz, CD₃OD): δ ppm 9.42 (s, 1H), 9.37 (s, 1H), 8.17-8.12 (m, 2H), 7.23 (d, J = 8.8 Hz, 1H), 5.65-5.58 (m, 1H), 4.83-4.77 (m, 3H), 4.66-4.50 (m, 3H), 4.32-4.26 (m, 1H), 4.20-4.14 (m, 1H), 4.07-3.96 (m, 1H), 3.10-3.00 (m, 1H), 1.83 (d, J = 8.0 Hz, 6H), 1.54 (s, 3H), 1.45 (s, 3H), 1.40 (d, J = 7.2 Hz, 3H). | Scheme 1, Intermediate 3 and Intermediate 56 |
| 78 | 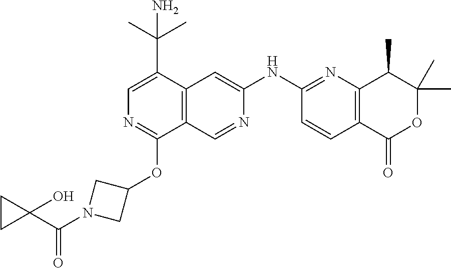 [SMILES: C[C@@H]1c2nc(Nc3cc4c(C(C)(C)N)cnc(OC5CN(C(=O)C6(O)CC6)C5)c4cn3)ccc2C(=O)OC1(C)C] | 547 | ¹H NMR (400 MHz, CD₃OD): δ ppm 9.45 (s, 1H), 9.38 (s, 1H), 8.18 (s, 1H), 8.13 (d, J = 8.8 Hz, 1H), 7.23 (d, J = 8.8 Hz, 1H), 5.66-5.57 (m, 1H), 5.13-5.01 (m, 1H), 4.74-4.67 (m, 1H), 4.54-4.45 (m, 1H), 4.22-4.09 (m, 1H), 3.08-3.00 (m, 1H), 1.81 (d, J = 7.6 Hz, 6H), 1.57-1.51 (m, 3H), 1.45 (s, 3H), 1.40 (d, J = 7.2 Hz, 3H), 1.26-1.20 (m, 2H), 0.98-0.91 (m, 2H). | Scheme 2, Intermediate 3, Intermediate 9, Step 1, and 1-hydroxy cyclopropane- 1-carboxylic acid |
| 79 | 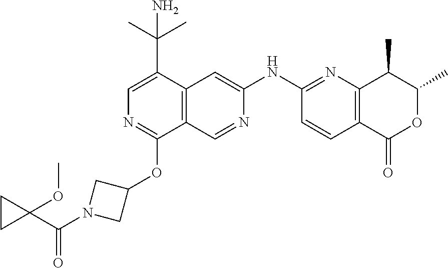 [SMILES: COC1(C(=O)N2CC(Oc3ncc(C(C)(C)N)c4cc(Nc5ccc6c(n5)[C@@H](C)[C@H](C)OC6=O)ncc34)C2)CC1] | 547 | ¹H NMR (400 MHz, CD₃OD): δ ppm 9.44 (s, 1H), 9.22 (s, 1H), 8.19 (s, 1H), 8.15 (d, J = 8.8 Hz, 1H), 7.31 (d, J = 8.8 Hz, 1H), 5.70-5.62 (m, 1H), 5.03-4.94 (m, 1H), 4.70-4.64 (m, 1H), 4.61-4.54 (m, 2H), 4.23-4.14 (m, 1H), 3.34 (s, 1H), 3.08-3.00 (m, 1H), 1.80 (d, J = 5.2 Hz, 6H), 1.51 (d, J = 7.2 Hz, 3H), 1.46 (d, J = 6.8 Hz, 3H), 1.19-1.17 (m, 2H), 1.08-1.02 (m, 2H). | Scheme 2, Intermediate 1, Intermediate 9, Step 1, and 1- methoxy cyclopropane- 1-carboxylic acid |
| 80 | 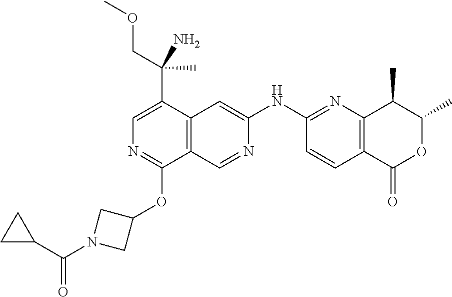 [SMILES: COC[C@@](C)(N)c1cnc(OC2CN(C(=O)C3CC3)C2)c2cnc(Nc3ccc4c(n3)[C@@H](C)[C@H](C)OC4=O)cc12] | 547 | ¹H NMR (400 MHz, CD₃OD) δ ppm 9.42 (s, 1H), 9.24 (s, 1H), 8.17 (s, 1H), 8.13 (d, J = 8.8 Hz, 1H), 7.32 (d, J = 8.8 Hz, 1H), 5.68-5.63 (m, 1H), 4.84-4.70 (m, 1H), 4.84- 4.67 (m, 1H), 4.52-4.49 (m, 2H), 3.97 (d, J = 3.6 Hz, 1H), 3.84 (m, 1H), 3.82 (m, 1H), 3.36 (s, 3H), 3.07- 3.04 (m, 1H), 1.75 (s, 3H), 1.64-1.54 (m, 1H), 1.52- 1.48 (m, 6H), 0.92-0.86 (m, 4H). | Scheme 1, Intermediate 1 and Intermediate 16 |
| 81 | 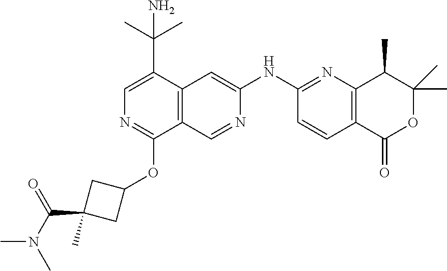 | 547 | ¹H NMR (400 MHz, CD₃OD): δ ppm 9.37 (d, J = 10.0 Hz, 2H), 8.19 (s, 1H), 8.15 (d, J = 8.8 Hz, 1H), 7.23 (d, J = 8.8 Hz, 1H), 5.53-5.39 (m, 1H), 3.10-3.04 (m, 1H), 3.03 (s, 3H), 2.96 (s, 3H), 2.76- 2.62 (m, 4H), 1.81 (d, J = 7.2 Hz, 6H), 1.55 (d, J = 3.6 Hz, 6H), 1.47 (s, 3H), 1.42 (d, J = 7.2 Hz, 3H) | Scheme 1, Intermediate 3 and Intermediate 70, 2nd eluting isomer by chiral SFC Column: Daicel Chiralpak OD (250mm* 30mm, 10um); Mobile Phase: [0.1% NH3H2O IPA]; B %: 55%-55%) |
| 82 | 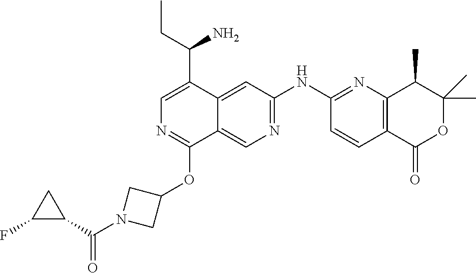 [SMILES: CC[C@@H](N)c1cnc(OC2CN(C(=O)[C@H]3C[C@H]3F)C2)c2cnc(Nc3ccc4c(n3)[C@@H](C)C(C)(C)OC4=O)cc12] | 548 | ¹H NMR (400 M Hz, CD₃OD): δ ppm 9.51 (d, J = 5.6 Hz, 1H), 8.83 (s, 1H), 8.12 (d, J = 8.8 Hz, 1H), 7.76-7.71 (m, 1H), 7.28 (d, J = 8.8 Hz, 1H), 6.79-6.71 (m, 1H), 5.41-5.31 (m, 1H), 4.97-4.92 (m, 1H), 4.82-4.73 (m, 1H), 4.64- 4.44 (m, 3H), 4.19-4.12 (m, 1H), 3.23-3.12 (m, 1H), 2.24-2.12 (m, 2H), 1.97- 1.84 (m, 1H), 1.77-1.63 (m, 1H), 1.56-1.49 (m, 6H), 1.45 (s, 3H), 1.22-1.07 (m, 1H), 1.01 (t, J = 7.4 Hz, 3H). | Scheme 5, Intermediate 3, Intermediate 39, and (1R,2R)-2- fluorocyclopro pane-1- carboxylic acid |
| | or | | | |
| | 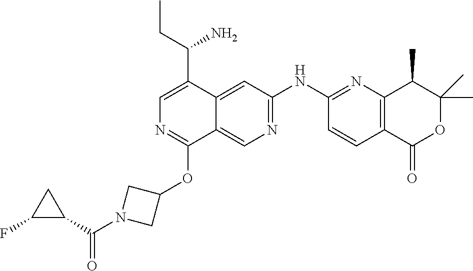 [SMILES: CC[C@H](N)c1cnc(OC2CN(C(=O)[C@H]3C[C@H]3F)C2)c2cnc(Nc3ccc4c(n3)[C@@H](C)C(C)(C)OC4=O)cc12] | | | |
| 83 | 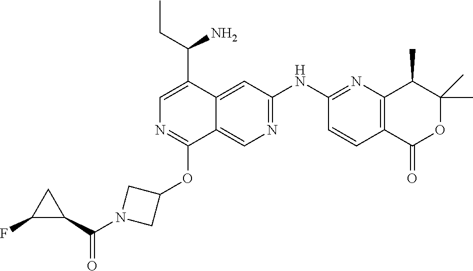 [SMILES: CC[C@@H](N)c1cnc(OC2CN(C(=O)[C@@H]3C[C@@H]3F)C2)c2cnc(Nc3ccc4c(n3)[C@@H](C)C(C)(C)OC4=O)cc12] | 548 | ¹H NMR (400 MHz, CD₃OD): δ ppm 9.43 (d, J = 4.4 Hz, 1H), 8.92 (s, 1H), 8.09 (d, J = 8.4 Hz, 1H), 7.72-7.62 (m, 1H), 7.20 (d, J = 8.8 Hz, 1H), 6.66 (d, J = 8.0 Hz, 1H), 5.38-5.29 (m, 1H), 4.97-4.90 (m, 1H), 4.81-4.70 (m, 1H), 4.65-4.42 (m, 3H), 4.22- 4.12 (m, 1H), 3.19-3.10 (m, 1H), 2.04-1.83 (m, 3H), 1.78-1.63 (m, 1H), 1.54- 1.47 (m, 6H), 1.45 (s, 3H), 1.18-1.07 (m, 1H), 0.97 (t, J = 7.4 Hz, 3H). | Scheme 5, Intermediate 3 and Intermediate 39 (15,2S)-2- fluorocyclopro pane-1- carboxylic acid |
| | or | | | |
| | 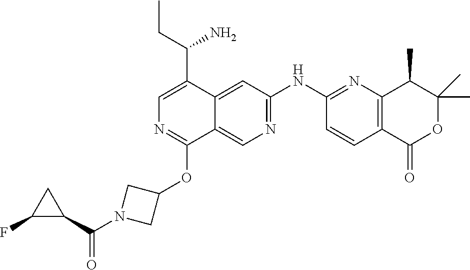 [SMILES: CC[C@H](N)c1cnc(OC2CN(C(=O)[C@@H]3C[C@@H]3F)C2)c2cnc(Nc3ccc4c(n3)[C@@H](C)C(C)(C)OC4=O)cc12] | | | |
| 84 | 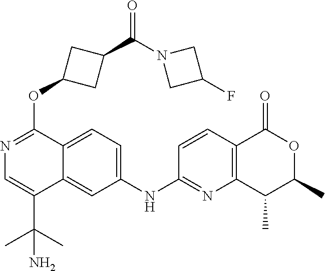 [SMILES: C[C@@H]1OC(=O)c2ccc(Nc3ccc4c(O[C@H]5C[C@@H](C(=O)N6CC(F)C6)C5)ncc(C(C)(C)N)c4c3)nc2[C@H]1C] | 549 | ¹H NMR (400 MHz, CD₃OD): δ ppm 9.43 (s, 1H), 9.07 (s, 1H), 8.53 (s, 2H), 8.18 (d, J = 8.8 Hz, 1H), 8.06 (s, 1H), 7.38 (d, J = 8.4 Hz, 1H), 5.47-5.35 (m, 2H), 4.70- 4.35 (m, 1H), 4.51-4.47 (m, 1H), 4.37-4.22 (m, 2H), 4.10- 3.98 (m, 1H), 3.02-2.92 (m, 2H), 2.83-3.76 (m, 2H), 2.50- 2.40 (m, 2H), 1.92 (d, J = 6.0 Hz, 6H), 1.51 (d, J = 7.2 Hz, 3H), 1.46 (d, J = 6.4 Hz, 3H). | Scheme 4, Intermediate 1, Intermediate 6, methyl (cis)-3- hydroxycyclob utane-1- carboxylate, and 3-fluoro azetidine |
| 85 | 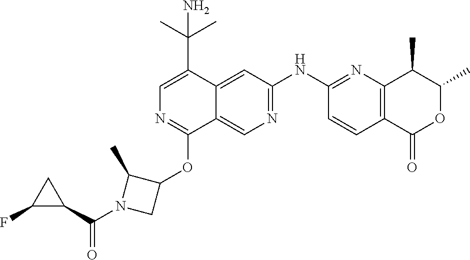 [SMILES: C[C@@H]1OC(=O)c2ccc(Nc3cc4c(C(C)(C)N)cnc(OC5CN(C(=O)[C@@H]6C[C@@H]6F)[C@H]5C)c4cn3)nc2[C@H]1C] | 549 | ¹H NMR (400 MHz, CD₃OD): δ ppm 9.50-9.39 (m, 1H), 9.24 (s, 1H), 8.23 (s, 1H), 8.17 (d, J = 8.8 Hz, 1H), 7.33 (d, J = 8.8 Hz, 1H), 5.29-5.19 (m, 1H), 4.98-4.91 (m, 1H), 4.81-4.75 (m, 1H), 4.71-4.63 (m, 1H), 4.61-4.44 (m, 1H), 4.43-4.06 (m, 1H), 3.10-2.97 (m, 1H), 1.92-1.82 (m, 2H), 1.81 (d, J = 4.8 Hz, 6H), 1.74- 1.61 (m, 3H), 1.57-1.44 (m, 6H), 1.18-1.06 (m, 1H) | Scheme 1, Intermediate 1 and Intermediate 12 |
| 86 | 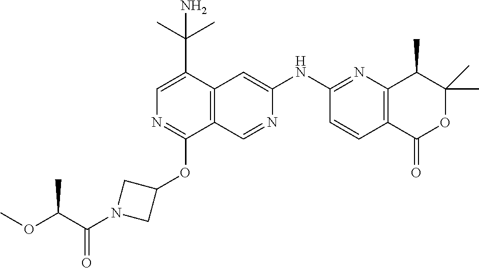 | 549 | ¹H NMR (400 MHz, CD₃OD): δ ppm 9.55 (s, 1H), 9.34 (s, 1H), 8.17 (s, 1H), 8.14 (s, 1H), 7.28 (d, J = 8.6 Hz, 1H), 5.67 (d, 1H), 4.81 (s, 1H), 4.65-4.45 (m, 2H), 4.21 (m, 1H), 3.96 (m, 1H), 3.38 (s, 3H), 3.05 (m, 1H), 1.91 (s, 3H), 1.89 (s, 3H), 1.56 (s, 3H), 1.47 (s, 3H), 1.43 (d, J = 7.2 Hz, 3H), 1.37 (m, 3H). | Scheme 1, Intermediate 3 and Intermediate 55, 2nd eluting isomer from chiral SFC Column: Daicel Chiralpak IC (250mm* 30mm, 10 um); Mobile Phase: [0.1% NH3H2O MEOH]; B%: 70%-70%) |
| | or | | | |
| | 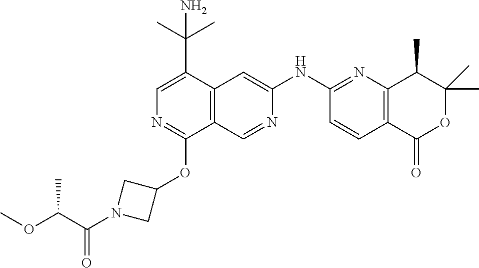 | | | |
| 87 | 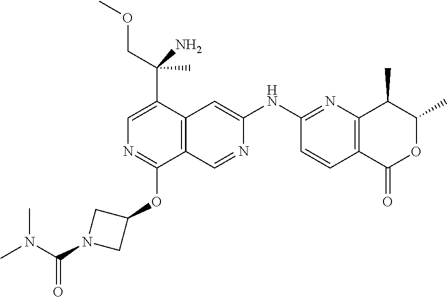 | 549 | ¹H NMR (DMSO-d₆, 400 MHz) δ ppm 10.66 (s, 1H), 9.46 (s, 1H), 9.29 (s, 1H), 8.08 (d, J = 8.0 Hz, 2H), 7.37 (d, J = 8.7 Hz, 1H), 5.32 (p, J = 7.7 Hz, 1H), 4.64 - 4.57 (m, 1H), 3.73 (dd, J = 20.9, 9.0 Hz, 2H), 3.26 (s, 3H), 3.16 - 3.06 (m, 1H), 3.00 (dd, J = 12.9, 6.8 Hz, 1H), 2.95 (s, 3H), 2.83 (s, 3H), 2.76 - 2.68 (m, 2H), 2.38 - 2.29 (m, 2H), 1.98 (s, 2H), 1.59 (s, 3H), 1.45 (d, J = 7.1 Hz, 3H), 1.38 (d, J = 6.5 Hz, 3H). | Scheme 1, Intermediate 1 and Intermediate 33 |
| 88 | 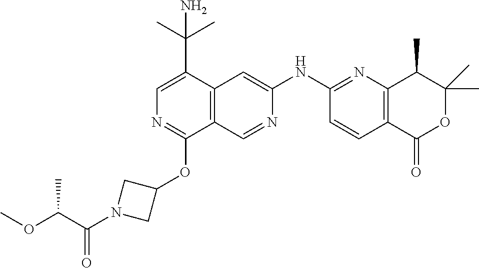 | 549 | ¹H NMR (400 MHz, CD₃OD): δ ppm 9.45 (s, 1H), 9.40 (s, 1H), 8.19 (s, 1H), 8.15 (d, J = 8.6 Hz, 1H), 7.25 (d, J = 8.6 Hz, 1H), 5.68-5.50 (m, 1H), 4.63-4.52 (m, 3H), , 4.26- 4.20 (m, 1H), 3.96 (m, 1H), 3.38 (s, 3H), 3.03-3.09 (m, 1H), 1.85 (s, 3H), 1.83 (s, 3H), 1.56 (s, 3H), 1.47 (s, 3H), 1.42 (m, 3H), 1.37 (m, 3H). | Scheme 1, Intermediate 3 and Intermediate 55, 1st eluting isomer from chiral SFC Column: Daicel Chiralpak IC (250 mm* 30 mm, 10 um); Mobile Phase: [0.1% NH3H2O MEOH]; B %: 70%-70%) |
| | or | | | |
| | 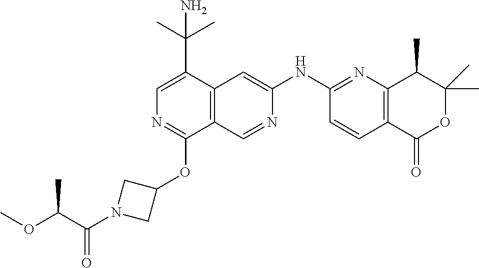 | | | |
| 89 | 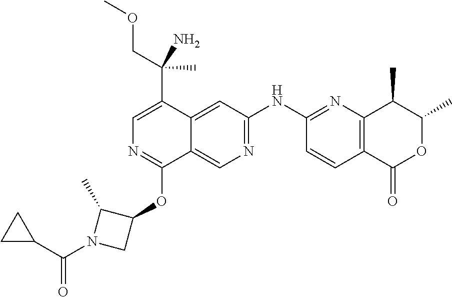 [SMILES: COC[C@@](C)(N)c1cnc(O[C@H]2CN(C(=O)C3CC3)[C@@H]2C)c2cnc(Nc3ccc4c(n3)[C@@H](C)[C@H](C)OC4=O)cc12] | 561 | ¹H NMR (DMSO-d₆, 400 MHz): δ ppm 10.68 (1H, s), 9.47-9.47 (1H, m), 9.39 (1H, s), 8.08 (2H, d, J = 7.3 Hz), 7.37 (1H, d, J = 8.7 Hz), 5.16 (1H, s), 4.66-4.72 (1H, m), 4.57-4.61 (1H, m), 4.25-4.31 (1H, m), 3.83-4.32 (1H, m), 3.69-3.76 (2H, m), 3.25 (3H, s), 2.96-3.02 (1H, m), 1.95- 2.10 (1H, m), 1.66-1.71 (1H, m), 1.59 (3H, s), 1.53 (2H, d, J = 6.6 Hz), 1.44 (3H, d, J = 7.0 Hz), 1.37 (3H, d, J = 6.5 Hz), 1.10-1.29 (2H, m), 0.71- 0.79 (4H, m). | Scheme 1, Intermediate 1 and Intermediate 30 |
| 90 | 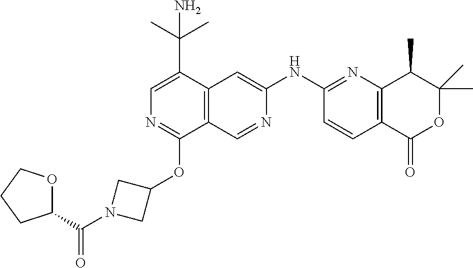 [SMILES: C[C@@H]1c2nc(Nc3cc4c(C(C)(C)N)cnc(OC5CN(C(=O)[C@@H]6CCCO6)C5)c4cn3)ccc2C(=O)OC1(C)C] | 561 | ¹H NMR (400 MHz, CD₃OD): δ ppm 9.48 (s, 1H), 9.26 (s, 1H), 8.18-8.15 (m, 1H), 8.08 (s, 1H), 7.29 (d, J = 8.6 Hz, 1H), 5.65 (d, J = 4.4 Hz, 1H), 4.58-4.46 (m, 4H), 4.18 (m, 1H), 4.01-3.83 (m, 2H), 3.06-3.00 (m, 1H), 2.13- 2.00 (m, 2H), 1.97 (s, 3H), 1.95 (s, 3H), 1.56 (s, 3H), 1.44 (s, 3H), 1.43 (d, J = 7.2 Hz, 3H). | Scheme 1, Intermediate 3 and Intermediate 59 |
| 91 | 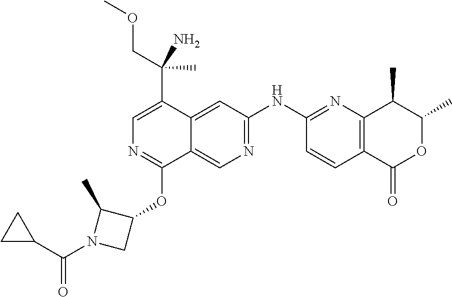 [SMILES: COC[C@@](C)(N)c1cnc(O[C@@H]2CN(C(=O)C3CC3)[C@H]2C)c2cnc(Nc3ccc4c(n3)[C@@H](C)[C@H](C)OC4=O)cc12] | 561 | ¹H NMR (DMSO-d₆, 400 MHz): δ ppm 10.78-10.81 (1H, m), 9.46 (1H, t, J = 1.4 Hz), 8.87-9.06 (1H, m), 8.87- 8.90 (1H, m), 8.11-8.14 (1H, m), 7.97-8.00 (1H, m), 7.47- 7.53 (1H, m), 5.16-5.21 (1H, m), 4.68-4.75 (1H, m), 4.60- 4.67 (1H, m), 4.37-4.42 (2H, m), 4.26-4.33 (1H, m), 3.92- 4.00 (1H, m), 3.85-3.91 (1H, m), 2.92-3.01 (1H, m), 1.75- 1.76 (2H, m), 1.66-1.73 (2H, m), 1.52-1.54 (2H, m), 1.40 (3H, d, J = 6.6 Hz), 1.38 (3H, d, J = 6.0 Hz), 1.20-1.26 (2H, m), 0.68-0.77 (4H, m). | Scheme 1, Intermediate 1 and Intermediate 31 |
| 92 | 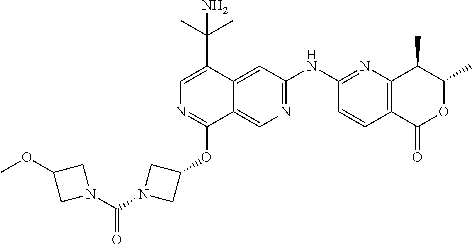 | 561 | ¹H NMR (400 MHz, CD₃OD): δ ppm 9.43 (s, 1H), 9.00 (s, 1H), 8.52 (s, 1H), 8.17 (d, J = 8.4 Hz, 1H), 8.00 (s, 1H), 7.38 (d, J = 8.4 Hz, 1H), 5.40-5.37 (m, 1H), 4.66- 4.64 (m, 1H), 4.40-4.35 (m, 1H), 4.26-4.24 (m, 1H), 4.18- 4.12 (m, 1H), 4.04-4.01 (m, 1H), 3.83-3.78 (m, 1H), 3.30 (s, 3H), 3.01-2.91 (m, 2H), 2.85-2.72 (m, 2H), 2.50-2.40 (m, 2H), 1.97 (d, J = 6.8 Hz, 6H), 1.50 (d, J = 7.2 Hz, 3H), 1.45 (d, J = 6.4 Hz, 3H). | Scheme 4, Intermediate1, Intermediate 6, methyl (cis)-3- hydroxycyclo- butane-1- carboxylate, and 3-methoxy azetidine |
| 93 | 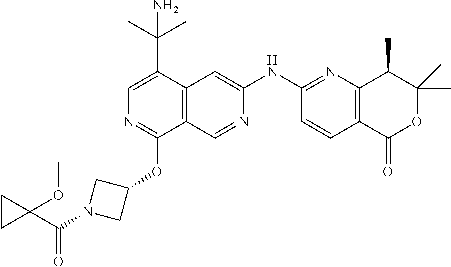 | 561 | ¹H NMR (400 MHz, CD₃OD): δ ppm 9.45 (d, J = 0.8 Hz, 1H), 9.41 (s, 1H), 8.21 (s, 1H), 8.14 (d, J = 8.8 Hz, 1H), 7.23 (d, J = 8.8 Hz, 1H), 5.72-5.61 (m, 1H), 4.02-4.96 (m, 1H), 4.61-4.53 (m, 2H), 4.27-4.11 (m, 1H), 3.35 (s, 3H), 3.10-3.00 (m, 1H), 1.81 (d, J = 7.50 Hz, 6H), 1.55 (s, 3H), 1.46 (s, 3H), 1.41 (d, J = 6.8 Hz, 3H), 1.21-1.18 (m, 2H), 1.09-1.04 (m, 2H). | Scheme 2, Intermediate 3, Intermediate 9, Step 1, and 1-methoxy cyclopropane- 1-carboxylic acid |
| 94 | 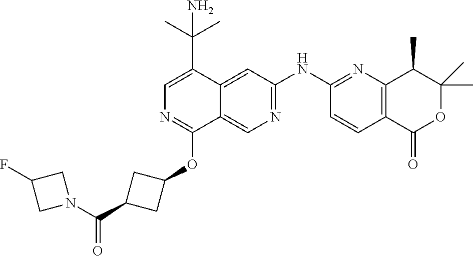 [SMILES: C[C@@H]1c2nc(Nc3cc4c(C(C)(C)N)cnc(O[C@H]5C[C@@H](C(=O)N6CC(F)C6)C5)c4cn3)ccc2C(=O)OC1(C)C] | 563 | ¹H NMR (400 MHz, CD₃OD): δ ppm 9.40 (s, 1H), 9.28 (s, 1H), 8.55 (s, 1H), 8.16 (d, J = 8.8 Hz, 1H), 8.10 (s, 1H), 7.26 (d, J = 8.8 Hz, 1H), 5.47-5.30 (m, 2H), 4.60- 4.50 (m, 1H), 4.35-4.25 (m, 2H), 4.09-4.00 (m, 1H), 3.03- 2.93 (m, 2H), 2.82-2.79 (m, 2H), 2.49-2.44 (m, 2H), 1.90 (d, J = 9.6 Hz, 6H), 1.54 (s, 3H), 1.45 (s, 3H), 1.41 (d, J = 7.2 Hz, 3H). | Scheme 4, Intermediate 3, Intermediate 6, methyl (cis)-3- hydroxycyclob utane-1- carboxylate, and 3- fluoroazetidine |
| 95 | 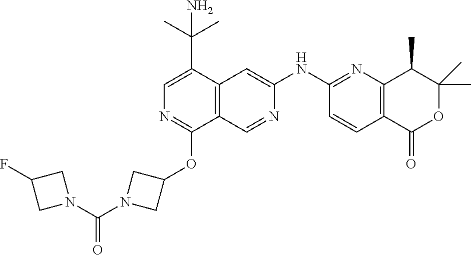 [SMILES: C[C@@H]1c2nc(Nc3cc4c(C(C)(C)N)cnc(OC5CN(C(=O)N6CC(F)C6)C5)c4cn3)ccc2C(=O)OC1(C)C] | 564 | ¹H-NMR (400 MHz, CD₃OD): δ ppm 9.32 (s, 1H), 9.29 (s, 1H), 8.07 (s, 1H), 8.04 (d, J = 8.8 Hz, 1H), 7.13 (d, J = 8.8 Hz, 1H), 5.54-5.44 (m, 1H), 5.32-5.10 (m, 1H), 4.49 (s, 1H), 4.40 (dd, J = 6.8, 9.6 Hz, 2H), 4.27-4.13 (m, 2H), 4.08-3.94 (m, 4H), 1.70 (d, J = 7.6 Hz, 6H), 1.44 (s, 3H), 1.35 (s, 3H), 1.30 (d, J = 7.2 Hz, 3H). | Scheme 1, Intermediate 3 and Intermediate 71 |
| 96 | 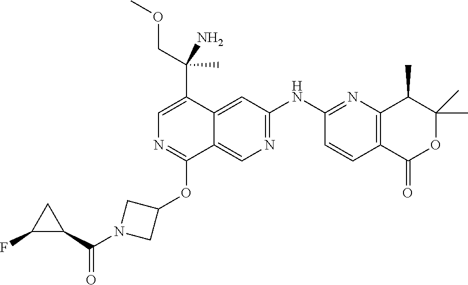 [SMILES: COC[C@@](C)(N)c1cnc(OC2CN(C(=O)[C@@H]3C[C@@H]3F)C2)c2cnc(Nc3ccc4c(n3)[C@@H](C)C(C)(C)OC4=O)cc12] | 565 | ¹H NMR (400 MHz, CD₃OD): δ ppm 9.47 (d, J = 3.2 Hz, 1H), 9.07 (d, J = 3.2 Hz, 1H), 8.16 (d, J = 8.4 Hz, 1H), 8.05 (s, 1H), 7.36 (d, J = 8.4 Hz, 1H), 5.70- 5.65 (m, 1H), 4.88-4.47 (m, 5H), 4.20-4.10 (m, 1H), 4.08- 3.95 (m, 2H), 3.38 (s, 3H), 3.10-2.95 (m, 1H), 1.95-1.85 (m, 4H), 1.80-1.64 (m, 1H), 1.60-1.40 (m, 6H), 1.16-1.07 (m, 1H). | Scheme 1, Intermediate 1 and Intermediate72 |
| 97 | 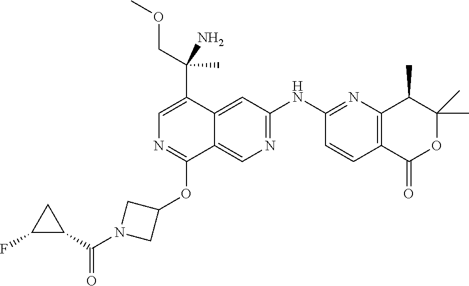 [SMILES: COC[C@@](C)(N)c1cnc(OC2CN(C(=O)[C@H]3C[C@H]3F)C2)c2cnc(Nc3ccc4c(n3)[C@@H](C)C(C)(C)OC4=O)cc12] | 565 | ¹H NMR (DMSO-d₆, 400 MHz): δ ppm 10.69 (1H, s), 9.41 (2H, t, J = 0.9 Hz), 8.06- 8.10 (2H, m), 7.39 (1H, d, J = 8.6 Hz), 5.58 (1H, t, J = 5.2 Hz), 4.76-4.81 (1H, m), 4.65- 4.68 (1H, m), 4.60 (1H, t, J = 6.2 Hz), 4.28-4.44 (2H, m), 4.04-4.07 (1H, m), 3.78 (1H, d, J = 9.1 Hz), 3.72 (1H, d, J = 9.1 Hz), 3.26 (3H, s), 2.99 (1H, t, J = 6.7 Hz), 1.83-1.88 (1H, m), 1.61-1.63 (3H, m), 1.48-1.56 (2H, m), 1.44 (3H, d, J = 7.0 Hz), 1.37 (3H, d, J = 6.4 Hz), 1.22-1.23 (1H, m), 1.00-1.05 (1H, m). | Scheme 1, Intermediate 1 and Intermediate 32 |
| 98 | 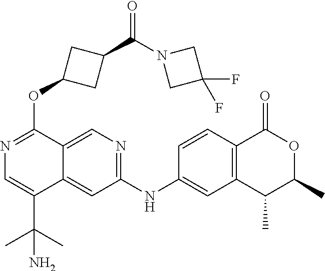 [SMILES: C[C@@H]1OC(=O)c2ccc(Nc3cc4c(C(C)(C)N)cnc(O[C@H]5C[C@@H](C(=O)N6CC(F)(F)C6)C5)c4cn3)cc2[C@H]1C] | 567 | ¹H NMR (400 MHz, CD₃OD): δ ppm 9.43 (s, 1H), 9.05 (s, 1H), 8.55 (s, 1H), 8.45 (d, J = 8.4 Hz, 1H), 8.02 (s, 1H), 7.40 (d, J = 8.4 Hz, 1H), 5.44 - 5.40 (m, 1H), 4.80- 4.75 (m, 1H), 4.70 - 4.67 (m, 2H), 4.36 (t, J = 12 Hz, 2H), 3.03 - 3.00 (m, 2H), 2.80-2.70 (m, 2H), 2.55-2.45 (m, 2H), 1.96 (d, J = 6.0 Hz, 6H), 1.53 (d, J = 7.2 Hz, 3H), 1.48 (d, J = 6.0 Hz, 3H). | Scheme 4, Intermediate 1, Intermediate 6, methyl (cis)-3- hydroxycyclo- butane-1- carboxylate, and 3,3-difluoro azetidine |
| 99 | 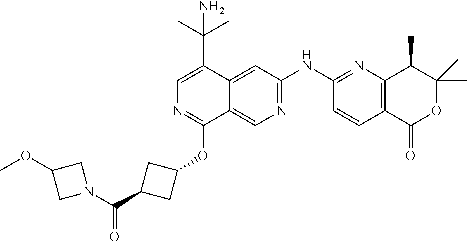 | 574 | ¹H NMR (400 MHz, CD₃OD): δ ppm 9.46 (s, 1H), 9.19 (s, 1H), 8.53 (s, 1H), 8.17 (d, J = 8.8 Hz, 1H), 8.02 (s, 1H), 7.29 (d, J = 8.8 Hz, 1H), 5.64-5.58 (m, 1H), 4.81 (m, 1H), 4.36-4.32 (m, 1H), 4.27-4.17 (m, 2H), 3.85-3.82 (m, 1H), 3.30 (s, 3H), 3.02- 2.97 (m, 1H), 2.80-2.74 (m, 2H), 2.59-2.52 (m, 2H), 1.97 (d, J = 11.6 Hz, 9H), 1.53 (s, 3H), 1.45 (s, 3H), 1.41 (d, J = 7.2 Hz, 3H). | Scheme 4, Intermediate 1, Intermediate 6, methyl (trans)- 3-hydroxy cyclobutane-1- carboxylate, and 3-methoxy azetidine |
| 100 | 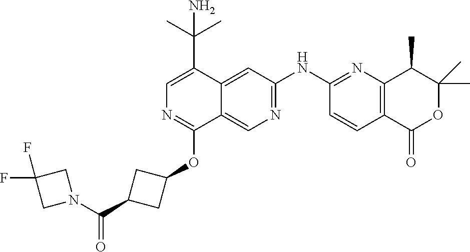 [SMILES: C[C@@H]1c2nc(Nc3cc4c(C(C)(C)N)cnc(O[C@H]5C[C@@H](C(=O)N6CC(F)(F)C6)C5)c4cn3)ccc2C(=O)OC1(C)C] | 581 | ¹H NMR (400 MHz, CD₃OD): δ ppm 9.40 (s, 1H), 9.27 (s, 1H), 8.55 (s, 1H), 8.16 (d, J = 8.8 Hz, 1H), 8.10 (s, 1H), 7.26 (d, J = 8.8 Hz, 1H), 5.46-5.36 (m, 1H), 4.67- 4.62 (m, 2H), 4.36 (t, J = 12.4 Hz, 2H), 3.08-2.95 (m, 2H), 2.85-2.81 (m, 2H), 2.55- 2.4 (m, 2H), 1.91(d, J = 9.6 Hz, 6H), 1.55 (s, 3H), 1.47 (s, 3H), 1.42 (d, J = 7.2 Hz, 3H). | Scheme 4, Intermediate 3, Intermediate 6, methyl (cis)-3- hydroxycyclo- butane-1- carboxylate and 3,3-difluoro azetidine |
| 101 | 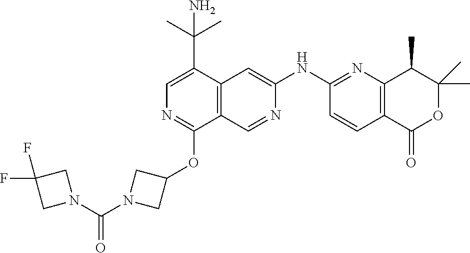 [SMILES: C[C@@H]1c2nc(Nc3cc4c(C(C)(C)N)cnc(OC5CN(C(=O)N6CC(F)(F)C6)C5)c4cn3)ccc2C(=O)OC1(C)C] | 582 | ¹H-NMR (400 MHz, CD₃OD): δ ppm 9.39 (d, J = 6.4 Hz, 2H), 8.17 (s, 1H), 8.12 (d, J = 8.8 Hz, 1H), 7.21 (d, J = 8.8 Hz, 1H), 5.64-5.56 (m, 1H), 4.52 (d, J = 6.8, 9.6 Hz, 2H), 4.35 (d, J = 12.4 Hz, 4H), 4.17 (d, J = 4.0, 10.0 Hz, 2H), 3.04 (d, J = 7.0 Hz, 1H), 1.79 (d, J = 7.2 Hz, 6H), 1.54 (s, 3H), 1.45 (s, 3H), 1.40 (d, J = 7.2 Hz, 3H) | Scheme 1, Intermediate 3 and Intermediate 73 |
| 102 | 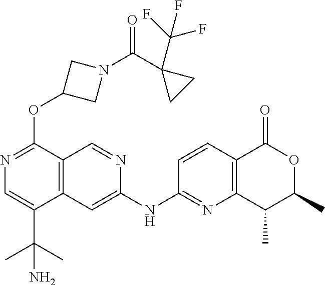 [SMILES: C[C@@H]1OC(=O)c2ccc(Nc3cc4c(C(C)(C)N)cnc(OC5CN(C(=O)C6(C(F)(F)F)CC6)C5)c4cn3)nc2[C@H]1C] | 585 | ¹H NMR (400 MHz, CD₃OD): δ ppm 9.46 (s, 1H), 9.15 (s, 1H), 8.54 (s, 1H), 8.18-8.15 (m, 1H), 8.11 (s, 1H), 7.34 (d, J = 8.8 Hz, 1H), 5.69-5.57 (m, 1H), 4.92-4.88 (m, 1H), 4.72-4.63 (m, 1H), 4.62-4.44 (m, 2H), 4.30-4.11 (m, 1H), 3.08-2.96 (m, 1H), 1.87 (d, J = 5.6 Hz, 6H), 1.52 (d, J = 7.2 Hz, 3H), 1.46 (d, J = 6.4 Hz, 3H), 1.31-1.24 (m, | Scheme 2, Intermediate 1, Intermediate 9, Step 1, and 1-(trifluoro methyl) cyclopropane- 1-carboxylic acid |
| 103 | 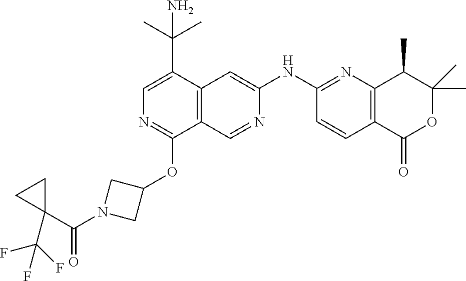 [SMILES: C[C@@H]1c2nc(Nc3cc4c(C(C)(C)N)cnc(OC5CN(C(=O)C6(C(F)(F)F)CC6)C5)c4cn3)ccc2C(=O)OC1(C)C] | 599 | ¹H NMR (400 MHz, CD₃OD): δ ppm 9.46 (d, J = 0.8 Hz, 1H), 9.30 (s, 1H), 8.15 (d, J = 8.8 Hz, 1H), 8.10 (s, 1H), 7.26 (d, J = 8.8 Hz, 1H), 5.68-5.59 (m, 1H), 5.01-4.91 (m, 1H), 4.63-4.44 (m, 2H), 4.32-4.12 (m, 1H), 3.06-2.98 (m, 1H), 1.92 (s, 3H), 1.89 (s, 3H), 1.54 (s, 3H), 1.45 (s, 3H), 1.41 (d, J = 7.2 Hz, 3H), 1.28 (d, J = 6.0 Hz, 4H). | Scheme 2, Intermediate 3, Intermediate 9, Step 1, and 1-(trifluoro methyl)cyclo propane-1- carboxylic acid |
| 104 | 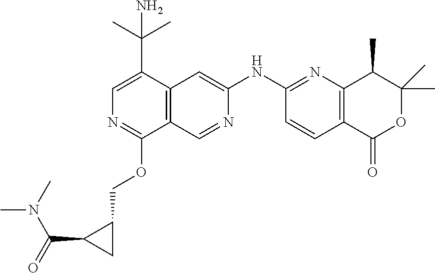 | 533 | ¹H NMR (400 MHz, CD₃OD): δ ppm 9.37 (s, 1H), 9.32 (s, 1H), 8.15 (s, 1H), 8.13 (d, J = 10.8 Hz, 1H), 7.23 (d, J = 8.8 Hz, 1H), 4.68-4.63 (m, 1H), 4.37-4.32 (m, 1H), 3.21 (s, 3H), 3.06-3.01 (m, 1H), 2.94 (s, 3H), 2.19-2.13 (m, 1H), 1.90 (s, 1H), 1.81 (d, J = 8.4 Hz, 6H), 1.54 (s, 3H), 1.45 (s, 3H), 1.40 (d, J = 7.2 Hz, 3H), 1.29-1.28 (m, 2H). | Scheme 1, Intermediate 3 and Intermediate 74, 2nd eluting isomer from chiral SFC Column: Daicel Chiralpak AD (250 mm* 30 mm, 10 um); Mobile Phase: [0.1% NH3H2O |
| | | | | IPA]; B %: |
| | | | | 40%-40%) |
| | or | | | |
| | 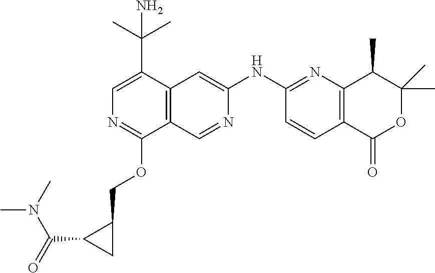 | | | |
| 105 | 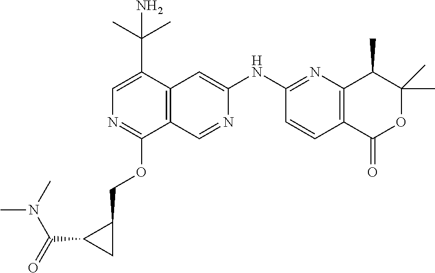 | 533 | ¹H NMR (400 MHz, CD₃OD): δ ppm 9.37 (s, 1H), 9.34 (s, 1H), 8.16 (s, 1H), 8.13 (d, J = 8.4 Hz, 1H), 7.23 (d, J = 8.8 Hz, 1H), 4.67-4.63 (m, 1H), 4.37-4.32 (m, 1H), 3.21 (s, 3H), 3.06-3.01 (m, 1H), 2.94 (s, 3H), 2.19-2.13 (m, 1H), 1.94-1.90 (m, 1H), 1.83 (d, J = 8 Hz, 6H), 1.53 (s, 3H), 1.45 (s, 3H), 1.40 (d, J = 7.2 Hz, 3H), 1.29-1.27 (m, 2H). | Scheme 1, Intermediate 3 and Intermediate 74, 1st eluting isomer from chiral SFC Column: Daicel Chiralpak AD (250 mm* 30 mm, 10 um); Mobile Phase: [0.1% NH3H2O |
| | | | | IPA]; B %: |
| | | | | 40%-40%) |
| | or | | | |
| | 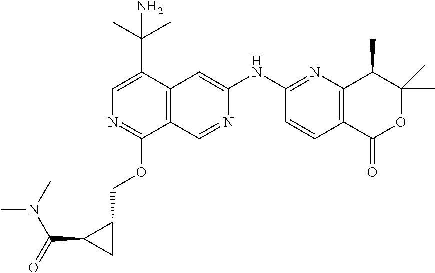 | | | |
| 106 | 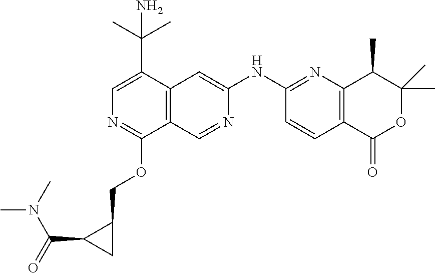 | 533 | ¹H NMR (400 MHz, CD₃OD): δ ppm 9.35 (s, 1H), 9.25 (s, 1H), 8.15 (d, J = 8.4 Hz, 1H), 8.07 (s, 1H), 7.26 (d, J = 8.8 Hz, 1H), 4.57 (s, 1H), 4.27-4.21 (m, 1H), 3.23 (s, 3H), 3.05-3.00 (m, 1H), 2.84 (s, 3H), 2.23-2.17 (m, 1H), 2.06-2.00 (m, 1H), 1.91 (s, 3H), 1.88 (s, 3H), 1.54 (s, 3H), 1.45 (s, 3H), 1.41 (d, J = 7.2 Hz, 3H), 1.31 (s, 1H), 1.16-1.11 (m, 1H). | Scheme 1, Intermediate 3 and Intermediate 75, 2nd eluting cis isomer from chiral SFC Column: Daicel Chiralpak IC (250 mm* 30 mm, 10 um); Mobile Phase: [0.1% NH3H2O |
| | | | | IPA]; B %: |
| | | | | 50%-50%) |
| | or | | | |
| | 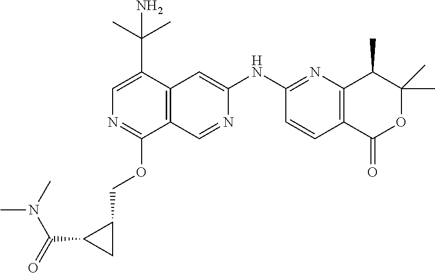 | | | |
| 107 | 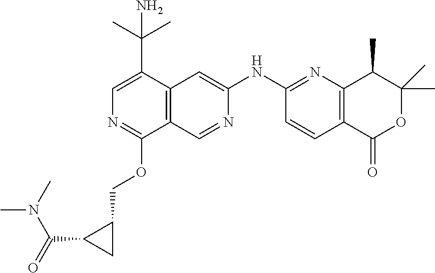 | 533 | ¹H NMR (400 MHz, CD₃OD): δ ppm 9.34 (s, 1H), 9.28 (s, 1H), 8.15 (d, J = 8.8 Hz, 1H), 8.09 (s, 1H), 7.25 (s, 1H), 4.56 (s, 1H), 4.26-4.20 (m, 1H), 3.22 (s, 3H), 3.05- 3.00 (m, 1H), 2.84 (s, 3H), 2.22-2.17 (m, 1H), 2.04-2.02 (m, 1H), 1.88 (s, 3H), 1.86 (s, 3H), 1.54 (s, 3H), 1.45 (s, 3H), 1.40 (d, J = 7.2 Hz, 3H), 1.32 (s, 1H), 1.15-1.12 (m, 1H). | Scheme 1, Intermediate 3 and Intermediate 75, 1st eluting cis isomer from chiral SFC Column: Daicel Chiralpak IC (250 mm* 30 mm, 10 um); Mobile Phase: [0.1% NH3H2O |
| | | | | IPA]; B %: |
| | | | | 50%-50%) |
| | or | | | |
| | 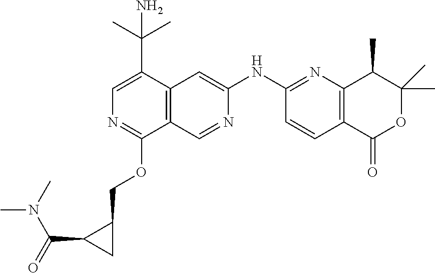 | | | |
| 108 | 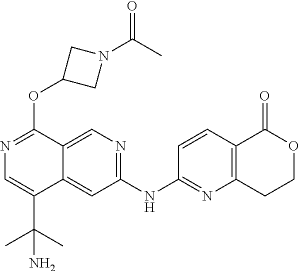 [SMILES: CC(=O)N1CC(Oc2ncc(C(C)(C)N)c3cc(Nc4ccc5c(n4)CCOC5=O)ncc23)C1] | 463 | ¹H NMR (400 MHz, CD₃OD): δ ppm 10.75 (s, 1H), 9.65 (s, 1H), 9.38 (s, 1H), 8.16 (s, 1H), 8.08 (d, J = 8.8 Hz, 1H), 7.29 (d, J = 8.8 Hz, 1H), 5.58-5.48 (m, 1H), 4.66-4.55 (m, 3H), 4.35-4.22 (m, 2H), 4.03-3.97 (m, 1H), 3.17-3.13 (m, 2H), 1.81 (s, 3H), 1.67 (s, 3H). | Scheme 1, Intermediate 19 and Intermediate 7 |
| 109 | 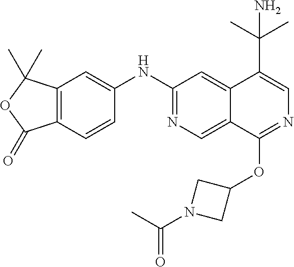 [SMILES: CC(=O)N1CC(Oc2ncc(C(C)(C)N)c3cc(Nc4ccc5c(c4)C(C)(C)OC5=O)ncc23)C1] | 477 | ¹H NMR (400 MHz, CD₃OD): δ ppm 9.43 (d, J = 8.0 Hz, 2H), 8.22 (s, 1H), 7.99 (d, J = 8.8 Hz, 1H), 7.26 (d, J = 8.4 Hz, 1H), 5.66-5.57 (m, 1H), 4.75-4.70 (m, 1H), 4.53- 4.45 (m, 1H), 4.42-4.35 (m, 1H), 4.17-4.08 (m, 1H), 1.94 (s, 3H), 1.82 (s, 6H), 1.76 (s, 6H). | Scheme 1, Intermediate 18 and Intermediate 7 |
| 110 | 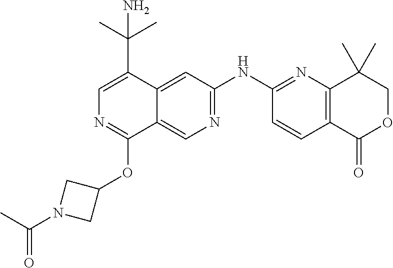 [SMILES: CC(=O)N1CC(Oc2ncc(C(C)(C)N)c3cc(Nc4ccc5c(n4)C(C)(C)COC5=O)ncc23)C1] | 491 | ¹H NMR (400 MHz, CDCl₃): δ ppm 9.74 (s, 1H), 8.44 (d, J = 8.8 Hz, 1H), 8.27 (s, 1H), 8.07 (s, 1H), 7.59 (d, J = 8.8 Hz, 1H), 5.73-5.67 (m, 1H), 4.79-4.72 (m, 1H), 4.55-4.43 (m, 2H), 4.42 (s, 2H), 4.20- 4.13 (m, 1H), 2.02 (s, 6H), 1.95 (s, 3H), 1.57 (s, 6H). | Scheme 1, Intermediate 26 and Intermediate 7 |
| 111 | 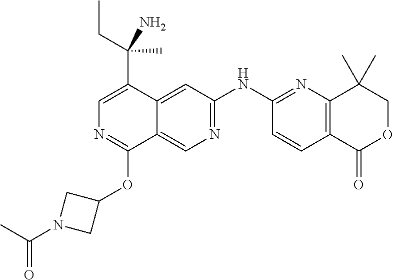 [SMILES: CC[C@@](C)(N)c1cnc(OC2CN(C(C)=O)C2)c2cnc(Nc3ccc4c(n3)C(C)(C)COC4=O)cc12] | 505 | ¹H NMR (400 M Hz, CD₃OD): δ ppm 9.46 (s, 1H), 8.72 (s, 1H), 8.17(d, J = 8.4 Hz, 1H), 8.14 (s, 1H), 7.53 (d, J = 8.8 Hz, 1H), 5.69-5.56 (m, 1H), 4.76-4.69 (m, 1H), 4.53- 4.47 (m, 1H), 4.44-4.36 (m, 1H), 4.32 (s, 2H), 4.18-4.11 (m, 1H), 2.47-2.31 (m, 1H), 2.20-2.04 (m, 1H), 1.95 (s, 3H), 1.74 (s, 3H), 1.47 (s, 6H), 0.72 (t, J = 7.4 Hz, 3H). | Scheme 1, Intermediate 26 and Intermediate45 |
| 112 | 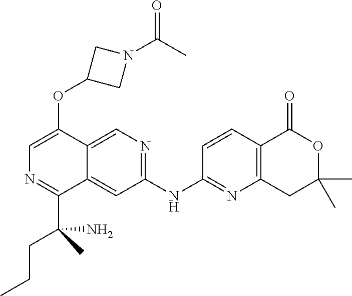 [SMILES: CCC[C@@](C)(N)c1ncc(OC2CN(C(C)=O)C2)c2cnc(Nc3ccc4c(n3)CC(C)(C)OC4=O)cc12] | 519 | ¹H NMR (400 MHz, CD₃OD): δ ppm 9.45 (s, 1H), 9.35 (s, 1H), 8.16 (d, J = 8.8 Hz, 1H), 8.05 (s, 1H), 7.22 (d, J = 8.8 Hz, 1H), 5.65-5.60 (m, 1H), 4.75- 4.70 (m, 1H), 4.52-4.47 (m, 1H), 4.42-4.39 (m, 1H), 4.15-4.11 (m, 1H), 3.23 (s, 2H), 2.52-2.44 (m, 1H), 2.23-2.15 (m, 1H), 1.94 (s, 3H), 1.86 (s, 3H), 1.54 (d, J = 2.8 Hz, 6H), 1.32-1.26 (m, 1H), 1.05-0.98 (m, 1H), 0.87-0.84 (m, 3H). | Scheme 1, Intermediate 17 and Intermediate 77 |
| | or | | | |
| | 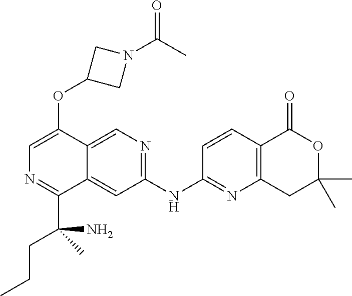 [SMILES: CCC[C@](C)(N)c1ncc(OC2CN(C(C)=O)C2)c2cnc(Nc3ccc4c(n3)CC(C)(C)OC4=O)cc12] | | | |
| 113 | 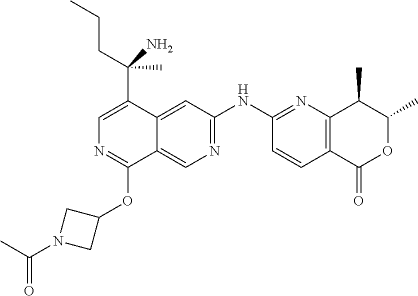 [SMILES: CCC[C@@](C)(N)c1cnc(OC2CN(C(C)=O)C2)c2cnc(Nc3ccc4c(n3)[C@@H](C)[C@H](C)OC4=O)cc12] | 519 | ¹H NMR (400 MHz, CD₃OD): δ ppm 9.44 (s, 1H), 9.25 (s, 1H), 8.16(d, J = 8.8 Hz, 1H), 8.12 (s, 1H), 7.31 (d, J = 8.8 Hz, 1H), 5.63-5.60 (m, 1H), 4.74- 4.69 (m, 2H), 4.52-4.47 (m, 1H), 4.41-4.38 (m, 1H), 4.15-4.12 (m, 1H), 3.08- 3.02 (m, 1H), 2.47-2.39 (m, 1H), 2.12-2.04 (m, 1H), 1.94 (s, 3H), 1.77 (s, 3H), 1.51 (d, J = 7.2 Hz, 3H), 1.45 (d, J = 6.4 Hz, 3H), 1.30-1.23 (m, 1H), 0.99- 0.92 (m, 1H), 0.84-0.80 (m, 3H). | Scheme 1, Intermediate 1 and Intermediate 77 |
| | or | | | |
| | 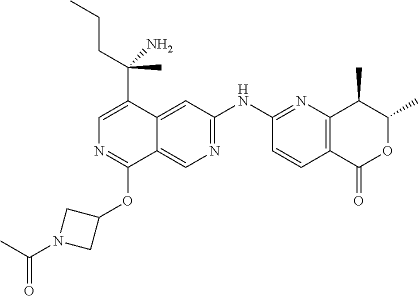 [SMILES: CCC[C@](C)(N)c1cnc(OC2CN(C(C)=O)C2)c2cnc(Nc3ccc4c(n3)[C@@H](C)[C@H](C)OC4=O)cc12] | | | |
| 114 | 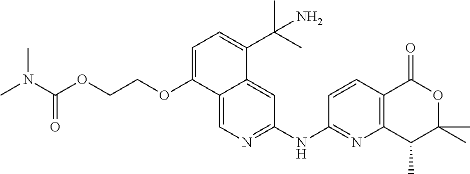 | 522 | ¹H NMR (400 MHz, CD₃OD): δ ppm 9.41 (s, 1H), 9.33 (s, 1H), 8.55 (s, 1H), 8.09 (d, J = 8.8 Hz, 1H), 7.65 (d, J = 8.4 Hz, 1H), 7.16 (d, J = 8.8 Hz, 1H), 6.86 (d, J = 8.4 Hz, 1H), 4.57 (t, J = 3.6 HZ, 2H), 4.43 (t, J = 4.8 HZ, 2H), 3.05-2.97 (m, 1H), 2.92 (s, 6H), 1.90 (d, J = 7.6 Hz, 6H), 1.54 (s, 3H), 1.44 (s, 3H), 1.38 (d, J = 7.2 Hz, 3H). | Scheme 1, Intermediate 3 and Intermediate41 |
| 115 | 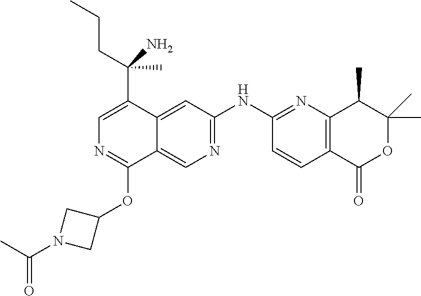 [SMILES: CCC[C@@](C)(N)c1cnc(OC2CN(C(C)=O)C2)c2cnc(Nc3ccc4c(n3)[C@@H](C)C(C)(C)OC4=O)cc12] | 533 | ¹H NMR (400 MHz, CD₃OD): δ ppm 9.44-9.43 (m, 2H), 8.14 (d, J = 8 Hz, 2H), 7.21 (d, J = 8.8 Hz, 1H), 5.64-5.69 (m, 1H), 4.74-4.70 (m, 1H), 4.51-4.47 (m, 1H), 4.41-4.37 (m, 1H), 4.15-4.12 (m, 1H), 3.09-3.03 (m, 1H), 2.44-2.36 (m, 1H), 2.16-2.07 (m, 1H), 1.94 (s, 3H), 1.75 (s, 3H), 1.55 (s, 3H), 1.44 (s, 3H), 1.40 (d, J = 7.6 Hz, 3H), 1.29-1.20 (m, 1H), 1.02-0.94 (m, 1H), 0.84-0.81 (m, 3H). | Scheme 1, Intermediate 3 and Intermediate77 |
| | or | | | |
| | 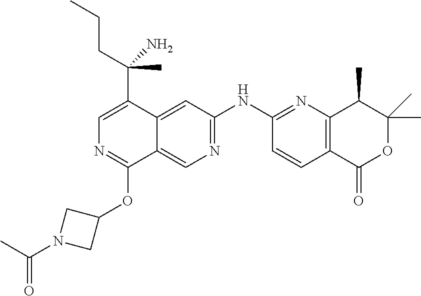 [SMILES: CCC[C@](C)(N)c1cnc(OC2CN(C(C)=O)C2)c2cnc(Nc3ccc4c(n3)[C@@H](C)C(C)(C)OC4=O)cc12] | | | |
| 116 | 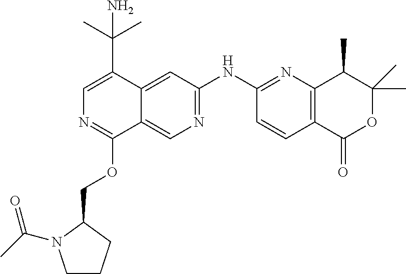 [SMILES: CC(=O)N1CCC[C@@H]1COc1ncc(C(C)(C)N)c2cc(Nc3ccc4c(n3)[C@@H](C)C(C)(C)OC4=O)ncc12] | 533 | ¹H NMR (400 MHz, CD₃OD): 9.37 (s, 1H), 9.20 (d, J = 13.2 Hz, 1H), 8.14 (d, J = 8.4 Hz, 1H), 8.05 (d, J = 14.4 Hz, 1H), 7.29-7.25 (m, 1H), 4.74-4.52 (m, 4H), 3.70- 3.54 (m, 2H), 3.04-2.98 (m, 1H), 2.26 (s, 1H), 2.22-2.12 (m, 3H), 2.09 (s, 2H), 2.07- 2.04 (m, 1H), 1.95 (d, J = 2.8 Hz, 3H), 1.92 (s, 3H), 1.53 (s, 3H), 1.45 (s, 3H), 1.40 (d, J = 7.2 Hz, 3H). | Scheme 1, Intermediate 3 and Intermediate 78 |
| 117 | 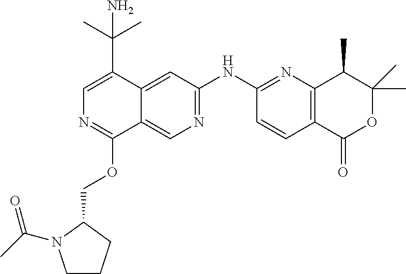 [SMILES: CC(=O)N1CCC[C@H]1COc1ncc(C(C)(C)N)c2cc(Nc3ccc4c(n3)[C@@H](C)C(C)(C)OC4=O)ncc12] | 533 | ¹H NMR (400 MHz, CD₃OD): δ ppm 9.39-9.35 (m, 2H), 8.20-8.12 (m, 2H), 7.24-7.21 (m, 1H), 4.74-4.68 (m, 1H), 4.63-4.49 (m, 3H), 3.70-3.45 (m, 2H), 3.07-3.02 (m, 1H), 2.27 (s, 1H), 2.23- 2.12 (m, 3H), 2.09 (s, 2H), 2.07-1.99 (m, 1H), 1.80-1.78 (m, 6H), 1.54 (s, 3H), 1.45 (s, 3H), 1.40 (d, J = 7.2 Hz, 3H). | Scheme 1, Intermediate 3 and Intermediate 79 |
| 118 | 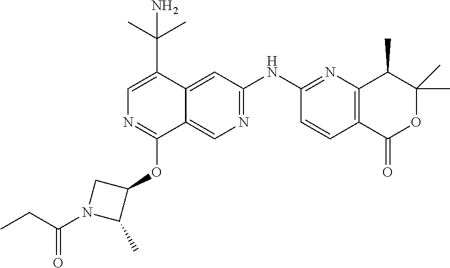 [SMILES: CCC(=O)N1C[C@@H](Oc2ncc(C(C)(C)N)c3cc(Nc4ccc5c(n4)[C@@H](C)C(C)(C)OC5=O)ncc23)[C@@H]1C] | 533 | ¹H NMR (400 MHz, CD₃OD): δ ppm 9.41 (s, 1H), 9.40 (s, 1H), 8.21 (s, 1H), 8.14 (d, J = 8.8 Hz, 1H), 7.23 (d, J = 8.8 Hz, 1H), 5.23-5.15 (m, 1H), 4.53-4.52(m, 1H), 4.23-4.10 (m, 1H), 3.08-3.05 (m, 1H), 2.27-2.23 (m, 2H), 1.80 (d, J = 7.2 Hz, 6H), 1.74- 1.66 (m, 3H), 1.54 (s, 3H), 1.45 (s, 3H), 1.40 (d, J = 3.2 Hz, 3H). | Scheme 1, Intermediate 3 and Intermediate62 |
| 119 | 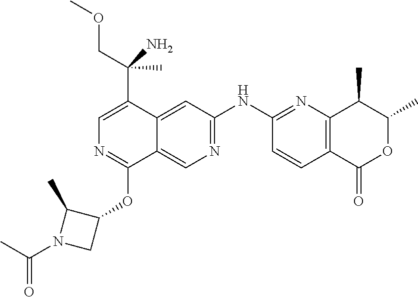 [SMILES: COC[C@@](C)(N)c1cnc(O[C@@H]2CN(C(C)=O)[C@H]2C)c2cnc(Nc3ccc4c(n3)[C@@H](C)[C@H](C)OC4=O)cc12] | 535 | ¹H NMR (400 MHz, CD3OD): δ ppm 9.21 (s, 1H), 9.06 (s, 1H), 8.06 (s, 1H), 7.98 (d, J = 8.8 Hz, 1H), 7.14 (d, J = 8.8 Hz, 1H), 5.07 - 4.97 (m, 1H), 4.58 - 4.53 (m, 1H), 4.41 - 4.27 (m, 1H), 4.17-4.11 (m, 1H), 3.97-3.91 (m, 1H), 3.90-3.84 (m, 1H), 3.73 - 3.67(m, 1H), 3.25 (s, 3H), 2.96 - 2.84 (m, 1H), 1.84 (d, J = 15.2 Hz, 3H), 1.65 - 1.52 (m, 6H), 1.44 - 1.33 (m, 6H). | Scheme 1, Intermediate 1 and Intermediate 80 |
| 120 | 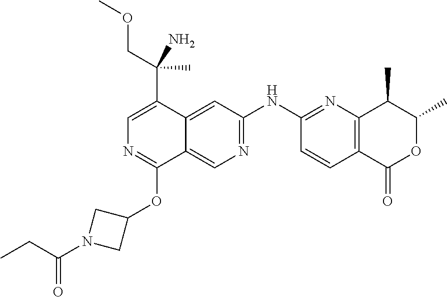 [SMILES: CCC(=O)N1CC(Oc2ncc([C@](C)(N)COC)c3cc(Nc4ccc5c(n4)[C@@H](C)[C@H](C)OC5=O)ncc23)C1] | 535 | ¹H NMR (400 MHz, CD3OD): δ ppm 9.44 (s, 1H), 9.10 (s, 1H), 8.50 (s, 1H), 8.16 (d, J = 8.8 Hz, 1H), 8.07 (s, 1H), 7.35 (d, J = 8.8 Hz, 1H), 5.68-5.60 (m, 1H), 4.78- 4.70 (m, 2H), 4.60-4.50 (m, 1H), 4.45-4.35 (m, 1H), 4.20- 4.10 (m, 1H), 4.05-3.90 (m, 2H), 3.37 (s, 3H), 3.08-3.00 (m, 1H), 2.30-2.20 (m, 2H), 1.86 (s, 3H), 1.55-1.43 (m, 6H), 1.18-1.10 (m, 3H). | Scheme 1, Intermediate 1 and Intermediate 81 |
| 121 | 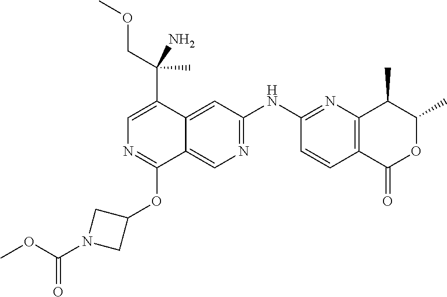 | 537 | ¹H NMR (400 MHz, 6d- DMSO): δ ppm 10.80-10.62 (m, 1H), 9.54-9.45 (m, 1H), 9.39 (s, 1H), 8.12-8.01 (m, 2H), 7.39 (d, J = 8.8 Hz, 1H), 5.62-5.48 (m, 1H), 4.67-4.54 (m, 1H), 4.40 (d, J = 7.2 Hz, 2H), 4.14-3.98 (m, 2H), 3.80- 3.74 (m, 1H), 3.72-3.67 (m, 1H), 3.59 (s, 3H), 3.28-3.23 (m, 3H), 3.06-2.94 (m, 1H), 1.59 (s, 3H), 1.50-1.42 (m, 3H), 1.38 (d, J = 6.8 Hz, 3H). | Scheme 1, Intermediate 1 and Intermediate 82 |
| 122 | 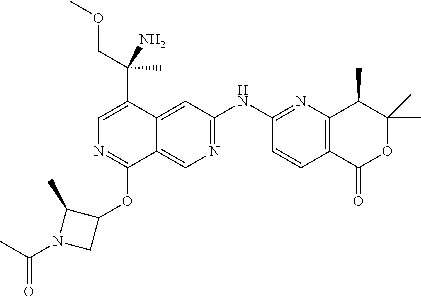 [SMILES: COC[C@@](C)(N)c1cnc(OC2CN(C(C)=O)[C@H]2C)c2cnc(Nc3ccc4c(n3)[C@@H](C)C(C)(C)OC4=O)cc12] | 549 | ¹H NMR (400 MHz, CD3OD): δ ppm 9.32 (s, 1H), 9.29 (s, 1H), 8.12 (s, 1H), 8.04 (d, J = 8.8 Hz, 1H), 7.11 (d, J = 8.4 Hz, 1H), 5.15 - 5.07 (m, 1H), 4.66 - 4.56 (m, 1H), 4.50 - 4.30 (m, 1H), 4.23-4.16 (m, 1H), 4.03 - 3.95 (m, 1H), 3.93 - 3.87 (m, 1H), 3.85 - 3.77 (m, 1H), 3.31 (s, 3H), 3.02-2.95 (m, 1H), 1.89 (d, J = 15.2 Hz, 3H), 1.71 - 1.59 (m, 6H), 1.49 (s, 3H), 1.40 (d, J = 6.0, 3H), 1.35 (t, J = 8.0, 3H ). | Scheme 1, Intermediate 3 and Intermediate 80 |
| 123 | 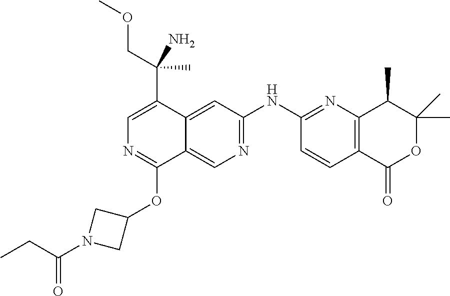 [SMILES: CCC(=O)N1CC(Oc2ncc([C@](C)(N)COC)c3cc(Nc4ccc5c(n4)[C@@H](C)C(C)(C)OC5=O)ncc23)C1] | 549 | ¹H NMR (400 MHz, CD3OD): δ ppm 9.43 (s, 1H), 9.38 (s, 1H), 8.16-8.10 (m, 2H), 7.23 (d, J = 8.4 Hz, 1H), 5.68-5.60 (m, 1H), 4.78-4.70 (m, 1H), 4.55-4.45 (m, 1H), 4.40-4.30 (m, 1H), 4.20-4.10 (m, 1H), 3.98-3.90 (m, 2H), 3.36 (s, 3H), 3.08-3.00 (m, 1H), 2.30-2.20 (m, 2H), 1.78 (s, 3H), 1.54 (s, 3H), 1.46 (s, 3H), 1.42 (d, J = 7.2 Hz, 3H), 1.15-1.10 (m, 3H). | Scheme 1, Intermediate 3 and Intermediate 81 |
| 124 | 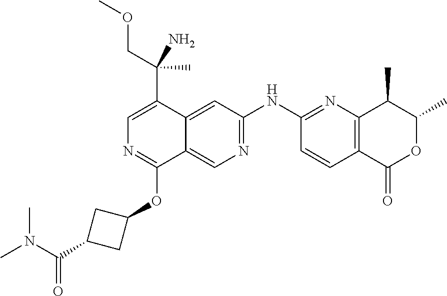 | 549 | ¹H NMR (400 MHz, DMSO- d6) δ 10.65 (s, 1H), 9.45 (s, 1H), 9.34 (s, 1H), 8.08 (d, J = 9.4 Hz, 2H), 7.39 (d, J = 8.8 Hz, 1H), 5.37 (p, J = 6.6 Hz, 1H), 4.64 - 4.57 (m, 1H), 3.72 (dd, J = 22.6, 9.0 Hz, 2H), 3.50 - 3.42 (m, 1H), 3.29 (s, 1H), 3.26 (s, 3H), 3.03 - 2.95 (m, 1H), 2.91 (s, 3H), 2.87 (s, 3H), 2.73 - 2.65 (m, 2H), 2.52- 2.42 (m, 1H), 2.01 (bs, 2H), 1.59 (s, 3H), 1.45 (d, J = 7.1 Hz, 3H), 1.38 (d, J = 6.4 Hz, 3H). | Scheme 1, Intermediate 1 and Intermediate 34 |
| 125 | 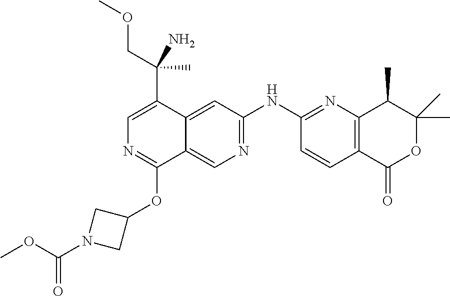 | 55 | ¹H NMR (400 MHz, 6d- DMSO): δ ppm 10.72 (s, 1H), 9.67-9.60 (m, 1H), 9.38 (s, 1H), 8.09-8.03 (m, 2H), 7.31 (d, J = 8.8 Hz, 1H), 5.60-5.48 (m, 1H), 4.41 (s, 2H), 4.08 (d, J = 6.4 Hz, 2H), 3.85-3.75 (m, 1H), 3.59 (s, 3H), 3.47-3.37 (m, 1H), 3.28-3.21 (m, 3H), 3.04-2.94 (m, 1H), 1.60 (s, 3H), 1.44 (s, 3H), 1.37 (s, 3H), 1.34-1.28 (m, 3H). | Scheme 1, Intermediate 3 and Intermediate 82 |
| 126 | 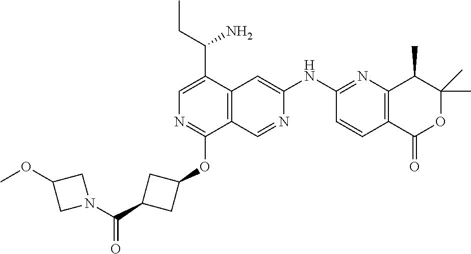 | 575 | ¹H NMR (400 MHz, CD3OD): δ ppm 9.33 (s, 1H), 8.78 (s, 1H), 8.18 - 8.04 (m, 2H), 7.25 (d, J = 8.8 Hz, 1H), 5.44 - 5.33 (m, 1H), 4.52 (t, J = 6.8 Hz, 1H), 4.40 - 4.34 (m, 1H), 4.29 - 4.22 (m, 1H), 4.19 - 4.11 (m, 1H), 4.05 - 3.98 (m, 1H), 3.85-3.77(m, 1H), 3.31 (s, 3H), 3.21-3.12 (m, 1H), 2.98 - 2.85 (m, 1H), 2.83 - 2.71 (m, 2H), 2.51 - 2.39 (m, 2H), 2.10 - 1.90 (m, 2H), 1.54 - 1.46 (m, 6H), 1.43 (s, 3H), 0.98 (t, J = | Scheme 3, Intermediate 3 and Intermediate 66, 2nd eluting isomer obtained from separating penultimate intermediate by prep-HPLC Column: 3_Phenomenex Luna C18 75*30 mm* |
| | | | 7.2 Hz, 3H). | 3 um; |
| | | | | Mobile Phase: |
| | | | | [water |
| | | | | (0.05% HCl)- |
| | | | | ACN]; B %: |
| | | | | 40%-60%, |
| | | | | 6.5 min) |
| | or | | | |
| | 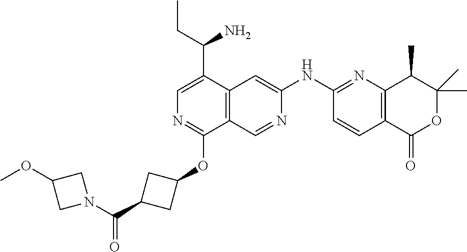 | | | |
| 127 | 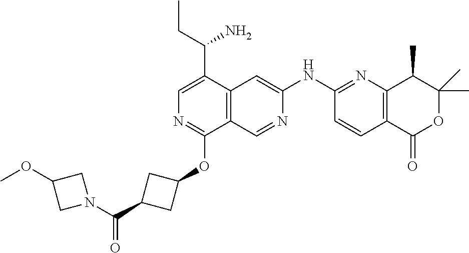 | 575 | ¹H NMR (400 MHz, CD3OD): δ ppm 9.33 (s, 1H), 8.74 (s, 1H), 8.18 - 8.07 (m, 2H), 7.27 (d, J = 8.2 Hz, 1H), 5.43 - 5.32 (m, 1H), 4.48 (t, J = 6.8 Hz, 1H), 4.42 - 4.34 (m, 1H), 4.30 - 4.23 (m, 1H), 4.19- 4.12 (m, 1H), 4.06 - 3.98 (m, 1H), 3.85-3.76 (m, 1H), 3.30 (s, 3H), 3.20 - 3.12 (m, 1H), 2.97 - 2.85 (m, 1H), 2.83 - 2.72 (m, 2H), 2.51 - 2.39 (m, 2H), 2.11 - 1.90 (m, 2H), 1.53- 1.47 (m, 6H), 1.43 (s, 3H), 1.00 (t, J = 7.6 Hz, 3H) | Scheme 3, Intermediate 3 and Intermediate 66, 1st eluting isomer obtained from separating penultimate intermediate by prep-HPLC Column: 3_Phenomenex Luna C18 75*30 mm* |
| | | | | 3 um; |
| | | | | Mobile Phase: |
| | | | | [water |
| | | | | (0.05% HCl)- |
| | | | | ACN]; B %: |
| | | | | 40%-60%, |
| | | | | 6.5 min) |
| | or | | | |
| | 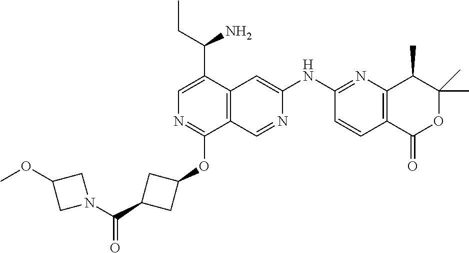 | | | |

Included in the present teachings are pharmaceutically acceptable salts of the compounds disclosed herein (including compounds 1-127 disclosed in Table 1 and Exemplification) as well as the corresponding charge neutral form e.g., free base.

Another embodiment of the disclosure is a compound disclosed herein, including a compound of Formulae I-III, IV(A), IV(B), V(A), V(B), VI(A), VI(B), VI(C), VII, VII(a), VII(B), VII(C), VIII(A), VIII(B), VIII(C), IX(A), IX(B), IX(C), X(A), X(B), X(C), XI, XII(A) and XII(B), or a compound in Table 1 or exemplification or a pharmaceutically acceptable salt of any of the foregoing, in which one or more hydrogen atoms is replaced with deuterium. The deuterium enrichment at any one of the sites where hydrogen has been replaced by deuterium is at least 50%, 75%, 85%, 90%, 95%, 98% or 99%. Deuterium enrichment is a mole percent and is obtained by dividing the number of compounds with deuterium enrichment at the site of enrichment with the number of compounds having hydrogen or deuterium at the site of enrichment.

### Definitions

As used herein, the term “pharmaceutically acceptable salt” refers to pharmaceutical salts that are, within the scope of sound medical judgment, suitable for use in contact with the tissues of humans and lower animals without undue toxicity, irritation, and allergic response, and are commensurate with a reasonable benefit/risk ratio. Pharmaceutically acceptable salts are well known in the art. For example, S. M. Berge et al. describes pharmacologically acceptable salts in J. Pharm. Sci. (1977) 66:1-19. Compounds of the present teachings with basic groups can form pharmaceutically acceptable salts with pharmaceutically acceptable acid(s). Suitable pharmaceutically acceptable acid addition salts of the compounds described herein include salts of inorganic acids (such as hydrochloric acid, hydrobromic, phosphoric, nitric, and sulfuric acids) and of organic acids (such as acetic acid, benzenesulfonic, benzoic, methanesulfonic, and p-toluenesulfonic acids). Compounds of the present teachings with acidic groups can form pharmaceutically acceptable salts with pharmaceutically acceptable base(s). Suitable pharmaceutically acceptable basic salts include ammonium salts, alkali metal salts (such as sodium and potassium salts) and alkaline earth metal salts (such as magnesium and calcium salts).

The term “alkyl” used alone or as part of a larger moiety, such as “alkoxy”, “hydroxyalkyl” and the like, means a saturated aliphatic straight-chain or branched monovalent hydrocarbon radical. Unless otherwise specified, an alkyl group typically has 1 to 6 carbon atoms (C₁₋₆ alkyl) (i.e., 1, 2, 3, 4, 5 or 6), alternatively, 1 to 3 carbon atoms (C₁₋₃ alkyl) (i.e., 1, 2 or 3). “C₁₋₆ alkyl” is means a radical having 1 to 6 carbon atoms in a linear or branched arrangement, such as methyl, ethyl, propyl, isopropyl, butyl, isobutyl, tert-butyl, and the like.

The term “alkylene” means a C₁-C₆ bivalent alkyl group, for example a group —(CH₂)n- where n is 1 to 6, unless otherwise specified.

The term “halogen” or “halo” means fluorine or fluoro (F), chlorine or chloro (Cl), bromine or bromo (Br), or iodine or iodo (I).

“Cycloalkyl” means a saturated aliphatic cyclic hydrocarbon ring radical. Unless otherwise specified, a cycloalkyl has 3 to 8 ring carbon atoms (C₃₋₈ cycloalkyl) (i.e., 3, 4, 5, 6, 7, or 8), alternatively, 3 to 6 ring carbon atoms (C₃₋₆ cycloalkyl) (i.e., 3, 4, 5, or 6), alternatively, 3 to 5 carbon atoms (C₃₋₅ cycloalkyl) (i.e., 3, 4, or 5). “C₃₋₆ Cycloalkyl” means a radical having from 3 to 6 carbon atoms arranged in a monocyclic ring. A C₃₋₆ cycloalkyl includes cyclopropyl, cyclobutyl, cyclopentyl, and cyclohexyl. A C₃₋₅ cycloalkyl includes cyclopropyl, cyclobutyl, and cyclopentyl.

The term “heterocycle” refers to a monocyclic non-aromatic ring radical containing unless otherwise specified, 3 to 8 ring atoms (i.e., “3, 4, 5, 6, 7, or 8 membered”) selected from carbon atom and 1 or 2 heteroatoms. Each heteroatom is independently selected from nitrogen, quaternary nitrogen, oxidized nitrogen (e.g., NO); oxygen; and sulfur, including sulfoxide and sulfone. For example, 4-6 membered heterocycle containing nitrogen refers to a monocyclic non-aromatic ring radical containing 2-5 carbon atoms and 1 or 2 nitrogen; 4-6 membered heterocycle containing oxygen refers to a monocyclic non-aromatic ring radical containing 2-5 carbon atoms and 1 or 2 oxygen. Representative heterocycles include azetidinyl, morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like.

The term “substituted”, whether preceded by the term “optionally” or not, refers to the replacement of a hydrogen substituent in a given structure with a non-hydrogen substituent. Thus, for example, a substituted alkyl is an alkyl wherein at least one non-hydrogen substituent is in the place of a hydrogen substituent on the alkyl group. To illustrate, monofluoroalkyl is an alkyl substituted with a fluoro substituent, and difluoroalkyl is an alkyl substituted with two fluoro substituents. It should be recognized that if there is more than one substitution on a substituent, each non-hydrogen substituent can be identical or different (unless otherwise stated).

If a group is described as “optionally substituted”, the group can be either (1) not substituted or (2) substituted.

If a group is described as optionally substituted with up to a particular number of non-hydrogen substituents, that group can be either (1) not substituted; or (2) substituted by up to that particular number of non-hydrogen substituents or by up to the maximum number of substitutable positions on the substituent, whichever is less. Thus, for example, if a group is described as a cycloalkyl optionally substituted with up to 3 non-hydrogen substituents, then any cycloalkyl with less than 3 substitutable positions would be optionally substituted by up to only as many non-hydrogen substituents as the cycloalkyl has substitutable positions.

Compounds having one or more chiral centers can exist in various stereoisomeric forms, i.e., each chiral center can have an R or S configuration or can be a mixture of both. Stereoisomers are compounds that differ only in their spatial arrangement. Stereoisomers include all diastereomeric and enantiomeric forms of a compound. Enantiomers are stereoisomers that are mirror images of each other. Diastereomers are stereoisomers having two or more chiral centers that are not identifcal and are not mirror images of each other.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9% (except when the designation “rac” or “racemate accompanies the structure or name, as explained in the following two paragraphs). “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When the stereochemical configuration at a chiral center in a compound is depicted by chemical name (e.g., where the configuration is indicated in the name by “R” or “S”) or structure (e.g., the configuration is indicated by “wedge” bonds) and the designation “rac” or “racemate” accompanies the structure or is designated in the chemical name, a racemic mixture is intended.

When two or more stereoisomers are depicted by their chemical names or structures, and the names or structures are connected by an “or”, one or the other of the two or more stereoisomers is intended, but not both. The enrichment of one stereoisomer relative to the other is as indicated above.

When a disclosed compound having a chiral center is depicted by a structure without showing a configuration at that chiral center, the structure is meant to encompass the compound with the S configuration at that chiral center, the compound with the R configuration at that chiral center, or the compound with a mixture of the R and S configuration at that chiral center. When a disclosed compound having a chiral center is depicted by its chemical name without indicating a configuration at that chiral center with “S” or “R”, the name is meant to encompass the compound with the S configuration at that chiral center, the compound with the R configuration at that chiral center or the compound with a mixture of the R and S configuration at that chiral center.

A racemic mixture means a mixture of 50% of one enantiomer and 50% of its corresponding enantiomer. The present teachings encompass all enantiomerically-pure, enantiomerically-enriched, diastereomerically pure, diastereomerically enriched, and racemic mixtures, and diastereomeric mixtures of the compounds described herein.

Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

“Peak 1” or “first eluting isomer” in Table 1 and in the Exemplification section refers to an intended reaction product compound obtained from a chromatography separation/purification that elutes earlier than a second intended reaction product compound from the same preceding reaction. The second intended product compound is referred to as “peak 2” or “second eluting isomer”.

When a compound is designated by a name or structure that indicates a single enantiomer, unless indicated otherwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that, unless otherwise indicated, one of the encompassed stereoisomers or any mixture of the encompassed stereoisomers are included. It is to be further understood that the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

### Use Embodiments

Compounds of the disclosure are MAP4K1 inhibitors. The use of the word “inhibitor” means that a compound or a pharmaceutically acceptable salt thereof inhibits activity of MAP4K1. By “inhibit” herein is meant to decrease the activity of the target enzyme as compared to the activity of that enzyme in the absence of the inhibitor. In some alternatives, the term “inhibit” means a decrease in MAP4K1 activity of at least 5%, at least 10%, at least 20%, at least 50%, at least 60%, at least 79%, at least 80%, at least 90% or at least 95%. In other alternatives, inhibit means a decrease in MAP4K1 activity of 5% to 25%, 25% to 50%, 50 to 70%, 75 to 100%. In some embodiments, inhibit means a decrease in MAP4K1 activity about 95% to 100%, e.g., a decrease in activity of 95%, 96%, 97%, 98%, 99%, or 100%. Such decreases can be measured using a variety of techniques that would be recognizable by one of skill in the art, including in vitro kinase assays.

Compounds of the disclosure are selective MAP4K1 inhibitors. As used herein, a “selective MAP4K1 inhibitor” refers to a compound or a pharmaceutically acceptable salt thereof, which has the ability to selectively inhibit MAP4K1 kinase over other targets. More specifically, a selective MAP4K1 inhibitor has the ability to selectively inhibit MAP4K1 over another kinase. A selective MAP4K1 inhibitor has the ability to selectively reduce target signaling activity relative to off-target signaling activity, via direct or indirect interaction with the target. The ability to selectively target MAP4K1 with a compound or pharmaceutically acceptable salt therof provides advantages in terms of improved potency, less off-target activity and an increased probability of clinical success in comparison with a non-selective compound or salt. A MAP4K1 inhibitor that selectively inhibits MAP4K1 may have an activity that is at least 2-fold relative to another kinase (e.g., at least 10-fold; at least 15-fold; at least 20-fold; at least 30-fold; at least 40-fold selectivity; at least 50-fold; at least 60-fold; at least 70-fold; at least 80-fold; at least 90-fold; at least 100-fold; at least 125-fold; at least 150-fold; at least 175-fold; or at least 200-fold. In some alternatives, a selective MAP4K1 inhibitor exhibits at least 15-fold selectivity over another kinase, e.g., LCK and MAP4K family members (MAP4K4 (HGK) and MAP4K3 (GLK)). In some alternatives, the selective MAP4K1 inhibitors are selective over EGFR and L858R/T790M EGFR. In some alternatives, the selective MAP4K1 inhibitors of the disclosure are selective over BTK. In some alternatives, the selective MAP4K1 inhibitors of the disclosure are selective over JNK.

The disclosure provides methods of modulating (e.g., inhibiting) MAP4K1 activity in a subject in need thereof, said method comprising administering to the subject a compound provided herein, or a pharmaceutically acceptable salt thereof. In certain embodiments, the compounds of the disclosure, or pharmaceutically acceptable salts thereof, are useful for therapeutic administration to enhance, stimulate and/or increase immunity in subjects in need thereof, e.g., in cancer patients or patients with viral infection. In some instances, the compounds of the disclosure, or pharmaceutically acceptable salts thereof reduce, inhibit, or otherwise diminish pSLP76. In some instances, the compounds of the disclosure, or pharmaceutically acceptable salts thereof, are useful for therapeutic administration to enhancing at least one of activation, priming, migration, proliferation, survival and cytolytic activity of T cells relative to prior to administration. In certain aspects, T cell activation is characterized by enhanced levels of IL-2, IFN-gamma, or granzyme B production by T cells relative to prior to administration of the compound or pharmaceutically acceptable salt thereof. In some instances, the compounds of the disclosure, or pharmaceutically acceptable salts thereof, are useful for therapeutic administration to induce a change in cell cycle or cell viability. In some instances, the compounds of the disclosure, or pharmaceutically acceptable salts thereof, are useful for improving function of T effector cells.

In some instances, the compounds of the disclosure, or pharmaceutically acceptable salts thereof, are useful for inhibiting the suppressive effects of T regulatory cells or improving the T cell response to immune suppressive factors including adenosine and PGE2. In some instances, the compounds of the disclosure, or pharmaceutically acceptable salts thereof, are useful for increasing the frequency of CD8+ tumor infiltrating lymphocytes (TILS). In some instances, the compounds of the disclosure, or pharmaceutically acceptable salts thereof, are useful for enhancing CD3+/Treg ratios. In some instances, the compounds of the disclosure, or pharmaceutically acceptable salts thereof, are useful for enhancing cytokines. In some instances, the compounds of the disclosure, or pharmaceutically acceptable salts thereof, are useful for enhanacing cytokines with no impact on IL-6. In some instances, the compounds of the disclosure, or pharmaceutically acceptable salts thereof, indirectly inhibit the growth of cancer cells. In some instances, the compounds of the disclosure, or pharmaceutically acceptable salts thereof, are useful for priming of the immune response (i.e., vaccines) to tumors or viruses for booting or generating anti-viral/anti-tunor immunity. In one instance, the compounds of the disclosure, or pharmaceutically acceptable salts thereof, are used for enhancing or boosting response to a vaccine (such as a cancer vaccine or a personalized cancer vaccine (PCV)) or a CAR-T cell therapy.

Methods of treating a MAP4K1-dependent disease or disorder can include administering to a subject in need thereof a therapeutically effective amount of a compound provided herein, or a pharmaceutically acceptable salt thereof. For example, the MAP4K1-dependent disease or disorder is a cancer. The term “cancer” encompasses all forms of cancer including, but not limited to, all forms of carcinoma, melanomas, blastomas, sarcomas, lymphomas, leukemias. In some embodiments, cancer includes metastatic forms. Additionally, the disclosure includes refractory or recurrent malignancies whose growth may be inhibited using the compounds of the disclosure. For the uses described herein, any of the compounds of the disclosure, or pharmaceutically acceptable salts thereof, may be used alone or in combination with other therapeutic agents.

In some embodiments, the treatment results in a sustained response in the subject after cessation of the treatment. “Sustained response” refers to the sustained effect on reducing tumor growth after cessation of a treatment. For example, the tumor size may remain the same or smaller as compared to the size at the beginning of the administration phase. In some embodiments, the sustained response has a duration at least the same as the treatment duration, at least 1.5×, 2.0×, 2.5×, or 3.0× length of the treatment duration.

The treatment methods disclosed herein may result in a partial or complete response. As used herein, “complete response” or “CR” refers to disappearance of all target lesions; “partial response” or “PR” refers to at least a 30% decrease in the sum of the longest diameters (SLD) of target lesions, taking as reference the baseline SLD; and“stable disease” or “SD” refers to neither sufficient shrinkage of target lesions to qualify for PR, nor sufficient increase to qualify for PD, taking as reference the smallest SLD since the treatment started. As used herein, “overall response rate” (ORR) refers to the sum of complete response (CR) rate and partial response (PR) rate.

The treatment methods disclosed herein can lead to an increase in progression free survival and overall survival of the subject administered the selective MAP4K1 inhibitor. As used herein, “progression free survival” (PFS) refers to the length of time during and after treatment during which the disease being treated (e.g., cancer) does not get worse. Progression-free survival may include the amount of time subjects have experienced a complete response or a partial response, as well as the amount of time subjects have experienced stable disease.

As used herein, “overall survival” (OS) refers to the percentage of subjects in a group who are likely to be alive after a particular duration of time.

In some embodiments, cancers treatable with compounds of the disclosure or pharmaceutically acceptable salt thereof, include colon cancer, pancreatic cancer, breast cancer, prostate cancer, lung cancer, ovarian cancer, cervical cancer, renal cancer, bladder cancer, stomach cancer, liver cancer, gastric cancer, cancer of the head and neck, lymphoma, leukemia, urothelial carcinoma, merkel cell carcinoma, gastroesophageal junction carcinoma, esophageal squamous cell carcinoma, skin squamous cell carcinoma and melanoma.

In some embodiments, cancers treatable with compounds of the disclosure or pharmaceutically acceptable salts thereof include colon cancer, pancreatic cancer, breast cancer, prostate cancer, lung cancer, ovarian cancer, cervical cancer, renal cancer, bladder cancer, stomach cancer, liver cancer, cancer of the head and neck, lymphoma, leukemia, and melanoma.

In some embodiments, cancers that are treatable using the compounds of the disclosure or pharmaceutically acceptable salts thereof include, but are not limited to, solid tumors, including prostate cancer, colon cancer, esophageal cancer, endometrial cancer, ovarian cancer, uterine cancer, renal cancer, hepatic cancer, pancreatic cancer, gastric cancer, breast cancer, lung cancer, head and neck cancer, thyroid cancer, brain cancer, and bladder cancer and hematological cancer, including lymphoma, leukemia (chronic and acute forms) such as ALL, AML, CLL, CML, DLBCL, mantle cell lymphoma, Non-Hodgkin's lymphoma (NHL), including relapsed or refractory NHL and recurrent follicular, Hodgkin's lymphoma and multiple myeloma, and myeloproliferative diseases.

In some embodiments, diseases and indications that are treatable using the compounds of the disclosure or pharmaceutically acceptable salts thereof include, but are not limited to hematological cancer, sarcomas, respiratory cancer, gastrointestinal cancer, genitourinary tract cancer, liver cancer, bone cancer, nervous system cancer, gynecological cancer, and skin cancer.

Exemplary hematological cancer includes, for example, lymphomas and leukemias such as ALL, AML, acute promyelocyte leukemia (APL), CLL, CML, DLBCL, mantle cell lymphoma, Non-Hodgkin lymphoma (NHL), including Primary mediastinal B-cell lymphoma (PMBCL), relapsed or refractory NHL, recurrent follicular, and primary CNS lymphoma, Hodgkin's lymphoma, myeloproliferative diseases, including, primary myelofibrosis (PMF), polycythemia vera (PV), essential thrombocytosis (ET), myelodysplasia syndrome (MDS), T-cell acute lymphoblastic lymphoma (T-ALL), multiple myeloma, cutaneous T-cell lymphoma, Waldenstrom's Macroglubulinemia, hairy cell lymphoma, chronic myelogenic lymphoma, and Burkitt's lymphoma.

Exemplary sarcoma includes, for example, chondrosarcoma, Ewing's sarcoma, Kaposi's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, sarcoma of the soft tissue, and teratoma.

Exemplary respiratory tract cancer includes, for example, lung cancer such as non-small cell lung cancer (NSCLC), small cell lung cancer, epidermoid cancer, bronchogenic carcinoma, including squamous cell, undifferentiated small cell, undifferentiated large cell, adenocarcinoma, alveolar (bronchiolar) carcinoma, bronchial adenoma, chondromatous hamartoma, mesothelioma, and pleuropulmonary blastoma.

Exemplary gastrointestinal cancer includes, for example, cancers of the esophagus, including squamous cell carcinoma, adenocarcinoma, leiomyosarcoma, and lymphoma; stomach, including carcinoma, lymphoma, and leiomyosarcoma; pancreas, including ductal adenocarcinoma, insulinoma, glucagonoma, gastrinoma, carcinoid tumors, and vipoma; small instestine, including adenocarcinoma, lymphoma, carcinoid tumors, Kaposi's sarcoma, leiomyoma, hemangioma, lipoma, neurofibroma, and fibroma; large intestine, including adenocarcinoma, tubular adenoma, villous adenoma, hamartoma, and leiomyoma; colon; and gall bladder, including adenocarcinoma; and intestinal type and diffuse type gastric adenocarcinoma, rectum carcinoma, familiar adenomatous polyposis carcinoma and hereditary non-polyposis colorectal cancer.

Exemplary genitourinary tract cancer includes, for example, cancers of the kidney, including adenocarcinoma, Wilm's tumor [nephroblastoma], renal cell carcinoma, urothelial carcinoma, juxtaglomerular cell tumor (reninoma), angiomyolipoma, renal oncocytoma, Bellinio duct carcinoma, clear-cell sarcoma of the kidney, and mesoblastic nephroma; adrenal gland; renal pelvis; bladder, including transitional cell carcinoma, squamous cell carcinoma, adenocarcinoma, sarcoma, and small cell carcinoma; urethra, including squamous cell carcinoma, transitional cell carcinoma, and adenocarcinoma; prostate, including adenocarcinoma, sarcoma, and carcinoma; testis, including seminoma, teratoma, embryonal carcinoma, teratocarcinoma, choriocarcinoma, sarcoma, interstitial cell carcinoma, fibroma, fibroadenoma, adenomatoid tumors, and lipoma; penis; and pancreas.

Exemplary liver cancer includes, for example, hepatoma, including hepatocellular carcinoma, cholangiocarcinoma, hepatoblastoma, angiosarcoma, hepatocellular adenoma, biliary tract cancer, and hemangioma.

Exemplary bone cancer includes, for example, osteogenic sarcoma, fibrosarcoma, malignant fibrous histiocytoma, chondrosarcoma, Ewing's sarcoma, malignant lymphoma, including reticulum cell sarcoma, multiple myeloma, malignant giant cell tumor chordoma, osteochronfroma, including osteocartilaginous exostoses, benign chondroma, chondroblastoma, chondromyxofibroma, osteoid osteoma, and giant cell tumors Exemplary nervous system cancer includes, for example, cancer of the skull, including osteoma, hemangioma, granuloma, xanthoma, and osteitis deformans; meninges including, meningioma, meningiosarcoma, and gliomatosis; brain, including astrocytoma, meduoblastoma, glioma, ependymoma, germinoma (pinealoma), neuroectodermal tumor, glioblastoma, glioblastoma multiform, oligodendroglioma, schwannoma, retinoblastoma, congenital tumors, brain stem and hypopthamic glioma; and spinal cord, including neurofibroma, meningioma, glioma, and sarcoma; as well as neuroblastoma and Lhermitte-Duclos disease.

Exemplary gynecological cancer includes, for example, cancer of the uterus, including endometrial carcinoma; cervix, including cervical carcinoma, pre-tumor cervical dysplasia, squamouse cell carcinoma, adenocarcinoma, adenosquamous carcinoma, small cell carcinoma, neuroendocrine tumor, glassy cell carcinoma and villoglandular adenocarcinoma; ovaries, including ovarian carcinoma (serous cystadenocarcinoma, mucinous cystadenocarcinoma, unclassified carcinoma, endometroid tumor), granulosa-thecal cell tumors, Sertoli-Leydig cell tumors, dysgerminoma, malignant teratoma, and arrhenoblastoma; vulva (squamous cell carcinoma, intraepithelial carcinoma, adenocarcinoma, fibrosarcoma, and melanoma; vagina, including clear cell carcinoma, squamous cell carcinoma, and botryoid sarcoma (embryonal rhabdomyosarcoma); labia; and fallopian tubes.

Exemplary skin cancer includes, for example, melanoma, sebaceous gland carcinoma, basal cell carcinoma, squamous cell carcinoma, Kaposi's sarcoma, Merkel cell skin cancer, moles dysplastic nevi, lipoma, angioma, dermatofibroma, and keloids.

Examples of breast cancer include, for example, ER+/HER2− breast cancer, triple-negative breast cancer (TNBC), invasive ductal carcinoma, invasive lobular carcinoma, ductal carcinoma in situ, and lobular carcinoma in situ.

Exemplary head and neck cancer includes, for example, glioblastoma, melanoma, rhabdosarcoma, lymphosarcoma, osteosarcoma, squamous cell carcinomas, adenocarcinomas, oral cancer, throat cancer, including oropharyngeal cancer, laryngeal cancer, nasopharyngeal cancer, nasal and paranasal cancer, salivary gland cancer, mouth cancer, eye cancer, acoustic neuroma, pituitary adenoma, hypopharngx, and thyroid (medullary and papillary) and parathyroid cancer.

Other cancers include, for example, sweat gland cancer, spinal axis tumor, chest cancer, sickle cell anemia, and environmentally induced cancers including those induced by asbestos.

In some instances, the MAP4K1-dependent disease or disorder is a viral infection, such as infection caused by hepatitis B virus (HBV), hepatitis C virus (HCV), human papilloma virus (HPV), cytomegalovirus (CMV), herpes simplex virus (HSV), Epstein-Barr virus (EBV), varicella zoster virus, coxsackie virus, and human immunodeficiency virus (HIV).

### Combination Therapies

Compounds of the disclosure or pharmaceutically acceptable salts thereof can be administered as the sole pharmaceutical agent or in combination with one or more other anti-cancer agents for the treatment of cancel, where the combination causes no unacceptable adverse effects. In some embodiments, the other anti-cancer agents are immune-oncology agent, anticancer agents that are enzyme/protein/receptor inhibitors, radiation or chemotherapy.

Compounds of the disclosure or pharmaceutically acceptable salts thereof can be co-formulated with an immuno-oncology agent. Immuno-oncology agents include, for example, a small molecule drug, antibody, or other biologic or small molecule. Examples of biologic immuno-oncology agents include, but are not limited to, cancer vaccines, antibodies, and cytokines. In one aspect, the antibody is a monoclonal antibody. In another aspect, the monoclonal antibody is humanized or human. In another aspect, the antibody is a bispecific antibody.

In one aspect, the immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses (often referred to as immune checkpoint regulators).

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fnl4, TWEAK, BAFFR, EDAR, XEDAR, TACI, APRIL, BCMA, LTfiR, LIGHT, DcR3, HVEM, VEGI/TL1A, TRAMP/DR3, EDAR, EDA1, XEDAR, EDA2, TNFR1, Lymphotoxin α/TNPβ, TNFR2, TNF a, LT R, Lymphotoxin a 1β2, FAS, FASL, RELT, DR6, TROY, NGFR.

In one aspect, T cell responses can be stimulated by a combination of a compound of the disclosure and one or more of (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4, and (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

Other agents that can be combined with compounds of the disclosure for the treatment of cancer include antagonists of inhibitory receptors on NK cells or agonists of activating receptors on NK cells. For example, compounds of the disclosure can be combined with antagonists of KIR, such as lirilumab.

Yet other agents for combination therapies include agents that inhibit or deplete macrophages or monocytes, including but not limited to CSF-1R antagonists such as CSF-1R antagonist antibodies including RG7155 or FPA-008.

In another aspect, compounds of the disclosure or pharmaceutically acceptable salts thereof can be used with one or more of agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell anergy or exhaustion) and agents that trigger innate immune activation and/or inflammation at tumor sites.

In some embodiments, the immuno-oncology agent is a CTLA-4 antagonist, such as an antagonistic CTLA-4 antibody. Suitable CTLA-4 antibodies include, for example, YERVOY (ipilimumab) or tremelimumab. In another aspect, the immuno-oncology agent is a PD-1 antagonist, such as an antagonistic PD-1 antibody. Suitable PD-1 antibodies include, for example, OPDIVO (nivolumab), KEYTRUDA (pembrolizumab), or MEDI-0680 (AMP-514; WO2012/145493). The immuno-oncology agent may also include pidilizumab (CT-011), though its specificity for PD-1 binding has been questioned. Another approach to target the PD-1 receptor is the recombinant protein composed of the extracellular domain of PD-L2 (B7-DC) fused to the Fc portion of IgG1, called AMP-224

In another aspect, the immuno-oncology agent is a PD-L1 antagonist, such as an antagonistic PD-L1 antibody. Suitable PD-L1 antibodies include, for example, TECENTRIQ (atezolizumab) (RG7446; WO2010/077634), durvalumab (MEDI4736), BMS-936559 (WO2007/005874), and MSB0010718C (WO2013/79174).

In another aspect, the immuno-oncology agent is a LAG-3 antagonist, such as an antagonistic LAG-3 antibody. Suitable LAG3 antibodies include, for example, BMS-986016 (WO10/19570, WO14/08218), or IMP-731 or IMP-321 (WO08/132601, WO09/44273).

In another aspect, the immuno-oncology agent is a CD137 (4-1BB) agonist, such as an agonistic CD137 antibody. Suitable CD137 antibodies include, for example, urelumab and PF-05082566 (WO12/32433).

In another aspect, the immuno-oncology agent is a GITR agonist, such as an agonistic GITR antibody. Suitable GITR antibodies include, for example, BMS-986153, BMS-986156, TRX-518 (WO06/105021, WO09/009116) and MK-4166 (WOl 1/028683).

In another aspect, the immuno-oncology agent is an IDO antagonist. Suitable IDO antagonists include, for example, INCB-024360 (WO2006/122150, WO07/75598, WO08/36653, WO08/36642), indoximod, or NLG-919 (WO09/73620, WO09/1156652, WOl1/56652, WO12/142237).

In another aspect, the immuno-oncology agent is an OX40 agonist, such as an agonistic OX40 antibody. Suitable OX40 antibodies include, for example, MEDI-6383 or MEDI-6469. In another aspect, the immuno-oncology agent is an OX40L antagonist, such as an antagonistic OX40 antibody. Suitable OX40L antagonists include, for example, RG-7888 (WO06/029879).

In another aspect, the immuno-oncology agent is a CD40 agonist, such as an agonistic CD40 antibody. In yet another embodiment, the immuno-oncology agent is a CD40 antagonist, such as an antagonistic CD40 antibody. Suitable CD40 antibodies include, for example, lucatumumab or dacetuzumab.

In another aspect, the immuno-oncology agent is a CD27 agonist, such as an agonistic CD27 antibody. Suitable CD27 antibodies include, for example, varlilumab.

In another aspect, the immuno-oncology agent is MGA271 (to B7H3) (WOl 1/109400).

The compounds of the disclosure or pharmaceutically acceptable salts thereof can be used in combination with anticancer agents that are enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

The compounds of the disclosure or pharmaceutically acceptable salts thereof can be used in combination with one or more other enzyme/protein/receptor inhibitors for the treatment of cancer. For example, the compounds of the disclosure can be combined with one or more inhibitors of the following kinases for the treatment of cancer: Akt1, Akt2, Akt3, TGF-βPv, PKA, PKG, PKC, CaM-kinase, phosphorylase kinase, MEKK, ERK, MAPK, mTOR, EGFR, HER2, HER3, HER4, INS-R, IGF-1R, IR-R, PDGFotR, PDGFpR, CSFIR, KIT, FLK-II, KDR/FLK-1, FLK-4, fit-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK, and B-Raf.

In some embodiments, the compounds of the disclosure or pharmaceutically acceptable salts thereof can be combined with one or more of the following inhibitors for the treatment of cancer. Non-limiting examples of inhibitors that can be combined with the compounds of the disclosure or pharmaceutically acceptable salts thereof for treatment of cancers include an FGFR inhibitor (FGFR1, FGFR2, FGFR3 or FGFR4, e.g., fisogatinib, AZD4547, BAY 1187982, ARQ087, BGJ398, BIBF1120, TKI258, lucitanib, dovitinib, TAS-120, J J-42756493, Debiol347, INCB54828, INCB62079, and INCB63904), a JAK inhibitor (JAK1 and/or JAK2, e.g., ruxolitinib, baricitinib, or itacitinib (INCB39110)), an IDO inhibitor (e.g., epacadostat and NLG919), an LSD1 inhibitor (e.g., GSK2979552, INCB59872 and INCB60003), a TDO inhibitor, a PI3K-delta inhibitor (e.g., INCB50797 and INCB50465), a PI3K-gamma inhibitor such as a PI3K-gamma selective inhibitor (eganelisib) or a dual PI3K-delta/gamma selective inhibitor (duvelisib), a CSF1R inhibitor (e.g., PLX3397 and LY3022855), a TAM receptor tyrosine kinases (Tyro-3, Axl, and Mer), an angiogenesis inhibitor (Such as Avastin (bevacizumab)), an interleukin receptor inhibitor, bromo and extra terminal family members inhibitors (for example, bromodomain inhibitors or BET inhibitors such as OTX015, CPI-0610, INCB54329, and INCB57643), and an adenosine receptor antagonist or combinations thereof. Inhibitors of HDAC such as panobinostat and vorinostat can be combined with the compounds of the disclosure. Inhibitors of c-Met such as onartumzumab, tivantnib, and capmatinib (INC-280) be combined with the compounds of the disclosure. Inhibitors of BTK such as ibrutinib can be combined with the compounds of the disclosure. Inhibitors of mTOR such as rapamycin, sirolimus, temsirolimus, and everolimus can be combined with the compounds of the disclosure. Inhibitors of Raf, such as vemurafenib and dabrafenib can be combined with the compounds of the disclosure. Inhibitors of MEK such as trametinib, selumetinib and GDC-0973 can be combined with the compounds of the disclosure. Inhibitors of KIT, including avapritinib, imatinib, sunitinib, regorafenib, ripritinib (DCC2618), PLX9486, PLX3397, crenolanib, CDX-0158, CDX-0159. Inhibitors of RET including pralsetinib, selperctinib, alectinib, levatinib, cabozantinib, BOS172738 (DS-5010), SL-1001, TPX-0046, sitravatinib (MGCD516), and RXDX-105. Inhibitors of Hsp90 (e.g., tanespimycin), cyclin dependent kinases (e.g., palbociclib), PARP (e.g., olaparib) and Pim kinases (LGH447, INCB053914, and SGI-1776) can also be combined with compounds of the disclosure.

Compounds of the disclosure or pharmaceutically acceptable salts thereof can be used in combination with one or more agents for the treatment of cancer. In some embodiments, the agent is an alkylating agent, a proteasome inhibitor, a corticosteroid, or an immunomodulatory agent. Examples of an alkylating agent include bendamustine, nitrogen mustards, ethylenimine derivatives, alkyl sulfonates, nitrosoureas and triazenes, uracil mustard, chlormethine, cyclophosphamide (CYTOXAN), ifosfamide, melphalan, chlorambucil, pipobroman, triethylene-melamine, triethylenethiophosphoramine, busulfan, carmustine, lomustine, streptozocin, dacarbazine, and temozolomide. In some embodiments, the proteasome inhibitor is carfilzomib. In some embodiments, the corticosteroid is dexamethasone (DEX).

The compounds of the disclosure or pharmaceutically acceptable salts thereof can be administered in combination with one or more anti-cancer drugs, such as a chemotherapeutics. Example chemotherapeutics include any of: abarelix, abiraterone, afatinib, aflibercept, aldesleukin, alemtuzumab, alitretinoin, allopurinol, altretamine, anastrozole, arsenic trioxide, asparaginase, axitinib, azacitidine, bevacizumab, bexarotene, baricitinib, bicalutamide, bleomycin, bortezombi, bortezomib, brivanib, buparlisib, busulfan intravenous, busulfan oral, calusterone, capecitabine, carmustine, cediranib, cetuximab, chlorambucil, cladribine, clofarabine, crizotinib, cyclophosphamide, cytarabine, dacarbazine, dacomitinib, dactinomycin, dalteparin sodium, dasatinib, dactinomycin, daunorubicin, decitabine, degarelix, denileukin, denileukin diftitox, deoxycoformycin, dexrazoxane, docetaxel, doxorubicin, droloxafine, dromostanolone propionate, eculizumab, enzalutamide, epidophyllotoxin, epirubicin, erlotinib, estramustine, etoposide phosphate, etoposide, exemestane, fentanyl citrate, filgrastim, floxuridine, fludarabine, fluorouracil, flutamide, fulvestrant, gefitinib, gemcitabine, gemtuzumab ozogamicin, goserelin acetate, histrelin acetate, ibritumomab tiuxetan, idarubicin, idelalisib, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mithramycin, mitomycin C, mitotane, mitoxantrone, nandrolone phenpropionate, navelbene, necitumumab, nelarabine, neratinib, nilotinib, nilutamide, nofetumomab, oserelin, paclitaxel, pamidronate, panitumumab, pazopanib, pegaspargase, pegfilgrastim, pemetrexed disodium, pentostatin, pilaralisib, pipobroman, plicamycin, cisplatin, carboplatin, oxaliplatin, ponatinib, prednisone, procarbazine, quinacrine, rasburicase, regorafenib, reloxafine, rituximab, ruxolitinib, sorafenib, streptozocin, sunitinib, sunitinib maleate, tamoxifen, tegafur, temozolomide, teniposide, testolactone, thalidomide, thioguanine, thiotepa, topotecan, toremifene, tositumomab, trastuzumab, tretinoin, triptorelin, uracil mustard, valrubicin, vandetanib, vinblastine, vincristine, vinorelbine, vorinostat, and zoledronate.

Other anti-cancer agent(s) include antibody therapeutics such as trastuzumab (Herceptin).

Compounds of the disclosure or pharmaceutically acceptable salts thereof can be administered as the sole pharmaceutical agent or in combination with one or more anti-viral agents for the treatment of chronic viral infections, where the combination causes no unacceptable adverse effects. Chronic viral infections include, but are not limited to, diseases caused by: hepatitis C virus (HCV), human papilloma virus (HPV), cytomegalovirus (CMV), herpes simplex virus (HSV), Epstein-Barr virus (EBV), varicella zoster virus, coxsackie virus, human immunodeficiency virus (HIV). Parasitic infections (e.g., malaria) may also be treated by the above methods wherein compounds known to treat the parasitic conditions are optionally added in place of the antiviral agents.

Suitable antiviral agents contemplated for use in combination with the compound of the disclosure or a pharmaceutically acceptable salt thereof can comprise nucleoside and nucleotide reverse transcriptase inhibitors (NRTIs), non-nucleoside reverse transcriptase inhibitors (NNRTIs), protease inhibitors and other antiviral drugs.

Examples of suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir (BMS-180194); BCH-I0652; emitricitabine [(−)-FTC]; beta-L-FD4 (also called beta-L-D4C and named beta-L-2′,3′-dicleoxy-5-fluoro-cytidene); DAPD, (( )-beta-D-2,6-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrirnidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1549. Other antiviral agents include hydroxyurea, ribavirin, IL-2, IL-12, pentafuside and Yissum Project No. 11607.

When more than one pharmaceutical agent is administered to a subject, they can be administered simultaneously, separately, sequentially, or in combination (e.g., for more than two agents). For examples, when administered together with an additional anti-cancer or antiviral agent, the disclosed compounds or pharmaceutically acceptable salts thereof can be administered simultaneously in the same pharmaceutical formulation or simultaneously in separate pharmaceutical formulations. Alternatively, when administered together with an additional anti-cancer or antiviral agent, the disclosed compounds or pharmaceutically acceptable salts thereof can be administered at separate times, depending the dosing requirements of the additional anti-cancer or antiviral agent.

Pharmaceutical compositions are disclosed that include one or more compounds provided herein (such as the compound of Formulae I-III, IV(A), IV(B), V(A), V(B), VI(A), VI(B), VI(C), VII, VII(a), VII(B), VII(C), VIII(A), VIII(B), VIII(C), IX(A), IX(B), IX(C), X(A), X(B), X(C), XI, XII(A) and XII(B)), and typically at least one additional substance, such as an excipient, a known therapeutic other than those of the disclosure, and combinations thereof. In some embodiments, the disclosed compounds or pharmaceutically acceptable salts thereof can be used in combination with other agents known to have beneficial activity targeting diseases or disorders listed above. For example, disclosed compounds or pharmaceutically acceptable salts thereof can be administered alone or in combination with one or more anti-cancer or antiviral agent.

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

A “subject” is a mammal in need of medical treatment, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

The precise amount of compound or pharmaceutically acceptable salt thereof administered to provide an “effective amount” to the subject will depend on the mode of administration, the type, and severity of the disease or condition, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-cancer or antiviral agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the disclosure or a pharmaceutically acceptable salt thereof being used by following, for example, dosages reported in the literature and recommended in the Physician's Desk Reference (57th ed., 2003).

The term “effective amount” means an amount when administered to the subject which results in beneficial or desired results, including clinical results, e.g., inhibits, suppresses or reduces the symptoms of the condition being treated in the subject as compared to a control. For example, a therapeutically effective amount can be given in unit dosage form (e.g., 0.1 mg to about 50 g per day, alternatively from 1 mg to about 5 grams per day; and in another alternatively from 10 mg to 1 gram per day).

The particular mode of administration and the dosage regimen will be selected by the attending clinician, taking into account the particulars of the case (e.g. the subject, the disease, the disease state involved, the particular treatment, and whether the treatment is prophylactic). Treatment can involve daily or multi-daily or less than daily (such as weekly or monthly etc.) doses over a period of a few days to months, or even years.

The pharmaceutical composition of the disclosure is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In preferred embodiments, the pharmaceutical composition is formulated for intravenous administration.

“Pharmaceutically acceptable excipient” and “pharmaceutically acceptable carrier” refer to a substance that aids the formulation and/or administration of an active agent to and/or absorption by a subject and can be included in the compositions of the disclosure without causing a significant adverse toxicological effect on the subject. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds.

### General Synthetic Methods and Intermediates

The reactions for preparing compounds of the disclosure can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the disclosure can involve the protection and deprotection of various chemical groups. The need for protection and deprotection, and the selection of appropriate protecting groups, can be readily determined by one skilled in the art. The chemistry of protecting groups can be found, for example, in Wuts and Greene, Protective Groups in Organic Synthesis, 5th ed., John Wiley & Sons: New Jersey, (2014), which is incorporated herein by reference in its entirety.

Reactions can be monitored according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC).

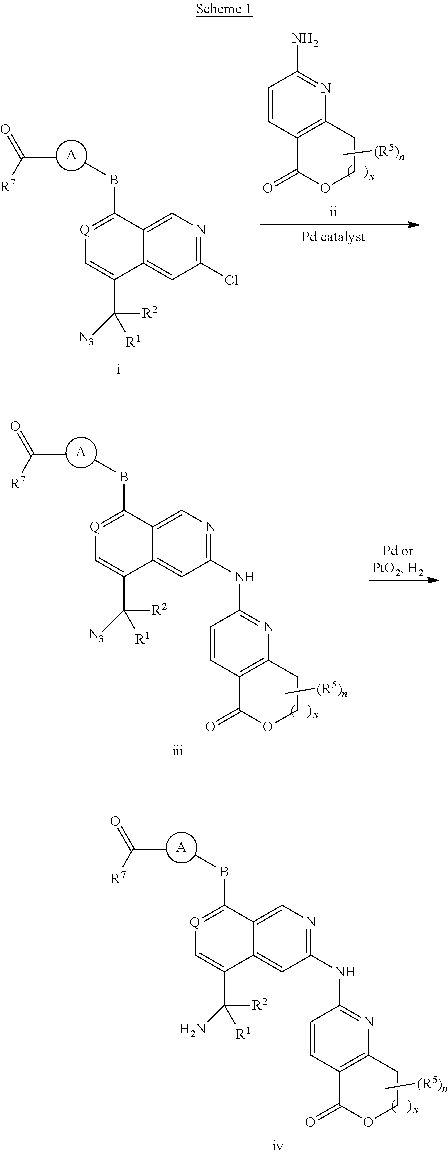

Scheme 1 Shows a Synthetic Protocol for the Preparation of Compounds of Formula iv

The azide-substituted chloro heterocyclic intermediates i can be coupled to the substituted anilines ii under Pd-catalyzed coupling conditions to give iii. The azide interediate iii can be reduced under catalytic hydrogenation conditions with a catalyst such as Pd/C or PtO₂ to give the amine compounds iv which are examples of MAP4K1 inhibitors described herein.

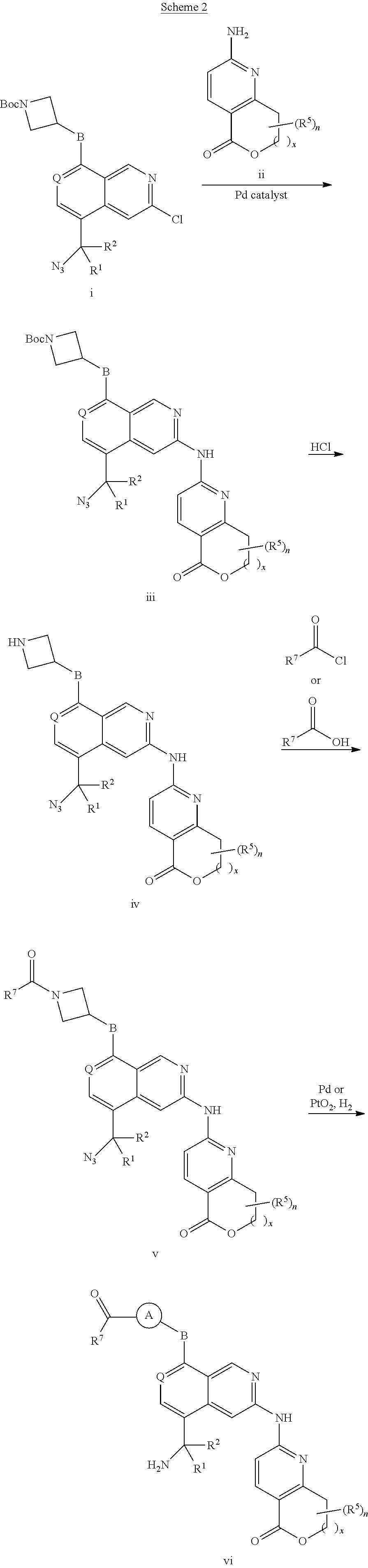

Scheme 2 Shows a Synthetic Protocol for the Preparation of Compounds of Formula vi

The Boc-protected azide-substituted chloro heterocyclic intermediates i can be coupled to the substituted anilines ii under Pd-catalyzed coupling conditions to give iii. In the scheme above, the intermediate is a Boc protected azetidine but this can be another acyclic or cyclic Boc protected amine. The Boc protecting group in intermediate iii can be removed under acidic conditions such as HCl to give the amine intermediate iv. The amine group of intermediate iv can be coupled to substituted carboxylic acids under amide coupling conditions such as HATU or coupled to substituted acid chlorides to give intermediate v. The azide group of intermediate v can be reduced under catalytic hydrogenation conditions with a catalyst such as Pd/C or PtO₂ to give the amine compounds vi which are examples of MAP4K1 inhibitors described herein.

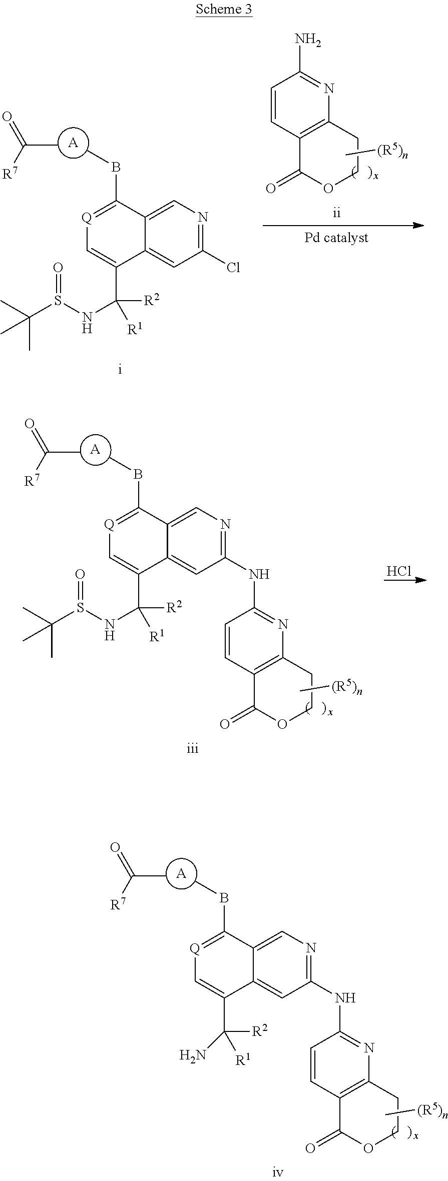

Scheme 3 Shows a Synthetic Protocol for the Preparation of Compounds of Formula iv

The sulfinamide-substituted chloro heterocyclic intermediates i can be coupled to the substituted anilines ii under Pd-catalyzed coupling conditions to give iii. The sulfinamide group of intermediate iii can converted to an amine under acidic conditions such as HCl to give amine compounds iv which are examples of MAP4K1 inhibitors described herein.

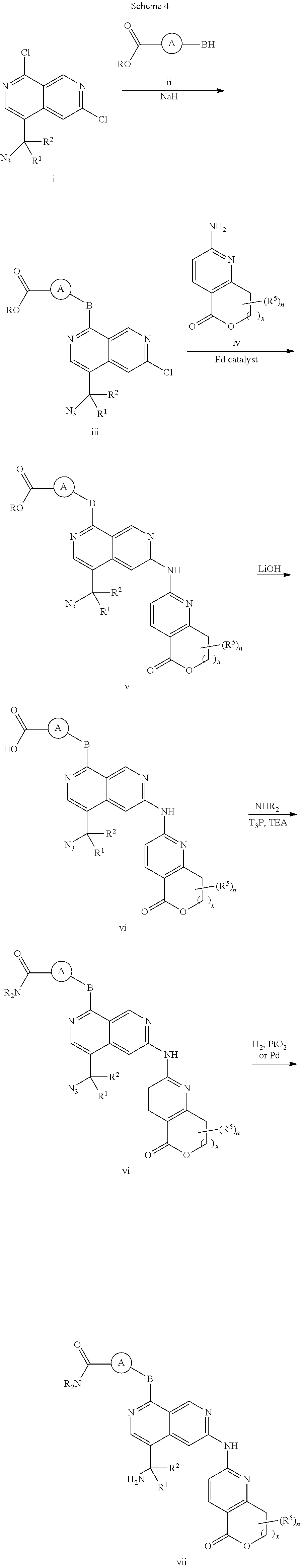

Scheme 4 Shows a Synthetic Protocol for the Preparation of Compounds of Formula vii

The dichloro heterocyclic intermediates i can be treated with alcohol or amine intermediates ii under SnAr conditions to give the ester-substituted chloro heterocyclic intermediates iii. Intermediate iii can be coupled to the substituted anilines iv under Pd-catalyzed coupling conditions to give v. The ester group in v can be hydrolyzed using a hydroxide base such as lithium hydroxide to give the carboxylic acid substituted intermediates vi. The carboxylic acid of intermediate vi can be coupled to primary and secondary amines to give the amide compounds vi. The azide group in intermediate v can be reduced under catalytic hydrogenation conditions with a catalyst such as Pd/C or PtO₂ to give the amine compounds vii which are examples of MAP4K1 inhibitors described herein.

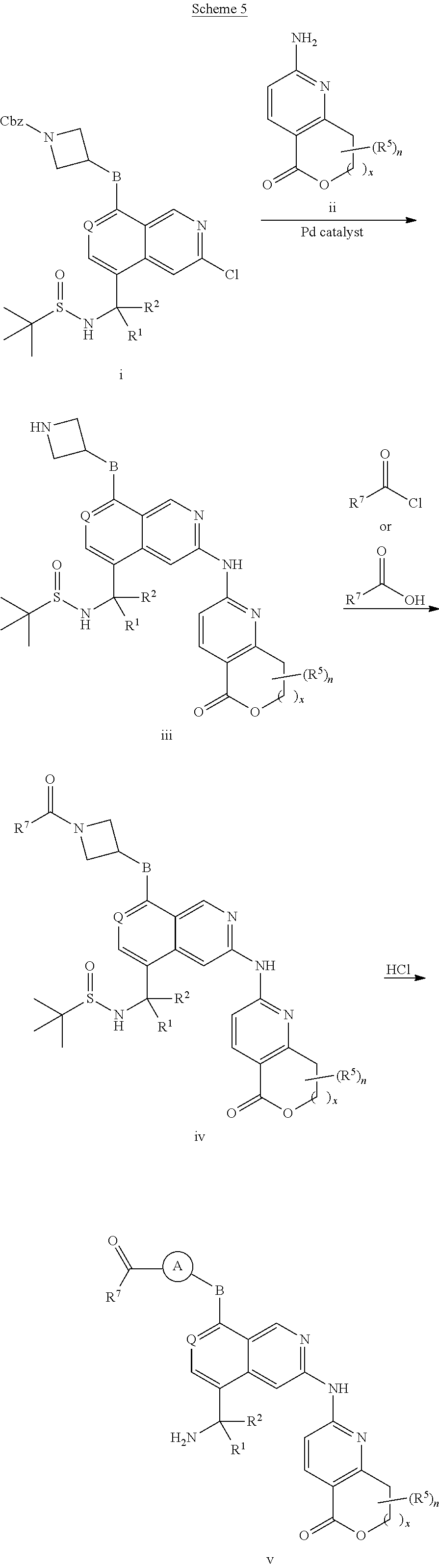

Scheme 5 Shows a Synthetic Protocol for the Preparation of Compounds of Formula iv

The sulfinamide-substituted chloro heterocyclic intermediates i that also contain a Cbz protected azetidine can be coupled to the substituted anilines ii under Pd-catalyzed coupling conditions to give iii which also has the Cbz removed under the reaction conditions. The amine group of intermediate iii can be coupled to substituted carboxylic acids under amide coupling conditions such as HATU or coupled to substituted acid chlorides to give intermediate iv. The sulfinamide group of intermediate iv can converted to an amine under acidic conditions such as HCl to give amine compounds v which are examples of MAP4K1 inhibitors described herein.

The following examples are intended to be illustrative and are not meant in any way to be limiting.

### EXEMPLIFICATION

### Abbreviations

ACN acetonitrile
ATP adenosine triphosphate
Boc₂O di-tert-butyl dicarbonate
BrettPhos Dicyclohexyl(2′,4′,6′-triisopropyl-3,6-dimethoxy-[1,1′-biphenyl]-2-yl)phosphine
t-BuONO tert-butyl nitrite
C Celsius
DBU diazabicyclo[5.4.0]undec-7-ene
DCE dichloroethane
DCM dichloromethane
DIPEA diisopropylamine
DMBNH₂ 2,4-dimethoxybenzylamine
DMF dimethyl formamide
DMSO dimethylsulfoxide
DTT dithiothreitol
EA ethyl acetate
EDCI 1-ethyl-3-(3-dimethylaminopropyl)carbodiimide
EDTA ethylenediaminetetracetic acid
FA formic acid
h hours
HATU (1-[Bis(dimethylamino)methylene]-1H-1,2,3-triazolo[4,5-b]pyridinium 3-oxide hexafluorophosphate)
HEPES 4-(2-hydroxyethyl)-1-piperazineethanesulfonic acid
HOBT hydroxybenzotriazole
HPLC high performance liquid chromatography
IBX 2-iodobenzoic acid
IC50 inhibitory concentration 50%
IPA isopropanol
KF potassium fluoride
LiHMDS lithium bis(trimethylsilyl)amide
min minutes
MTBE methyl tert-butyl ether
MeCN acetonitrile
MeI methyl iodide
MeOH methanol
NBS N-bromosuccinimide
NEt₃ triethylamine
NMO N-methylmorpholine N-oxide
NMP N-methyl-2-pyrrolidone
Pd(dppf)Cl₂ [1,1′-bis(diphenylphosphino)ferrocene]dichloropalladium (II)
Pd(PPh₃)₄ tetrakis(triphenylphosphine)palladium (0)
PE petroleum ether
SFC supercritical fluid chromatography
T₃P Propylphosphonic anhydride
TEA trimethylamine
TFA trifluoroacetic acid
TfOH trifluoromethanesulfonic acid
THF tetrahydrofuran
Ti(i-PrO)₄ titanium (IV) isopropoxide
TMEDA tetramethylenediamine
TMSN₃ trimethylsilyl azide

Methods for preparing compounds of the disclosure can be carried out in suitable solvents which can be readily selected by one of skill in the art of organic synthesis. Suitable solvents can be substantially non-reactive with the starting materials (reactants), intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the disclosure can involve the protection and deprotection of various chemical groups. The need for protection and deprotection, and the selection of appropriate protecting groups, can be readily determined by one skilled in the art. The chemistry of protecting groups can be found, for example, in Wuts and Greene, Protective Groups in Organic Synthesis, 5th ed., John Wiley & Sons: New Jersey, (2014), which is incorporated herein by reference in its entirety.

Reactions can be monitored according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC). Analytical instruments and methods for compound characterization: LC-MS: Unless otherwise indicated, all liquid chromatography-mass spectrometry

(LC-MS) data (sample analyzed for purity and identity) were obtained with an Agilent model-1260 LC system using an Agilent model 6120 mass spectrometer utilizing ES-API ionization fitted with an Agilent Poroshel 120 (EC-C18, 2.7 um particle size, 3.0×50 mm dimensions) reverse-phase column at 22.4 degrees Celsius. The mobile phase consisted of a mixture of solvent 0.1% FA in water and 0.1% FA in acetonitrile. A constant gradient from 95% aqueous/5% organic to 5% aqueous/95% organic mobile phase over the course of 4 minutes was utilized. The flow rate was constant at 1 mL/min.

Prep LC-MS: Preparative HPLC was performed on a Shimadzu Discovery VP® Preparative system fitted with a Luna 5u C18(2) 100A, AXIA packed, 250×21.2 mm reverse-phase column at 22.4 degrees Celsius. The mobile phase consisted of a mixture of solvent 0.1% FA in water and 0.1% FA in ACN. A constant gradient from 95% aqueous/5% organic to 5% aqueous/95% organic mobile phase over the course of 25 minutes was utilized. The flow rate was constant at 20 mL/min. Reactions carried out in a microwave were done so in a Biotage Initiator microwave unit.

Silica gel chromatography: Silica gel chromatography was performed on either a Teledyne Isco CombiFlash® Rf unit or a Biotage® Isolera Four unit.

Proton NMR: Unless otherwise indicated, all ¹H NMR spectra were obtained with a Varian 400 MHz Unity Inova 400 MHz NMR instrument (acquisition time=3.5 seconds with a 1 second delay; 16 to 64 scans). Where characterized, all protons were reported in DMSO-d6 solvent as parts-per million (ppm) with respect to residual DMSO (2.50 ppm).

One of ordinary skill in the art will recognize that modifications of the gradient, column length, and flow rate are possible and that some conditions may be more suitable for compound characterization than others, depending on the chemical species being analyzed.

### Example 1: Synthesis of Arylamine Intermediates

### Example 1a

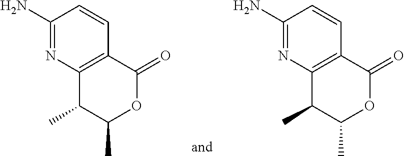

### Intermediates 1 and 2: (7S,8R)-2-Amino-7,8-dimethyl-7,8-dihydro-5H-pyrano[4,3-b]pyridin-5-one (Intermediate 1) and (7R,8S)-2-amino-7,8-dimethyl-7,8-dihydro-5H-pyrano[4,3-b]pyridin-5-one (Intermediate 2)

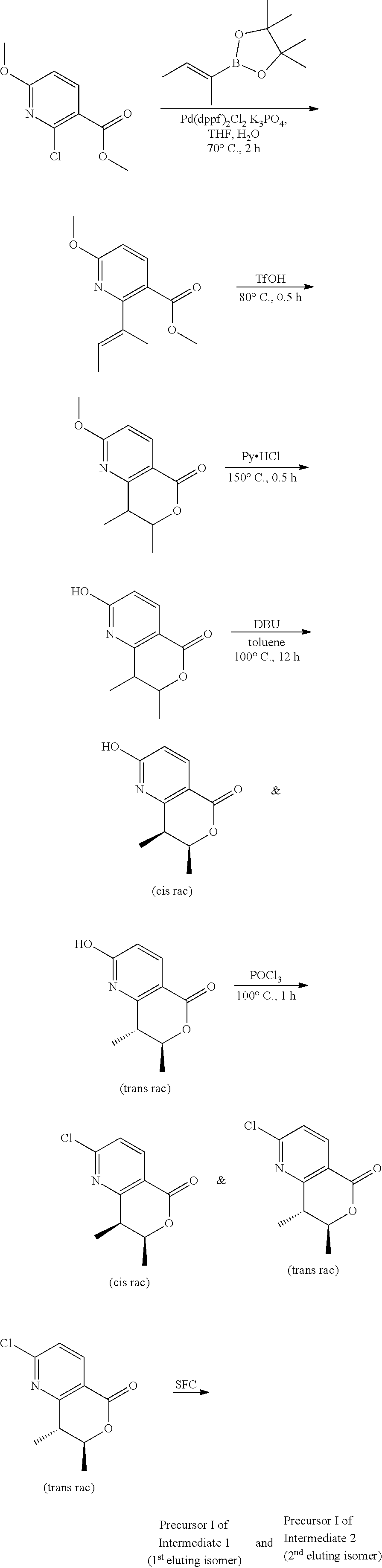 [0219] each of which is represented by the structures shown below:

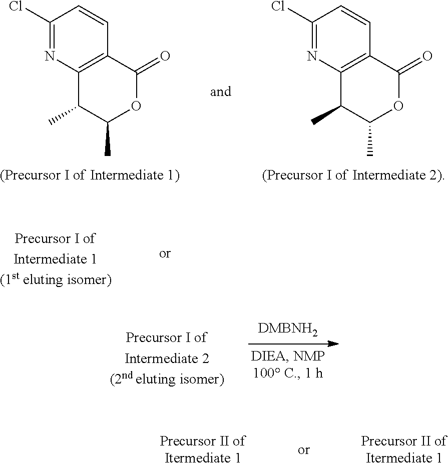

each of which is represented by the structures shown below:

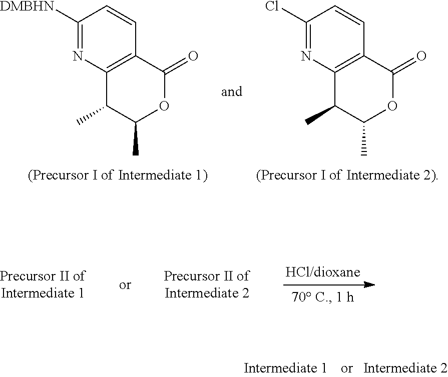

each of which is represented by the structures shown below:

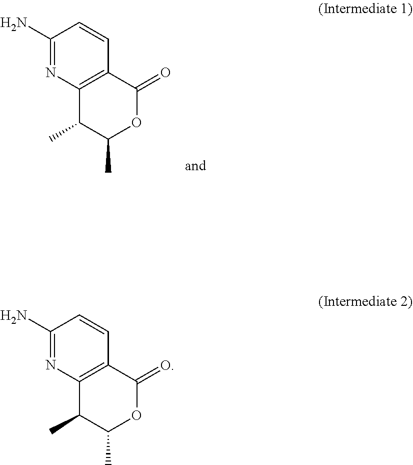

### Step 1: Methyl (E)-2-(but-2-en-2-yl)-6-methoxynicotinate

K₃PO₄ (120 g, 565 mmol, 3.00 eq) and Pd(dppf)Cl₂—CH₂Cl₂ (7.70 g, 9.42 mmol, 0.05 eq) were added to a solution of methyl 2-chloro-6-methoxynicotinate (38.0 g, 188 mmol, 1.00 eq) and (Z)-2-(but-2-en-2-yl)-4,4,5,5-tetramethyl-1,3,2-dioxaborolane (44.6 g, 245 mmol, 1.30 eq) in THF (320 mL) and H₂O (80.0 mL). The reaction mixture was stirred under N₂ at 70° C. for 2 h. The reaction mixture was diluted with water (300 mL) and extracted with EA (250 mL×3). The organic layers were combined and dried over sodium sulfate, then filtered and concentrated in vacuo to give the residue. The residue was purified by prep-HPLC (ACN-H₂O gradient with 0.1% TFA additive). The product-containing fractions were adjusted to pH=8-9 with solid sodium carbonate and the mixture was extracted with EA (300 mL×3). The combined organic layers were washed with brine (500 mL), dried over Na₂SO₄, filtered and concentrated to give the title compound (37.0 g, 167 mmol, 88.7% yield) as a yellow oil.

### Step 2: 2-Methoxy-7,8-dimethyl-7,8-dihydro-5H-pyrano[4,3-b]pyridin-5-one

A solution of methyl (E)-2-(but-2-en-2-yl)-6-methoxynicotinate (37.0 g, 167 mmol, 1.00 eq) in TfOH (171 g, 1.15 mol, 101 mL, 6.85 eq) was stirred at 80° C. for 0.5 h. The mixture was then cooled to ambient temperature, poured into saturated aqueous NaHCO₃ solution (1000 mL) and extracted with EA (300 mL×5). The organic layers were dried over sodium sulfate, filtered, and concentrated to give a residue. The residue was purified by flash-column chromatography on silica gel (gradient elution, 1% to 25% EA-PE) to give the title compound (30.0 g, 144 mmol, 86.6% yield) as a yellow oil.

### Steps 3: 2-Hydroxy-7,8-dimethyl-7,8-dihydro-5H-pyrano[4,3-b]pyridin-5-one

A mixture of 2-methoxy-7,8-dimethyl-7,8-dihydro-5H-pyrano[4,3-b]pyridin-5-one (30.0 g, 144 mmol, 1.00 eq) and pyridine-hydrochloride (41.8 g, 361 mmol, 2.50 eq) was stirred at 150° C. for 0.5 h. The reaction mixture was purified directly by flash-column chromatography on silica gel (gradient elution, 2% to 10% MeOH-DCM) to give the title compound (26.0 g, 134 mmol, 92.9% yield) as a yellow solid.

### Step 4: rac-(7S,8S)-2-Hydroxy-7,8-dimethyl-7,8-dihydro-5H-pyrano[4,3-b]pyridin-5-one and rac-(7S,8R)-2-hydroxy-7,8-dimethyl-7,8-dihydro-5H-pyrano[4,3-b]pyridin-5-one

DBU (60.8 mL, 403 mmol, 3.00 eq) was added to a solution of 2-hydroxy-7,8-dimethyl-7,8-dihydro-5H-pyrano[4,3-b]pyridin-5-one (26.0 g, 134 mmol, 1.00 eq) in toluene (290 mL). The reaction mixture was stirred at 100° C. for 12 h, then was cooled to ambient temperature and concentrated under vacuum. The residue was purified by flash-column chromatography on silica gel (gradient elution, 1% to 10% MeOH-DCM) to afford the title compounds as a mixture of isomers that were used in the next step without further purification.

### Step 5: rac-(7S,8S)-2-Chloro-7,8-dimethyl-7,8-dihydro-5H-pyrano[4,3-b]pyridin-5-one and rac-(7S,8R)-2-chloro-7,8-dimethyl-7,8-dihydro-5H-pyrano[4,3-b]pyridin-5-one

A mixture of rac-(7S,8S)-2-hydroxy-7,8-dimethyl-7,8-dihydro-5H-pyrano[4,3-b]pyridin-5-one and rac-(7S,8R)-2-hydroxy-7,8-dimethyl-7,8-dihydro-5H-pyrano[4,3-b]pyridin-5-one (26.0 g, 134 mmol, 1 equiv) in POCl₃ (150 mL, 1.61 mol, 11.9 equiv) was stirred at 100° C. for 1 h. The reaction mixture was then cooled to ambient temperature and poured into saturated aqueous NaHCO₃ solution (2 L) at 0-10° C. The quenched mixture was extracted with EA (300 mL×3) and the combined organic layers were washed with brine, dried over sodium sulfate, filtered, and concentrated. The cis- and trans-racemic isomers were separated by prep-HPLC (column: Phenomenex luna C18 250*50 mm*10 um; mobile phase: [water (0.1% TFA)-ACN]; B %: 35% ACN-55% CAN over 20 min). rac-(7S,8S)-2-Chloro-7,8-dimethyl-7,8-dihydro-5H-pyrano[4,3-b]pyridin-5-one was the first compound to elute and was obtained as a white solid. MS (ES+) C₁₀H₁₀ClNO₂ requires: 211, found: 212[M+H]+. ¹H NMR: 400 MHz, CDCl₃ δ 8.29 (d, J=8.2 Hz, 1H), 7.39 (d, J=8.2 Hz, 1H), 4.83 (dq, J=3.2, 6.6 Hz, 1H), 3.09 (dq, J=3.2, 7.2 Hz, 1H), 1.49 (d, J=6.5 Hz, 3H), 1.30 (d, J=7.2 Hz, 3H). rac-(7S,8R)-2-Chloro-7,8-dimethyl-7,8-dihydro-5H-pyrano[4,3-b]pyridin-5-one was the second compound to elute and was obtained as a white solid. MS (ES+) C₁₀H₁₀ClNO₂ requires: 211, found: 212[M+H]⁺. ¹H NMR: 400 MHz, CDCl₃ δ 8.28 (d, J=8.2 Hz, 1H), 7.38 (d, J=8.2 Hz, 1H), 4.58-4.43 (m, 1H), 3.05 (quin, J=7.2 Hz, 1H), 1.56-1.40 (m, 6H).

### Step 6: (7S,8R)-2-Chloro-7,8-dimethyl-7,8-dihydro-5H-pyrano[4,3-b]pyridin-5-one and (7R,8S)-2-chloro-7,8-dimethyl-7,8-dihydro-5H-pyrano[4,3-b]pyridin-5-one

rac-(7S,8R)-2-Chloro-7,8-dimethyl-7,8-dihydro-5H-pyrano[4,3-b]pyridin-5-one was separated by SFC (column: DAICEL CHIRALPAK AD (250 mm×50 mm, 10 um); mobile phase: MeOH in CO₂) to give the first eluting isomer (peak 1) as a white solid and second eluting isomer (peak 2) as a white solid.

### Step 7: (7S,8R)-2-((2,4-Dimethoxybenzyl)amino)-7,8-dimethyl-7,8-dihydro-5H-pyrano[4,3-b]pyridin-5-one

DIPEA (7.48 g, 57.8 mmol, 10.1 mL, 2.50 eq) and DMBNH₂ (5.03 g, 30.1 mmol, 4.53 mL, 1.30 eq) were added to a solution of (7S,8R)-2-chloro-7,8-dimethyl-7,8-dihydro-5H-pyrano[4,3-b]pyridin-5-one (1^{st} eluting isomer (peak 1) from Step 6 above) (4.90 g, 23.1 mmol, 1.00 eq) in NMP (50.0 mL). The reaction mixture was stirred at 100° C. for 1 h, then was poured into water (500 mL) and extracted with EA (100 mL×3). The combined organic layers were washed with brine (200 mL), dried over sodium sulfate, filtered and concentrated to give the title compound (7.93 g, crude) as a yellow oil that was used directly in the next step. MS (ES+) C₁₉H₁₁N₂O₄ requires: 342, found: 343[M+H]⁺.

### Step 8: (7S,8R)-2-Amino-7,8-dimethyl-7,8-dihydro-5H-pyrano[4,3-b]pyridin-5-one

A solution of (7S,8R)-2-((2,4-Dimethoxybenzyl)amino)-7,8-dimethyl-7,8-dihydro-5H-pyrano[4,3-b]pyridin-5-one (7.93 g, 23.1 mmol, 1.00 eq) in HCl/dioxane (4.00 M, 50.0 mL, 8.64 eq) was stirred at 70° C. for 1 h. The reaction mixture was then concentrated and partitioned between saturated aqueous sodium bicarbonate solution (100 mL) and was extracted with DCM (100 mL×3). The combined organic layer was washed with brine (200 mL), dried over sodium sulfate, filtered and concentrated. The residue was triturated in MTBE (50 mL) for 10 mins and filtered to get a yellow solid. The yellow solid was dried under vacuum to give the title compound, Intermediate 1(3.23 g, 16.4 mmol, 71.2% yield, 98.1% purity). MS (ES+) C₁₀H₁₂N₂O₂ requires: 192, found: 193[M+H]⁺. ¹H NMR: 400 MHz, DMSO-d6 δ 7.77 (d, J=8.6 Hz, 1H), 6.97 (s, 2H), 6.40 (d, J=8.6 Hz, 1H), 4.43-4.21 (m, 1H), 2.88-2.65 (m, 1H), 1.35 (d, J=6.4 Hz, 3H), 1.25 (d, J=7.0 Hz, 3H). The absolute stereochemistry of Intermediate 1 was determined by X-ray crystal structure.

### Steps 9 and 10: (7R,8S)-2-Amino-7,8-dimethyl-7,8-dihydro-5H-pyrano[4,3-b]pyridin-5-one

The title compound (Intermediate 2) was prepared separately from (7R,8S)-2-chloro-7,8-dimethyl-7,8-dihydro-5H-pyrano[4,3-b]pyridin-5-one (the second eluting isomer (peak 2) of Step 6) using the same two-step procedure as described in Steps 7 and 8 for Intermediate 2. MS (ES+) C₁₀H₁₂N₂O₂ requires: 192, found: 193[M+H]⁺. ¹H NMR: 400 MHz, DMSO-d₆ δ 7.77 (d, J=8.6 Hz, 1H), 6.97 (s, 2H), 6.40 (d, J=8.6 Hz, 1H), 4.43-4.21 (m, 1H), 2.88-2.65 (m, 1H), 1.35 (d, J=6.4 Hz, 3H), 1.25 (d, J=7.0 Hz, 3H).

### Example 1b

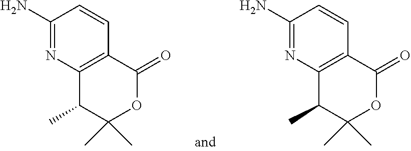

### Intermediates 3 and 4: (R)-2-amino-7,7,8-trimethyl-7,8-dihydro-5H-pyrano[4,3-b]pyridin-5-one and (S)-2-amino-7,7,8-trimethyl-7,8-dihydro-5H-pyrano[4,3-b]pyridin-5-one

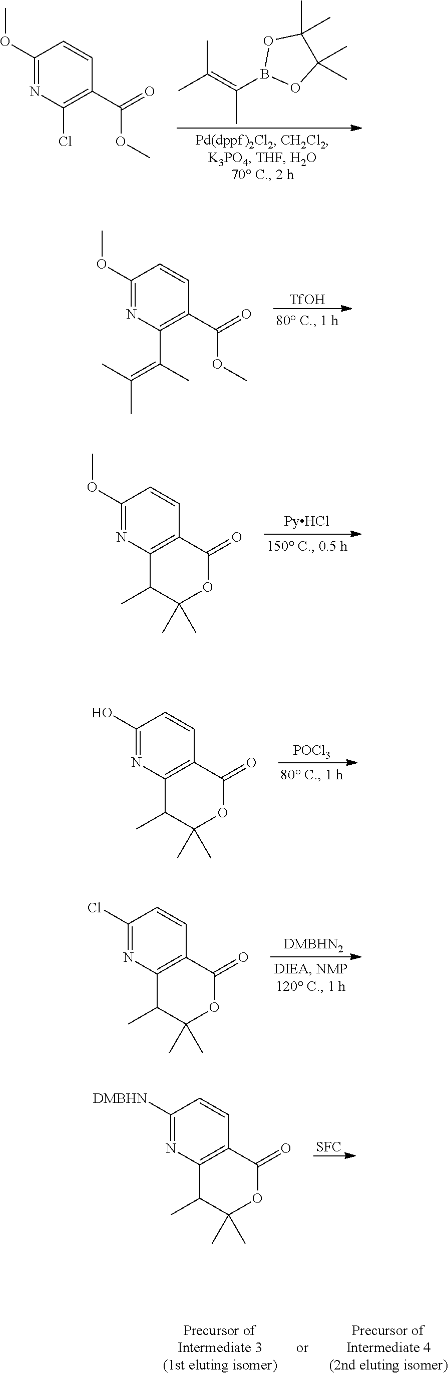 [0233] each of which is represented by the structure shown below:

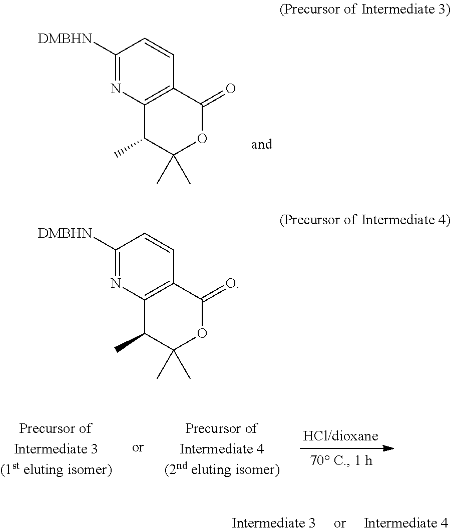

each of which is represented by the structure shown below:

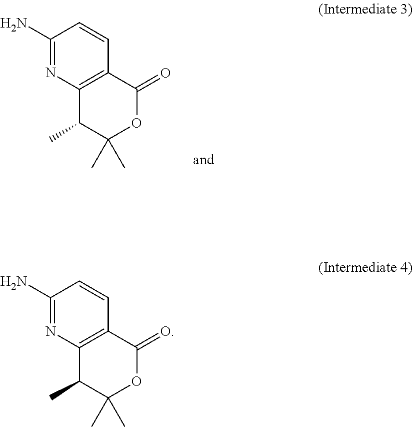

### Steps 1-5: rac 2-((2,4-Dimethoxybenzyl)amino)-7,7,8-trimethyl-7,8-dihydro-5H-pyrano[4,3-b]pyridin-5-one

The title compound was prepared from methyl 2-chloro-6-methoxynicotinate and 4,4,5,5-tetramethyl-2-(3-methylbut-2-en-2-yl)-1,3,2-dioxaborolane using similar procedures as described above in Steps 1-3 and 5-7 for Intermediate 1.

### Step 6: (R)-2-((2,4-Dimethoxybenzyl)amino)-7,7,8-trimethyl-7,8-dihydro-5H-pyrano[4,3-b]pyridin-5-one and (S)-2-((2,4-Dimethoxybenzyl)amino)-7,7,8-trimethyl-7,8-dihydro-5H-pyrano[4,3-b]pyridin-5-one

Rac 2-((2,4-Dimethoxybenzyl)amino)-7,7,8-trimethyl-7,8-dihydro-5H-pyrano[4,3-b]pyridin-5-one was separated by SFC (column: DAICEL CHIRALPAK AS-H (250 mm*30 mm, 5 um); mobile phase: [0.1% NH₄OH MeOH in CO₂]) to give (R)-2-((2,4-dimethoxybenzyl)amino)-7,7,8-trimethyl-7,8-dihydro-5H-pyrano[4,3-b]pyridin-5-one (1^{st} eluting isomer (Precursor to Intermediate 3), 0.55 g, 79% yield) and (S)-2-((2,4-dimethoxybenzyl)amino)-7,7,8-trimethyl-7,8-dihydro-5H-pyrano[4,3-b]pyridin-5-one (2^{nd} eluting isomer (Precursor to Intermediate 4), 0.55 g, 79% yield). Each intermediate was isolated as a yellow oil.

### Step 7 and 8: (R)-2-amino-7,7,8-trimethyl-7,8-dihydro-5H-pyrano[4,3-b]pyridin-5-one and (S)-2-amino-7,7,8-trimethyl-7,8-dihydro-5H-pyrano[4,3-b]pyridin-5-one

The title compounds (Intermediates 3 and 4) were prepared separately from the 1^{st} and 2^{nd} eluting isomers, i.e., (R)-2-((2,4-dimethoxybenzyl)amino)-7,7,8-trimethyl-7,8-dihydro-5H-pyrano[4,3-b]pyridin-5-one and (S)-2-((2,4-Dimethoxybenzyl)amino)-7,7,8-trimethyl-7,8-dihydro-5H-pyrano[4,3-b]pyridin-5-one, using the same procedure as described in Step 8 of Intermediate 1. Intermediate 3, (R)-2-amino-7,7,8-trimethyl-7,8-dihydro-5H-pyrano[4,3-b]pyridin-5-one, was obtained as a yellow solid. MS (ES+) C₁₁H₁₄N₂O₂ requires: 206, found: 207[M+H]⁺. ¹H-NMR (400 MHz, CD₃OD): δ ppm 7.89 (d, J=8.8 Hz, 1H), 6.50 (d, J=8.8 Hz, 1H), 2.85-2.80 (m, 1H), 1.41 (s, 6H), 1.27 (d, J=7.2 Hz, 3H). Intermediate 4, (S)-2-Amino-7,7,8-trimethyl-7,8-dihydro-5H-pyrano[4,3-b]pyridin-5-one, was obtained as a yellow solid. MS (ES+) C₁₁H₁₄N₂O₂ requires: 206, found: 207[M+H]⁺. ¹H-NMR (400 MHz, CD₃OD): δ ppm 7.89 (d, J=8.8 Hz, 1H), 6.50 (d, J=8.8 Hz, 1H), 2.85-2.80 (m, 1H), 1.41 (s, 6H), 1.27 (d, J=7.2 Hz, 3H). The stereochemistry of Intermediate 3 was determined in the context of compound 16 using an X-ray crystal structure.

### Example 1c

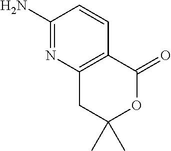

### Intermediate 17: 2-Amino-7,7-dimethyl-7,8-dihydro-5H-pyrano[4,3-b]pyridin-5-one

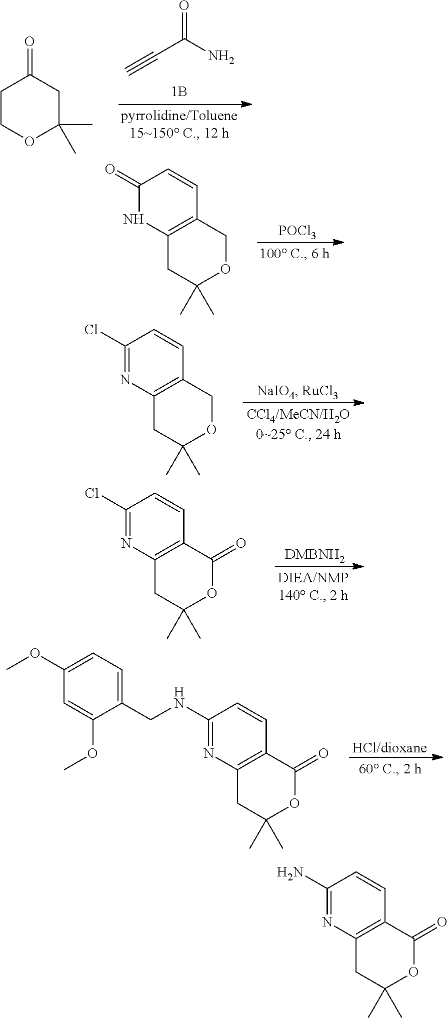

### Step 1: 7,7-Dimethyl-1,5,7,8-tetrahydro-2H-pyrano[4,3-b]pyridin-2-one

A mixture of 2,2-dimethyltetrahydro-4H-pyran-4-one (500 g, 3.90 mol, 1.00 eq) and pyrrolidine (391 mL, 4.68 mol, 1.20 eq) in toluene (4.00 L) was heated at 145° C. with a Dean-Stark trap for 2 h. The water layer (˜16 mL) was removed from the Dean-Stark trap and the reaction mixture was cooled to 15° C. After cooling, prop-2-ynamide (539 g, 7.80 mol, 2.00 eq) was added and the reaction mixture was heated to 150° C. The reaction mixture was heated at 150° C. for 10 h, then was cooled to ambient temperature. The cooled reaction mixture was filtered, and the filtrate was concentrated under reduced pressure to give a residue. The residue was purified by flash-column chromatography on silica gel (10% methanol-dichloromethane) to give the title compound (560 g, 62% yield) as a yellow solid.

### Step 2: 2-Chloro-7,7-dimethyl-7,8-dihydro-5H-pyrano[4,3-b]pyridine

A solution of 7,7-dimethyl-1,5,7,8-tetrahydro-2H-pyrano[4,3-b]pyridin-2-one (500 g, 2.23 mol, 1 eq) in POCl₃ (350 mL, 3.77 mol, 9.64 eq) was heated to 100° C. for 6 h. The reaction mixture then cooled to ambient temperature and concentrated under vacuum. The residue was poured over ice-water (1.00 L). The mixture was extracted with EA (750 mL×2). The combined organic layers were dried over Na₂SO₄, filtered and concentrated under vacuum to give the title compound (363 g, 82.2% yield) as a brown oil.

### Step 3: 2-Chloro-7,7-dimethyl-7,8-dihydro-5H-pyrano[4,3-b]pyridin-5-one

A solution of NaIO₄ (487 g, 2.28 mol, 3.00 eq) in water (1.20 L) as added to a mixture of 2-chloro-7,7-dimethyl-7,8-dihydro-5H-pyrano[4,3-b]pyridine (150 g, 759 mmol, 1.00 eq) in MeCN (50.0 mL) and CCl4 (2.70 L). The mixture was cooled to 0° C., and then RuCl₃ (11.0 g, 53.1 mmol, 0.07 eq) was added. The reaction mixture was stirred at 0° C. for 0.5 h, then was warmed to 20° C. for 11.5 h. Saturated aqueous sodium sulfite solution (1.00 L) was added, and the mixture was filtered. The filtrate was extracted with EA (500 mL×3), and the organic layers were combined. The combined organic layer was washed with brine (1.00 L), dried over Na₂SO₄, filtered and concentrated to give the title compound (132 g, 624 mmol, 82.1% yield) as a yellow solid.

### Step 4: 2-((2,4-Dimethoxybenzyl)amino)-7,7-dimethyl-7,8-dihydro-5H-pyrano[4,3-b]pyridin-5-one

(2,4-Dimethoxyphenyl) methanamine (160 g, 957 mmol, 1.50 eq) was added to a solution of 2-chloro-7,7-dimethyl-7,8-dihydro-5H-pyrano[4,3-b]pyridin-5-one (135 g, 638 mmol, 1.00 eq) and DIPEA (222 mL, 1.28 mol, 2.00 eq) in NMP (1.08 L) at ambient temperature. The reaction mixture was heated to 140° C. for 2 h, and then was cooled to ambient temperature. The reaction mixture was then partitioned between water (700 mL) and EA. The layers were separated, and the aqueous layer was further extracted with EA (500 mL×3). The organic layers were combined and washed with brine (400 mL×3), dried over anhydrous sodium sulfate, filtered and concentrated to afford the title compound as a yellow solid (160 g). The crude product was used for next step directly.

### Step 5: 2-Amino-7,7-dimethyl-7,8-dihydro-5H-pyrano[4,3-b]pyridin-5-one

HCl (4.0 M in dioxane, 1.20 L, 11.0 equiv) was added to: 2-((2,4-dimethoxybenzyl)amino)-7,7-dimethyl-7,8-dihydro-5H-pyrano[4,3-b]pyridin-5-one (150 g, 438 mmol, 1.00 eq) at 20° C. The reaction mixture was heated to 60° C. for 2 h, then was cooled to ambient temperature and concentrated under vacuum. The residue was poured into saturated NaHCO₃ aqueous solution (1.00 L) and extracted with EA (500 mL×4). The combined organic layer was washed with brine (500×2), dried over Na₂SO₄, filtered and concentrated. The residue was dissolved in EA (300 mL) and petroleum ether (150 mL) was added drop wise to get yellow slurry. The solids were filtered and collected to give the title compound (52.0 g, 60.9% yield) as a yellow solid. MS (ES+) C₁₀H₁₂N₂O₂ requires: 192, found: 193[M+H]⁺. ¹H NMR (400 MHz, DMSO-d6) δ 7.76 (d, J=8.4 Hz, 1H), 6.98 (s, 2H), 6.39 (d, J=8.8 Hz, 1H), 2.89 (s, 2H), 1.37 (s, 6H).

### Example 1d

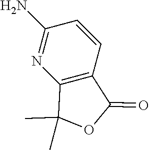

### Intermediate 18: 2-Amino-7,7-dimethylfuro[3,4-b]pyridin-5(7H)-one

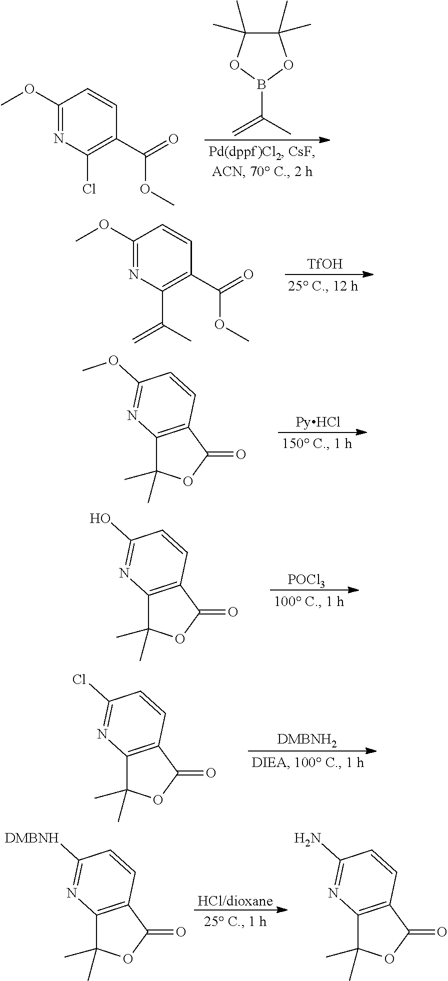

### Step 1: Methyl 6-methoxy-2-(prop-1-en-2-yl)nicotinate

Pd(dppf)Cl₂ (544 mg, 744 umol, 0.500 equiv) and cesium fluoride (4.52 g, 29.8 mmol, 2.00 equiv) were added to a mixture of methyl 2-chloro-6-methoxynicotinate (3.00 g, 14.9 mmol) and 4,4,5,5-tetramethyl-2-(prop-1-en-2-yl)-1,3,2-dioxaborolane (3.75 g, 22.3 mmol) in MeCN (50 mL). The mixture was stirred at 70° C. for 2 h under nitrogen atmosphere, then was cooled to ambient temperature. The reaction mixture was then poured onto water (200 mL) and extracted with EA (50 mL×3). The combined organic layers were dried over anhydrous sodium sulfate, filtered and concentrated in vacuo to give a residue. The residue was purified by flash-column chromatography on silica gel (gradient elution, 1% to 2% EA-petroleum ether) to give the title compound (3.0 g, crude) as a colorless oil.

### Step 2: 2-Methoxy-7,7-dimethylfuro[3,4-b]pyridin-5(7H)-one

A solution of Methyl 6-methoxy-2-(prop-1-en-2-yl)nicotinate (3.00 g, 14.5 mmol) in TfOH (17.0 g, 113 mmol, 10 mL) was stirred at 25° C. for 12 h. The reaction mixture was then poured over water (50 mL) and saturated aqueous sodium bicarbonate solution was added to adjust the pH to 7. The mixture was extracted with EA (30 mL×3). The combined organic layers were dried over anhydrous sodium sulfate, filtered and concentrated in vacuo to give a residue. The residue was purified by flash-column chromatography on silica gel (gradient elution, 10% to 33% EA-petroleum ether) to give the title compound (2.3 g, 82% yield) as a yellow solid.

### Steps 3-6: 2-Amino-7,7-dimethylfuro[3,4-b]pyridin-5(7H)-one

The title compound was prepared from 2-Methoxy-7,7-dimethylfuro[3,4-b]pyridin-5(7H)-one using the four-step procedure described in Steps 3, 5, 7 and 8 for Intermediate 1. MS (ES+) C₉H₁₀N₂O₂ requires: 178, found: 179[M+H]⁺.

### Example 1e

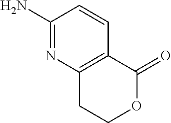 [SMILES: Nc1ccc2c(n1)CCOC2=O]

### Intermediate 19: 2-Amino-7,8-dihydro-5H-pyrano[4,3-b]pyridin-5-one

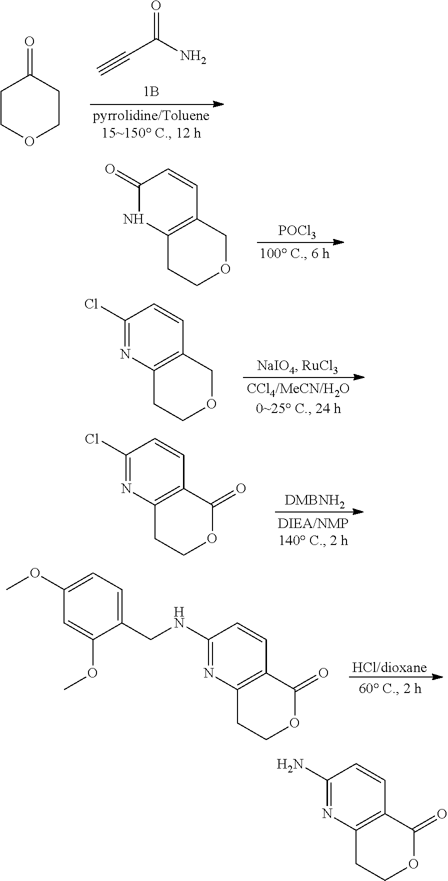

### Step 1: 2-Amino-7,8-dihydro-5H-pyrano[4,3-b]pyridin-5-one

The title compound was prepared from tetrahydro-4H-pyran-4-one using the same five-step procedure described in Steps 1-5 for Intermediate 17. MS (ES+) C₈H₈N₂O₂ requires: 164, found: 165[M+H]⁺. ¹H NMR, 400 MHz, DMSO-d6, δ=7.77 (d, J=8.8 Hz, 1H), 7.01 (s, 2H), 6.41 (d, J=8.8 Hz, 1H), 4.44-4.41 (m, 2H), 2.88-2.85 (m, 2H).

### Example 1f

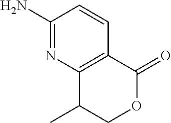

### Intermediate 20: 2-Amino-8-methyl-7,8-dihydro-5H-pyrano[4,3-b]pyridin-5-one

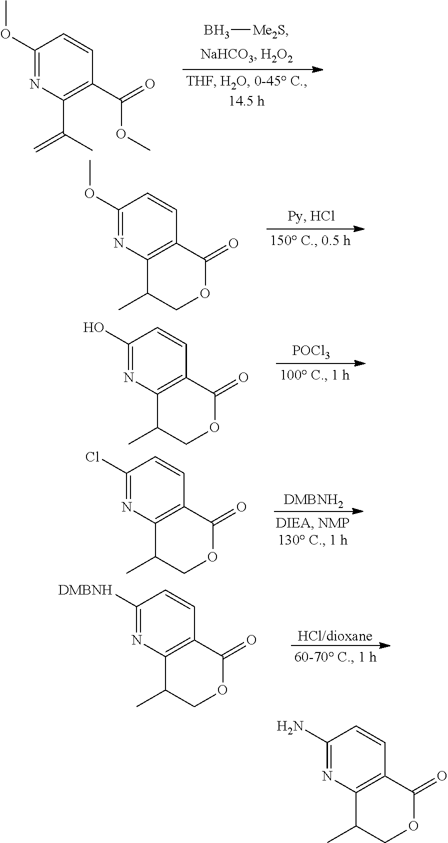

### Step 1: 2-Methoxy-8-methyl-7,8-dihydro-5H-pyrano[4,3-b]pyridin-5-one

BH₃-Me₂S (10 M, 5.31 mL, 53.1 mmol, 1.10 equiv) was added dropwise to a solution of methyl 6-methoxy-2-(prop-1-en-2-yl)nicotinate (10.0 g, 48.3 mmol, 1.00 eq) in THF (100 mL) at 0° C. The mixture was warmed to 20° C. and stirred at that temperature for 2 h. The reaction mixture was then cooled to 0° C. and NaHCO₃(20.3 g, 241 mmol, 5.00 eq) in water (35.0 mL) and H₂O₂(30% in water, 69.6 mL, 724 mmol, 15.0 eq) were added dropwise. The reaction mixture was stirred at 20° C. for 30 min and at 30-45° C. for 12 h. The reaction mixture was then poured into saturated aqueous Na₂SO₃ solution (200 mL) and extracted with EA (50.0 mL×3). The organic layers were combined and washed with brine (200 mL), dried over Na₂SO₄, filtered and concentrated. The residue was purified by flash-column chromatography on silica gel (gradient elution, 5% to 15% EA-petroleum ether) to give the title compound (15.0 g, 77.6 mmol, 80.4% yield) as yellow solid.

### Steps 2-5: -Amino-8-methyl-7,8-dihydro-5H-pyrano[4,3-b]pyridin-5-one

The title compound was prepared from 2-methoxy-8-methyl-7,8-dihydro-5H-pyrano[4,3-b]pyridin-5-one using the four-step procedure described in Steps 3, 5, 7 and 8 for Intermediate 1. MS (ES+) C₉H₁₀N₂O₂ requires: 178, found: 179[M+H]⁺. ¹H NMR: 400 MHz, DMSO-d6 δ 7.76 (d, J=8.6 Hz, 1H), 7.01 (s, 2H), 6.40 (d, J=8.6 Hz, 1H), 4.44 (dd, J=11.2 Hz, 4.4 Hz, 1H), 4.12 (dd, J=11.0 Hz, 6.8 Hz, 1H), 2.93 (td, J=7.0 Hz, 4.4 Hz, 1H), 1.20 (d, J=7.0 Hz, 3H).

### Example 1g

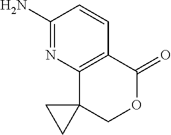 [SMILES: Nc1ccc2c(n1)C1(CC1)COC2=O]

### Intermediate 21: 2′-Amino-5′H,7′H-spiro[cyclopropane-1,8′-pyrano[4,3-b]pyridin]-5′-one

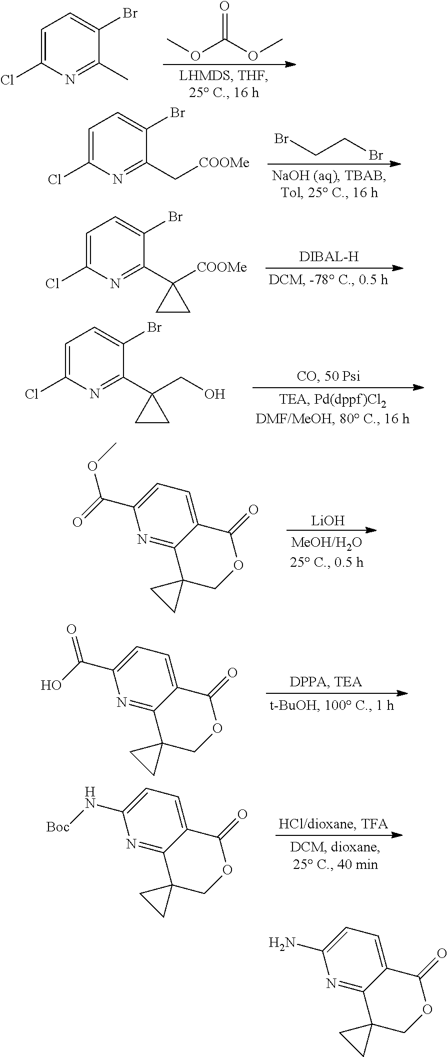

### Step 1: Methyl 2-(3-bromo-6-chloropyridin-2-yl)acetate

LiHMDS (1 M, 388 mL) was added to a solution of 3-bromo-6-chloro-2-methylpyridine (20.0 g, 96.9 mmol) in THF (300 mL) at 25° C. under nitrogen. After 2.5 h, dimethyl carbonate (14.0 g, 155 mmol) was added to the mixture and stirred at 25° C. for 13.5 h. The reaction mixture was then was added to saturated aqueous NH₄Cl (1000 mL) and extracted with EA (60 mL×3). The combined organic layers were dried over anhydrous sodium sulfate, filtered and concentrated to give a residue. The residue was purified by flash-column chromatography on silica gel (gradient elution, 0% to 10% EA-petroleum ether) to give the title compound (18.0 g, 70% yield) as a yellow oil. ¹H NMR (400 MHz, CDCl3): δ ppm 7.81 (d, J=8.4 Hz, 1H), 7.16 (d, J=8.0 Hz, 1H), 4.03 9s, 2H), 3.74 (s, 3H).

### Step 2: Methyl 1-(3-bromo-6-chloropyridin-2-yl)cyclopropane-1-carboxylate

Tetrabutylammonium bromide (2.44 g, 7.56 mmol) and NaOH (50 mL, 50 wt % in water) were added to a solution of 1,2-dibromoethane (10.7 g, 56.7 mmol) and methyl 2-(3-bromo-6-chloropyridin-2-yl)acetate (10.0 g, 37.8 mmol) in toluene (50 mL) at 25° C. The reaction mixture was stirred at 25° C. for 16 h, then was diluted with water (300 mL) and extracted with EA (200 mL×3). The combined organic layers were dried over anhydrous sodium sulfate, filtered and concentrated to give a residue. The residue was purified by flash-column chromatography on silica gel (gradient elution, 0% to 10% EA-petroleum ether) to give the title compound (6.10 g, 56% yield) as a yellow solid. ¹H NMR (400 MHz, CDCl₃): δ ppm 7.81 (d, J=8.4 Hz, 1H), 7.15 (d, J=8.0 Hz, 1H), 3.66 (s, 3H), 1.81-1.75 (m, 2H), 1.46-1.41 (m, 2H).

### Step 3: (1-(3-Bromo-6-chloropyridin-2-yl)cyclopropyl)methanol

Diisobutylaluminium hydride (1 M, 56 mL) was added to a solution of methyl 1-(3-bromo-6-chloropyridin-2-yl)cyclopropane-1-carboxylate (5.40 g, 18.6 mmol) in DCM (80 mL) at −78° C. under nitrogen. The reaction mixture was stirred at −78° C. for 0.5 h, then was quenched by addition of aqueous saturated NH₄Cl solution (50 mL), diluted with water (200 mL) and extracted with EA (200 mL×3). The combined organic layers were dried over anhydrous sodium sulfate, filtered and concentrated to give the crude title compound (5.00 g, crude) as a yellow solid which was used in the next step without further purification.

### Step 4: Methyl 5′-oxo-5′H,7′H-spiro[cyclopropane-1,8′-pyrano[4,3-b]pyridine]-2′-carboxylate

Triethylamine (2.31 g, 22.9 mmol) and Pd(dppf)Cl₂ (557 mg, 762 μmol) were added to a solution of (1-(3-Bromo-6-chloropyridin-2-yl)cyclopropyl)methanol in MeOH (25 mL) and DMF (25 mL) under nitrogen atmosphere. The suspension was degassed under vacuum and purged with carbon monoxide several times. The mixture was stirred under carbon monoxide (50 psi) at 80° C. for 16 h. The reaction mixture was then concentrated to remove methanol, diluted with water (100 mL) and extracted with EA (60 mL×3). The combined organic layers were washed with brine (100 mL×3), dried over anhydrous sodium sulfate, filtered and concentrated to give a residue. The title compound (1.8 g, crude) was obtained as a yellow solid and used in the next step without further purification. MS (ES+) C₁₀H₁₀N₂O₂ requires: 233, found: 234[M+H]⁺.

### Step 5: 5′-Oxo-5′H,7′H-spiro[cyclopropane-1,8′-pyrano[4,3-b]pyridine]-2′-carboxylic acid

Lithium hydroxide (555 mg, 23.2 mmol) was added to a solution of methyl 5′-oxo-5′H,7′H-spiro[cyclopropane-1,8′-pyrano[4,3-b]pyridine]-2′-carboxylate (1.80 g, 7.72 mmol) in methanol (30 mL) and water (10 mL). The reaction mixture was stirred at 25° C. for 0.5 h, then was concentrated to remove the methanol. The mixture was diluted with water (60 mL) and extracted with EA (50 mL×3). The aqueous layer was acidified by addition aqueous hydrochloric acid solution (6 M, 5 mL), then the mixture was extracted with EA (50 mL×3). The combined organic layers were dried over anhydrous sodium sulfate, filtered and concentrated to give the title compound (1.20 g, 71% yield) as a brown solid that was used without further purification.

### Step 6: tert-Butyl (5′-oxo-5′H,7′H-spiro[cyclopropane-1,8′-pyrano[4,3-b]pyridin]-2′-yl)carbamate

Triethylamine (831 mg, 8.21 mmol) and diphenyl phosphoryl azide (2.26 g, 8.21 mmol) were added to a solution of 5′-oxo-5′H,7′H-spiro[cyclopropane-1,8′-pyrano[4,3-b]pyridine]-2′-carboxylic acid (1.20 g, 5.47 mmol) in tert-butanol (20 mL). The reaction mixture was stirred at 100° C. for 1 h, then was cooled to ambient temperature, diluted with water (60 mL), and extracted with EA (50 mL×3). The combined organic layers were dried over anhydrous sodium sulfate, filtered and concentrated to give a residue. The residue was purified by flash-column chromatography on silica gel (gradient elution, 0% to 50% EA-petroleum ether) to give the title compound (330 mg, 19% yield) as a yellow solid and 2′-amino-5′H,7′H-spiro[cyclopropane-1,8′-pyrano[4,3-b]pyridin]-5′-one (420 mg, 28% yield) as a yellow oil.

### Step 7: 2′-Amino-5′H,7′H-spiro[cyclopropane-1,8′-pyrano[4,3-b]pyridin]-5′-one

HCl in dioxane (4.0 M, 0.5 mL) was added to a solution of tert-butyl (5′-oxo-5′H,7′H-spiro[cyclopropane-1,8′-pyrano[4,3-b]pyridin]-2′-yl)carbamate (100 mg, 344 μmol) in dioxane (1.5 mL) at 25° C. The reaction mixture was stirred for 10 min, then was concentrated. DCM (2 mL) and TFA (1 mL, 13.5 mmol) were added to the residue, and the reaction mixture was stirred at 25° C. for 30 min. The reaction mixture was then concentrated and EA (5 mL) was added to the residue. The mixture was neutralized by addition of saturated aqueous NaHCO₃(20 mL) and extracted with EA (15 mL×3). The combined organic layers were dried over anhydrous sodium sulfate, filtered and concentrated under to give the title compound (60.0 mg, 92% yield) as a yellow oil that was used in the next step without further purification. MS (ES+) C₁₂H₁₁NO₄ requires: 190, found: 191[M+H]⁺.

### Example 1h

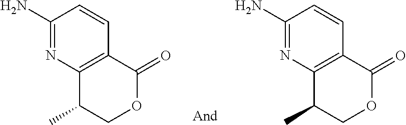

### Intermediates 22 and 23: (R)-2-Amino-8-methyl-7,8-dihydro-5H-pyrano[4,3-b]pyridin-5-one and (S)-2-amino-8-methyl-7,8-dihydro-5H-pyrano[4,3-b]pyridin-5-one

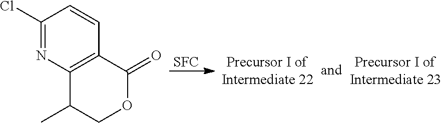 [0268] each of which is represented by one of the structures shown below:

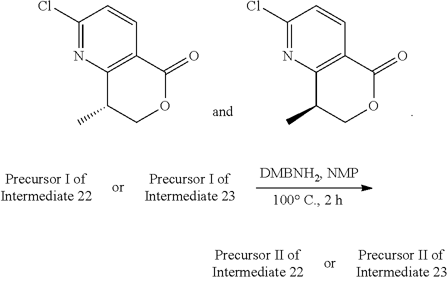

each of which is represented by one of the structures shown below:

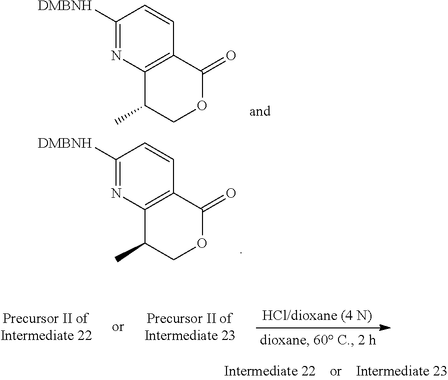

each of which is represented by one of the structures shown below:

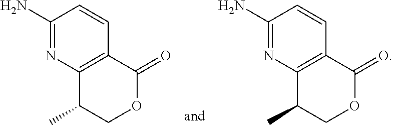

### Step 1: (R)-2-chloro-8-methyl-7,8-dihydro-5H-pyrano[4,3-b]pyridin-5-one and (S)-2-chloro-8-methyl-7,8-dihydro-5H-pyrano[4,3-b]pyridin-5-one

rac-2-Chloro-8-methyl-7,8-dihydro-5H-pyrano[4,3-b]pyridin-5-one (title compound from Step 3 of Intermediate 20, 700 mg, 3.54 mmol) was separated by SFC (Daicel Chiralpak IG, MeOH gradient in CO₂ with 0.1% NH₄OH) to give two peaks separately. The first eluting isomer (330 mg, 47% yield) and second eluting isomer (330 mg, 47% yield) were obtained as yellow solids.

### Steps 2 and 3: One of (R or S)-2-amino-8-methyl-7,8-dihydro-5H-pyrano[4,3-b]pyridin-5-one

The title compound (Intermediate 22) was prepared from one of (R or S)-2-chloro-8-methyl-7,8-dihydro-5H-pyrano[4,3-b]pyridin-5-one (first eluting isomer from step 1) using the same two-step procedure as described in Steps 4 and 5 for Intermediate 17. MS (ES+) C₉H₁₀N₂O₂ requires: 178, found: 179[M+H]⁺.

### Steps 3 and 4: The remaining one of (R or S)-2-amino-8-methyl-7,8-dihydro-5H-pyrano[4,3-b]pyridin-5-one

The title compound (Intermediate 23) was prepared from the remaining one of (R or S)-2-chloro-8-methyl-7,8-dihydro-5H-pyrano[4,3-b]pyridin-5-one (second eluting isomer from step 1) using the same two-step procedure as described in Steps 4 and 5 for Intermediate 17. MS (ES+) C₉H₁₀N₂O₂ requires: 178, found: 179[M+H]⁺.

### Example 1i

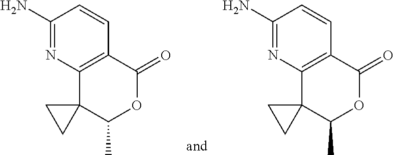

### Intermediates 24 and 25: (R)-2′-Amino-7′-methyl-5′H,7′H-spiro[cyclopropane-1,8′-pyrano[4,3-b]pyridin]-5′-one and (S)-2′-Amino-7′-methyl-5′H,7′H-spiro[cyclopropane-1,8′-pyrano[4,3-b]pyridin]-5′-one

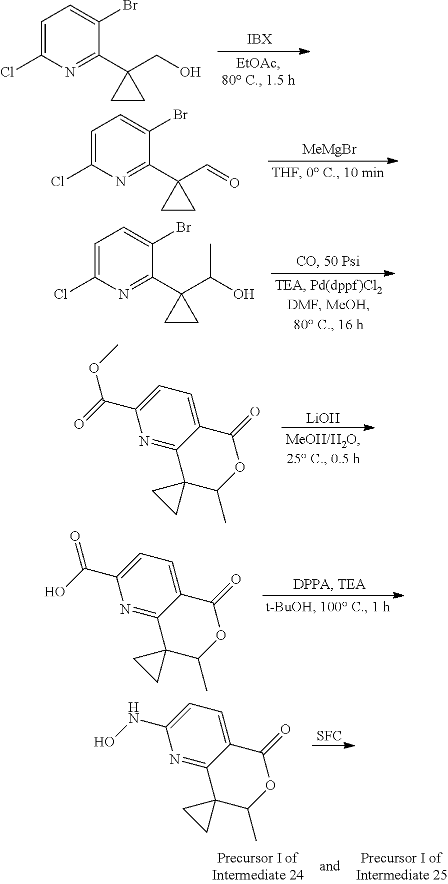 [0276] each of which is represented by one of the structures shown below:

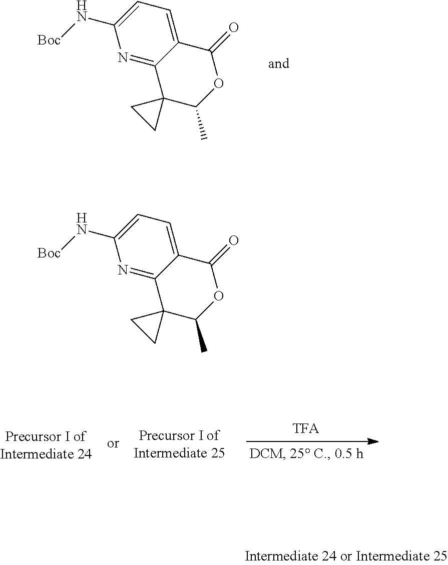 [SMILES: C[C@H]1OC(=O)c2ccc(NC(=O)OC(C)(C)C)nc2C12CC2 ; C[C@@H]1OC(=O)c2ccc(NC(=O)OC(C)(C)C)nc2C12CC2]

each which is represented by one of the structures shown below:

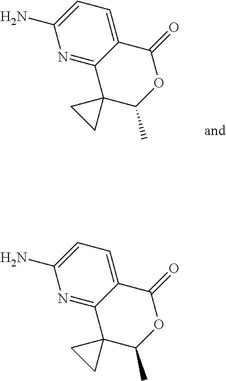

### Step 1: 1-(3-Bromo-6-chloropyridin-2-yl)cyclopropane-1-carbaldehyde

IBX (6.50 g, 10.7 mmol, 46% purity) was added to a solution of (1-(3-bromo-6-chloropyridin-2-yl)cyclopropyl)methanol (2.65 g, 10.1 mmol) in EA (80 mL). The reaction mixture was stirred at 80° C. for 1 h, then additional IBX (2.00 g, 3.29 mmol, 46% purity) was added. The reaction mixture was stirred at 80° C. for 0.5 h, then was filtered and concentrated to give the title compound (2.60 g, crude) as a yellow solid that was used without further purification. MS (ES+) C₉H₇BrClNO requires: 261, found: 262 [M+H]⁺.

### Step 2: 1-(1-(3-Bromo-6-chloropyridin-2-yl)cyclopropyl)ethan-1-ol

Methylmagnesium bromide (3 M, 17 mL) was added to a solution of 1-(3-bromo-6-chloropyridin-2-yl)cyclopropane-1-carbaldehyde (2.60 g, 9.98 mmol) in THF (80 mL) at 0° C. The reaction mixture was stirred for 10 min, then was quenched by addition of aqueous saturated NH₄Cl solution (80 mL), diluted with water (40 mL) and extracted with EA (80 mL×3). The combined organic layers were dried over anhydrous sodium sulfate, filtered and concentrated to give the title compound (2.70 g, crude) as a yellow oil that was used without further purification. MS (ES+) C₁₀H₁₁N₂O₂ requires: 277, found: 278 [M+H]⁺.

### Steps 3-5: tert-Butyl (7′-methyl-5′-oxo-5′H,7′H-spiro[cyclopropane-1,8′-pyrano[4,3-b]pyridin]-2′-yl)carbamate

The title compound was prepared from 1-(1-(3-bromo-6-chloropyridin-2-yl)cyclopropyl)ethan-1-ol using a similar procedure as described in Steps 4-6 for Intermediate 21 above. ¹H NMR (400 MHz, CDCl₃): δ ppm 8.29 (d, J=8.8 Hz, 1H), 7.90 (d, J=8.8 Hz, 1H), 7.31 (s, 1H), 4.63-4.53 (m, 1H), 1.61 (s, 3H), 1.53 (s, 9H), 1.38-1.35 (m, 1H), 1.09-1.00 (m, 2H).

### Step 6: tert-Butyl (R)-(7′-methyl-5′-oxo-5′H,7′H-spiro[cyclopropane-1,8′-pyrano[4,3-b]pyridin]-2′-yl)carbamate and tert-butyl (S)-(7′-methyl-5′-oxo-5′H,7′H-spiro[cyclopropane-1,8′-pyrano[4,3-b]pyridin]-2′-yl)carbamate

tert-Butyl (7′-methyl-5′-oxo-5′H,7′H-spiro[cyclopropane-1,8′-pyrano[4,3-b]pyridin]-2′-yl)carbamate (400 mg) was separated by SFC (column: REGIS (s,s) WHELK-01 (250 mm×50 mm, 10 um), EtOH gradient in CO₂ with 0.1% NH₄OH) to give two separate peaks. The first eluting isomer (100 mg, 24% yield) and second eluting isomer (140 mg, 34% yield) were obtained as yellow solids.

### Step 7: One of (R or S)-2′-amino-7′-methyl-5′H,7′H-spiro[cyclopropane-1,8′-pyrano[4,3-b]pyridin]-5′-one

TFA (2.31 g, 20.3 mmol) was added to a solution of one of tert-butyl (R or S)-(7′-methyl-5′-oxo-5′H,7′H-spiro[cyclopropane-1,8′-pyrano[4,3-b]pyridin]-2′-yl)carbamate (first eluting isomer from Step 6, 100 mg) in DCM (6 mL). The reaction mixture was stirred at 25° C. for 30 min, then was quenched with saturated aqueous NaHCO₃ solution (30 mL) and extracted with DCM (20 mL×3). The combined organic layers were dried over anhydrous sodium sulfate, filtered and concentrated to give the title compound (Intermediate 24, 70 mg, crude) as a yellow oil that was used without further purification. MS (ES+) C₁₁H₁₂N₂O₂ requires: 204, found: 205[M+H]⁺.

### Step 8: The remaining one of (R or S)-2′-amino-7′-methyl-5′H,7′H-spiro[cyclopropane-1,8′-pyrano[4,3-b]pyridin]-5′-one

The title compound (Intermediate 25) was prepared from one of tert-butyl (R or S)-(7′-methyl-5′-oxo-5′H,7′H-spiro[cyclopropane-1,8′-pyrano[4,3-b]pyridin]-2′-yl)carbamate (second eluting isomer from Step 6) using the same procedure as described in Step 7 for Intermediate 24. MS (ES+) C₁₁H₁₂N₂O₂ requires: 204, found: 205[M+H]⁺.

### Example 1j

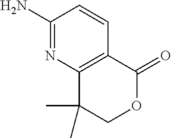

### Intermediate 26: 2-Amino-8,8-dimethyl-7,8-dihydro-5H-pyrano[4,3-b]pyridin-5-one

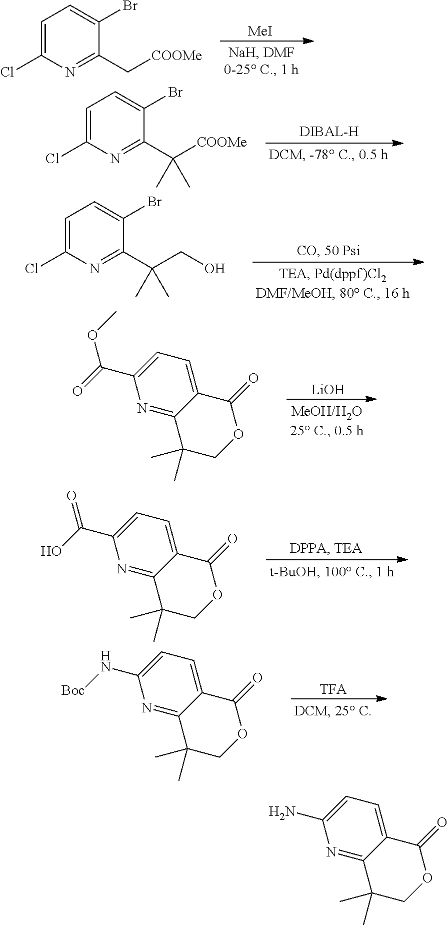

### Step 1: Methyl 2-(3-bromo-6-chloropyridin-2-yl)-2-methylpropanoate

Sodium hydride (2.91 g, 72.8 mmol, 60% purity) was added to a solution of methyl 2-(3-bromo-6-chloropyridin-2-yl)acetate (5.50 g, 20.8 mmol) in THF (20 mL) at 0° C. The reaction mixture was stirred for 15 minutes at 0° C., then iodomethane (7.38 g, 51.9 mmol) was added. The reaction mixture was warmed to 25° C. and stirred for 45 min, then was quenched with water (30 mL) and extracted with EA (30 mL×2). The combined organic layers were concentrated to give a residue. The residue was purified by flash-column chromatography on silica gel (gradient elution, 0% to 10% EA-petroleum ether) to give the title compound (5.5 g, 90% yield) as a yellow oil.

¹H NMR (400 MHz, CD₃OD): δ ppm 7.97 (d, J=8.4 Hz, 1H), 7.20 (d, J=8.4 Hz, 1H), 3.95 (s, 2H), 1.50 (s, 6H).

### Steps 2-6: 2-Amino-8,8-dimethyl-7,8-dihydro-5H-pyrano[4,3-b]pyridin-5-one

The title compound was prepared using similar procedures as described in Steps 3-6 of Intermediate 21 and Step 7 of Intermediate 24. MS (ES+) C₁₀H₁₂N₂O₂ requires: 192, found: 193 [M+H]⁺. ¹H NMR (400 MHz, 6d-DMSO): δ ppm 7.77 (d, J=8.8 Hz, 1H), 7.01 (s, 2H), 6.40 (d, J=8.8 Hz, 1H), 4.15 (s, 2H), 1.21 (s, 6H).

### Example 1k

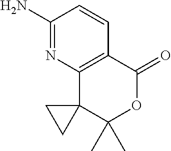 [SMILES: CC1(C)OC(=O)c2ccc(N)nc2C12CC2]

### Intermediate 27: 2′-Amino-7′,7′-dimethyl-5′H,7′H-spiro[cyclopropane-1,8′-pyrano[4,3-b]pyridin]-5′-one

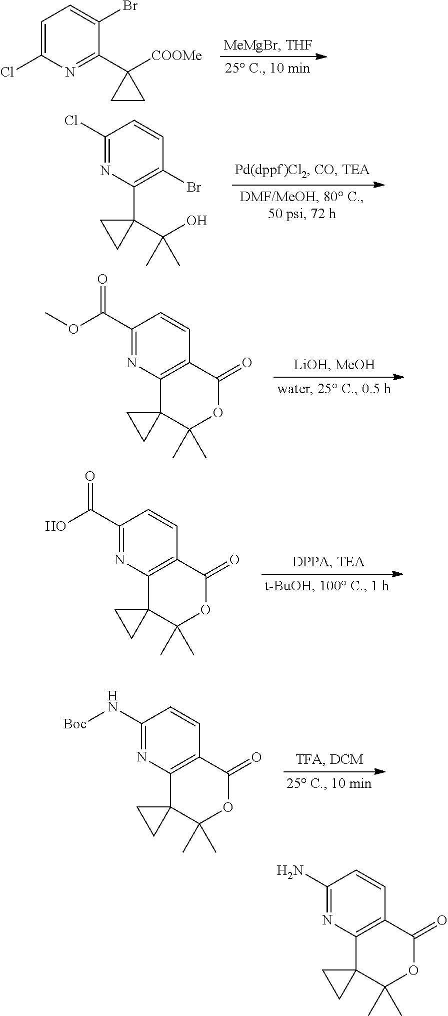

### Step 1: 2-(1-(3-Bromo-6-chloropyridin-2-yl)cyclopropyl)propan-2-ol

To a solution of methyl 1-(3-bromo-6-chloropyridin-2-yl)cyclopropane-1-carboxylate (1.3 g, 4.47 mmol) in THF (10 mL) was added MeMgBr (3 M, 14.9 mL) at 25° C. The reaction mixture was stirred at 25° C. for 10 min, then was poured into water (20 mL) and extracted with EA (50 mL×3). The organic layers were combined and dried over sodium sulfate, filtered, and concentrated to give a residue. The residue was purified by column chromatography (SiO₂, PE/EA=1/0 to 50/1) to give the title compound (500 mg, 38% yield) as colorless oil.

### Steps 2-5: 2′-Amino-7′,7′-dimethyl-5′H,7′H-spiro[cyclopropane-1,8′-pyrano[4,3-b]pyridin]-5′-one

The title compound was prepared from 2-(1-(3-Bromo-6-chloropyridin-2-yl)cyclopropyl)propan-2-ol using a similar procedure as described in Steps 4-6 for Intermediate 21 and Step 7 of Intermediate 24 above. ¹H NMR (400 MHz, CDCl₃): δ ppm 8.05 (d, J=8.4 Hz, 1H), 6.36 (d, J=8.8 Hz, 1H), 4.85 (s, 2H), 1.42-1.32 (m, 8H), 1.06-1.03 (m, 2H).

### Example 2: Synthesis of Arylchloride Intermediates

### Example 2a

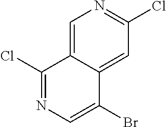 [SMILES: Clc1cc2c(Br)cnc(Cl)c2cn1]

### Intermediate 5: 4-Bromo-1,6-dichloro-2,7-naphthyridine

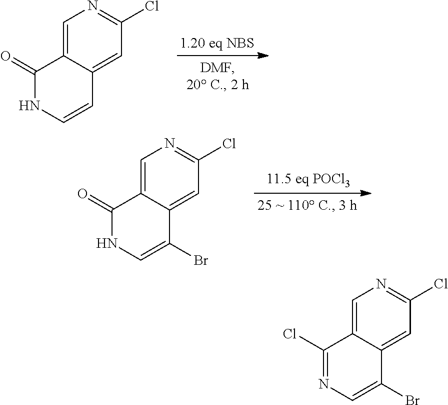 [SMILES: O=c1[nH]ccc2cc(Cl)ncc12 ; O=c1[nH]cc(Br)c2cc(Cl)ncc12 ; Clc1cc2c(Br)cnc(Cl)c2cn1]

### Step 1: 4-Bromo-6-chloro-2,7-naphthyridin-1(2H)-one

NBS (70.9 g, 398 mmol, 1.20 eq) was added to a solution of 6-chloro-2,7-naphthyridin-1(2H)-one (60.0 g, 332 mmol, 1.00 eq) in DMF (600 mL). The reaction mixture was stirred at 20° C. for 2 h, then was poured into water (1 L) and filtered. The filter cake was dried under vacuum to give 4-bromo-6-chloro-2,7-naphthyridin-1(2H)-one (90.8 g, crude) as a brown solid. MS (ES+) C₈H₄BrClN₂O requires: 260, found: 261[M+H]⁺.

### Step 2: 4-Bromo-1,6-dichloro-2,7-naphthyridine

4-Bromo-6-chloro-2,7-naphthyridin-1(2H)-one (70.8 g, 272 mmol, 1.00 eq) was added in portions to POCl₃ (484 g, 3.16 mol, 293 mL, 11.5 eq) at 25° C. The reaction mixture was then stirred at 110° C. for 3 h. The reaction mixture was then concentrated under vacuum, and the residue was adjusted to pH=8 with saturated aqueous Na₂CO₃ at 25° C. The mixture was extracted with DCM (500 mL×3), washed with brine (500 mL), dried over Na₂SO₄, filtered and concentrated under vacuum to give the title compound (75.0 g, 269 mmol, 98.9% yield) as a yellow solid. MS (ES+) C₈H₃BrCl₂N₂ requires: 278, found: 279[M+H]⁺.

### Example 2b

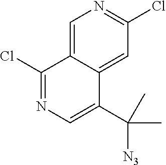 [SMILES: CC(C)(N=[N+]=[N-])c1cnc(Cl)c2cnc(Cl)cc12]

### Intermediate 6: 4-(2-Azidopropan-2-yl)-1,6-dichloro-2,7-naphthyridine

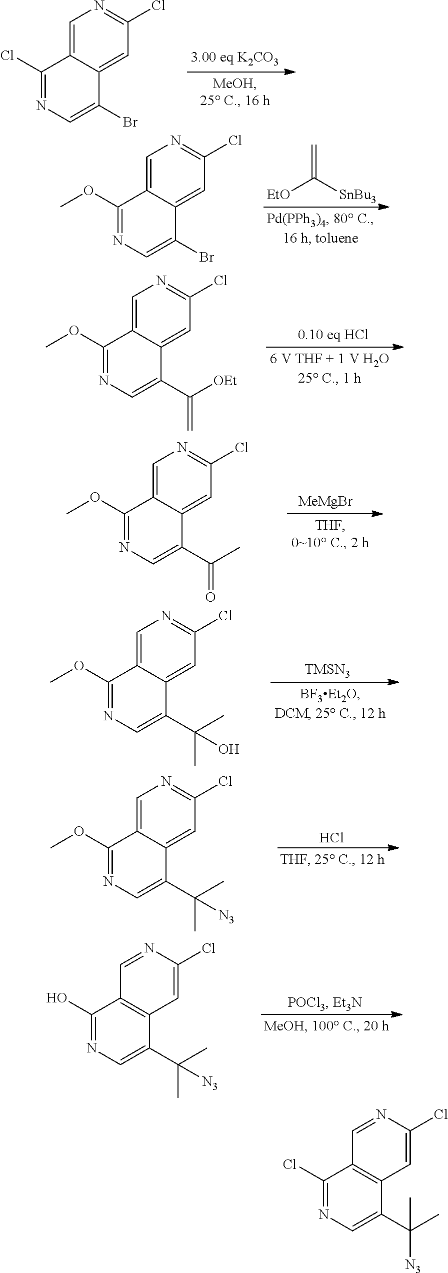

### Step 1: 4-Bromo-6-chloro-1-methoxy-2,7-naphthyridine

A suspension of 4-bromo-1,6-dichloro-2,7-naphthyridine (75.0 g, 269 mmol, 1.00 eq), K₂CO₃ (111 g, 809 mmol, 3.00 eq) in MeOH (3 L) was stirred at 25° C. for 16 h. The reaction mixture was then concentrated under vacuum, and the residue was dissolved in H₂O (300 mL) and extracted with DCM (100 mL×2). The combined organic layers were concentrated under vacuum to give a residue. The residue was triturated in PE/EA (40 mL 20:1) and filtered. The filter cake was dried under vacuum to give the title compound (47.0 g, 171 mmol, 63.6% yield) as a yellow solid.

### Step 2: 6-Chloro-4-(1-ethoxyvinyl)-1-methoxy-2,7-naphthyridine

A solution of 4-bromo-6-chloro-1-methoxy-2,7-naphthyridine (47.0 g, 171 mmol, 1.00 eq), tributyl(1-ethoxyvinyl)stannane (74.4 g, 206 mmol, 69.6 mL, 1.20 eq) and Pd(PPh₃)₄(19.8 g, 17.1 mmol, 0.10 eq) in toluene (500 mL) was stirred at 80° C. for 16 h under N₂. The reaction mixture was then cooled to 20° C. and poured into saturated aqueous KF solution (500 mL) and stirred for 1 h. The aqueous mixture was extracted with EA (300 mL×3), and the organic layers were combined. The combined organic layer was concentrated under vacuum to give the title compound (64.0 g, crude) as a yellow oil. MS (ES+) C₁₃H₁₃ClN₂O₂ requires: 264, found: 265[M+H]⁺.

### Step 3: 1-(6-Chloro-1-methoxy-2,7-naphthyridin-4-yl)ethan-1-one

Aqueous HCl (1.50 M, 20.1 mL, 0.10 eq) was added to a solution of 6-chloro-4-(1-ethoxyvinyl)-1-methoxy-2,7-naphthyridine (80.0 g, 302 mmol, 1.00 eq) in THF (480 mL) and H₂O (80 mL). The reaction mixture was stirred at 25° C. for 1 h. The reaction mixture was then poured into saturated aqueous NaHCO₃ solution (500 mL) and extracted with EA (300 mL×2). The organic layers were combined and concentrated under vacuum. The residue was purified by flash-column chromatography on silica gel (gradient elution, 5% to 50% EA-PE) to give the title compound (28.0 g, 118 mmol, 39.1% yield) as a white solid. MS (ES+) C₁₁H₉ClN₂O₂ requires: 236, found: 237[M+H]⁺.

### Step 4: 2-(6-Chloro-1-methoxy-2,7-naphthyridin-4-yl)propan-2-ol

MeMgBr (3.0 M in diethyl ether, 118 mL, 3.00 eq) was added to a solution of 1-(6-chloro-1-methoxy-2,7-naphthyridin-4-yl)ethan-1-one (28.0 g, 118 mmol, 1.00 eq) in THF (300 mL) at 0˜10° C. The mixture was stirred at 0-10° C. for 2 h, and then was poured into saturated aqueous NH₄Cl solution (300 mL) and extracted with EA (200 mL×2). The organic layers were combined and concentrated under vacuum to give the title compound (33 g, crude) as a yellow oil. MS (ES+) C₁₂H₁₃ClN₂O₂ requires: 252, found: 253[M+H]⁺.

### Step 5: 4-(2-Azidopropan-2-yl)-6-chloro-1-methoxy-2,7-naphthyridine

TMSN₃ (14.4 g, 125 mmol, 16.5 mL, 2.50 eq) was added to a solution of 2-(6-chloro-1-methoxy-2,7-naphthyridin-4-yl)propan-2-ol (28.0 g, 50.3 mmol, 45.4% purity, 1.00 eq) and BF₃·Et₂O (15.5 g, 50.3 mmol, 13.5 mL, 46.0% purity, 1.00 eq) in DCM (280 mL) at 25° C. The reaction mixture was stirred at 25° C. for 12 h, then was partitioned between saturated aqueous NaHCO₃ solution (350 mL) and DCM (200 mL). The layers were separated, and the aqueous layer was further extracted with DCM (200 mL). The organic layers were combined and washed with brine (200 mL), dried over sodium sulfate, filtered, and then concentrated. The residue was purified by flash-column chromatography on silica gel (gradient elution, 2% to 50% EA-PE) to give the title compound (20.0 g, 65.% yield, 91% purity) as an off-white solid. MS (ES+) C₁₂H₁₂ClN₅O requires: 277, found: 278[M+H]⁺.

### Step 6: 4-(2-Azidopropan-2-yl)-6-chloro-2,7-naphthyridin-1-ol

Aqueous HCl (2 M, 163 mL, 5.00 eq) was added to a solution of 4-(2-azidopropan-2-yl)-6-chloro-1-methoxy-2,7-naphthyridine (20.0 g, 65.5 mmol, 91% purity, 1.00 eq) in THF (200 mL) at 25° C. The reaction mixture was stirred at 25° C. for 12 h, then was poured into saturated aqueous NaHCO₃(1.00 L) and extracted with EA (500 mL*2). The organic layers were combined and washed with brine (500 mL), dried over sodium sulfate, filtered, and concentrated to give the title compound (19.0 g, 96% yield, 88% purity) as a yellow solid. MS (ES+) C₁₁H₁₀ClN₅O requires: 263, found: 264[M+H]⁺.

### Step 7: 4-(2-Azidopropan-2-yl)-1,6-dichloro-2,7-naphthyridine

POCl₃ (2.54 g, 16.5 mmol, 1.54 mL, 4.37 eq) and Et₃N (1.01 g, 9.99 mmol, 1.39 mL, 2.63 eq) were added to a mixture of 4-(2-azidopropan-2-yl)-6-chloro-2,7-naphthyridin-1-ol 7 (1.00 g, 3.79 mmol, 1.00 eq) in ACN (20.0 mL). The reaction mixture was then heated to 100° C. for 20 h. The reaction mixture was then concentrated and the residue was diluted with EA. The diluted residue was quenched with water, and the mixture was adjusted to pH=8 with aqueous sodium carbonate. The mixture was then extracted with EA, and the organic layer was washed with brine. The washed organic layer was dried over sodium sulfate, filtered, and concentrated. The residue was purified by flash-column chromatography on silica gel (gradient elution, 0% to 5% EA-PE) to give the title compound (14.2 g, 84% yield) as a light yellow solid. MS (ES+) C₁₁H₉Cl₂N₂ requires: 281, found: 282[M+H]⁺.

¹H-NMR (400 MHz, DMSO-d₆): δ ppm 9.57 (s, 1H), 8.61 (s, 1H), 8.45 (s, 1H), 1.83 (s, 6H).

### Example 2c

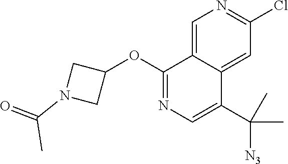 [SMILES: CC(=O)N1CC(Oc2ncc(C(C)(C)N=[N+]=[N-])c3cc(Cl)ncc23)C1]

### Intermediate 7: 1-(3-((4-(2-Azidopropan-2-yl)-6-chloro-2,7-naphthyridin-1-yl)oxy)azetidin-1-yl)ethan-1-one

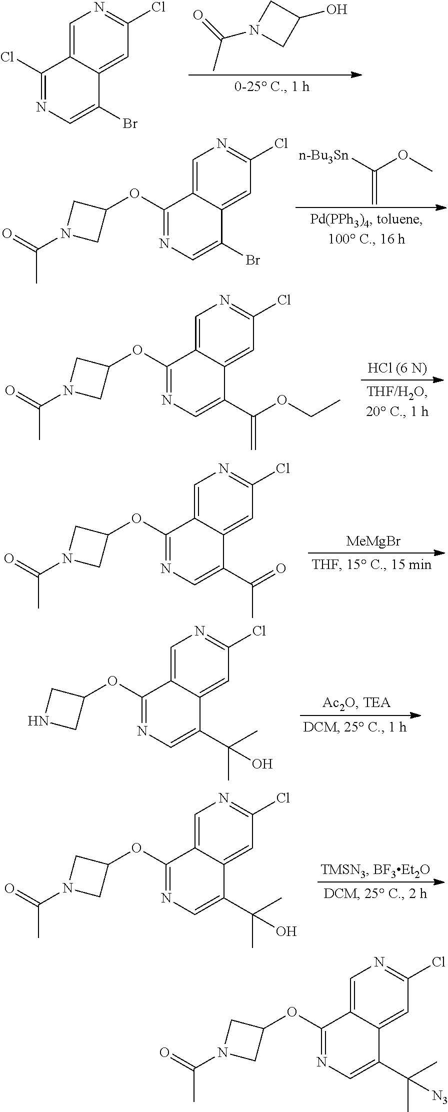

### Step 1: 1-(3-((4-Bromo-6-chloro-2,7-naphthyridin-1-yl)oxy)azetidin-1-yl)ethan-1-one

Sodium hydride (2.15 g, 53.8 mmol, 60 wt %) was added to a solution of 1-(3-hydroxyazetidin-1-yl)ethan-1-one (Combi-Blocks #ST-8959) (5.16 g, 44.8 mmol) in THF (200 mL) at 0° C. The cooling bath was removed, the reaction mixture was stirred for 0.5 h, and then 4-bromo-1,6-dichloro-2,7-naphthyridine (Intermediate 5) (12.4 g, 44.8 mmol) was added. The reaction mixture was stirred at 25° C. for 1 h in MeOH, then was quenched by addition of water (70 mL). The quenched reaction mixture was extracted with EA, dried over sodium sulfate, filtered, and concentrated to give the title compound (10.0 g, 63% yield).

### Step 2: 1-(3-((6-Chloro-4-(1-ethoxyvinyl)-2,7-naphthyridin-1-yl)oxy)azetidin-1-yl)ethan-1-one

A mixture of compound tributyl(1-ethoxyvinyl)stannane (9.14 g, 25.3 mmol 1-(3-((4-bromo-6-chloro-2,7-naphthyridin-1-yl)oxy)azetidin-1-yl)ethan-1-one (8.20 g, 23.0 mmol) and Pd(PPh₃)₄(2.66 g, 2.30 mmol) in toluene (150 mL) was degassed and purged with nitrogen 3 times, and then the mixture was stirred at 100° C. for 16 h. The reaction mixture was cooled to ambient temperature, quenched by addition aqueous saturated KF (300 mL), diluted with water (100 mL), and extracted with EA (200 mL×3). The combined organic layers were dried over anhydrous sodium sulfate, filtered and concentrated to give a residue. The residue was purified by flash-column chromatography on silica gel to give the title compound (7.50 g, 94% yield) as a yellow solid.

### Step 3: 1-(3-((4-Acetyl-6-chloro-2,7-naphthyridin-1-yl)oxy)azetidin-1-yl)ethan-1-one

Aqueous hydrochloric acid (6 M, 0.5 mL) was added to a solution of 1-(3-((6-chloro-4-(1-ethoxyvinyl)-2,7-naphthyridin-1-yl)oxy)azetidin-1-yl)ethan-1-one (6.90 g, 19.8 mmol) in THF (105 mL) and water (35 mL) at 20° C. The reaction mixture was stirred at 20° C. for 1 h, then was diluted with water (300 mL) and extracted with EA (200 mL×3). The combined organic layers were dried over sodium sulfate, filtered and concentrated. The residue was purified by flash-column chromatography on silica gel (gradient elution, 0% to 100% EA-PE) to give the title compound (5.00 g, 79% yield) as a yellow solid.

### Steps 4 and 5: 1-(3-((6-Chloro-4-(2-hydroxypropan-2-yl)-2,7-naphthyridin-1-yl)oxy)azetidin-1-yl)ethan-1-one

Methylmagnesium bromide (3 M in diethyl ether, 14 mL) was added to a solution of 1-(3-((4-acetyl-6-chloro-2,7-naphthyridin-1-yl)oxy)azetidin-1-yl)ethan-1-one (4.50 g, 14.1 mmol) in THF (200 mL) at 0° C. The reaction mixture was stirred at 0° C. for 15 min, then was quenched with water (200 mL) and extracted with EA (150 mL×3). The combined organic layers were dried over sodium sulfate, filtered and concentrated to give a mixture of 1-(3-((6-chloro-4-(2-hydroxypropan-2-yl)-2,7-naphthyridin-1-yl)oxy)azetidin-1-yl)ethan-1-one and 2-(1-(azetidin-3-yloxy)-6-chloro-2,7-naphthyridin-4-yl)propan-2-ol (4.10 g, 99% yield) as a yellow oil. The mixture was dissolved in DCM (50 mL) and TEA (3.53 g, 34.9 mmol) and acetic anhydride (2.14 g, 20.9 mmol) were added. The reaction mixture was stirred at 25° C. for 1 h, then was diluted with water (200 mL) and extracted with DCM (150 mL×3). The combined organic layers were dried over sodium sulfate, filtered and concentrated to give a residue. The residue was purified by flash-column chromatography on silica gel (gradient elution, 50% to 100% EA-PE, then 5% to 10% MeOH-EA) to give the title compound (4.10 g, 87% yield) as a yellow oil.

### Step 6: 1-(3-((4-(2-Azidopropan-2-yl)-6-chloro-2,7-naphthyridin-1-yl)oxy)azetidin-1-yl)ethan-1-one

Azidotrimethylsilane (6.69 g, 58.1 mmol) and boron trifluoride diethyl etherate (6.59 g, 46.5 mmol) were added to a solution of 1-(3-((6-chloro-4-(2-hydroxypropan-2-yl)-2,7-naphthyridin-1-yl)oxy)azetidin-1-yl)ethan-1-one (3.90 g, 11.6 mmol) in DCM (100 mL). The reaction mixture was stirred at 25° C. for 2 h, then was diluted with water (200 mL) and extracted with DCM (150 mL×3). The combined organic layers were dried over sodium sulfate, filtered and concentrated to give a residue. The residue was purified by flash-column chromatography on silica gel (gradient elution, 0% to 100% EA-PE) to give the title compound (3.70 g, 88% yield) as a yellow oil. ¹H-NMR (400 MHz, CDCl₃): δ ppm 9.47 (s, 1H), 8.39 (s, 1H), 8.15 (s, 1H), 5.64-5.57 (m, 1H), 4.68-4.60 (m, 1H), 4.56-4.48 (m, 1H), 4.36-4.27 (m, 1H), 4.25-4.17 (m, 1H), 1.95 (s, 3H), 1.81 (d, J=8.0 Hz, 6H).

### Example 2d

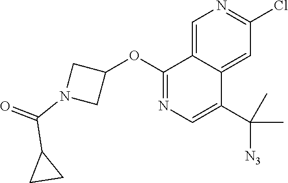 [SMILES: CC(C)(N=[N+]=[N-])c1cnc(OC2CN(C(=O)C3CC3)C2)c2cnc(Cl)cc12]

### Intermediate 8: (3-((4-(2-Azidopropan-2-yl)-6-chloro-2,7-naphthyridin-1-yl)oxy)azetidin-1-yl)(cyclopropyl)methanone

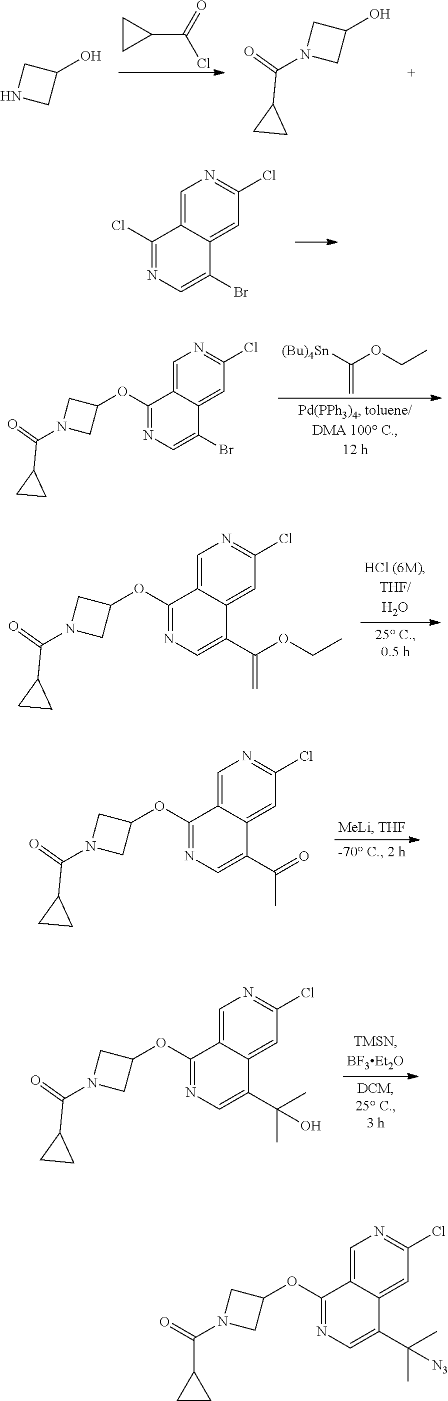

### Step 1: Cyclopropyl(3-hydroxyazetidin-1-yl)methanone

Cyclopropanecarbonyl chloride (83.0 g, 757 mmol, 1.00 eq) in DCM (800 mL) was added dropwise to a solution of azetidin-3-ol (HCl salt, 63.3 g, 606 mmol, 55.0 mL, 0.80 eq) and TEA (191 g, 1.89 mol, 263 mL, 2.50 eq) in DCM (800 mL) at 0° C. The mixture was stirred at 25° C. for 2 hr, then was filtered. The filtrate was concentrated to give a residue (150 g, crude). The residue was triturated with EA to give the title compound (32 g, 30% yield) as a white solid.

### Steps 2-4: 1-(6-Chloro-1-((1-(cyclopropanecarbonyl)azetidin-3-yl)oxy)-2,7-naphthyridin-4-yl)ethan-1-one

The title compound was prepared from cyclopropyl(3-hydroxyazetidin-1-yl)methanone and 4-Bromo-1,6-dichloro-2,7-naphthyridine (Intermediate 5) using the same 3-step procedure described in Steps 1-3 of Intermediate 7.

### Step 5: (3-((6-Chloro-4-(2-hydroxypropan-2-yl)-2,7-naphthyridin-1-yl)oxy)azetidin-1-yl)(cyclopropyl)methanone

MeLi (1.6 M, 10.9 mL) was added dropwise to a solution of 1-(6-chloro-1-((1-(cyclopropanecarbonyl)azetidin-3-yl)oxy)-2,7-naphthyridin-4-yl)ethan-1-one (4 g, 11.6 mmol) in THF (64 mL) at −70° C. The reaction mixture was stirred at −70° C. for 2 h, then was quenched with the saturated aqueous NH₄Cl (100 mL) and extracted with EA (300 mL). The combined organic layers were dried over sodium sulfate, filtered and concentrated to give the title compound (4.0 g, crude) as a yellow semisolid, which was used in the next step without further purification. MS (ES+) C₁₈H₂₀ClN₃O₃ requires: 361, found: 362[M+H]⁺.

### Step 6: (3-((4-(2-Azidopropan-2-yl)-6-chloro-2,7-naphthyridin-1-yl)oxy)azetidin-1-yl)(cyclopropyl)methanone

The title compound was prepared from (3-((6-chloro-4-(2-hydroxypropan-2-yl)-2,7-naphthyridin-1-yl)oxy)azetidin-1-yl)(cyclopropyl)methanone using the same procedure as described in Step 6 of Intermediate 7. ¹H-NMR (400 MHz, CDCl₃): δ ppm 9.49 (s, 1H), 8.39 (s, 1H), 8.16 (s, 1H), 5.66-5.61 (m, 1H), 4.80-4.73 (m, 1H), 4.57-4.51 (m, 1H), 4.48-4.42 (m, 1H), 4.24-4.20 (m, 1H), 1.81 (br d, J=5.6 Hz, 6H), 1.49-1.43 (m, 1H), 1.08-1.01 (m, 2H), 0.82-0.78 (m, 2H).

### Example 2e

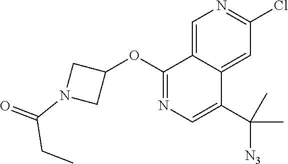 [SMILES: CCC(=O)N1CC(Oc2ncc(C(C)(C)N=[N+]=[N-])c3cc(Cl)ncc23)C1]

### Intermediate 9: 1-(3-((4-(2-Azidopropan-2-yl)-6-chloro-2,7-naphthyridin-1-yl)oxy)azetidin-1-yl)propan-1-one

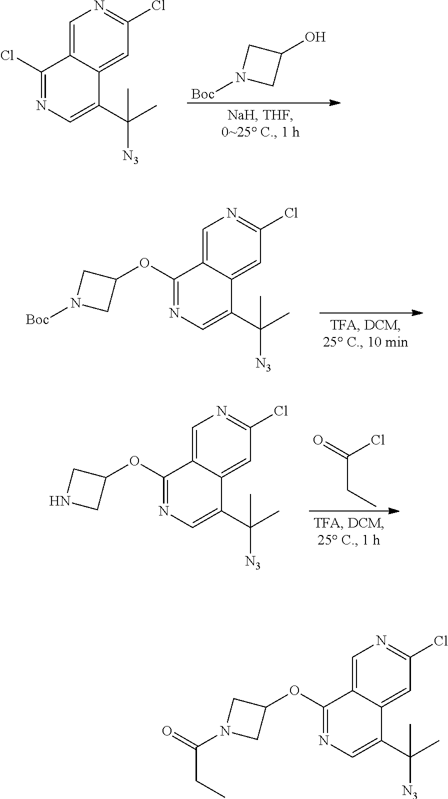

### Step 1: tert-Butyl 3-((4-(2-azidopropan-2-yl)-6-chloro-2,7-naphthyridin-1-yl)oxy)azetidine-1-carboxylate

Sodium hydride (170 mg, 4.25 mmol, 60 wt %) was added to a solution of tert-butyl 3-hydroxyazetidine-1-carboxylate (Combi-Blocks #AM-2061)(675 mg, 3.90 mmol) in THF (12 mL) at 0° C. The reaction mixture was stirred at 0° C. for 30 min, then 4-(2-azidopropan-2-yl)-1,6-dichloro-2,7-naphthyridine (Intermediate 6) (1 g, 3.54 mmol) was added and the cooling bath was removed. The reaction mixture was stirred for 30 min, then was quenched with water (30 mL) and extracted with EA (40 mL×3). The combined organic layers were dried over sodium sulfate, filtered and concentrated to give a residue. The residue was purified by flash-column chromatography on silica gel (gradient elution, 0% to 60% EA-PE) to give the title compound (1.1 g, 63% yield) as a yellow solid. MS (ES+) C₁₉H₂₃ClN₆O₃ requires: 418, found: 419[M+H]⁺.

### Step 2: 1-(Azetidin-3-yloxy)-4-(2-azidopropan-2-yl)-6-chloro-2,7-naphthyridine

A solution of tert-butyl 3-((4-(2-azidopropan-2-yl)-6-chloro-2,7-naphthyridin-1-yl)oxy)azetidine-1-carboxylate (400 mg, 955 umol) in DCM (2 mL) was added to a solution of trifluoroacetic acid (6.16 g, 54.0 mmol, 4 mL) in DCM (2 mL) at 25° C. The reaction mixture was stirred at 25° C. for 10 min, then was concentrated to give a residue (350 mg, crude) which was used in the next step without further purification. MS (ES+) C₁₄H₁₅ClN₆O requires: 318, found: 319[M+H]⁺.

### Step 3: 1-(3-((4-(2-Azidopropan-2-yl)-6-chloro-2,7-naphthyridin-1-yl)oxy)azetidin-1-yl)propan-1-one

Propionyl chloride (203 mg, 2.20 mmol, 203 uL) and TEA (278 mg, 2.75 mmol) were added to a solution of 1-(azetidin-3-yloxy)-4-(2-azidopropan-2-yl)-6-chloro-2,7-naphthyridine (350 mg, 1.1 mmol) in DCM (10 mL) at at 0° C. The reaction mixture was stirred at 0° C. for 1 h, then was diluted with water (30 mL) and extracted with EA (30 mL×3). The combined organic layers were dried over sodium sulfate, filtered and concentrated to give a residue. The residue was purified by flash-column chromatography on silica gel (gradient elution, 0% to 100% EA-PE) to give the title compound (118 mg, 24% yield) as a white solid. MS (ES+) C₁₇H₁₉ClN₆O₂ requires: 374, found: 375[M+H]⁺.

### Example 2f

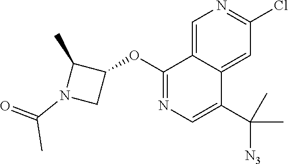 [SMILES: CC(=O)N1C[C@@H](Oc2ncc(C(C)(C)N=[N+]=[N-])c3cc(Cl)ncc23)[C@@H]1C]

### Intermediate 10: 1-((2S,3R)-3-((4-(2-Azidopropan-2-yl)-6-chloro-2,7-naphthyridin-1-yl)oxy)-2-methylazetidin-1-yl)ethan-1-one

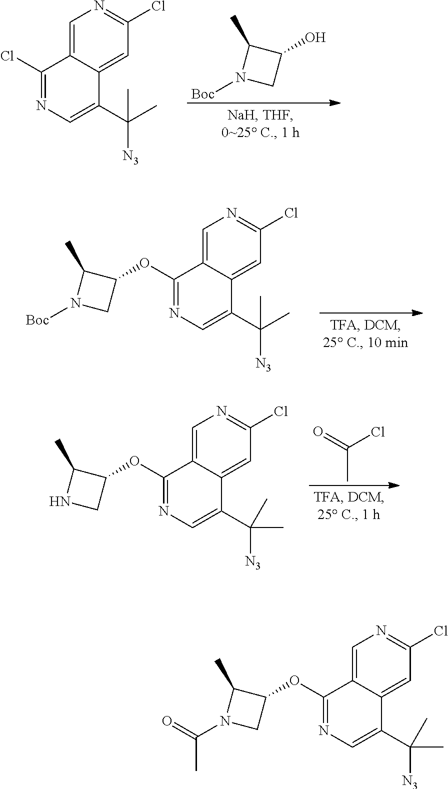

### Steps 1-3: 1-((2S,3R)-3-((4-(2-Azidopropan-2-yl)-6-chloro-2,7-naphthyridin-1-yl)oxy)-2-methylazetidin-1-yl)ethan-1-one

The title compound was prepared from 4-(2-azidopropan-2-yl)-1,6-dichloro-2,7-naphthyridine (Intermediate 6) and tert-butyl (2S,3R)-3-hydroxy-2-methylazetidine-1-carboxylate (Combi-Blocks #HD-7336) using the same 3-Step procedure described for Steps 1-3 of Intermediate 9 above. In Step 3, acetyl chloride was used instead of propionyl chloride. C₁₇H₁₉ClN₆O₂ requires: 374, found: 375[M+H]⁺.

### Example 2g

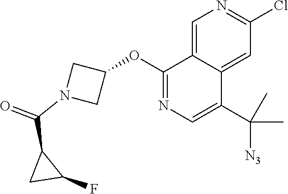

### Intermediate 11 and its Enantiomer: (3-((4-(2-Azidopropan-2-yl)-6-chloro-2,7-naphthyridin-1-yl)oxy)azetidin-1-yl)((1S,2S)-2-fluorocyclopropyl)methanone and (3-((4-(2-Azidopropan-2-yl)-6-chloro-2,7-naphthyridin-1-yl)oxy)azetidin-1-yl)((1R,2R)-2-fluorocyclopropyl)methanone

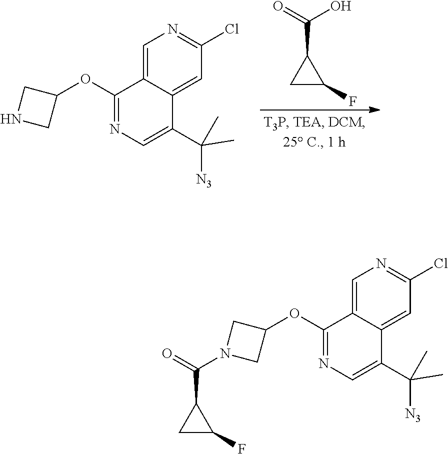 [SMILES: CC(C)(N=[N+]=[N-])c1cnc(OC2CNC2)c2cnc(Cl)cc12 ; O=C(O)[C@@H]1C[C@@H]1F ; CC(C)(N=[N+]=[N-])c1cnc(OC2CN(C(=O)[C@@H]3C[C@@H]3F)C2)c2cnc(Cl)cc12]

### Step 1: (3-((4-(2-Azidopropan-2-yl)-6-chloro-2,7-naphthyridin-1-yl)oxy)azetidin-1-yl)((1S,2S)-2-fluorocyclopropyl)methanone

T₃P (998 mg, 1.57 mmol, 50% purity) and TEA (158 mg, 1.57 mmol) were added to a solution of 1-(azetidin-3-yloxy)-4-(2-azidopropan-2-yl)-6-chloro-2,7-naphthyridine (100 mg, 313 μmol) (Intermediate 9, Step 2) and (1S,2S)-2-fluorocyclopropane-1-carboxylic acid (Enamine #EN300-1654606) 48.9 mg, 470 μmol) in DCM (3 mL). The reaction mixture was stirred at 25° C. for 1 h, then was concentrated to give a residue. The residue was purified by flash-column chromatography on silica gel (gradient elution, 5% to 50% EA-PE) to give the title compound (80.0 mg, 63% yield) as a yellow solid. MS (ES+) C₁₈H₁₈ClFN₆O₂ requires: 404, found: 405[M+H]⁺. The enantiomer of Intermediate 11 is made using a procedure that is similar to the procedure for Intermediate 11, except that (1R,2R)-2-fluorocyclopropane-1-carboxylic acid (Combi-Blocks #QM-4290) is used.

### Example 2h

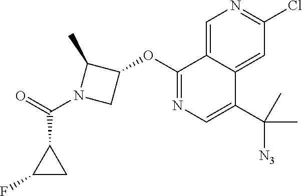 [SMILES: C[C@H]1[C@H](Oc2ncc(C(C)(C)N=[N+]=[N-])c3cc(Cl)ncc23)CN1C(=O)[C@@H]1C[C@@H]1F]

### Intermediate 12: ((2S,3R)-3-((4-(2-Azidopropan-2-yl)-6-chloro-2,7-naphthyridin-1-yl)oxy)-2-methylazetidin-1-yl)((1S,2S)-2-fluorocyclopropyl)methanone

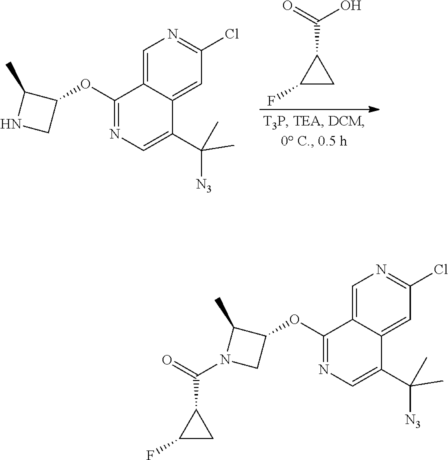 [SMILES: C[C@@H]1NC[C@H]1Oc1ncc(C(C)(C)N=[N+]=[N-])c2cc(Cl)ncc12 ; O=C(O)[C@@H]1C[C@@H]1F ; C[C@H]1[C@H](Oc2ncc(C(C)(C)N=[N+]=[N-])c3cc(Cl)ncc23)CN1C(=O)[C@@H]1C[C@@H]1F]

### Step 1: ((2S,3R)-3-((4-(2-Azidopropan-2-yl)-6-chloro-2,7-naphthyridin-1-yl)oxy)-2-methylazetidin-1-yl)((1S,2S)-2-fluorocyclopropyl)methanone

The title compound was prepared from 4-(2-azidopropan-2-yl)-6-chloro-1-(((2S,3R)-2-methylazetidin-3-yl)oxy)-2,7-naphthyridine (Intermediate 10, Step 2) and (1S,2S)-2-fluorocyclopropane-1-carboxylic acid using a similar procedure described above for Intermediate 11, except that the reaction is carried out at 0° C. for 0.5 h.

### Example 2i

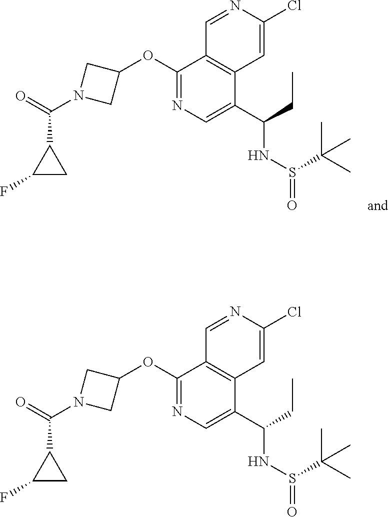

### Intermediates 13 and 14: (S)—N—((R)-1-(6-chloro-1-((1-((1S,2S)-2-fluorocyclopropane-1-carbonyl)azetidin-3-yl)oxy)-2,7-naphthyridin-4-yl)propyl)-2-methylpropane-2-sulfinamide and (S)—N—((S)-1-(6-chloro-1-((1-((1S,2S)-2-fluorocyclopropane-1-carbonyl)azetidin-3-yl)oxy)-2,7-naphthyridin-4-yl)propyl)-2-methylpropane-2-sulfinamide

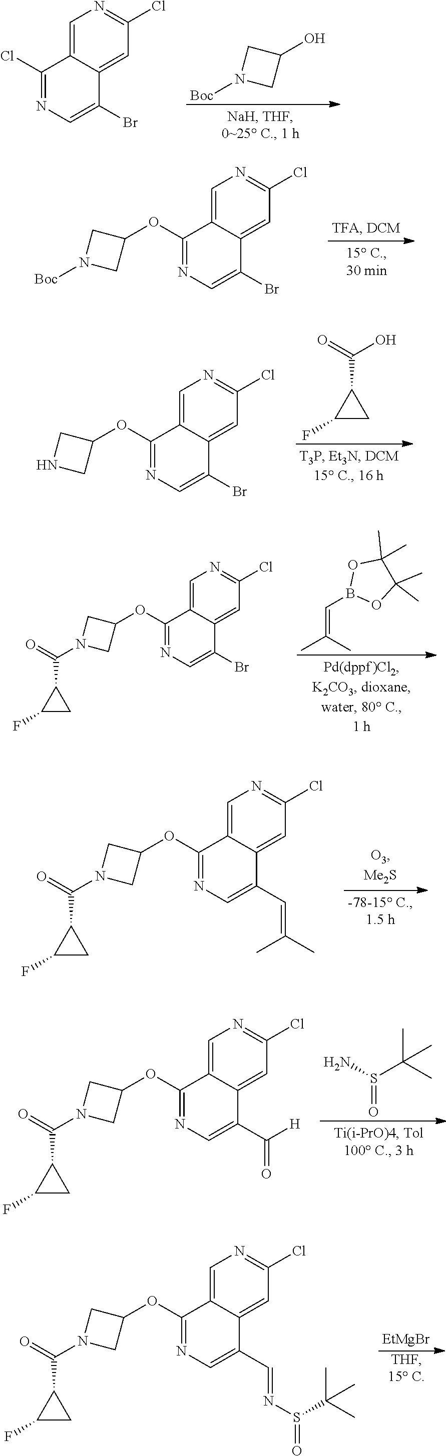

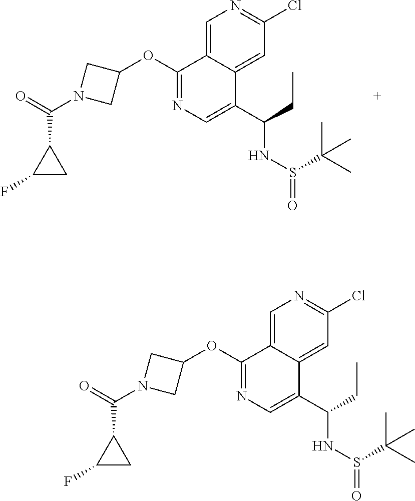 [SMILES: CC[C@@H](N[S@@](=O)C(C)(C)C)c1cnc(OC2CN(C(=O)[C@@H]3C[C@@H]3F)C2)c2cnc(Cl)cc12 ; CC[C@H](N[S@@](=O)C(C)(C)C)c1cnc(OC2CN(C(=O)[C@@H]3C[C@@H]3F)C2)c2cnc(Cl)cc12]

### Step 1: tert-Butyl 3-((4-bromo-6-chloro-2,7-naphthyridin-1-yl)oxy)azetidine-1-carboxylate

The title compound was prepared from 4-bromo-1,6-dichloro-2,7-naphthyridine (Intermediate 5) and tert-butyl 3-hydroxyazetidine-1-carboxylate using the same procedure described in Step 1 of Intermediate 7.

### Step 2: 1-(Azetidin-3-yloxy)-4-bromo-6-chloro-2,7-naphthyridine

A solution of tert-butyl 3-((4-bromo-6-chloro-2,7-naphthyridin-1-yl)oxy)azetidine-1-carboxylate (1 g, 2.41 mmol) in TFA (5.50 g, 3.57 mL) and DCM (2 mL) was stirred at 15° C. for 30 min. The reaction mixture was then concentrated under reduced pressure at 15° C. to remove the solvent and give the title compound (750 mg, crude) as yellow oil that was used without further purification.

### Step 3: (3-((4-Bromo-6-chloro-2,7-naphthyridin-1-yl)oxy)azetidin-1-yl)((1S,2S)-2-fluorocyclopropyl)methanone

T₃P (2.28 g, 7.15 mmol) was added to a solution of 1-(azetidin-3-yloxy)-4-bromo-6-chloro-2,7-naphthyridine (750 mg, 2.38 mmol), (1S,2S)-2-fluorocyclopropane-1-carboxylic acid (273 mg, 2.62 mmol), and TEA (1.21 g, 11.9 mmol) in DCM (3 mL). The reaction mixture was stirred at 15° C. for 16 h, then was concentrated to give a residue. The residue was purified by flash-column chromatography on silica gel (50% EA-PE) to give the title compound (900 mg, 94% yield) as colorless oil.

### Step 4: (3-((6-Chloro-4-(2-methylprop-1-en-1-yl)-2,7-naphthyridin-1-yl)oxy)azetidin-1-yl)((1S,2S)-2-fluorocyclopropyl)methanone

Pd(dppf)Cl₂ (73.1 mg, 99.8 μmol) and potassium carbonate (552 mg, 3.99 mmol) were added to a solution of (3-((4-bromo-6-chloro-2,7-naphthyridin-1-yl)oxy)azetidin-1-yl)((1S,2S)-2-fluorocyclopropyl)methanone (800 mg, 2.00 mmol) and 4,4,5,5-tetramethyl-2-(2-methylprop-1-en-1-yl)-1,3,2-dioxaborolane (382 mg, 2.10 mmol) in dioxane (10 mL) and water (2 mL). The reaction mixture was stirred at 80° C. for 1 h under nitrogen, then was cooled to ambient temperature and partitioned between EA and water. The organic layer was dried over sodium sulfate, filtered, and concentrated to give a residue. The residue was purified by flash-column chromatography on silica gel (50% EA-PE) to give the title compound (700 mg, 93% yield) as yellow solid.

### Step 5: 6-Chloro-1-((1-((1S,2S)-2-fluorocyclopropane-1-carbonyl)azetidin-3-yl)oxy)-2,7-naphthyridine-4-carbaldehyde

Ozone was bubbled into a solution of (3-((6-chloro-4-(2-methylprop-1-en-1-yl)-2,7-naphthyridin-1-yl)oxy)azetidin-1-yl)((1S,2S)-2-fluorocyclopropyl)methanone (700 mg, 1.86 mmol) in DCM (20 mL) and MeOH (2 mL) at −78° C. for 20 min. Excess ozone was purged by nitrogen, dimethylsulfide (231 mg, 3.73 mmol) was added at 0° C. over 10 min, and the reaction mixture was stirred at 15° C. for 1 h. The reaction mixture was concentrated, and the residue was purified by flash-column chromatography on silica gel (gradient elution, 50% to 75% EA-PE) to give the title compound (320 mg, 49% yield) as white solid.

### Step 6: (S)—N-((E)-(6-chloro-1-((1-((1S,2S)-2-fluorocyclopropane-1-carbonyl)azetidin-3-yl)oxy)-2,7-naphthyridin-4-yl)methylene)-2-methylpropane-2-sulfinamide

Ti(i-PrO)₄ (1.56 g, 5.49 mmol) was added to a solution of (S)-2-methylpropane-2-sulfinamide (333 mg, 2.74 mmol) and 6-chloro-1-((1-((1S,2S)-2-fluorocyclopropane-1-carbonyl)azetidin-3-yl)oxy)-2,7-naphthyridine-4-carbaldehyde (320 mg, 915 μmol) in toluene (10 mL). The reaction mixture was stirred at 100° C. for 3 h, then was poured into EA (200 mL) and water (50 mL), filtered, and extracted with EA (100 mL×3). The organic layers were combined and dried over sodium sulfate, filtered and concentrated to give a residue. The residue was purified by flash-column chromatography on silica gel (gradient elution, 50% to 75% EA-PE) to give the title compound (200 mg, 48% yield) as a white solid.

### Step 7: (S)—N—((R)-1-(6-chloro-1-((1-((1S,2S)-2-fluorocyclopropane-1-carbonyl)azetidin-3-yl)oxy)-2,7-naphthyridin-4-yl)propyl)-2-methylpropane-2-sulfinamide and (S)—N—((S)-1-(6-chloro-1-((1-((1S,2S)-2-fluorocyclopropane-1-carbonyl)azetidin-3-yl)oxy)-2,7-naphthyridin-4-yl)propyl)-2-methylpropane-2-sulfinamide

Ethylmagnesium bromide (3 M, 883 μL) was added to a solution of (S)—N—((E)-(6-chloro-1-((1-((1S,2S)-2-fluorocyclopropane-1-carbonyl)azetidin-3-yl)oxy)-2,7-naphthyridin-4-yl)methylene)-2-methylpropane-2-sulfinamide (200 mg, 441 μmol) in THF (2 mL) at 15° C. The reaction mixture was stirred at 15° C. for 10 min, then was poured into water (5 mL) and extracted with EA (10 mL×3). The organic layers were combined and dried over sodium sulfate, filtered and concentrated to give a residue. The residue was purified by prep-HPLC (column: Waters Xbridge 150*25 mm*5 um; mobile phase: [water (10 mM NH₄HCO₃)-ACN]; B %: 28%-58%, over 10 min) to give the title compounds separately. The stereochemistry was arbitrarily assigned.

First eluting, Peak 1 (Intermediate 13): 20 mg, 9.38% yield) as yellow solid. ¹H-NMR (400 MHz, CDCl₃): δ ppm 9.47 (s, 1H), 8.15 (d, J=3.2 Hz, 1H), 8.04 (d, J=10.0 Hz, 1H), 5.67-5.65 (m, 1H), 4.82-4.26 (m, 7H), 3.58-3.50 (m, 1H), 2.07-1.67 (m, 8H), 1.21 (s, 9H), 1.15-1.10 (m, 2H), 0.95-0.91 (m, 4H).

Second eluting, Peak 2 (Intermediate 14): 20 mg, 9.38% yield) as yellow solid. ¹H-NMR (400 MHz, CDCl₃): δ ppm 9.46 (s, 1H), 8.20 (m, 1H), 7.99 (d, J=11.2 Hz, 1H), 5.66-5.63 (m, 1H), 4.79-4.27 (m, 7H), 3.61-3.58 (m, 1H), 2.21-1.88 (m, 7H), 1.25 (s, 9H), 1.10-1.08 (m, 2H), 0.94-0.88 (m, 4H).

### Example 2j

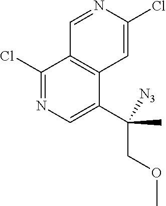 [SMILES: COC[C@@](C)(N=[N+]=[N-])c1cnc(Cl)c2cnc(Cl)cc12]

### Intermediate 15: (S)-4-(2-Azido-1-methoxypropan-2-yl)-1,6-dichloro-2,7-naphthyridine

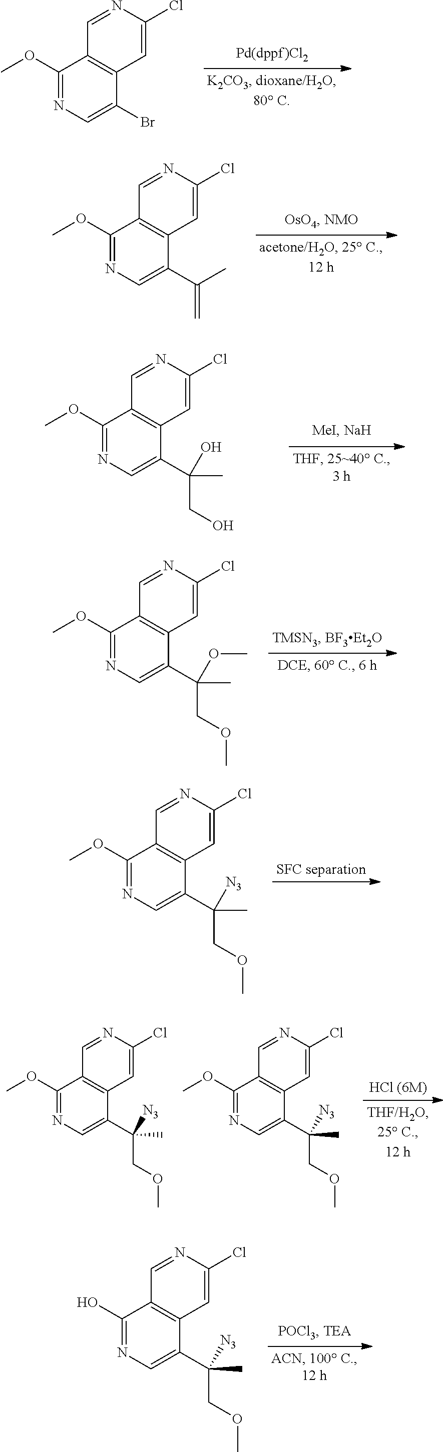

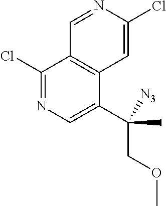 [SMILES: COC[C@@](C)(N=[N+]=[N-])c1cnc(Cl)c2cnc(Cl)cc12]

### Step 1: 6-Chloro-1-methoxy-4-(prop-1-en-2-yl)-2,7-naphthyridine

The title compound was prepared from 4-bromo-6-chloro-1-methoxy-2,7-naphthyridine (Intermediate 6, Step 1) and 2-isopropenyl-4,4,5,5-tetramethyl-1,3,2-dioxaborolane using the same procedure described in Step 4 of Intermediates 13 and 14.

### Step 2: 2-(6-Chloro-1-methoxy-2,7-naphthyridin-4-yl)propane-1,2-diol

(1.02 g, 4.01 mmol) was added to a mixture of 6-chloro-1-methoxy-4-(prop-1-en-2-yl)-2,7-naphthyridine (9.4 g, 40.0 mmol) and NMO (9.38 g, 80.1 mmol) in acetone (160 mL) and H₂O (40 mL). The reaction mixture was stirred at 25° C. for 12 h, then was quenched saturated aqueous KF solution (150 mL) and filtered. The solution was extracted with EA (2×300 mL). The organic phase was dried over Na₂SO₄, filtered and concentrated to give the title compound (9.3 g, 86% yield) as yellow oil which was used in the next step without further purification.

### Step 3: 6-Chloro-4-(1,2-dimethoxypropan-2-yl)-1-methoxy-2,7-naphthyridine

NaH (4.85 g, 121 mmol, 60% purity) was added to a solution of 2-(6-chloro-1-methoxy-2,7-naphthyridin-4-yl)propane-1,2-diol (9.3 g, 34.6 mmol) in THF (150 mL). The reaction mixture was stirred at 25° C. for 0.5 h, then MeI (12.3 g, 86.5 mmol) was added. The reaction mixture was stirred at 25° C. for 0.5 h, and then stirred at 40° C. for 2 h. The reaction mixture was then added into a stirring solution of the saturated aqueous NH₄Cl (50 mL) and extracted with EA (300 mL). The organic layer was washed with saturated aqueous NH₄Cl (100 mL×3), dried over Na₂SO₄, filtered and concentrated to give the title compound (10 g, 85% yield) as a yellow oil which was used in the next step without further purification.

### Step 4: 4-(2-Azido-1-methoxypropan-2-yl)-6-chloro-1-methoxy-2,7-naphthyridine

BF₃·Et₂O (8.80 g, 62.0 mmol) was added to a mixture of 6-chloro-4-(1,2-dimethoxypropan-2-yl)-1-methoxy-2,7-naphthyridine (9.2 g, 31.0 mmol), TMSN₃ (17.9 g, 155 mmol) in DCE (150 mL) at 25° C. The reaction mixture heated to 60° C. for 6 h under N₂. The reaction mixture was then added into a stirring solution of aqueous saturated NaHCO₃(300 mL) and extracted with EA (300 mL). The organic layer was dried over Na₂SO₄, filtered and concentrated to give a residue. The residue was purified by flash-column chromatography on silica gel (20% EA-PE) to give the title compound (8 g, 73% yield) as a colorless oil.

### Step 4: (R)-4-(2-Azido-1-methoxypropan-2-yl)-6-chloro-1-methoxy-2,7-naphthyridine and (S)-4-(2-azido-1-methoxypropan-2-yl)-6-chloro-1-methoxy-2,7-naphthyridine

The title compounds were prepared by chiral SFC separation of 4-(2-azido-1-methoxypropan-2-yl)-6-chloro-1-methoxy-2,7-naphthyridine (column: Daicel ChiralPak IG (250*30 mm, 10 um); mobile phase: [15% (IPA with 0.1% NH₄OH)] to give the title two isomers. The first eluting isomer was (R)-4-(2-Azido-1-methoxypropan-2-yl)-6-chloro-1-methoxy-2,7-naphthyridine and the second eluting isomer was (S)-4-(2-azido-1-methoxypropan-2-yl)-6-chloro-1-methoxy-2,7-naphthyridine. The isomers were determined by X-ray crystal structure of Compound 16, which was derived from the second eluting isomer.

### Step 5: (S)-4-(2-Azido-1-methoxypropan-2-yl)-6-chloro-2,7-naphthyridin-1-ol

Aqueous HCl (6 M, 5.41 mL) was added to a solution of (S)-4-(2-azido-1-methoxypropan-2-yl)-6-chloro-1-methoxy-2,7-naphthyridine (3 g, 9.75 mmol) in THF (240 mL). The mixture was stirred at 25° C. for 12 h, then was adjusted to pH ˜8 with addition of solid NaHCO₃. The mixture was extracted with EA (2×150 mL) and the organic layer was dried over Na₂SO₄, filtered and concentrated to give the title compound (2.7 g, 83% yield, 88% purity) as a white solid which was used in the next step without further purification.

### Step 6: (S)-4-(2-Azido-1-methoxypropan-2-yl)-1,6-dichloro-2,7-naphthyridine

POCl₃ (3.34 g, 21.8 mmol) was added to a mixture of (S)-4-(2-azido-1-methoxypropan-2-yl)-6-chloro-2,7-naphthyridin-1-ol (1.28 g, 4.36 mmol) and TEA (1.16 g, 11.5 mmol) in ACN (20 mL). The mixture was heated to 100° C. for 12 h. The reaction mixture was then added to saturated aqueous NH₄Cl (50 mL) and extracted with EA (100 mL×3). The organic layers were combined and dried over Na₂SO₄, filtered, and concentrated to give a residue. The residue was purified by flash-column chromatography on silica gel (25% EA-PE) to give the title compound (1.2 g, 88% yield) as a yellow oil. ¹H NMR (400 MHz, CDCl₃): δ ppm 9.61 (s, 1H), 8.57 (s, 1H), 8.48 (s, 1H), 3.98 (d, J=9.6 Hz, 1H), 3.78 (d, J=9.6 Hz, 1H), 3.41 (s, 3H), 1.80 (s, 3H).

### Example 2k

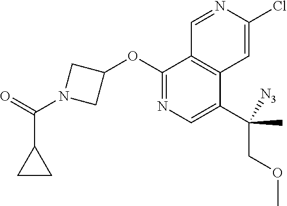 [SMILES: COC[C@@](C)(N=[N+]=[N-])c1cnc(OC2CN(C(=O)C3CC3)C2)c2cnc(Cl)cc12]

### Intermediate 16: (S)-(3-((4-(2-Azido-1-methoxypropan-2-yl)-6-chloro-2,7-naphthyridin-1-yl)oxy)azetidin-1-yl)(cyclopropyl)methanone

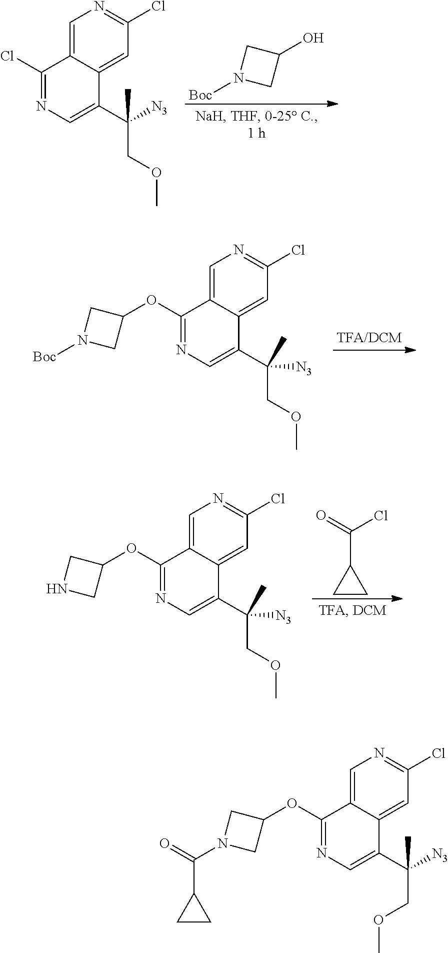 [SMILES: COC[C@@](C)(N=[N+]=[N-])c1cnc(Cl)c2cnc(Cl)cc12 ; CC(C)(C)OC(=O)N1CC(O)C1 ; COC[C@@](C)(N=[N+]=[N-])c1cnc(OC2CN(C(=O)OC(C)(C)C)C2)c2cnc(Cl)cc12 ; COC[C@@](C)(N=[N+]=[N-])c1cnc(OC2CNC2)c2cnc(Cl)cc12 ; O=C(Cl)C1CC1 ; COC[C@@](C)(N=[N+]=[N-])c1cnc(OC2CN(C(=O)C3CC3)C2)c2cnc(Cl)cc12]

### Steps 1-3: (S)-(3-((4-(2-Azido-1-methoxypropan-2-yl)-6-chloro-2,7-naphthyridin-1-yl)oxy)azetidin-1-yl)(cyclopropyl)methanone

The title compound was prepared from (S)-4-(2-azido-1-methoxypropan-2-yl)-1,6-dichloro-2,7-naphthyridine and cycloproponyl chloride using the same procedure as described in Steps 1-3 of Intermediate 9. MS (ES+) C₁₉H₂₁ClN₆O₃ requires: 416, found: 417 [M+H]⁺. ¹H NMR (400 MHz, CDCl₃) δ ppm 9.41 (s, 1H), 8.34 (s, 1H), 8.11 (s, 1H), 5.61-5.51 (m, 1H), 4.75-4.65 (m, 1H), 4.45 (dd, J=6.8, 10.9 Hz, 1H), 4.41-4.27 (m, 1H), 4.17-4.09 (m, 1H), 3.84 (dd, J=10.0, 15.0 Hz, 1H), 3.71-3.60 (m, 1H), 3.36 (d, J=5.6 Hz, 3H), 1.69 (d, J=13.9 Hz, 3H), 1.42-1.34 (m, 1H), 0.97-0.92 (m, 2H), 0.72 (dd, J=2.9, 7.8 Hz, 2H).

### Example 21

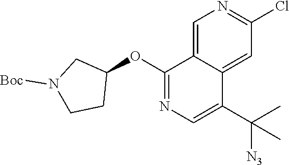 [SMILES: CC(C)(C)OC(=O)N1CC[C@H](Oc2ncc(C(C)(C)N=[N+]=[N-])c3cc(Cl)ncc23)C1]

### Intermediate 28: tert-Butyl (S)-3-((4-(2-azidopropan-2-yl)-6-chloro-2,7-naphthyridin-1-yl)oxy)pyrrolidine-1-carboxylate

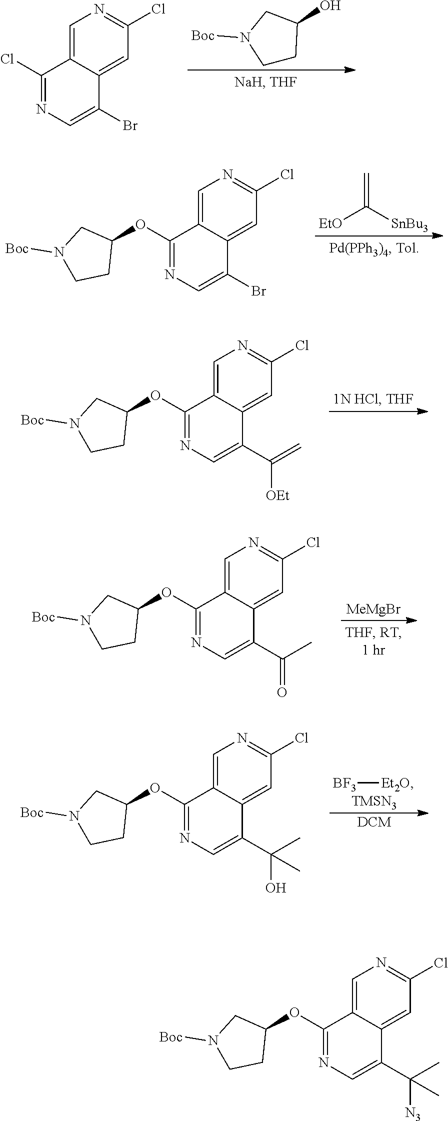

### Steps 1-2: tert-Butyl (S)-3-((6-chloro-4-(1-ethoxyvinyl)-2,7-naphthyridin-1-yl)oxy)pyrrolidine-1-carboxylate

The title compound was prepared from tert-butyl (S)-3-hydroxypyrrolidine-1-carboxylate and 4-bromo-1,6-dichloro-2,7-naphthyridine (Intermediate 5) using the same procedure as described in Steps 1-2 of Intermediate 7.

### Step 3: tert-Butyl (S)-3-((4-acetyl-6-chloro-2,7-naphthyridin-1-yl)oxy)pyrrolidine-1-carboxylate

A mixture of tert-butyl (S)-3-((6-chloro-4-(1-ethoxyvinyl)-2,7-naphthyridin-1-yl)oxy)pyrrolidine-1-carboxylate (5.00 g, 11.91 mmol, 1.00 eq) in THF (50 mL) and HCl (1 N, 50 mL) was stirred at 25° C. for 0.5 h. The reaction mixture was then quenched by addition of saturated aqueous NaHCO₃ solution (25) mL at 0° C. and concentrated to remove THF. The mixture was then extracted with EA (40 mL×3) and the combined organic layers were washed with brine (50 mL), dried over Na₂SO₄, filtered and concentrated to give a residue. The residue was purified by column chromatography (SiO₂, PE:EA=1:1 to 0:1) to give the title compound (2.20 g, yield: 47%) as a yellow oil.

### Step 4: tert-Butyl (S)-3-((6-chloro-4-(2-hydroxypropan-2-yl)-2,7-naphthyridin-1-yl)oxy)pyrrolidine-1-carboxylate

To a solution of MeMgBr (5.00 M in n-hexane, 1.79 mL, 5.00 eq) in THF (5.00 mL) was added a solution of tert-butyl (S)-3-((4-acetyl-6-chloro-2,7-naphthyridin-1-yl)oxy)pyrrolidine-1-carboxylate (700 mg, 1.79 mmol, 1.00 eq) in THF (5.00 mL). The reaction mixture was stirred at 25° C. for 1 h, then was quenched by addition of saturated aqueous NH₄Cl solution (15 mL) at 0° C. and extracted with EA (20.00 mL×3). The combined organic layers were washed with brine (15 mL), dried over Na₂SO₄, filtered and concentrated to give a residue. The residue was purified by column chromatography (SiO₂, PE:EA=1:1 to 0:1) to give the title compound (450 mg, yield: 62%) as a yellow oil.

### Step 5: tert-Butyl (S)-3-((4-(2-azidopropan-2-yl)-6-chloro-2,7-naphthyridin-1-yl)oxy)pyrrolidine-1-carboxylate

To a solution of tert-butyl (S)-3-((6-chloro-4-(2-hydroxypropan-2-yl)-2,7-naphthyridin-1-yl)oxy)pyrrolidine-1-carboxylate (1.30 g, 3.19 mmol, 1.00 eq) in DCM (20 mL) was added TMSN₃ (1.05 mL, 7.97 mmol, 2.50 eq) and BF₃·Et₂O (865 uL, 7.01 mmol, 2.20 eq) under N₂ atmosphere. The reaction mixture was stirred at 25° C. for 3 h, then was quenched by addition of H₂O (15 mL) and extracted with EA (20.00 mL×3). The combined organic layers were washed with brine (20 mL), dried over Na₂SO₄, filtered and concentrated to give a residue. The residue was purified by column chromatography (SiO₂, PE:EA=1:1 to 0:1) to give the title compound (0.90 g, yield: 65%) as a yellow oil. MS (ES+) C₂₀H₂₅ClN₆O₃ requires: 432, found: 433 [M+H]⁺.

### Example 2m

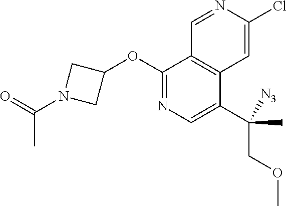 [SMILES: COC[C@@](C)(N=[N+]=[N-])c1cnc(OC2CN(C(C)=O)C2)c2cnc(Cl)cc12]

### Intermediate 29: (S)-1-(3-((4-(2-Azido-1-methoxypropan-2-yl)-6-chloro-2,7-naphthyridin-1-yl)oxy)azetidin-1-yl)ethan-1-one

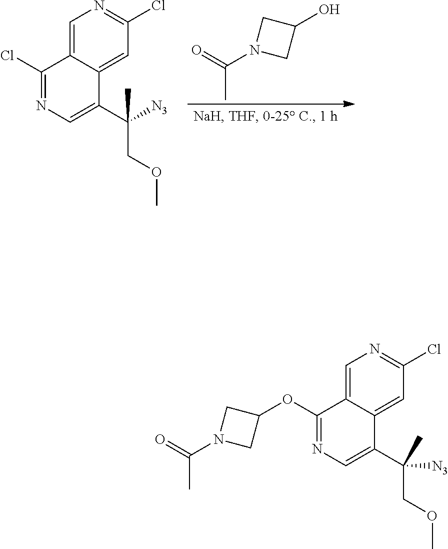

### Step 1: (S)-(3-((4-(2-Azido-1-methoxypropan-2-yl)-6-chloro-2,7-naphthyridin-1-yl)oxy)azetidin-1-yl)(cyclopropyl)methanone

The title compound was prepared from (S)-4-(2-azido-1-methoxypropan-2-yl)-1,6-dichloro-2,7-naphthyridine and 1-(3-hydroxyazetidin-1-yl)ethan-1-one (Intermediate 15) using a similar procedure as described in Step 1 of Intermediate 9. MS (ES+) C₁₇H₁₉ClN₆O₃ requires: 390, found: 391 [M+H]⁺.

### Example 2n

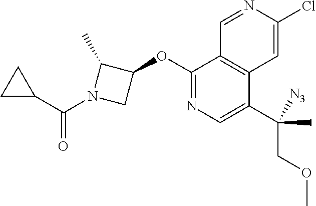 [SMILES: COC[C@@](C)(N=[N+]=[N-])c1cnc(O[C@H]2CN(C(=O)C3CC3)[C@@H]2C)c2cnc(Cl)cc12]

### Intermediate 30: ((2R,3S)-3-((4-((S)-2-Azido-1-methoxypropan-2-yl)-6-chloro-2,7-naphthyridin-1-yl)oxy)-2-methylazetidin-1-yl)(cyclopropyl)methanone

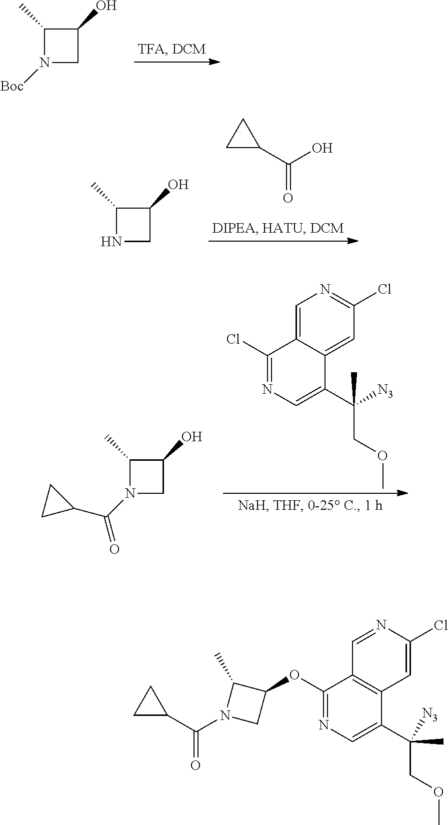

### Step 1: (2R,3S)-2-Methylazetidin-3-ol

TFA (500 μL, 6.53 mmol, 4.89 eq) was added to a solution of tert-butyl (2R,3S)-3-hydroxy-2-methylazetidine-1-carboxylate (250 mg, 1.34 mmol) in DCM (3.90 mL) at 0° C. The resulting solution was stirred at 23° C. for 2 h, then was concentrated to give a residue. Et₂O (10 mL) and toluene (10 mL) were added to the residue and the mixture was concentrated to give the title compound (TFA salt, 269 mg, 100%) as a colorless oil.

### Step 2: Cyclopropyl((2R,3S)-3-hydroxy-2-methylazetidin-1-yl)methanone

DIPEA (693 μL, 3.98 mmol, 4.0 eq) was added to a suspension of cyclopropanecarboxylic acid (79.2 μL, 994 μmol, 1.0 eq) and HATU (378 mg, 994 μmol, 1.0 eq) in anhydrous DMF (5.00 mL) at 23° C. followed by (2R,3S)-2-methylazetidin-3-ol 2,2,2-trifluoroacetate (200 mg, 994 μmol). The resulting reaction mixture was stirred at 23° C. for 5 h. The reaction mixture was concentrated, and the residue was purified by flash column chromatography on silica gel (0-10% MeOH/DCM) to give the title compound (117 mg, 76%) as a colorless oil. ¹H NMR (DMSO-d₆, 400 MHz): δ 5.68 (1H, s), 4.34-4.18 (1H, m), 3.92 (2H, s), 3.79-3.42 (1H, m), 1.42 (2H, s), 1.26 (2H, s), 0.66 (4H, m).

### Step 3: ((2R,3S)-3-((4-((S)-2-Azido-1-methoxypropan-2-yl)-6-chloro-2,7-naphthyridin-1-yl)oxy)-2-methylazetidin-1-yl)(cyclopropyl)methanone

The title compound was prepared from (S)-4-(2-azido-1-methoxypropan-2-yl)-1,6-dichloro-2,7-naphthyridine (Intermediate 15) and cyclopropyl((2R,3S)-3-hydroxy-2-methylazetidin-1-yl)methanone using a similar procedure as described in Step 1 of Intermediate 9.

### Example 20

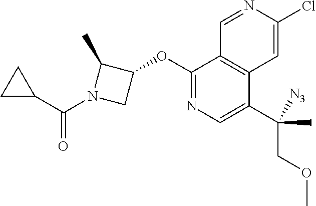 [SMILES: COC[C@@](C)(N=[N+]=[N-])c1cnc(O[C@@H]2CN(C(=O)C3CC3)[C@H]2C)c2cnc(Cl)cc12]

### Intermediate 31: ((2S,3R)-3-((4-((S)-2-Azido-1-methoxypropan-2-yl)-6-chloro-2,7-naphthyridin-1-yl)oxy)-2-methylazetidin-1-yl)(cyclopropyl)methanone

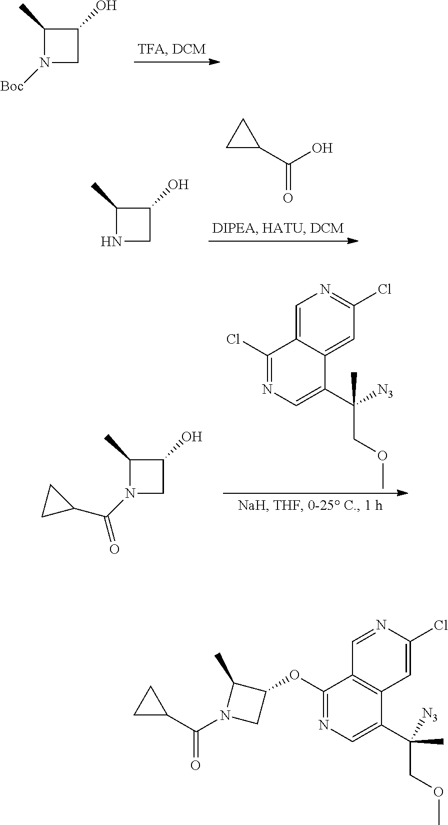

### Steps 1-3: ((2S,3R)-3-((4-((S)-2-Azido-1-methoxypropan-2-yl)-6-chloro-2,7-naphthyridin-1-yl)oxy)-2-methylazetidin-1-yl)(cyclopropyl)methanone

The title compound was prepared from tert-butyl (2S,3R)-3-hydroxy-2-methylazetidine-1-carboxylate and (S)-4-(2-azido-1-methoxypropan-2-yl)-1,6-dichloro-2,7-naphthyridine using a similar procedure as described in Steps 1-2 of Intermediate 30 and Step 1 of Intermediate 9.

### Example 2p

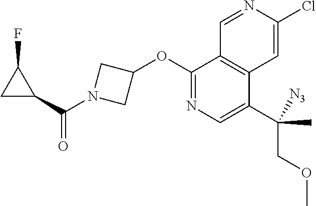 [SMILES: COC[C@@](C)(N=[N+]=[N-])c1cnc(OC2CN(C(=O)[C@H]3C[C@H]3F)C2)c2cnc(Cl)cc12]

### Intermediate 32: (3-((4-((S)-2-Azido-1-methoxypropan-2-yl)-6-chloro-2,7-naphthyridin-1-yl)oxy)azetidin-1-yl)((1R,2R)-2-fluorocyclopropyl)methanone

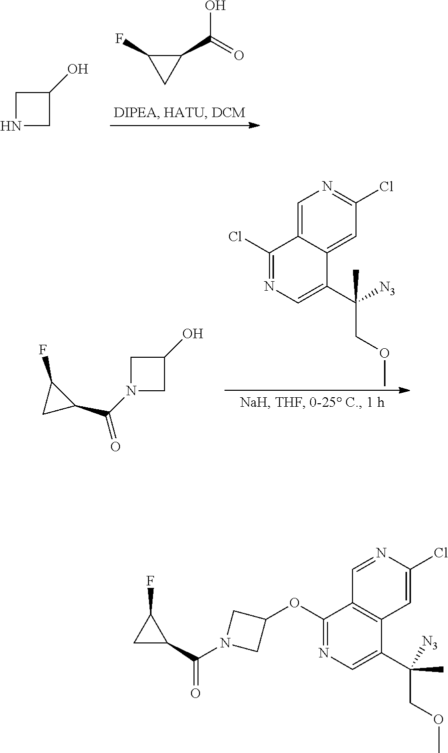 [SMILES: OC1CNC1 ; O=C(O)[C@H]1C[C@H]1F ; O=C([C@H]1C[C@H]1F)N1CC(O)C1 ; COC[C@@](C)(N=[N+]=[N-])c1cnc(Cl)c2cnc(Cl)cc12 ; COC[C@@](C)(N=[N+]=[N-])c1cnc(OC2CN(C(=O)[C@H]3C[C@H]3F)C2)c2cnc(Cl)cc12]

### Steps 1-2: (3-((4-((S)-2-Azido-1-methoxypropan-2-yl)-6-chloro-2,7-naphthyridin-1-yl)oxy)azetidin-1-yl)((1R,2R)-2-fluorocyclopropyl)methanone

The title compound was prepared from (1R,2R)-2-fluorocyclopropane-1-carboxylic acid and (S)-4-(2-azido-1-methoxypropan-2-yl)-1,6-dichloro-2,7-naphthyridine using a similar procedure as described in Step 2 of Intermediate 30 and Step 1 of Intermediate 9.

### Example 2q

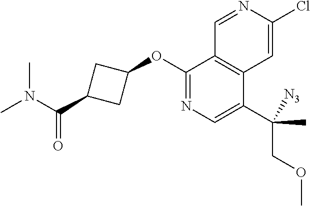 [SMILES: COC[C@@](C)(N=[N+]=[N-])c1cnc(O[C@H]2C[C@@H](C(=O)N(C)C)C2)c2cnc(Cl)cc12]

### Intermediate 33: cis-3-((4-((S)-2-Azido-1-methoxypropan-2-yl)-6-chloro-2,7-naphthyridin-1-yl)oxy)-N,N-dimethylcyclobutane-1-carboxamide

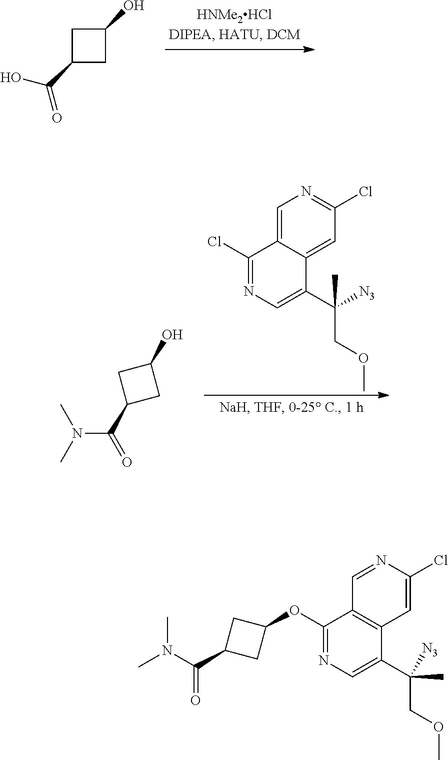

### Step 1: cis-3-Hydroxy-N,N-dimethylcyclobutane-1-carboxamide

DIPEA (1.16 mL, 6.63 mmol, 3.89 eq) was added to a solution of cis-3-hydroxycyclobutanecarboxylic acid (200 mg, 1.72 mmol) HATU (655 mg, 1.72 mmol, 1.0 eq) dimethylamine hydrochloride (169 mg, 2.07 mmol, 1.20 eq) in anhydrous DMF (5.00 mL) at 23° C. The resulting reaction mixture was stirred at 23° C. for 4 h, then was concentrated and purified by flash column chromatography on silica gel (gradient elution with MeOH/DCM) to give the title compound (222 mg, 86%) as a while solid.

### Step 2: cis-3-((4-((S)-2-Azido-1-methoxypropan-2-yl)-6-chloro-2,7-naphthyridin-1-yl)oxy)-N,N-dimethylcyclobutane-1-carboxamide

The title compound was prepared from cis-3-hydroxy-N,N-dimethylcyclobutane-1-carboxamide and (S)-4-(2-azido-1-methoxypropan-2-yl)-1,6-dichloro-2,7-naphthyridine using a similar procedure as described in Step 1 of Intermediate 9.

### Example 2r

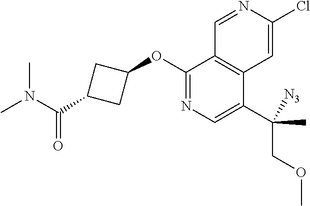 [SMILES: COC[C@@](C)(N=[N+]=[N-])c1cnc(O[C@H]2C[C@H](C(=O)N(C)C)C2)c2cnc(Cl)cc12]

### Intermediate 34: trans-3-((4-((S)-2-Azido-1-methoxypropan-2-yl)-6-chloro-2,7-naphthyridin-1-yl)oxy)-N,N-dimethylcyclobutane-1-carboxamide

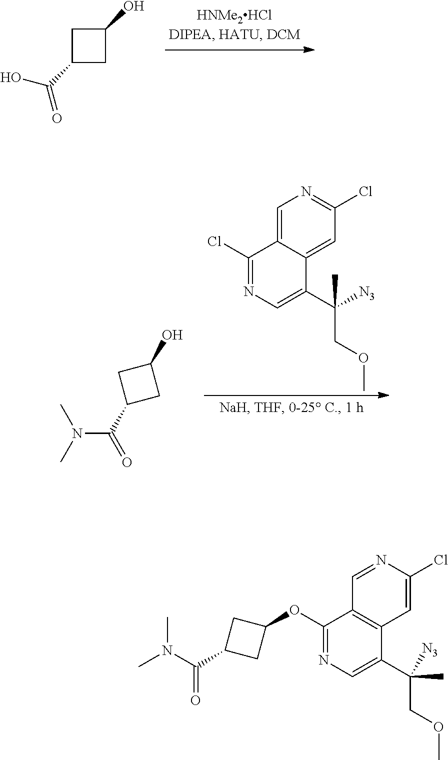

### Steps 1-2: trans-3-((4-((S)-2-Azido-1-methoxypropan-2-yl)-6-chloro-2,7-naphthyridin-1-yl)oxy)-N,N-dimethylcyclobutane-1-carboxamide

The title compound was prepared from trans-3-hydroxycyclobutane-1-carboxylic acid and (S)-4-(2-azido-1-methoxypropan-2-yl)-1,6-dichloro-2,7-naphthyridine using a similar procedure as described in Step 1 of Intermediate 33 and Step 1 of Intermediate 9.

### Example 2s

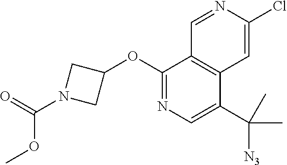 [SMILES: COC(=O)N1CC(Oc2ncc(C(C)(C)N=[N+]=[N-])c3cc(Cl)ncc23)C1]

### Intermediate 35: Methyl 3-((4-(2-azidopropan-2-yl)-6-chloro-2,7-naphthyridin-1-yl)oxy)azetidine-1-carboxylate

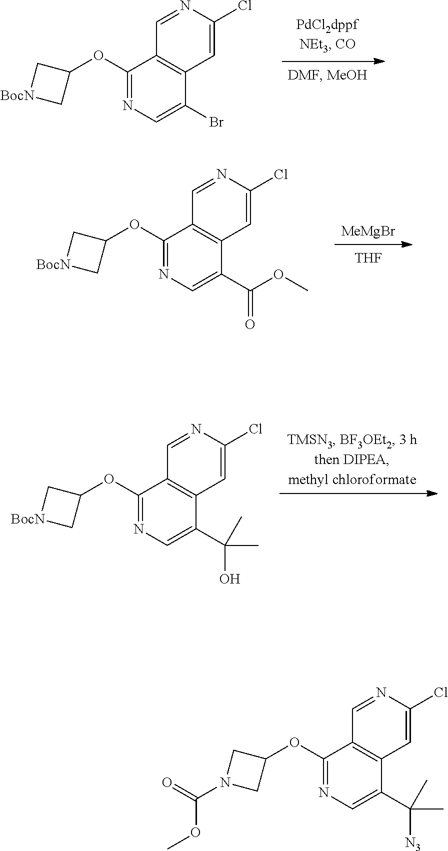

### Step 1: Methyl 1-((1-(tert-butoxycarbonyl)azetidin-3-yl)oxy)-6-chloro-2,7-naphthyridine-4-carboxylate

tert-Butyl 3-((4-bromo-6-chloro-2,7-naphthyridin-1-yl)oxy)azetidine-1-carboxylate (1.3 g, 3.13 mmol), Pd(dppf)Cl₂ (0.256 g, 0.313 mmol) and NEt₃ (0.524 ml, 3.76 mmol) was stirred in MeOH (8.36 ml) and DMF (4.18 ml). The resulting mixture was subjected to alternating vacuum and CO gas 3 times then stirred under 30 psi CO gas for 3 h at 50° C. The mixture was then subjected to alternating vacuum and N₂ gas 3 times, cooled to 23° C., and filtered through celite with DCM/MeOH (1:1). The filtrate was concentrated, and the residue was purified by flash-column chromatography on silica gel (20-100% EA:Hexanes) to give the title compound (0.6 g, 49% yield).

### Step 2: tert-Butyl 3-((6-chloro-4-(2-hydroxypropan-2-yl)-2,7-naphthyridin-1-yl)oxy)azetidine-1-carboxylate

MeMgBr (2.54 ml, 7.62 mmol) was added dropwise to a solution of methyl 1-((1-(tert-butoxycarbonyl)azetidin-3-yl)oxy)-6-chloro-2,7-naphthyridine-4-carboxylate (0.6 g, 1.524 mmol) in THF (6.09 ml) at 23° C. The reaction mixture was stirred for 30 min, then saturated aqueous NH₄Cl solution was slowly added until gas evolution ceased. The reaction mixture was then extracted with EA, and the organic layer was dried over sodium sulfate, filtered, and concentrated. The residue was purified by flash-column chromatography on silica gel (20-100% EA:Hexanes) to give the title compound (0.5 g, 83% yield).

### Step 3: Methyl 3-((4-(2-azidopropan-2-yl)-6-chloro-2,7-naphthyridin-1-yl)oxy)azetidine-1-carboxylate

To a solution of tert-butyl 3-((6-chloro-4-(2-hydroxypropan-2-yl)-2,7-naphthyridin-1-yl)oxy)azetidine-1-carboxylate (0.12 g, 0.305 mmol) in DCM (1.5 ml) at 23° C. was added TMSN₃ (0.100 ml, 0.762 mmol) followed by BF₃OEt₂ (0.085 ml, 0.670 mmol). The reaction mixture was stirred for 3 h, then DIPEA (0.532 ml, 3.05 mmol) was added to the reaction mixture followed immediately by methyl chloroformate (0.118 ml, 1.523 mmol). The reaction mixture was stirred for 1 h, then was diluted with EA and washed saturated aqueous sodium bicarbonate solution and brine. The organic layer was dried over sodium sulfate, filtered, and concentrated. The residue was purified by flash-column chromatography on silica gel (20-100% EA:Hexanes) to give the title compound (50 mg, 43.6% yield). MS (ES+) C₁₆H₁₇ClN₆O₃ requires: 376, found: 377 [M+H]⁺.

### Example 2t

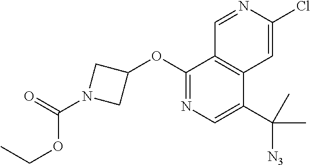 [SMILES: CCOC(=O)N1CC(Oc2ncc(C(C)(C)N=[N+]=[N-])c3cc(Cl)ncc23)C1]

### Intermediate 36: Ethyl 3-((4-(2-azidopropan-2-yl)-6-chloro-2,7-naphthyridin-1-yl)oxy)azetidine-1-carboxylate

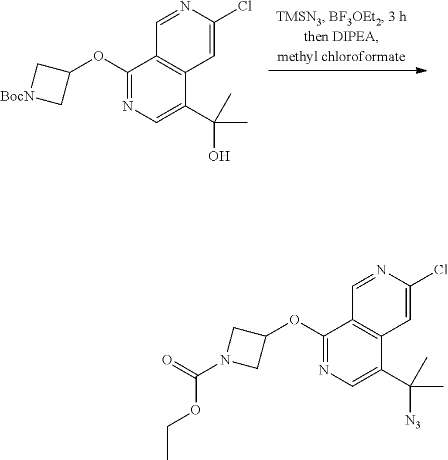 [SMILES: CC(C)(C)OC(=O)N1CC(Oc2ncc(C(C)(C)O)c3cc(Cl)ncc23)C1 ; "TMSN3, BF3OEt2, 3 hthen DIPEA,methyl chloroformate" ; CCOC(=O)N1CC(Oc2ncc(C(C)(C)N=[N+]=[N-])c3cc(Cl)ncc23)C1]

### Step 1: Ethyl 3-((4-(2-azidopropan-2-yl)-6-chloro-2,7-naphthyridin-1-yl)oxy)azetidine-1-carboxylate

The title compound was prepared from tert-butyl 3-((6-chloro-4-(2-hydroxypropan-2-yl)-2,7-naphthyridin-1-yl)oxy)azetidine-1-carboxylate and ethyl chloroformate using a similar procedure as described in Step 3 of Intermediate 35. MS (ES+) C₁₇H₁₉ClN₆O₃ requires: 390, found: 391 [M+H]⁺.

### Example 2u

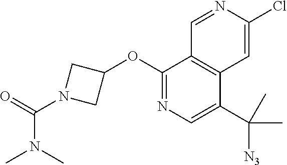 [SMILES: CN(C)C(=O)N1CC(Oc2ncc(C(C)(C)N=[N+]=[N-])c3cc(Cl)ncc23)C1]

### Intermediate 37: 3-((4-(2-Azidopropan-2-yl)-6-chloro-2,7-naphthyridin-1-yl)oxy)-N,N-dimethylazetidine-1-carboxamide

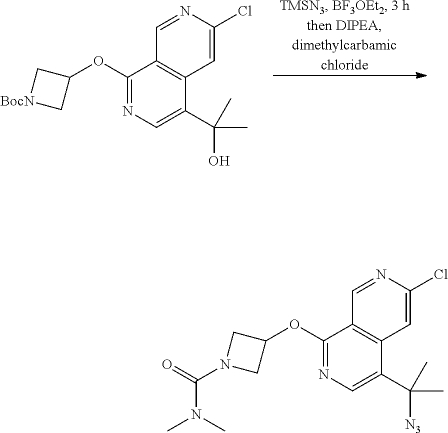 [SMILES: CC(C)(C)OC(=O)N1CC(Oc2ncc(C(C)(C)O)c3cc(Cl)ncc23)C1 ; "TMSN3, BF3OEt2, 3 hthen DIPEA,dimethylcarbamicchloride" ; CN(C)C(=O)N1CC(Oc2ncc(C(C)(C)N=[N+]=[N-])c3cc(Cl)ncc23)C1]

### Step 1: Ethyl 3-((4-(2-azidopropan-2-yl)-6-chloro-2,7-naphthyridin-1-yl)oxy)azetidine-1-carboxylate

The title compound was prepared from tert-butyl 3-((6-chloro-4-(2-hydroxypropan-2-yl)-2,7-naphthyridin-1-yl)oxy)azetidine-1-carboxylate and dimethylcarbamic chloride using a similar procedure as described in Step 3 of Intermediate 35. MS (ES+) C₁₇H₂₀ClN₇O₂ requires: 389, found: 390 [M+H]⁺.

### Example 2v

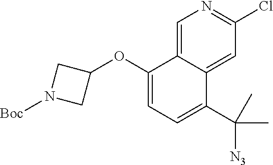 [SMILES: CC(C)(C)OC(=O)N1CC(Oc2ccc(C(C)(C)N=[N+]=[N-])c3cc(Cl)ncc23)C1]

### Intermediate 38: tert-Butyl 3-((5-(2-azidopropan-2-yl)-3-chloroisoquinolin-8-yl)oxy)azetidine-1-carboxylate

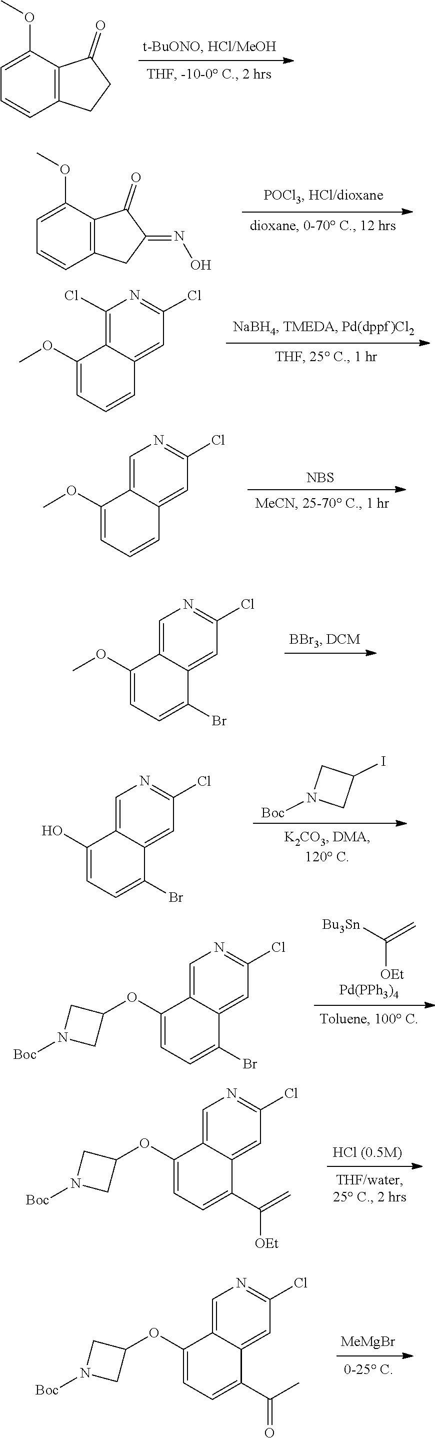

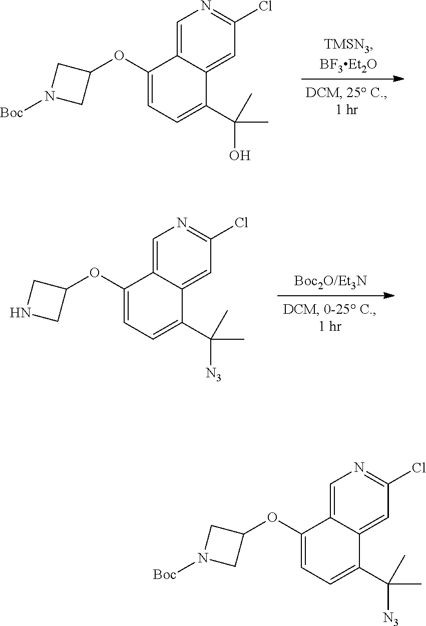

### Step 1: (E)-2-(Hydroxyimino)-7-methoxy-2,3-dihydro-1H-inden-1-one

t-BuONO (67.9 g, 659 mmol, 78.4 mL, 1.10 eq) was added to a solution of 7-methoxy-2,3-dihydro-1H-inden-1-one (99.0 g, 599 mmol, 1.00 eq) in THF (500 mL) at −10-0° C., followed by dropwise addition of HCl (4 M in MeOH, 15.0 mL, 0.10 eq) to the mixture at −10-0° C. The reaction mixture was stirred at 0° C. for 2 h, then was concentrated to give a residue. The residue was slurried in PE/EA=20:1 (200 mL) and filtered to give the title compound (107 g, 87% yield) as yellow solid. MS (ES+) C₁₀H₉NO₃ requires: 191, found: 192[M+H]⁺.

### Step 2: 1,3-Dichloro-8-methoxyisoquinoline

To a solution of (E)-2-(hydroxyimino)-7-methoxy-2,3-dihydro-1H-inden-1-one (107 g, 522 mmol, 1.00 eq) in dioxane (500 mL) was added POCl₃ (126 g, 827 mmol, 76.9 mL, 1.59 eq) and HCl (4 M in dioxane, 1.31 mL, 0.01 eq) at 0-10° C. The reaction mixture was stirred at 70° C. for 12 h, then was cooled to 25° C. and quenched with water (2.00 L). The quenched mixture was extracted with DCM (500 mL×4) and the organic layers were washed with brine (500 mL×2), dried over Na₂SO₄, filtered and concentrated to give a residue. The residue was purified by column chromatography (SiO₂, PE/EA=20/1 to 10/1) to give the title compound (48.8 g, 40.9% yield) as light yellow solid. MS (ES+) C₁₀H₇Cl₂NO requires: 227, found: 228[M+H]⁺.

### Step 3: 3-Chloro-8-methoxyisoquinoline

To a solution of 1,3-dichloro-8-methoxyisoquinoline (48.8 g, 213 mmol, 1.00 eq) in THF (250 mL) was added TMEDA (37.3 g, 320 mmol, 48.4 mL, 1.50 eq) and Pd(dppf)Cl₂ (1.57 g, 2.14 mmol, 0.01 eq) at 25° C. Then NaBH₄ (17.2 g, 456 mmol, 2.13 eq) was slowly added to the reaction mixture and the reaction mixture was stirred at 25° C. for 1 h. The reaction mixture was poured into 1N HCl (1.00 L), extracted with EA (200 mL×3). The combined organic layers were filtered through Celite® and the filtrate was washed with brine (500 mL), dried over Na₂SO₄, filtered and concentrated to give a residue. The residue was purified by column chromatography (SiO₂, PE/EA=20/1 to 5/1) to give the title compound (26.7 g, 64.3% yield) as a light yellow solid. MS (ES+) C₁₀H₈ClNO requires: 193, found: 194[M+H]⁺.

### Step 4: 5-Bromo-3-chloro-8-methoxyisoquinoline

To a solution of 3-chloro-8-methoxyisoquinoline (26.7 g, 137 mmol, 1.00 eq) in MeCN (300 mL) was added NBS (29.3 g, 165 mmol, 1.20 eq) at 25° C. The reaction mixture was stirred at 70° C. for 1 h, then was cooled to 25° C. The mixture was filtered, and the filter cake was washed with MeCN (100 mL). The filter cake was collected and dried under vacuum. The filtrate was purified by column chromatography (SiO₂, PE/EA=1:0 to 1:1, Rf=0.45) to give the title compound (26.17 g, 69.6% yield) as an off-white solid. MS (ES+) C₁₀H₇BrClNO requires: 273, found: 274[M+H]⁺.

### Step 5: 5-Bromo-3-chloroisoquinolin-8-ol

To a solution of 5-bromo-3-chloro-8-methoxyisoquinoline (3.00 g, 11.01 mmol) in DCM (50 mL) was added BBr₃ (13.8 g, 55.0 mmol) in one portion. The reaction mixture was stirred at 50° C. for 12 h, then was quenched with MeOH (15 mL) and concentrated to give a residue. The residue was purified by column chromatography (PE/EA=10:1 to 1:1) to give the title compound (2.5 g, 88% yield) as a yellow solid. ¹H NMR (400 MHz, 6d-DMSO): δ ppm 11.3 (s, 1H), 9.21 (s, 1H), 7.84 (d, J=8.4 Hz, 1H), 7.72 (s, 1H), 8.85 (d, J=8.4 Hz, 1H).

### Step 6: tert-Butyl 3-((5-bromo-3-chloroisoquinolin-8-yl)oxy)azetidine-1-carboxylate

A mixture of 5-bromo-3-chloroisoquinolin-8-ol (1.40 g, 5.4 mmol), tert-butyl 3-iodoazetidine-1-carboxylate (2.30 g, 8.12 mmol) and K₂CO₃ (1.50 g, 10.8 mmol) in DMA (40 mL) was stirred at 120° C. for 2 h. The reaction mixture was then diluted with water (30 mL) and extracted with EA (20 mL×3). The combined organic layers were concentrated to give a residue. The residue was purified by flash silica gel chromatography (Eluent of 0˜50% EA/PE) to give the title compound (2.22 g, 99% yield) as an off-white solid.

### Steps 7-9: tert-Butyl 3-((3-chloro-5-(2-hydroxypropan-2-yl)isoquinolin-8-yl)oxy)azetidine-1-carboxylate

The title compound was prepared from tert-butyl 3-((5-bromo-3-chloroisoquinolin-8-yl)oxy)azetidine-1-carboxylate using a similar procedure as described in Steps 2-4 of Intermediate 6.

### Step 10: 8-(Azetidin-3-yloxy)-5-(2-azidopropan-2-yl)-3-chloroisoquinoline

To a solution of tert-butyl 3-((3-chloro-5-(2-hydroxypropan-2-yl)isoquinolin-8-yl)oxy)azetidine-1-carboxylate (560 mg, 1.43 mmol) and TMSN₃ (821 mg, 7.13 mmol) in DCM (10 ml) was added BF₃Et₂O (405 mg, 2.85 mmol) at 25° C. The reaction mixture was stirred at 25° C. for 1 h, then was quenched by addition of saturated sodium bicarbonate solution (50 mL) and extracted with EA (50 ml×3). The combined organic layers were washed with brine, dried over sodium sulfate, filtered, and concentrated to give the title compound (600 mg, crude) as a yellow oil.

### Step 11: tert-Butyl 3-((5-(2-azidopropan-2-yl)-3-chloroisoquinolin-8-yl)oxy)azetidine-1-carboxylate

To a solution of 8-(azetidin-3-yloxy)-5-(2-azidopropan-2-yl)-3-chloroisoquinoline (580 mg, 1.83 mmol) and NEt₃ (148 mg, 1.46 mmol) in DCM (10 ml) was added Boc₂O (239 mg, 1.10 mmol), then the reaction mixture was stirred at 25° C. for 1 h. The reaction mixture was then concentrated, and the residue was purified by flash silica gel chromatography (Eluent of 0˜30% EA/PE) to give the title compound (510 mg) as an off-white solid.

### Example 2w

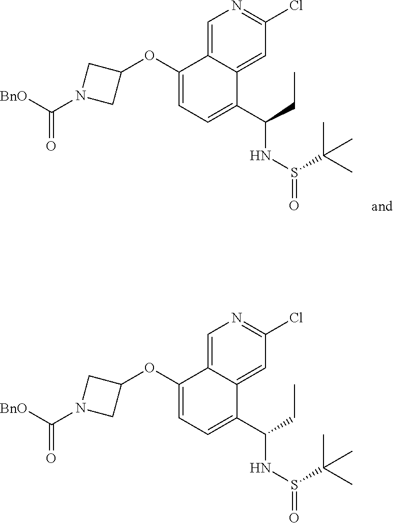 [SMILES: CC[C@@H](N[S@@](=O)C(C)(C)C)c1ccc(OC2CN(C(=O)OCc3ccccc3)C2)c2cnc(Cl)cc12 ; CC[C@H](N[S@@](=O)C(C)(C)C)c1ccc(OC2CN(C(=O)OCc3ccccc3)C2)c2cnc(Cl)cc12]

### Intermediates 39 and 40: Benzyl 3-((5-((R)-1-(((S)-tert-butylsulfinyl)amino)propyl)-3-chloroisoquinolin-8-yl)oxy)azetidine-1-carboxylate and benzyl 3-((5-((S)-1-(((S)-tert-butylsulfinyl)amino)propyl)-3-chloroisoquinolin-8-yl)oxy)azetidine-1-carboxylate

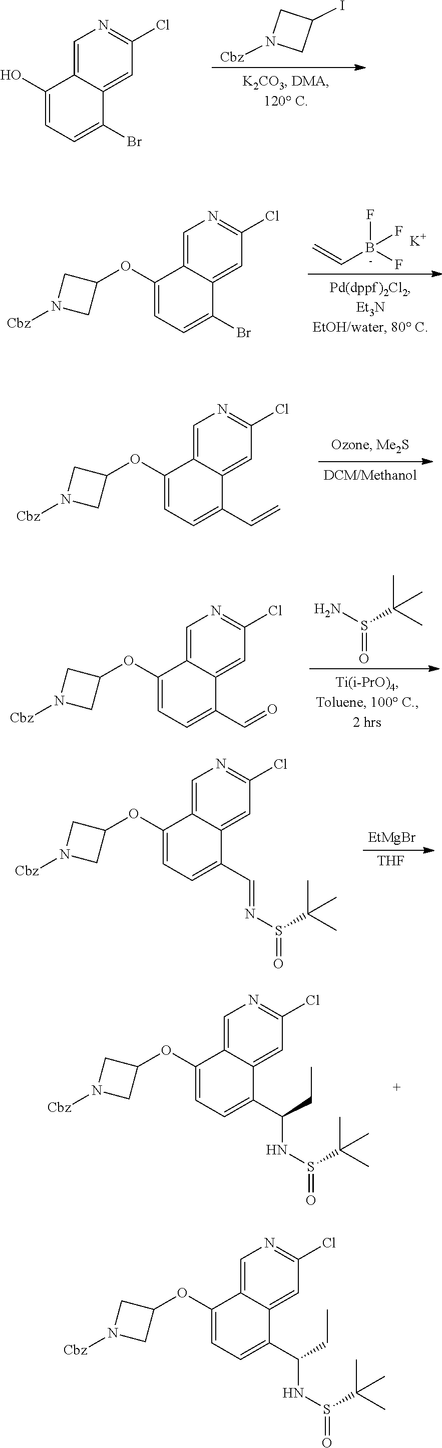

### Step 1: Benzyl 3-((5-bromo-3-chloroisoquinolin-8-yl)oxy)azetidine-1-carboxylate

The title compound was prepared from 5-bromo-3-chloroisoquinolin-8-ol and benzyl 3-iodoazetidine-1-carboxylate using a procedure similar to the one described in Steps 1-6 of Intermediate 38.

### Step 2: Benzyl 3-((3-chloro-5-vinylisoquinolin-8-yl)oxy)azetidine-1-carboxylate

A solution of benzyl 3-((5-bromo-3-chloroisoquinolin-8-yl)oxy)azetidine-1-carboxylate (3.00 g, 6.70 mmol), trifluoro(vinyl)borate (1.17 g, 8.71 mmol), Pd(dppf)Cl₂ (490 mg, 670 mol) and Et₃N (1.36 g, 13.4 mmol) in EtOH (250 mL) and water (1 mL) was stirred at 80° C. for 2 h. The reaction mixture was then concentrated, and the residue was purified by flash silica gel chromatography (eluent of 0-60% EA/PE) to give the title compound (2.20 g, 83% yield) as an off-white solid. MS (ES+) C₂₂H₁₉ClN₂O₃ requires: 394, found: 395[M+H]⁺.

### Steps 3-5: Benzyl 3-((5-((R)-1-(((S)-tert-butylsulfinyl)amino)propyl)-3-chloroisoquinolin-8-yl)oxy)azetidine-1-carboxylate and benzyl 3-((5-((S)-1-(((S)-tert-butylsulfinyl)amino)propyl)-3-chloroisoquinolin-8-yl)oxy)azetidine-1-carboxylate

The title compounds were prepared from benzyl 3-((3-chloro-5-vinylisoquinolin-8-yl)oxy)azetidine-1-carboxylate using a procedure similar to that described in Steps 5-7 of Intermediates 13 and 14. The mixture of two compounds was purified by prep-HPLC (column: Phenomenex luna C18 150*40 mm*15 um; mobile phase: [water (0.225% FA)-ACN]; B %: 58%-88%, 10 min) to give the title compounds separately. The stereochemistry was assigned arbitrarily.

First eluting isomer, Peak 1 (Intermediate 39): 400 mg, 42% yield was obtained as a yellow solid. MS (ES+) C₂₇H₃₂ClN₃O₄S requires: 529, found: 530[M+H]⁺.

Second eluting isomer, Peak 2 (Intermediate 40): (370 mg, 39% yield) was obtained as a yellow solid. MS (ES+) C₂₇H₃₂ClN₃O₄S requires: 529, found: 530[M+H]⁺.

### Example 2x

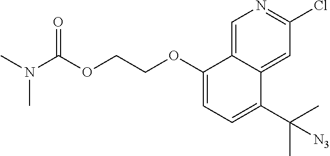 [SMILES: CN(C)C(=O)OCCOc1ccc(C(C)(C)N=[N+]=[N-])c2cc(Cl)ncc12]

### Intermediate 41: 2-((5-(2-Azidopropan-2-yl)-3-chloroisoquinolin-8-yl)oxy)ethyl dimethylcarbamate

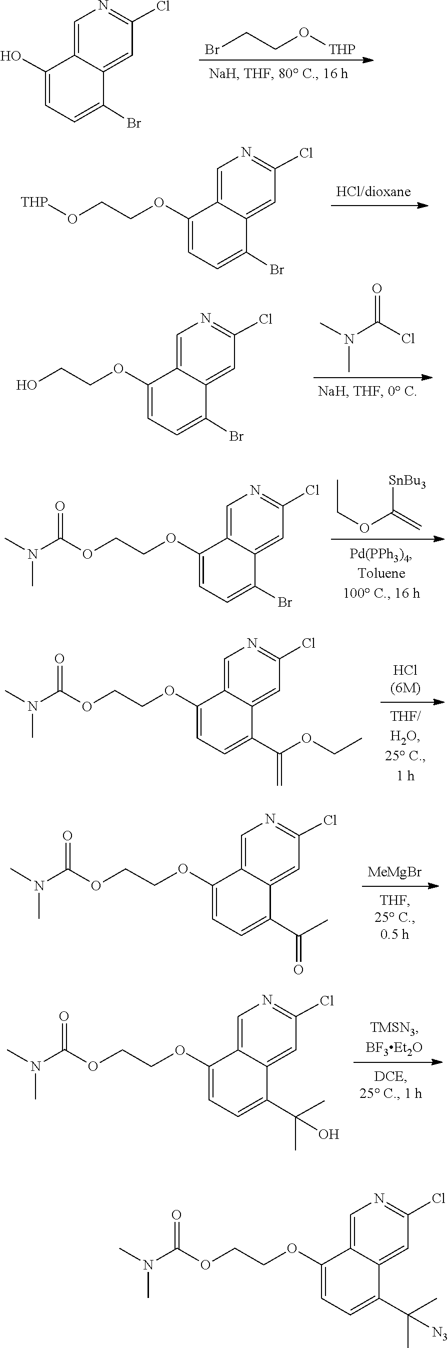

### Step 1: 5-Bromo-3-chloro-8-(2-((tetrahydro-2H-pyran-2-yl)oxy)ethoxy)isoquinoline

To a solution of 5-bromo-3-chloroisoquinolin-8-ol (title compound from Step 5 of Intermediate 38, 1 g, 3.87 mmol) in THF (100 mL) was added NaH (232.11 mg, 5.80 mmol, 60% purity, 1.5 eq). The reaction mixture was stirred at 25° C. for 0.5 h, then 2-(2-bromoethoxy)tetrahydro-2H-pyran (2.43 g, 11.61 mmol,) was added and the mixture was heated to 80° C. for 71.5 h. The reaction mixture was then removed from heat and diluted with water (50 mL) and extracted with EA (20 mL×3). The combined organic layers were dried over anhydrous sodium sulfate, filtered and concentrated to give a residue. The residue was purified by silica gel column chromatography (PE:EA=1:0 to 3:1) to give the title compound (1.4 g, 93.59% yield) as a yellow solid.

### Step 2: 2-((5-Bromo-3-chloroisoquinolin-8-yl)oxy)ethan-1-ol

To a solution of 5-bromo-3-chloro-8-(2-((tetrahydro-2H-pyran-2-yl)oxy)ethoxy) isoquinoline (1.3 g, 3.36 mmol, 1 eq) in dioxane (8 mL) was added HCl/dioxane (4 M, 3.88 mL). The mixture was stirred at 25° C. for 10 min, then was concentrated to give the title compound (1.0 g, 3.31 mmol, 98% yield) as a yellow solid that was used in the next step without further purification.

### Step 3: 2-((5-Bromo-3-chloroisoquinolin-8-yl)oxy)ethyl dimethylcarbamate

To a solution of N,N-dimethylcarbamoyl chloride (426.5 mg, 3.97 mmol) in THF (15 mL) was added NaH (158.65 mg, 3.97 mmol, 60% purity) at 0° C. The mixture was stirred for 15 min, then 2-((5-bromo-3-chloroisoquinolin-8-yl)oxy)ethan-1-ol (1.0 g, 3.31 mmol, 1 eq) was added and the mixture was stirred at 25° C. for 45 min. The reaction mixture was quenched with water (100 mL) and extracted with EA (50 mL×3). The combined organic layers were dried over anhydrous sodium sulfate, filtered and concentrated to give the title compound (1.03 g, 83.41% yield) as a yellow solid that was used in the next step without further purification.

### Steps 4-7: 2-((5-(2-Azidopropan-2-yl)-3-chloroisoquinolin-8-yl)oxy)ethyl dimethylcarbamate

The title compound was prepared from 2-((5-bromo-3-chloroisoquinolin-8-yl)oxy)ethyl dimethylcarbamate using a procedure similar to that described in Steps 2-5 of Intermediate 6.

MS (ES+) C₁₇H₂₀ClN₅O₃ requires: 377, found: 378[M+H]⁺.

### Example 2y

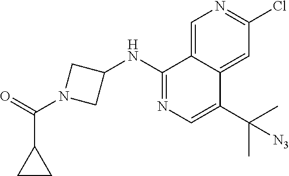 [SMILES: CC(C)(N=[N+]=[N-])c1cnc(NC2CN(C(=O)C3CC3)C2)c2cnc(Cl)cc12]

### Intermediate 42: (3-((4-(2-Azidopropan-2-yl)-6-chloro-2,7-naphthyridin-1-yl)amino)azetidin-1-yl)(cyclopropyl)methanone

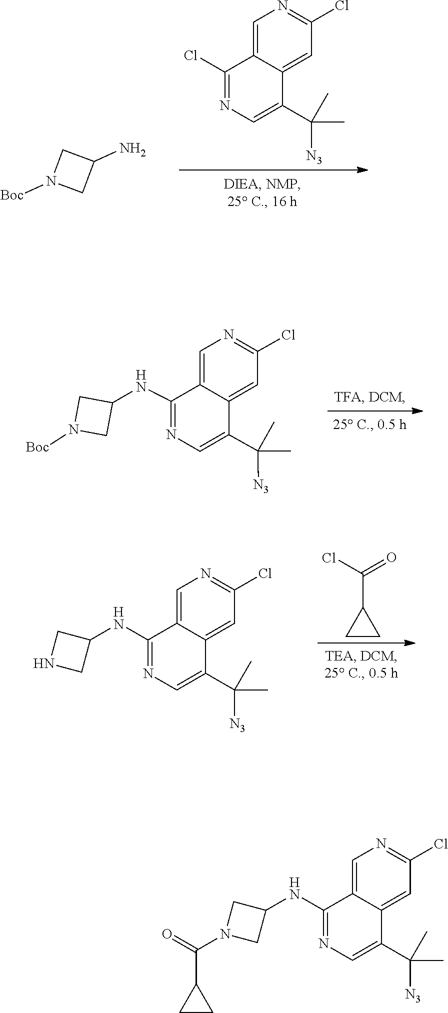 [SMILES: CC(C)(C)OC(=O)N1CC(N)C1 ; CC(C)(N=[N+]=[N-])c1cnc(Cl)c2cnc(Cl)cc12 ; CC(C)(C)OC(=O)N1CC(Nc2ncc(C(C)(C)N=[N+]=[N-])c3cc(Cl)ncc23)C1 ; CC(C)(N=[N+]=[N-])c1cnc(NC2CNC2)c2cnc(Cl)cc12 ; O=C(Cl)C1CC1 ; CC(C)(N=[N+]=[N-])c1cnc(NC2CN(C(=O)C3CC3)C2)c2cnc(Cl)cc12]

### Step 1: tert-Butyl 3-((4-(2-azidopropan-2-yl)-6-chloro-2,7-naphthyridin-1-yl)amino)azetidine-1-carboxylate

To a solution of 4-(2-azidopropan-2-yl)-1,6-dichloro-2,7-naphthyridine (Intermediate 6, 200 mg, 708 μmol) and tert-butyl 3-aminoazetidine-1-carboxylate (183 mg, 1.06 mmol) in NMP (2 mL) was added DIPEA (137 mg, 1.06 mmol). The mixture was stirred at 25° C. for 16 h, then was diluted with water (40 mL) and extracted with EA (40 mL×3). The combined organic layers were dried over anhydrous sodium sulfate, filtered and concentrated to give a residue. The residue was purified by column chromatography on silica gel (PE/EA=10/1 to 1/1) to give the title compound (200 mg, 67% yield) as a yellow solid.

### Step 2: N-(Azetidin-3-yl)-4-(2-azidopropan-2-yl)-6-chloro-2,7-naphthyridin-1-amine

To a solution of TFA (436 mg, 3.83 mmol) in DCM (0.5 mL) was added tert-butyl 3-((4-(2-azidopropan-2-yl)-6-chloro-2,7-naphthyridin-1-yl)amino)azetidine-1-carboxylate (80.0 mg, 191 μmol). The reaction mixture was stirred at 25° C. for 0.5 h, then was concentrated to give the title compound (60.0 mg, 98% yield) as yellow oil.

### Step 3: (3-((4-(2-Azidopropan-2-yl)-6-chloro-2,7-naphthyridin-1-yl)amino)azetidin-1-yl)(cyclopropyl)methanone

To a solution of N-(azetidin-3-yl)-4-(2-azidopropan-2-yl)-6-chloro-2,7-naphthyridin-1-amine (60.0 mg, 188 μmol) and Et₃N (38.2 mg, 377 μmol) in DCM (3 mL) was added cyclopropanecarbonyl chloride (19.7 mg, 188 μmol). The reaction mixture was stirred at 25° C. for 0.5 h, then was diluted with water (30 mL) and extracted with EA (20 mL×3). The combined organic layers were dried over anhydrous sodium sulfate, filtered and concentrated to give a residue. The residue was purified by prep-TLC (PE/EA=1/1) to give the title compound (60.0 mg, 82% yield) as a yellow solid.

### Example 2z

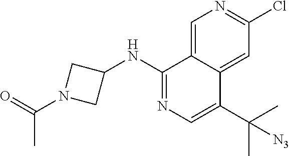 [SMILES: CC(=O)N1CC(Nc2ncc(C(C)(C)N=[N+]=[N-])c3cc(Cl)ncc23)C1]

### Intermediate 43: 1-(3-((4-(2-Azidopropan-2-yl)-6-chloro-2,7-naphthyridin-1-yl)amino)azetidin-1-yl)ethan-1-one

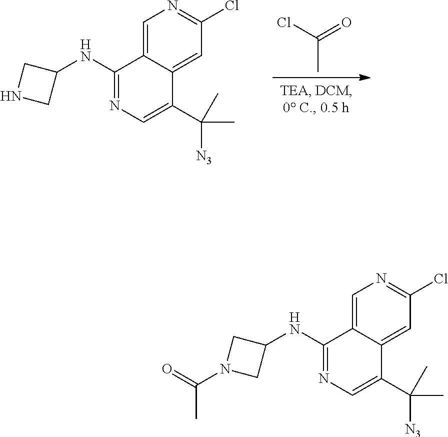

### Step 1: 1-(3-((4-(2-Azidopropan-2-yl)-6-chloro-2,7-naphthyridin-1-yl)amino)azetidin-1-yl)ethan-1-one

To a solution of N-(azetidin-3-yl)-4-(2-azidopropan-2-yl)-6-chloro-2,7-naphthyridin-1-amine (Intermediate 42, Step 2) (70.0 mg, 220 μmol) and Et₃N (44.5 mg, 440 μmol) in DCM (1 mL) was added acetyl chloride (17.2 mg, 220 μmol). The reaction mixture was stirred at 0° C. for 0.5 h, then was diluted with water (20 mL) and extracted with EA (20 mL×3). The combined organic layers were dried over anhydrous sodium sulfate, filtered and concentrated to give a residue. The residue was purified by prep-TLC (PE/EA=1/1) to give the title compound (70.0 mg, 88% yield) as yellow oil.

### Example 2ab

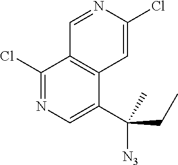 [SMILES: CC[C@@](C)(N=[N+]=[N-])c1cnc(Cl)c2cnc(Cl)cc12]

### Intermediate 44: (R)-4-(2-Azidobutan-2-yl)-1,6-dichloro-2,7-naphthyridine

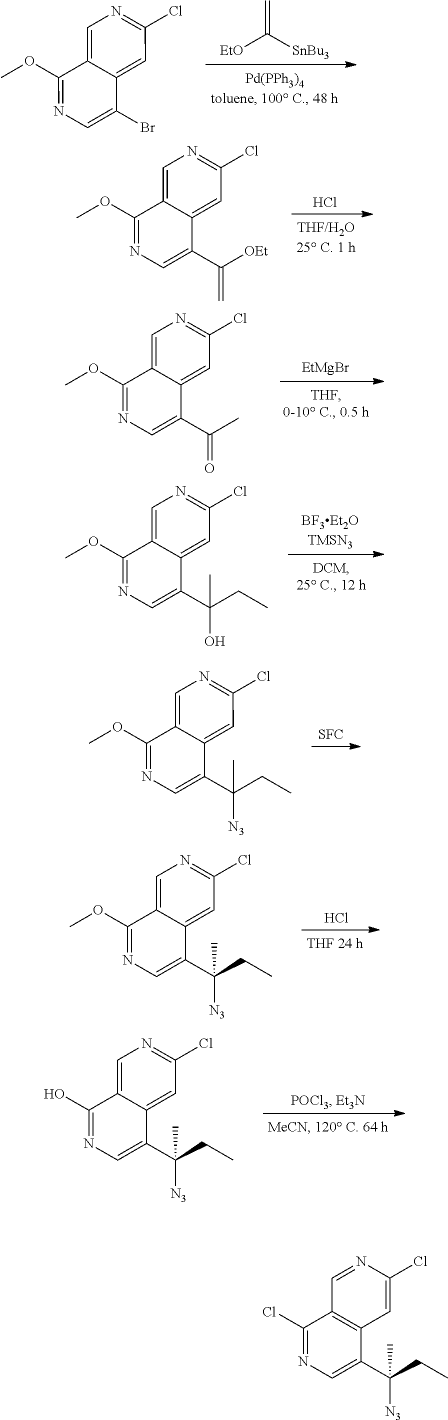

### Step 1: 6-Chloro-4-(1-ethoxyvinyl)-1-methoxy-2,7-naphthyridine

A suspension of 4-bromo-6-chloro-1-methoxy-2,7-naphthyridine (title compound from Step 1 of Intermediate 6, 220 g, 804 mmol, 1.00 eq), tributyl(1-ethoxyvinyl)stannane (264 g, 731 mmol, 246 mL, 0.90 eq), Pd(PPh₃)₄(46.4 g, 40.2 mmol, 0.05 eq) in toluene (2.20 L) was stirred at 100° C. under N₂ for 36 h. The mixture was cooled to 25° C. and more tributyl(1-ethoxyvinyl)stannane (0.35 eq) was added to above solution under N₂. The mixture was stirred at 100° C. for 12 h. The mixture was cooled to 25° C. and poured into saturated aqueous KF solution (2.00 L). The mixture was filtered through a pad of celite and the filtrate was extracted with EA (1.00 L×2). The combined organic layer was concentrated, and the residue was purified by column chromatography (SiO₂, PE/EA=20/1-10/1) to give the title compound (157 g, 668 mmol, 83.1% yield) as a white solid.

### Step 2: 1-(6-Chloro-1-methoxy-2,7-naphthyridin-4-yl)ethan-1-one

To a solution of 6-chloro-4-(1-ethoxyvinyl)-1-methoxy-2,7-naphthyridine (157 g, 593 mmol, 1.00 eq) in THF (942 mL) and H₂O (157 mL) was added HCl (1.50 M, 39.5 mL, 0.10 eq), then the suspension was stirred 25° C. for 1 h. The mixture was poured into saturated aqueous NaHCO₃ solution (1.50 L) and extracted with EA (1.50 L×2). The combined organic layer was concentrated to give a residue. The residue was slurried in PE/EA=10:1 (550 mL) at 20-25° C. for 10 mins, then the suspension was filtered, and the filter cake dried to give the title compound (114 g) as a white solid. MS (ES+) C₁₁H₉ClN₂O₂ requires: 236, found: 237[M+H]⁺.

### Step 3: 2-(6-Chloro-1-methoxy-2,7-naphthyridin-4-yl)butan-2-ol

A solution of 1-(6-chloro-1-methoxy-2,7-naphthyridin-4-yl)ethan-1-one (114 g, 481 mmol, 1.00 eq) in THF (2.28 L) was added to a mixture of EtMgBr (3.00 M, 481 mL, 3.00 eq) at 0˜10° C. The reaction mixture was stirred at 0-10° C. for 0.5 h, then was poured into saturated aqueous NH₄Cl solution (1.50 L) and extracted with EA (1.00 L*2). The combined organic layer was washed with brine (1.00 L), dried over with Na₂SO₄, filtered, and concentrated. The residue was purified by silica gel chromatography (PE/EA=5/1 to 2/1) to give the title compound (100 g) as a yellow oil. MS (ES+) C₁₃H₁₅ClN₂O₂ requires: 266, found: 267[M+H]⁺.

### Step 4: 4-(2-Azidobutan-2-yl)-6-chloro-1-methoxy-2,7-naphthyridine

To a solution of 2-(6-chloro-1-methoxy-2,7-naphthyridin-4-yl)butan-2-ol (100 g, 374 mmol, 1.00 eq) and BF₃·Et₂O (49.2 mL, 187 mmol, 47.0% purity, 0.50 eq) in DCM (1.00 L) was added TMSN₃ (123 mL, 937 mmol, 2.50 eq) at 25° C. The reaction mixture was stirred at 25° C. for 12 hrs. The reaction mixture was then added slowly to saturated aqueous NaHCO₃ solution (1.00 L) and extracted with DCM (200 mL×2). The organic layer was washed with brine (1.00 L), dried over with Na₂SO₄, filtered, and concentrated. The residue was purified by silica gel chromatography (PE/EA=100/1˜5/1) to give a solid. The solid was slurried in PE/EA=8/1 (90.0 mL) at 25° C. for 10 min, then was filtered and the filter cake was dried to give the title compound (46.0 g, 155 mmol, 41.5% yield) as a white solid. MS (ES+) C₁₃H₁₄ClN₅O requires: 291, found: 292[M+H]⁺.

### Step 5: (R)-4-(2-Azidobutan-2-yl)-6-chloro-1-methoxy-2,7-naphthyridine

4-(2-Azidobutan-2-yl)-6-chloro-1-methoxy-2,7-naphthyridine (46.0 g, 157 mmol, 98.7% purity, 1.00 eq) was separated by SFC (column: Daicel ChiralPak IG (250*30 mm, 10 um); mobile phase: [0.1% NH₃H₂O MEOH]; B %: 20%-20%). The second eluting peak was concentrated to give the title compound (21.5 g, 46.7% yield) as a white solid. The absolute stereochemistry of the title compound was determined by X-ray crystal structure of a final compound prepared from this intermediate. MS (ES+) C₁₃H₁₄ClN₅O requires: 291, found: 292[M+H]⁺.

### Step 6: (R)-4-(2-Azidobutan-2-yl)-6-chloro-2,7-naphthyridin-1-ol

To a solution of (R)-4-(2-azidobutan-2-yl)-6-chloro-1-methoxy-2,7-naphthyridine (21.5 g, 72.7 mmol, 1.00 eq) in THF (215 mL) was added HCl (3.00 M, 122 mL, 5.07 eq) at 25° C., then the suspension was stirred at 25° C. for 12 h, followed by stirring at 30° C. for 12 h. The reaction mixture poured into saturated aqueous NaHCO₃ (500 mL) and extracted with EA (150 mL×2), washed with brine (200 mL), dried over Na₂SO₄, filtered, and concentrated. The residue was slurried in PE (100 mL) at 25° C. for 10 mins, then was filtered and the filter cake was dried to give the title compound (18.5 g) as a white solid. MS (ES+) C₁₂H₁₂ClN₅O requires: 277, found: 278[M+H]⁺.

### Step 7: (R)-4-(2-Azidobutan-2-yl)-1,6-dichloro-2,7-naphthyridine

The reaction described below was carried out in parallel in triplicate and the resulting crude products were combined for purification. To a solution of (R)-4-(2-azidobutan-2-yl)-6-chloro-2,7-naphthyridin-1-ol (6.16 g, 22.1 mmol, 1.00 eq) in MeCN (190 mL) was added POCl₃ (10.3 mL, 110 mmol, 5.00 eq) and Et₃N (8.03 mL, 57.6 mmol, 2.60 eq). The reaction mixture was stirred at 120° C. for 64 h, then was concentrated to give a residue. The residue was diluted with ethyl acetate (500 mL), then was quenched with water (500 mL) at 20-30° C. and stirred at 30° C. for 30 min. The mixture was adjusted pH=8 with addition of saturated aqueous Na₂CO₃ solution at 25° C., then was extracted with EA (500 mL×2). The combined organic layer was washed with brine (500 mL), dried over with Na₂SO₄, filtered, and concentrated. The residue was purified by silica gel chromatography (PE/EA=1/0˜8/1) to give the title compound (18.0 g, 88.4% yield) as a light yellow solid. MS (ES+) C₁₂H₁₁Cl₂N₅ requires: 295, found: 296[M+H]⁺. ¹H NMR (400 MHz, CDCl₃): δ 9.62 (s, 1H), 8.52 (s, 1H), 8.43 (s, 1H), 2.03-2.16 (m, 2H), 1.61-1.89 (m, 3H), 0.85-0.89 (m, 3H).

### Example 2ac

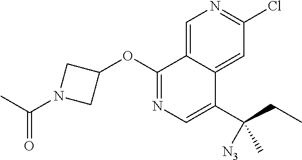 [SMILES: CC[C@@](C)(N=[N+]=[N-])c1cnc(OC2CN(C(C)=O)C2)c2cnc(Cl)cc12]

### Intermediate 45: (R)-1-(3-((4-(2-Azidobutan-2-yl)-6-chloro-2,7-naphthyridin-1-yl)oxy)azetidin-1-yl)ethan-1-one

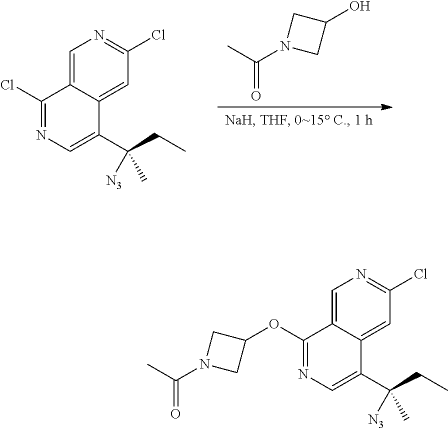

### Step 1: (R)-1-(3-((4-(2-Azidobutan-2-yl)-6-chloro-2,7-naphthyridin-1-yl)oxy)azetidin-1-yl)ethan-1-one

The title compound was prepared from (R)-4-(2-Azidobutan-2-yl)-1,6-dichloro-2,7-naphthyridine (Intermediate 44) and 1-(3-hydroxyazetidin-1-yl)ethan-1-one using a procedure similar to that described in Step 1 of Intermediate 9. ¹H-NMR (400 MHz, CDCl₃): δ ppm 9.47 (s, 1H), 8.40 (s, 1H), 8.18 (s, 1H), 5.63-5.59 (m, 1H), 4.64-4.61 (m, 1H), 4.52-4.50 (m, 1H), 4.33-4.32 (m, 1H), 4.31-4.32 (m, 1H), 2.10-2.06 (m, 1H), 1.95 (s, 3H), 1.84-1.79 (m, 3H), 0.89-0.83 (m, 3H).

### Example 2ad

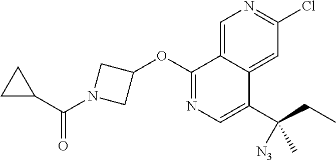 [SMILES: CC[C@@](C)(N=[N+]=[N-])c1cnc(OC2CN(C(=O)C3CC3)C2)c2cnc(Cl)cc12]

### Intermediate 46: (R)-(3-((4-(2-Azidobutan-2-yl)-6-chloro-2,7-naphthyridin-1-yl)oxy)azetidin-1-yl)(cyclopropyl)methanone

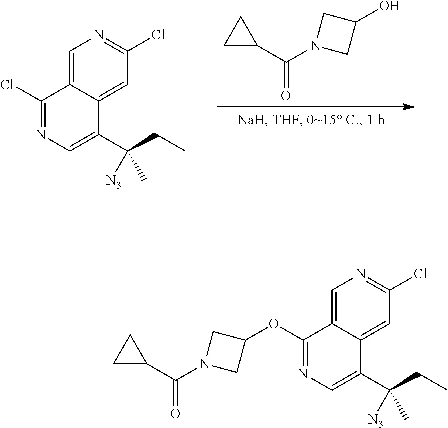 [SMILES: CC[C@@](C)(N=[N+]=[N-])c1cnc(Cl)c2cnc(Cl)cc12 ; O=C(C1CC1)N1CC(O)C1 ; CC[C@@](C)(N=[N+]=[N-])c1cnc(OC2CN(C(=O)C3CC3)C2)c2cnc(Cl)cc12]

### Step 1: (R)-(3-((4-(2-Azidobutan-2-yl)-6-chloro-2,7-naphthyridin-1-yl)oxy)azetidin-1-yl)(cyclopropyl)methanone

The title compound was prepared from (R)-4-(2-Azidobutan-2-yl)-1,6-dichloro-2,7-naphthyridine (Intermediate 44) and cyclopropyl(3-hydroxyazetidin-1-yl)methanone using a procedure similar to that described in Step 1 of Intermediate 9.

### Example 2ae

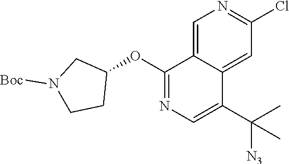 [SMILES: CC(C)(C)OC(=O)N1CC[C@@H](Oc2ncc(C(C)(C)N=[N+]=[N-])c3cc(Cl)ncc23)C1]

### Intermediate 47: tert-Butyl (R)-3-((4-(2-azidopropan-2-yl)-6-chloro-2,7-naphthyridin-1-yl)oxy)pyrrolidine-1-carboxylate

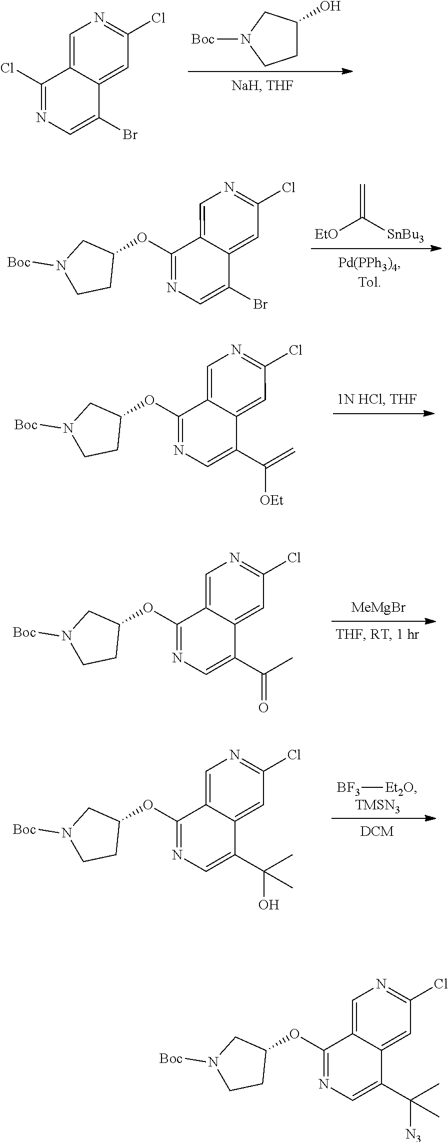

### Steps 1-5: tert-Butyl (R)-3-((4-(2-azidopropan-2-yl)-6-chloro-2,7-naphthyridin-1-yl)oxy)pyrrolidine-1-carboxylate

The title compound was prepared from tert-butyl (R)-3-hydroxypyrrolidine-1-carboxylate and 4-bromo-1,6-dichloro-2,7-naphthyridine using the same procedure as described in Steps 1-5 of Intermediate 28.

### Example 2af

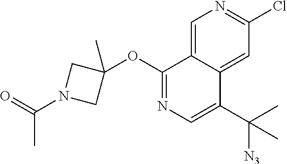 [SMILES: CC(=O)N1CC(C)(Oc2ncc(C(C)(C)N=[N+]=[N-])c3cc(Cl)ncc23)C1]

### Intermediate 48: 1-(3-((4-(2-Azidopropan-2-yl)-6-chloro-2,7-naphthyridin-1-yl)oxy)-3-methylazetidin-1-yl)ethan-1-one

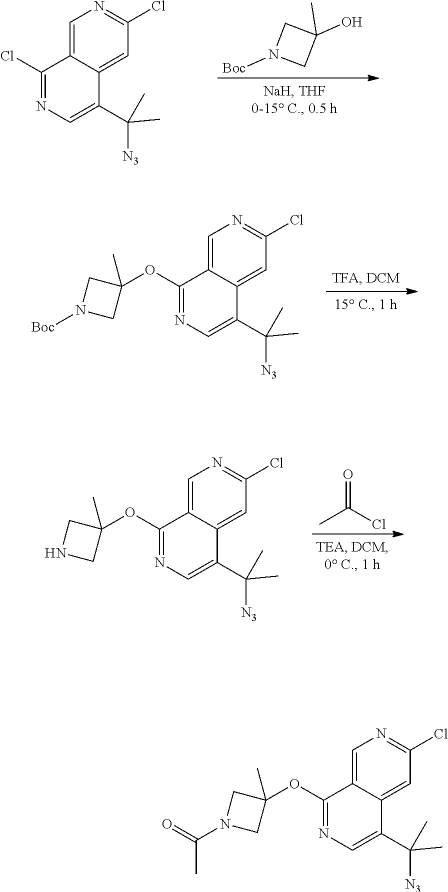

### Steps 1-3: 1-(3-((4-(2-Azidopropan-2-yl)-6-chloro-2,7-naphthyridin-1-yl)oxy)-3-methylazetidin-1-yl)ethan-1-one

The title compound was prepared from 4-(2-azidopropan-2-yl)-1,6-dichloro-2,7-naphthyridine and tert-butyl 3-hydroxy-3-methylazetidine-1-carboxylate using a procedure similar to that described in Steps 1-3 of Intermediate 9. MS (ES+) C₁₇H₁₉ClN₆O₂ requires: 374, found: 375[M+H]⁺.

### Example 2ag

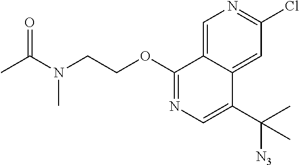 [SMILES: CC(=O)N(C)CCOc1ncc(C(C)(C)N=[N+]=[N-])c2cc(Cl)ncc12]

### Intermediate 49: N-(2-((4-(2-Azidopropan-2-yl)-6-chloro-2,7-naphthyridin-1-yl)oxy)ethyl)-N-methylacetamide

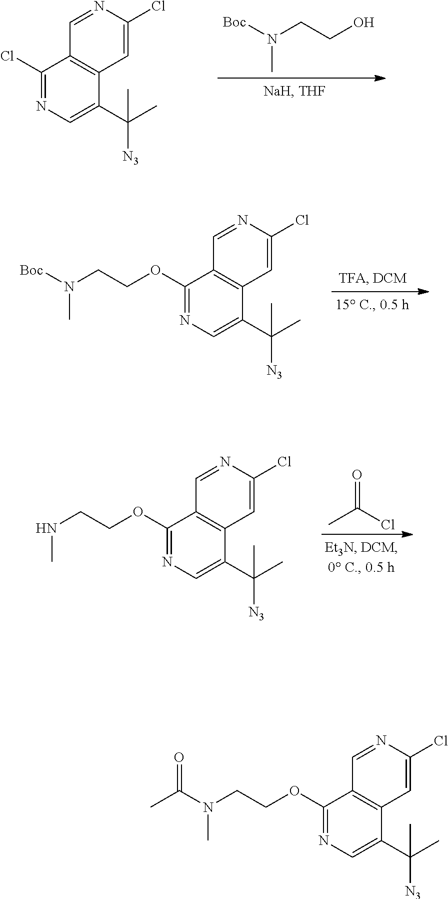

### Steps 1-3: N-(2-((4-(2-Azidopropan-2-yl)-6-chloro-2,7-naphthyridin-1-yl)oxy)ethyl)-N-methylacetamide

The title compound was prepared from 4-(2-azidopropan-2-yl)-1,6-dichloro-2,7-naphthyridine and tert-butyl (2-hydroxyethyl)(methyl)carbamate using a procedure similar to that described in Steps 1-3 of Intermediate 9.

### Example 2ah

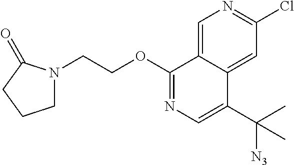 [SMILES: CC(C)(N=[N+]=[N-])c1cnc(OCCN2CCCC2=O)c2cnc(Cl)cc12]

### Intermediate 50: 1-(2-((4-(2-Azidopropan-2-yl)-6-chloro-2,7-naphthyridin-1-yl)oxy)ethyl)pyrrolidin-2-one

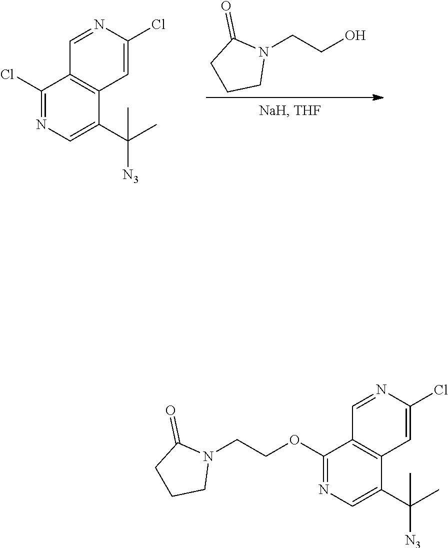 [SMILES: CC(C)(N=[N+]=[N-])c1cnc(Cl)c2cnc(Cl)cc12 ; O=C1CCCN1CCO ; CC(C)(N=[N+]=[N-])c1cnc(OCCN2CCCC2=O)c2cnc(Cl)cc12]

### Steps 1: 1-(2-((4-(2-Azidopropan-2-yl)-6-chloro-2,7-naphthyridin-1-yl)oxy)ethyl)pyrrolidin-2-one

The title compound was prepared from 4-(2-azidopropan-2-yl)-1,6-dichloro-2,7-naphthyridine and 1-(2-hydroxyethyl)pyrrolidin-2-one using a procedure similar to that described in Step 1 of Intermediate 9.

### Example 2ai

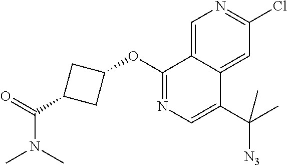 [SMILES: CN(C)C(=O)[C@H]1C[C@@H](Oc2ncc(C(C)(C)N=[N+]=[N-])c3cc(Cl)ncc23)C1]

### Intermediate 51: cis-3-((4-(2-Azidopropan-2-yl)-6-chloro-2,7-naphthyridin-1-yl)oxy)-N,N-dimethylcyclobutane-1-carboxamide

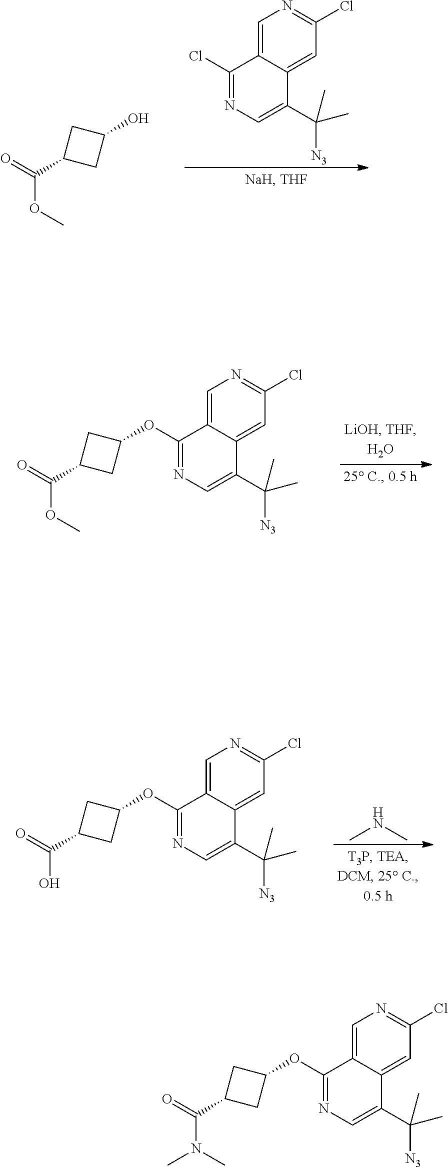

### Step 1: Methyl cis-3-((4-(2-azidopropan-2-yl)-6-chloro-2,7-naphthyridin-1-yl)oxy)cyclobutane-1-carboxylate

The title compound was prepared from 4-(2-azidopropan-2-yl)-1,6-dichloro-2,7-naphthyridine and methyl cis-3-hydroxycyclobutane-1-carboxylate using a procedure similar to that described in Step 1 of Intermediate 9.

### Step 2: cis-3-((4-(2-Azidopropan-2-yl)-6-chloro-2,7-naphthyridin-1-yl)oxy)cyclobutane-1-carboxylic acid

To a solution of methyl cis-3-((4-(2-azidopropan-2-yl)-6-chloro-2,7-naphthyridin-1-yl)oxy)cyclobutane-1-carboxylate (60 mg, 160 μmol) in water (1 mL) and THF (3 mL) was added LiOH (11.5 mg, 479 μmol). The reaction mixture was stirred at 15° C. for 0.5 h, then was acidified to pH=5 with aqueous hydrochloric acid (2 M) and extracted with EA (30 mL×3). The organic layers were dried with sodium sulfate, filtered and concentrated to give the title compound (55 mg, crude) as a white solid.

### Step 3: cis-3-((4-(2-Azidopropan-2-yl)-6-chloro-2,7-naphthyridin-1-yl)oxy)-N,N-dimethylcyclobutane-1-carboxamide

To a solution of cis-3-((4-(2-Azidopropan-2-yl)-6-chloro-2,7-naphthyridin-1-yl)oxy)cyclobutane-1-carboxylic acid (55 mg, 152 μmol), dimethylamine hydrochloride (24.8 mg, 304 μmol) and Et₃N (76.9 mg, 760 μmol) in DCM (10 mL) was added T₃P (145 mg, 456 mol). The reaction mixture was stirred at 15° C. for 1 h, then was concentrated to give a residue. The residue was purified by prep-TLC (SiO₂, PE/EA=2/1) to give the title compound (55 mg, 93% yield) as a white solid. ¹H NMR (400 MHz, CD₃OD): δ ppm 9.44 (s, 1H), 8.52 (s, 1H), 8.30 (s, 1H), 5.50-5.40 (s, 1H), 3.27-3.07 (m, 1H), 2.97 (s, 3H), 2.89 (s, 3H), 2.89-2.86 (m, 2H), 2.52-2.49 (m, 2H), 1.82 (s, 6H).

### Example 2aj

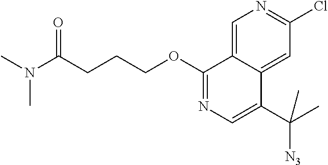 [SMILES: CN(C)C(=O)CCCOc1ncc(C(C)(C)N=[N+]=[N-])c2cc(Cl)ncc12]

### Intermediate 52: 4-((4-(2-Azidopropan-2-yl)-6-chloro-2,7-naphthyridin-1-yl)oxy)-N,N-dimethylbutanamide

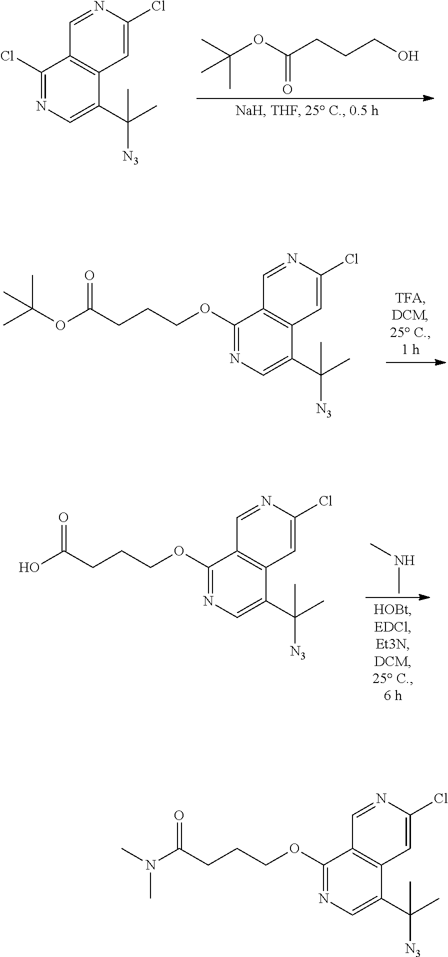

### Step 1: tert-Butyl 4-((4-(2-azidopropan-2-yl)-6-chloro-2,7-naphthyridin-1-yl)oxy)butanoate

The title compound was prepared from 4-(2-azidopropan-2-yl)-1,6-dichloro-2,7-naphthyridine and tert-butyl 4-hydroxybutanoate using a procedure similar to that described in Step 1 of Intermediate 9.

### Step 2: 4-((4-(2-Azidopropan-2-yl)-6-chloro-2,7-naphthyridin-1-yl)oxy)butanoic acid

To a solution of tert-butyl 4-((4-(2-azidopropan-2-yl)-6-chloro-2,7-naphthyridin-1-yl)oxy)butanoate (140 mg, 345 umol) in DCM (10 mL) was added TFA (2 mL), and the reaction mixture was stirred at 25° C. for 1 h. The reaction mixture was diluted with water (30 mL) and extracted with EA (30 mL×3). The combined organic layers were dried over anhydrous sodium sulfate, filtered and concentrated to give a residue. The residue was purified by prep-TLC (PE:EA=1:1) to give the title compound (20 mg, 16% yield) as a colorless oil.

### Step 3: 4-((4-(2-Azidopropan-2-yl)-6-chloro-2,7-naphthyridin-1-yl)oxy)-N,N-dimethylbutanamide

To a solution of 4-((4-(2-Azidopropan-2-yl)-6-chloro-2,7-naphthyridin-1-yl)oxy)butanoic acid (15 mg, 42.9 umol) and dimethylamine hydrochloride (9.67 mg, 119 umol) in DCM (2 mL) was added HOBt (6.95 mg, 51.5 umol), EDCI (12.3 mg, 64.3 umol) and Et₃N (5.97 uL, 42.9 umol). The reaction mixture was stirred at 25° C. for 6 h, then was diluted with water (30 mL) and extracted with EA (30 mL×3). The combined organic layers were dried over anhydrous sodium sulfate, filtered and concentrated to give the title compound (10 mg, 58% yield) as a white solid that was used without further purification. MS (ES+) C₁₇H₂₁ClN₆O₂ requires: 376, found: 377[M+H]⁺.

### Example 2ak

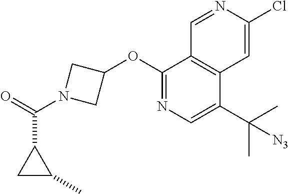

### Intermediate 53: (3-((4-(2-Azidopropan-2-yl)-6-chloro-2,7-naphthyridin-1-yl)oxy)azetidin-1-yl)((1S,2R)-2-methylcyclopropyl)methanone

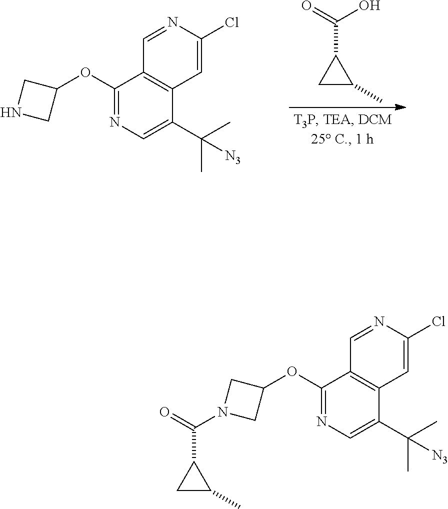

### Step 1: (3-((4-(2-Azidopropan-2-yl)-6-chloro-2,7-naphthyridin-1-yl)oxy)azetidin-1-yl)((1S,2R)-2-methylcyclopropyl)methanone

To a solution of (1S,2R)-2-methylcyclopropane-1-carboxylic acid (42.0 mg, 416 umol) and 1-(azetidin-3-yloxy)-4-(2-azidopropan-2-yl)-6-chloro-2,7-naphthyridine (title compound from Step 2 of Intermediate 9, 150 mg, 347 umol, TFA) in DCM (5 mL) was added Et₃N (175 mg, 1.73 mmol) and T₃P (441 mg, 693 umol, 50% purity). The reaction mixture was stirred at 25° C. for 1 h, then was diluted with water (30 mL) and extracted with DCM (20 mL×3). The combined organic layers were dried over anhydrous sodium sulfate, filtered and concentrated to give a residue. The residue was purified by prep-TLC (PE/EA=1:1) to give the title compound (100 mg, 72% yield) as a white solid.

### Example 2al

Intermediates 54-61: Intermediates were made using a similar procedure as described for Intermediate 53, except that a different reagent was used as noted below in Step 1.

| | | Reagent used in Step 1 of |
|---|---|---|
| Intermediate | Structure | prep for Intermediate |
| 54 | 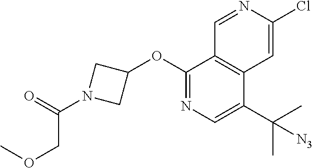 [SMILES: COCC(=O)N1CC(Oc2ncc(C(C)(C)N=[N+]=[N-])c3cc(Cl)ncc23)C1] | 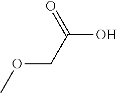 |
| 55 | 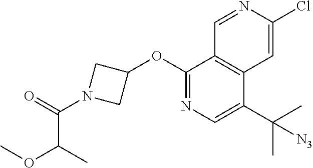 [SMILES: COC(C)C(=O)N1CC(Oc2ncc(C(C)(C)N=[N+]=[N-])c3cc(Cl)ncc23)C1] | 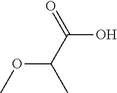 |
| 56 | 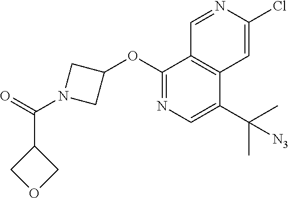 [SMILES: CC(C)(N=[N+]=[N-])c1cnc(OC2CN(C(=O)C3COC3)C2)c2cnc(Cl)cc12] | 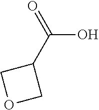 |
| 57 | 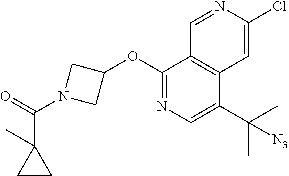 [SMILES: CC1(C(=O)N2CC(Oc3ncc(C(C)(C)N=[N+]=[N-])c4cc(Cl)ncc34)C2)CC1] | 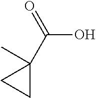 |
| 58 | 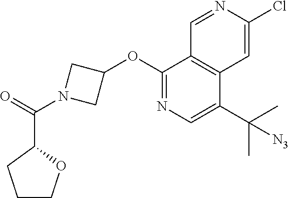 [SMILES: CC(C)(N=[N+]=[N-])c1cnc(OC2CN(C(=O)[C@H]3CCCO3)C2)c2cnc(Cl)cc12] | 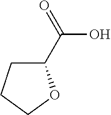 |
| 59 | 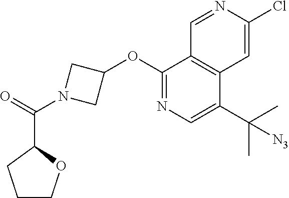 [SMILES: CC(C)(N=[N+]=[N-])c1cnc(OC2CN(C(=O)[C@@H]3CCCO3)C2)c2cnc(Cl)cc12] | 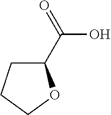 |
| 60 | 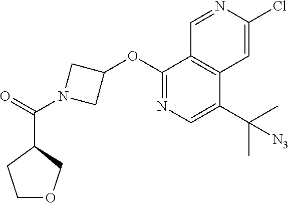 [SMILES: CC(C)(N=[N+]=[N-])c1cnc(OC2CN(C(=O)[C@@H]3CCOC3)C2)c2cnc(Cl)cc12] | 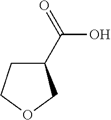 |
| 61 | 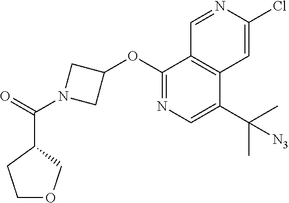 [SMILES: CC(C)(N=[N+]=[N-])c1cnc(OC2CN(C(=O)[C@H]3CCOC3)C2)c2cnc(Cl)cc12] | 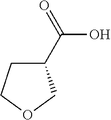 |

### Example 2am

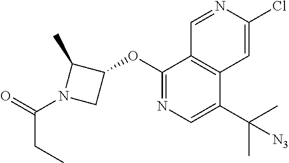 [SMILES: CCC(=O)N1C[C@@H](Oc2ncc(C(C)(C)N=[N+]=[N-])c3cc(Cl)ncc23)[C@@H]1C]

### Intermediate 62: 1-((2S,3R)-3-((4-(2-Azidopropan-2-yl)-6-chloro-2,7-naphthyridin-1-yl)oxy)-2-methylazetidin-1-yl)propan-1-one

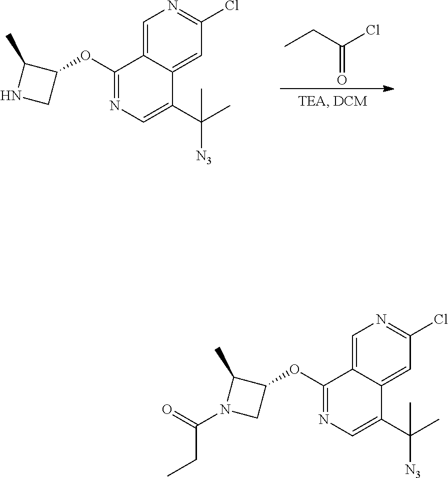

### Step 1: 1-((2S,3R)-3-((4-(2-Azidopropan-2-yl)-6-chloro-2,7-naphthyridin-1-yl)oxy)-2-methylazetidin-1-yl)propan-1-one

The title compound was prepared from 4-(2-azidopropan-2-yl)-6-chloro-1-(((2S,3R)-2-methylazetidin-3-yl)oxy)-2,7-naphthyridine (title compound from Step 2 of Intermediate 10) and propionyl chloride using a similar procedure as described in Step 3 of Intermediate 9 above. C₁₈H₂₁ClN₆O₂ requires: 388, found: 389[M+H]⁺.

### Example 2an

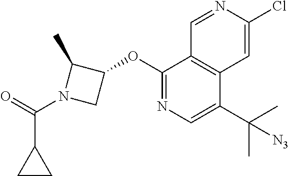 [SMILES: C[C@H]1[C@H](Oc2ncc(C(C)(C)N=[N+]=[N-])c3cc(Cl)ncc23)CN1C(=O)C1CC1]

### Intermediate 63: ((2S,3R)-3-((4-(2-Azidopropan-2-yl)-6-chloro-2,7-naphthyridin-1-yl)oxy)-2-methylazetidin-1-yl)(cyclopropyl)methanone

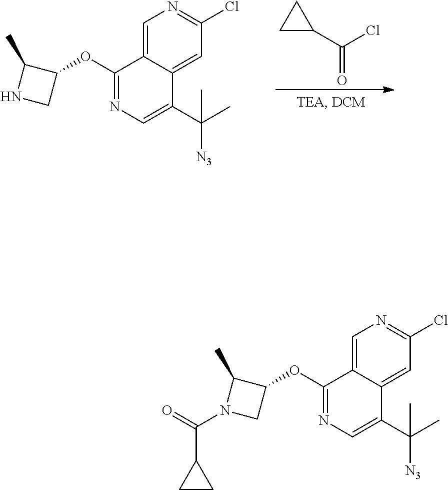 [SMILES: C[C@@H]1NC[C@H]1Oc1ncc(C(C)(C)N=[N+]=[N-])c2cc(Cl)ncc12 ; O=C(Cl)C1CC1 ; C[C@H]1[C@H](Oc2ncc(C(C)(C)N=[N+]=[N-])c3cc(Cl)ncc23)CN1C(=O)C1CC1]

### Step 1: ((2S,3R)-3-((4-(2-Azidopropan-2-yl)-6-chloro-2,7-naphthyridin-1-yl)oxy)-2-methylazetidin-1-yl)(cyclopropyl)methanone

The title compound was prepared from 4-(2-azidopropan-2-yl)-6-chloro-1-(((2S,3R)-2-methylazetidin-3-yl)oxy)-2,7-naphthyridine (title compound from Step 2 of Intermediate 10) and cyclopropanecarbonyl chloride using a similar procedure as described in Step 3 of Intermediate 9 above. C₁₉H₂₁ClN₆O₂ requires: 400, found: 401[M+H]⁺.

### Example 2ao

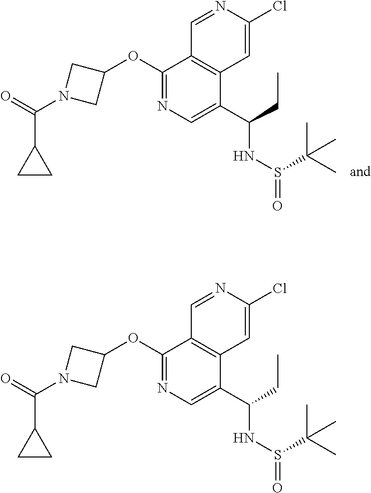 [SMILES: CC[C@@H](N[S@@](=O)C(C)(C)C)c1cnc(OC2CN(C(=O)C3CC3)C2)c2cnc(Cl)cc12 ; CC[C@H](N[S@@](=O)C(C)(C)C)c1cnc(OC2CN(C(=O)C3CC3)C2)c2cnc(Cl)cc12]

### Intermediates 64 and 65: (S)—N—((R)-1-(6-Chloro-1-((1-(cyclopropanecarbonyl)azetidin-3-yl)oxy)-2,7-naphthyridin-4-yl)propyl)-2-methylpropane-2-sulfinamide and (S)—N—((S)-1-(6-chloro-1-((1-(cyclopropanecarbonyl)azetidin-3-yl)oxy)-2,7-naphthyridin-4-yl)propyl)-2-methylpropane-2-sulfinamide

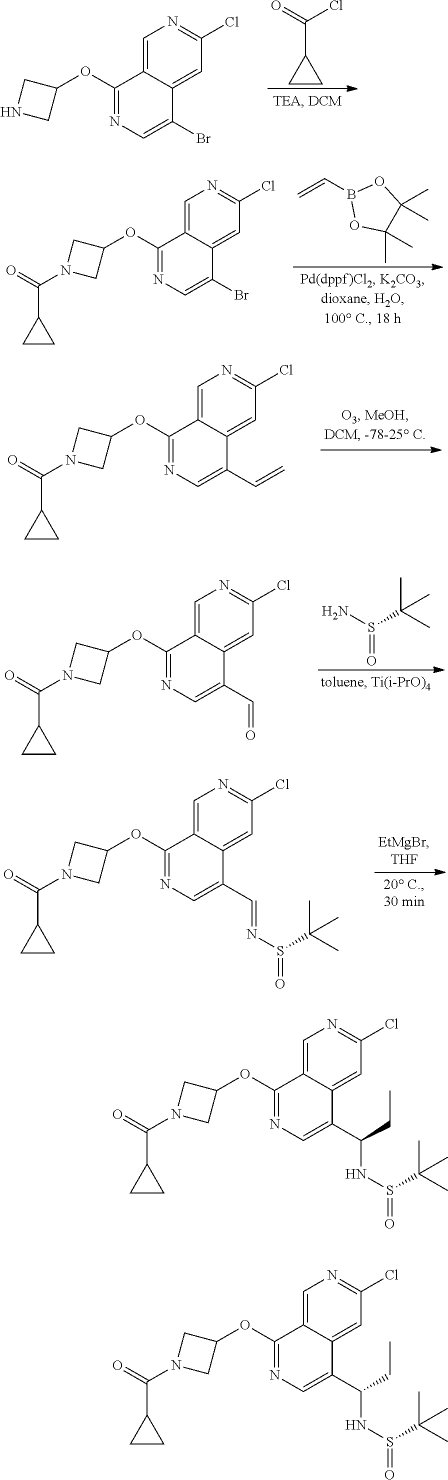

### Step 1: (3-((4-Bromo-6-chloro-2,7-naphthyridin-1-yl)oxy)azetidin-1-yl)(cyclopropyl)methanone

To a solution of 1-(azetidin-3-yloxy)-4-bromo-6-chloro-2,7-naphthyridine (title compound from Step 2 of Intermediate 13, 0.75 g, 2.38 mmol) and Et₃N (724 mg, 7.15 mmol) in DCM (8 mL) was added cyclopropanecarbonyl chloride (374 mg, 3.58 mmol) at 0° C. The reaction mixture was stirred at 0° C. for 1 h, then was partitioned between EA (80 mL) and brine (50 mL). The water layer was extracted with EA (50 mL×3), and the combined organic layers were dried over sodium sulfate, filtered and concentrated to give a residue. The residue was triturated with EA (10 mL) at 25° C. for 30 min to give the title compound (420 mg, 42% yield) as a yellow solid.

### Step 2: (3-((6-Chloro-4-vinyl-2,7-naphthyridin-1-yl)oxy)azetidin-1-yl)(cyclopropyl)methanone

To a solution of (3-((4-bromo-6-chloro-2,7-naphthyridin-1-yl)oxy)azetidin-1-yl)(cyclopropyl)methanone (5 g, 13.1 mmol) and 4,4,5,5-tetramethyl-2-vinyl-1,3,2-dioxaborolane (1.81 g, 11.8 mmol, 1.99 mL) in dioxane (20 mL) and water (5 mL) was added Pd(dppf)Cl₂ (956 mg, 1.31 mmol) and K₂CO₃ (3.61 g, 26.13 mmol). The reaction mixture was stirred at 80° C. for 18 h, then was partitioned between EA (200 mL) and brine (100 mL). The aqueous layer was extracted with EA (150 mL×3), and the combined organic layers were dried over sodium sulfate, filtered and concentrated to give a residue. The residue was purified by column chromatography (SiO₂, PE/EA=100/1 to 1/2) to give the title compound (2.6 g, 35% yield) as an off-white solid. MS (ES+) C₁₇H₁₆ClN₃O₂ requires: 329, found: 330[M+H]⁺.

### Steps 3-5: (S)—N—((R)-1-(6-Chloro-1-((1-(cyclopropanecarbonyl)azetidin-3-yl)oxy)-2,7-naphthyridin-4-yl)propyl)-2-methylpropane-2-sulfinamide and (S)—N—((S)-1-(6-chloro-1-((1-(cyclopropanecarbonyl)azetidin-3-yl)oxy)-2,7-naphthyridin-4-yl)propyl)-2-methylpropane-2-sulfinamide

The title compounds were prepared from (3-((6-Chloro-4-vinyl-2,7-naphthyridin-1-yl)oxy)azetidin-1-yl)(cyclopropyl)methanone using a procedure similar to that described in Steps 5-7 of Intermediates 13 and 14. The mixture of two compounds was purified by prep-HPLC (column: Waters Xbridge 150*25 mm*5 um; mobile phase: [water (0.05% ammonia hydroxide v/v)-ACN]; B %: 32%-52%, 10 min) to give the title compounds separately. The stereochemistry was assigned arbitrarily.

First eluting isomer, Peak 1 (Intermediate 64): 25 mg, 12% yield was obtained as a yellow solid. MS (ES+) C₂₂H₂₉ClN₄O₃S requires: 464, found: 465[M+H]⁺.

Second eluting isomer, Peak 2 (Intermediate 65): 80 mg, 39% yield was obtained as a yellow solid. MS (ES+) C₂₂H₂₉ClN₄O₃S requires: 464, found: 465[M+H]⁺.

### Example 2ap

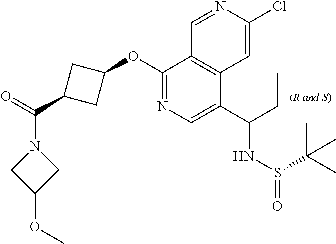 [SMILES: CCC(N[S@@](=O)C(C)(C)C)c1cnc(O[C@H]2C[C@@H](C(=O)N3CC(OC)C3)C2)c2cnc(Cl)cc12]

### Intermediate 66: (S)—N-(1-(6-Chloro-1-(cis-3-(3-methoxyazetidine-1-carbonyl)cyclobutoxy)-2,7-naphthyridin-4-yl)propyl)-2-methylpropane-2-sulfinamide

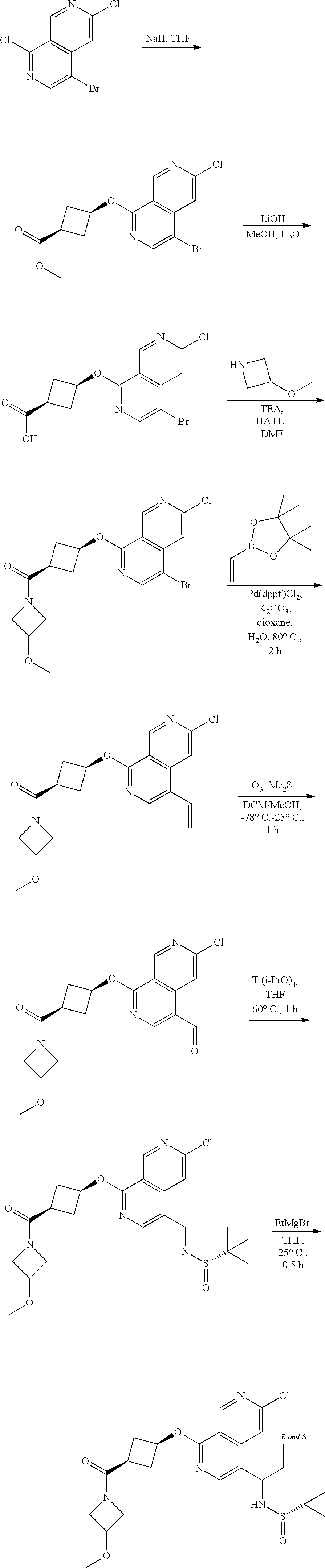

### Step 1: Methyl cis-3-((4-bromo-6-chloro-2,7-naphthyridin-1-yl)oxy)cyclobutane-1-carboxylate

The title compound was prepared from 4-bromo-1,6-dichloro-2,7-naphthyridine and methyl cis-3-hydroxycyclobutane-1-carboxylate using a similar procedure as described in Step 1 of Intermediate 9.

### Step 2: cis-3-((4-Bromo-6-chloro-2,7-naphthyridin-1-yl)oxy)cyclobutane-1-carboxylic acid

To a solution of methyl cis-3-((4-bromo-6-chloro-2,7-naphthyridin-1-yl)oxy)cyclobutane-1-carboxylate (3.5 g, 9.42 mmol, 1 eq) in MeOH (45 mL) and water (15 mL) was added LiOH (676.66 mg, 28.26 mmol, 3 eq). The reaction mixture was stirred at 25° C. for 1 h, then was concentrated and diluted with water (100 mL). The mixture was acidified with aqueous HCl solution (6 M, 1 mL), extracted with EA (100 mL×3), and the combined organic layers were concentrated to give the title compound (1.7 g, 4.75 mmol, 50.48% yield) as a white solid that was used in the next step without further purification.

### Step 3: (cis-3-((4-Bromo-6-chloro-2,7-naphthyridin-1-yl)oxy)cyclobutyl)(3-methoxyazetidin-1-yl)methanone

To a solution of cis-3-((4-bromo-6-chloro-2,7-naphthyridin-1-yl)oxy)cyclobutane-1-carboxylic acid (2.7 g, 7.55 mmol, 1 eq) and 3-methoxyazetidine (1.40 g, 11.33 mmol, 1.5 eq, HCl) in DMF (100 mL) was added Et₃N (5.25 mL, 37.75 mmol, 5 eq) and HATU (5.74 g, 15.10 mmol, 2 eq). The reaction mixture was stirred at 25° C. for 1 h, then was diluted with water (100 mL) and extracted with EA (75 mL×3). The combined organic layers were washed with brine (80 mL×3), dried over anhydrous sodium sulfate, filtered and concentrated to give a residue. The residue was purified by column chromatography (SiO₂, PE/EA=10/1 to 1/1) to give the title compound as a white solid.

### Steps 4-7: (S)—N-(1-(6-Chloro-1-(cis-3-(3-methoxyazetidine-1-carbonyl)cyclobutoxy)-2,7-naphthyridin-4-yl)propyl)-2-methylpropane-2-sulfinamide

The title compound was prepared from (cis-3-((4-bromo-6-chloro-2,7-naphthyridin-1-yl)oxy)cyclobutyl)(3-methoxyazetidin-1-yl)methanone using a procedure similar to that described in Step 2 of Intermediate 65 and Steps 5-7 of Intermediates 13 and 14.

### Example 2aq

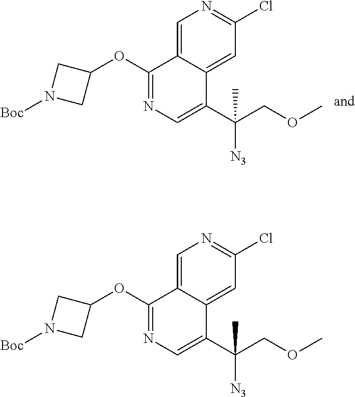 [SMILES: COC[C@@](C)(N=[N+]=[N-])c1cnc(OC2CN(C(=O)OC(C)(C)C)C2)c2cnc(Cl)cc12 ; COC[C@](C)(N=[N+]=[N-])c1cnc(OC2CN(C(=O)OC(C)(C)C)C2)c2cnc(Cl)cc12]

### Intermediates 67 and 68: tert-Butyl (S)-3-((5-(2-azido-1-methoxypropan-2-yl)-3-chloroisoquinolin-8-yl)oxy)azetidine-1-carboxylate and tert-butyl (R)-3-((5-(2-azido-1-methoxypropan-2-yl)-3-chloroisoquinolin-8-yl)oxy)azetidine-1-carboxylate

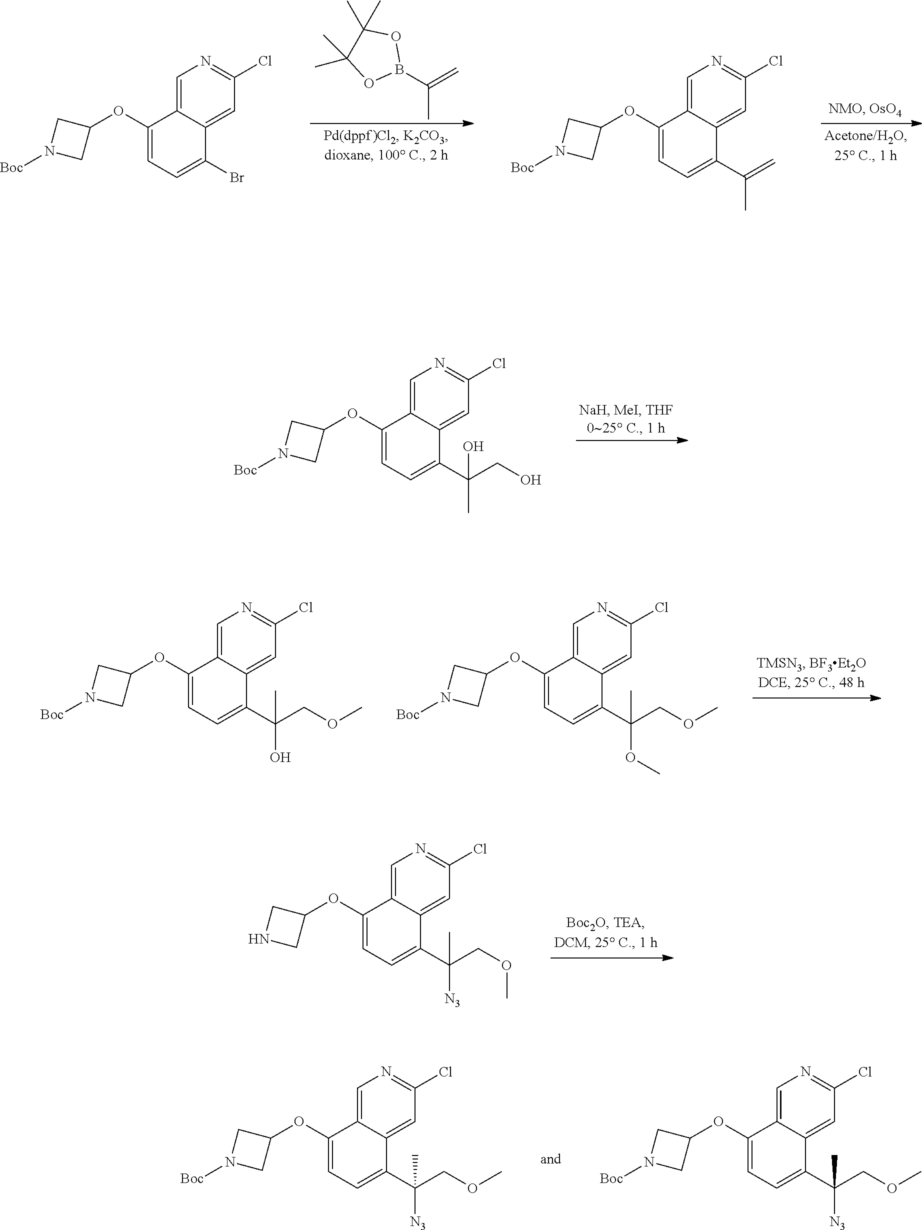

### Step 1: tert-Butyl 3-((3-chloro-5-(prop-1-en-2-yl)isoquinolin-8-yl)oxy)azetidine-1-carboxylate

To a mixture of tert-butyl 3-((5-bromo-3-chloroisoquinolin-8-yl)oxy)azetidine-1-carboxylate (title compound from Step 6 of Intermediate 38, 3 g, 7.25 mmol) and 4,4,5,5-tetramethyl-2-(prop-1-en-2-yl)-1,3,2-dioxaborolane (1.34 g, 7.98 mmol) in dioxane (40 mL), water (8 mL) was added Pd(dppf)Cl₂ (530 mg, 725 umol) and K₂CO₃ (2.00 g, 14.5 mmol). The reaction mixture was stirred at 100° C. for 2 h, then was removed from heat, diluted with water (40 mL), and extracted with EA (60 mL×3). The combined organic layers were dried over anhydrous sodium sulfate, filtered and concentrated to give a residue. The residue was purified by flash silica gel chromatography (Eluent of 0˜20% EA/PE) to give the title compound (2.1 g, 64% yield) as a yellow oil. MS (ES+) C₂₀H₂₃ClN₂O₃ requires: 374, found: 375[M+H]⁺.

### Step 2: tert-Butyl 3-((3-chloro-5-(1,2-dihydroxypropan-2-yl)isoquinolin-8-yl)oxy)azetidine-1-carboxylate

To a solution of tert-butyl 3-((3-chloro-5-(prop-1-en-2-yl)isoquinolin-8-yl)oxy)azetidine-1-carboxylate (1.9 g, 5.07 mmol) in Acetone (40 mL) and water (10 mL) was added OsO₄ (12.9 mg, 50.7 umol) and NMO (1.19 g, 10.1 mmol). The reaction mixture was stirred at 25° C. for 2 h, then was quenched by addition of saturated aqueous sodium sulfite (40 mL). The mixture was diluted with water (20 mL) and extracted with EA (60 mL×3). The combined organic layers were dried over anhydrous sodium sulfate, filtered and concentrated to give a residue. The residue was purified by flash silica gel chromatography (Eluent of 0˜80% EA/PE) to give the title compound (1.4 g, 68% yield) as a white solid.

### Step 3: tert-Butyl 3-((3-chloro-5-(2-hydroxy-1-methoxypropan-2-yl)isoquinolin-8-yl)oxy)azetidine-1-carboxylate and tert-butyl 3-((3-chloro-5-(1,2-dimethoxypropan-2-yl)isoquinolin-8-yl)oxy)azetidine-1-carboxylate

To a solution of tert-butyl 3-((3-chloro-5-(1,2-dihydroxypropan-2-yl)isoquinolin-8-yl)oxy)azetidine-1-carboxylate (1.3 g, 3.18 mmol) in THF (20 mL) was added NaH (153 mg, 3.82 mmol) at 0° C. After stirring for 0.5 h at 0° C., MeI (496 mg, 3.50 mmol) was added to the reaction mixture and the cooling bath was removed. The reaction mixture was stirred at 25° C. for 0.5 h, then was quenched with water (70 mL) and extracted with EA (50 mL×3). The combined organic layers were dried over anhydrous sodium sulfate, filtered and concentrated to give a residue. The residue was purified by flash silica gel chromatography (Eluent of 0˜60% EA/PE) to give tert-butyl 3-((3-chloro-5-(2-hydroxy-1-methoxypropan-2-yl)isoquinolin-8-yl)oxy)azetidine-1-carboxylate (790 mg, 57% yield) as a white solid and tert-butyl 3-((3-chloro-5-(1,2-dimethoxypropan-2-yl)isoquinolin-8-yl)oxy)azetidine-1-carboxylate (440 mg, 31% yield) as a white solid.

### Step 4: 8-(Azetidin-3-yloxy)-5-(2-azido-1-methoxypropan-2-yl)-3-chloroisoquinoline

To a solution of tert-butyl 3-((3-chloro-5-(2-hydroxy-1-methoxypropan-2-yl)isoquinolin-8-yl)oxy)azetidine-1-carboxylate (720 mg, 1.70 mmol) and TMSN₃ (981 mg, 8.51 mmol) in 1,2-DCE (25 mL) was added BF₃-Et₂O (483 mg, 3.41 mmol). The reaction mixture was stirred at 25° C. for 1 h, then was quenched by addition of saturated aqueous sodium bicarbonate solution (10 mL). The mixture was diluted with water (30 mL) was extracted with EA (30 mL×3). The combined organic layers were dried over anhydrous sodium sulfate, filtered and concentrated to give the crude title compound (410 mg, 55% yield) as a yellow oil that was used directly in the next step without further purification.

### Step 5: tert-Butyl (S)-3-((5-(2-azido-1-methoxypropan-2-yl)-3-chloroisoquinolin-8-yl)oxy)azetidine-1-carboxylate and tert-butyl (R)-3-((5-(2-azido-1-methoxypropan-2-yl)-3-chloroisoquinolin-8-yl)oxy)azetidine-1-carboxylate

A solution of 8-(azetidin-3-yloxy)-5-(2-azido-1-methoxypropan-2-yl)-3-chloroisoquinoline (680 mg, 1.96 mmol), Boc₂O (1.71 g, 7.82 mmol) and Et₃N (396 mg, 3.91 mmol) in DCM (25 mL) was stirred at 25° C. for 1 h. The reaction mixture was then diluted with water (100 mL) and extracted with EA (100 mL×3). The combined organic layers were dried over anhydrous sodium sulfate, filtered and concentrated to give a residue. The residue was purified by flash silica gel chromatography (Eluent of 0˜25% EA/PE) to give a racemic mixture of the title compounds. The mixture (400 mg, 38% yield) was separated chiral SFC (column: Daicel Chiralpak AD (250 mm×30 mm, 10 m); mobile phase: [0.1% NH₃H₂O MEOH]; B %: 35%-35%) to give the first of tert-butyl (S)-3-((5-(2-azido-1-methoxypropan-2-yl)-3-chloroisoquinolin-8-yl)oxy)azetidine-1-carboxylate or tert-butyl (R)-3-((5-(2-azido-1-methoxypropan-2-yl)-3-chloroisoquinolin-8-yl)oxy)azetidine-1-carboxylate (peak 1, Intermediate 67, 93 mg, 23% yield) as a colorless oil and the second of tert-butyl (S)-3-((5-(2-azido-1-methoxypropan-2-yl)-3-chloroisoquinolin-8-yl)oxy)azetidine-1-carboxylate or tert-butyl (R)-3-((5-(2-azido-1-methoxypropan-2-yl)-3-chloroisoquinolin-8-yl)oxy)azetidine-1-carboxylate (peak 2, Intermediate 68, 100 mg, 25% yield) as a colorless oil. MS (ES+) C₂₁H₂₆ClN₅O₄ requires: 447, found: 448[M+H]⁺.

### Example 2ar

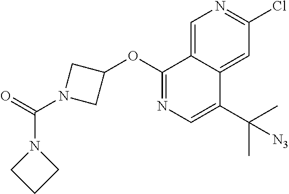 [SMILES: CC(C)(N=[N+]=[N-])c1cnc(OC2CN(C(=O)N3CCC3)C2)c2cnc(Cl)cc12]

### Intermediate 69: Azetidin-1-yl(13-((4-(2-azidopropan-2-yl)-6-chloro-2,7-naphthyridin-1-yl)oxy)azetidin-1-yl)methanone

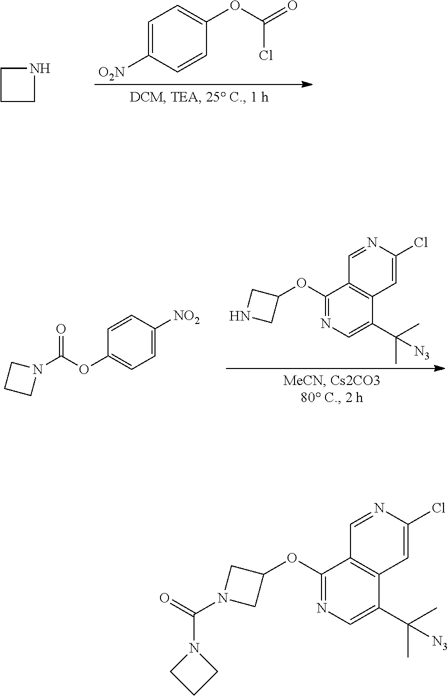

### Step 1: 4-Nitrophenyl azetidine-1-carboxylate

To a solution of azetidine (250 mg, 4.38 mmol) in DCM (10 mL) was added Et₃N (2.66 g, 26.3 mmol) and 4-nitrophenyl carbonochloridate (971 mg, 4.82 mmol). The reaction mixture was stirred at 25° C. for 1 h, then was partitioned between EA (80 mL) and saturated aqueous sodium chloride solution (50 mL). The layers were separated, and the aqueous layer was extracted with EA (20 mL×3). The combined organic layers were dried over sodium sulfate, filtered and concentrated to give a residue. The residue was purified by column chromatography (SiO₂, PE/EA=10/1 to 2/1) to give the title compound (770 mg, 76% yield) as a yellow solid. MS (ES+) C₁₀H₁₀N₂O₄ requires: 222, found: 223[M+H]⁺.

### Step 2: Azetidin-1-yl(3-((4-(2-azidopropan-2-yl)-6-chloro-2,7-naphthyridin-1-yl)oxy)azetidin-1-yl)methanone

To a solution of 4-nitrophenyl azetidine-1-carboxylate (153 mg, 690 umol) and 1-(azetidin-3-yloxy)-4-(2-azidopropan-2-yl)-6-chloro-2,7-naphthyridine (title compound from Step 2 of Intermediate 9, 220 mg, 690 umol) in acetonitrile (6 mL) was added Cs₂CO₃ (675 mg, 2.07 mmol). The reaction mixture was stirred at 80° C. for 2 h, then was partitioned between EA (80 mL) and saturated aqueous sodium chloride solution (50 mL). The organic layer was separated, and the water layer was extracted with EA (20 mL×3). The combined organic layers were dried over sodium sulfate, filtered and concentrated to give a residue. The residue was purified by prep-TLC (SiO₂, PE/EA=1:2) to give the title compound (120 mg, 32% yield) as a yellow solid. MS (ES+) C₁₈H₂₀ClN₇O₂ requires: 401, found: 402[M+H]⁺.

### Example 2as

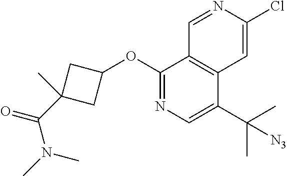 [SMILES: CN(C)C(=O)C1(C)CC(Oc2ncc(C(C)(C)N=[N+]=[N-])c3cc(Cl)ncc23)C1]

### Intermediate 70: 3-((4-(2-Azidopropan-2-yl)-6-chloro-2,7-naphthyridin-1-yl)oxy)-N,N,1-trimethylcyclobutane-1-carboxamide

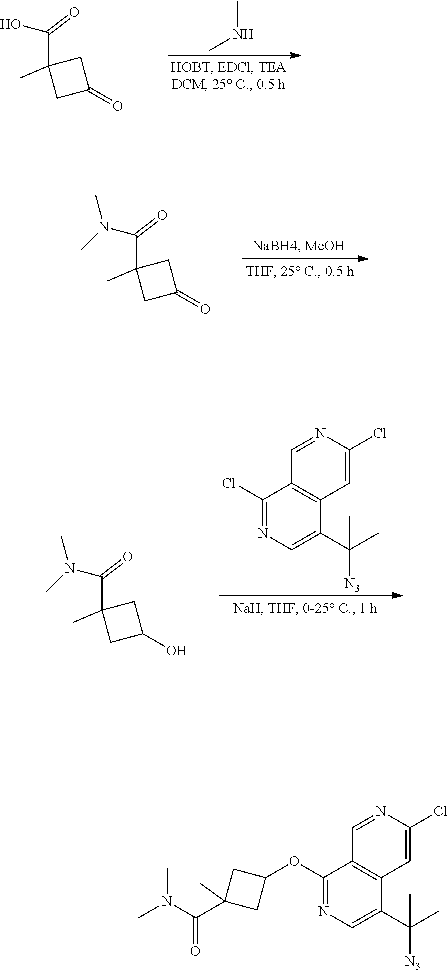

### Step 1: N,N,1-Trimethyl-3-oxocyclobutane-1-carboxamide

To a solution of 1-methyl-3-oxocyclobutane-1-carboxylic acid (400 mg, 3.12 mmol) and dimethylamine hydrochloride (1.27 g, 15.6 mmol) in DCM (10 mL) was added HOBt (505 mg, 3.74 mmol), EDCI (897 mg, 4.68 mmol) and Et₃N (473 mg, 4.68 mmol). The reaction mixture was stirred at 25° C. for 0.5 h, then was diluted with water (40 mL) and extracted with EA (40 mL×3). The combined organic layers were dried over anhydrous sodium sulfate, filtered and concentrated to give the title compound (400 mg, 82% yield) as a yellow oil.

### Step 2: 3-Hydroxy-N,N,1-trimethylcyclobutane-1-carboxamide

To a solution of N,N,1-trimethyl-3-oxocyclobutane-1-carboxamide (200 mg, 1.29 mmol) in MeOH (0.2 mL) and THF (2 mL) was added NaBH₄ (73.1 mg, 1.93 mmol). The reaction mixture was stirred at 25° C. for 0.5 h, then was quenched by addition of water (3 mL) and extracted with EA. The combined organic layers were dried over anhydrous sodium sulfate, filtered and concentrated to give the title compound (180 mg, 88% yield) as yellow oil.

### Step 3: 3-((4-(2-Azidopropan-2-yl)-6-chloro-2,7-naphthyridin-1-yl)oxy)-N,N,1-trimethylcyclobutane-1-carboxamide

The title compound was prepared from 4-bromo-1,6-dichloro-2,7-naphthyridine and 3-hydroxy-N,N,1-trimethylcyclobutane-1-carboxamide using a similar procedure as described in Step 1 of Intermediate 9.

### Example 2at

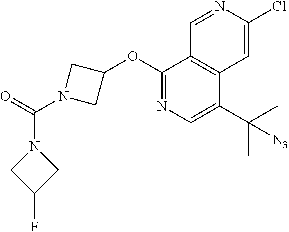 [SMILES: CC(C)(N=[N+]=[N-])c1cnc(OC2CN(C(=O)N3CC(F)C3)C2)c2cnc(Cl)cc12]

### Intermediate 71: (3-((4-(2-Azidopropan-2-yl)-6-chloro-2,7-naphthyridin-1-yl)oxy)azetidin-1-yl)(3-fluoroazetidin-1-yl)methanone

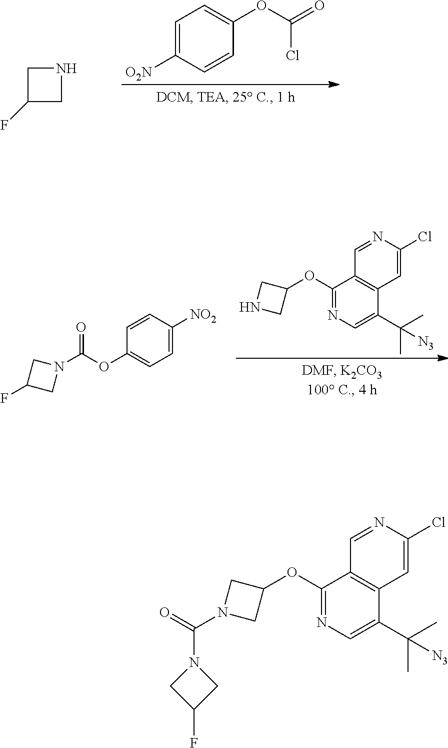 [SMILES: FC1CNC1 ; O=C(Cl)Oc1ccc([N+](=O)[O-])cc1 ; O=C(Oc1ccc([N+](=O)[O-])cc1)N1CC(F)C1 ; CC(C)(N=[N+]=[N-])c1cnc(OC2CNC2)c2cnc(Cl)cc12 ; CC(C)(N=[N+]=[N-])c1cnc(OC2CN(C(=O)N3CC(F)C3)C2)c2cnc(Cl)cc12]

### Steps 1-2: (3-((4-(2-Azidopropan-2-yl)-6-chloro-2,7-naphthyridin-1-yl)oxy)azetidin-1-yl)(3-fluoroazetidin-1-yl)methanone

The title compound was prepared from 3-fluoroazetidine and 1-(azetidin-3-yloxy)-4-(2-azidopropan-2-yl)-6-chloro-2,7-naphthyridine using a procedure similar to that described in Steps 1-2 of Intermediate 69, except that in Step 2 the solvent was DMF, base was K₂CO₃ and the temperature and time were 100° C. for 4 h. MS (ES+) C₁₈H₁₉ClFN₇O₂ requires: 419, found: 420[M+H]⁺.

### Example 2u

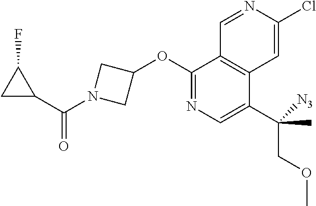 [SMILES: COC[C@@](C)(N=[N+]=[N-])c1cnc(OC2CN(C(=O)C3C[C@@H]3F)C2)c2cnc(Cl)cc12]

### Intermediate 72: (3-((4-((S)-2-Azido-1-methoxypropan-2-yl)-6-chloro-2,7-naphthyridin-1-yl)oxy)azetidin-1-yl)((1S,2S)-2-fluorocyclopropyl)methanone

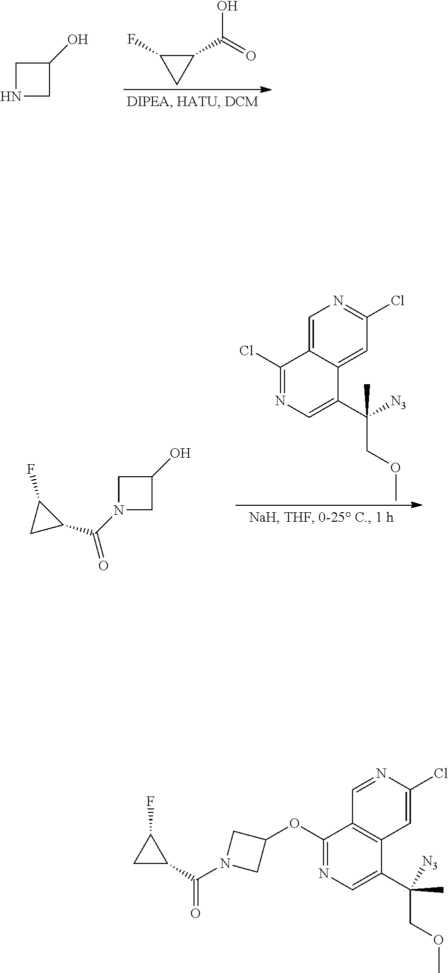 [SMILES: OC1CNC1 ; O=C(O)[C@@H]1C[C@@H]1F ; O=C([C@@H]1C[C@@H]1F)N1CC(O)C1 ; COC[C@@](C)(N=[N+]=[N-])c1cnc(Cl)c2cnc(Cl)cc12 ; COC[C@@](C)(N=[N+]=[N-])c1cnc(OC2CN(C(=O)[C@@H]3C[C@@H]3F)C2)c2cnc(Cl)cc12]

### Steps 1-2: (3-((4-((S)-2-Azido-1-methoxypropan-2-yl)-6-chloro-2,7-naphthyridin-1-yl)oxy)azetidin-1-yl)((1S,2S)-2-fluorocyclopropyl)methanone

The title compound was prepared from (1S,2S)-2-fluorocyclopropane-1-carboxylic acid and (S)-4-(2-azido-1-methoxypropan-2-yl)-1,6-dichloro-2,7-naphthyridine using a similar procedure as described in Step 2 of Intermediate 30 and Step 1 of Intermediate 9.

### Example 2av

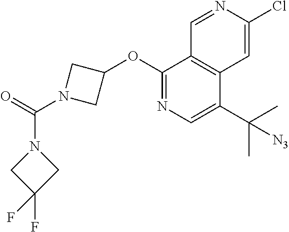 [SMILES: CC(C)(N=[N+]=[N-])c1cnc(OC2CN(C(=O)N3CC(F)(F)C3)C2)c2cnc(Cl)cc12]

### Intermediate 73: (3-((4-(2-Azidopropan-2-yl)-6-chloro-2,7-naphthyridin-1-yl)oxy)azetidin-1-yl)(3,3-difluoroazetidin-1-yl)methanone

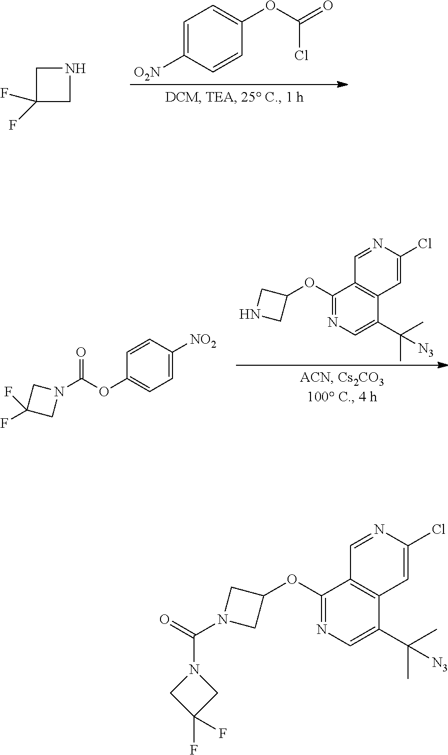 [SMILES: FC1(F)CNC1 ; O=C(Cl)Oc1ccc([N+](=O)[O-])cc1 ; O=C(Oc1ccc([N+](=O)[O-])cc1)N1CC(F)(F)C1 ; CC(C)(N=[N+]=[N-])c1cnc(OC2CNC2)c2cnc(Cl)cc12 ; CC(C)(N=[N+]=[N-])c1cnc(OC2CN(C(=O)N3CC(F)(F)C3)C2)c2cnc(Cl)cc12]

### Steps 1-2: (3-((4-(2-Azidopropan-2-yl)-6-chloro-2,7-naphthyridin-1-yl)oxy)azetidin-1-yl)(3,3-difluoroazetidin-1-yl)methanone

The title compound was prepared from 3-fluoroazetidine and 1-(azetidin-3-yloxy)-4-(2-azidopropan-2-yl)-6-chloro-2,7-naphthyridine using a procedure similar to that described in Steps 1-2 of Intermediate 69, except that in Step 2 the temperature and time were 100° C. for 4 h. MS (ES+) C₁₈H₁₈ClF₂N₇O₂ requires: 437, found: 438[M+H]⁺.

### Example 2aw

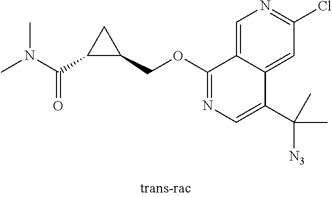 [SMILES: CN(C)C(=O)[C@@H]1C[C@H]1COc1ncc(C(C)(C)N=[N+]=[N-])c2cc(Cl)ncc12]

### Intermediate 74: (1R,2R and 1S,2S)-2-(((4-(2-Azidopropan-2-yl)-6-chloro-2,7-naphthyridin-1-yl)oxy)methyl)-N,N-dimethylcyclopropane-1-carboxamide

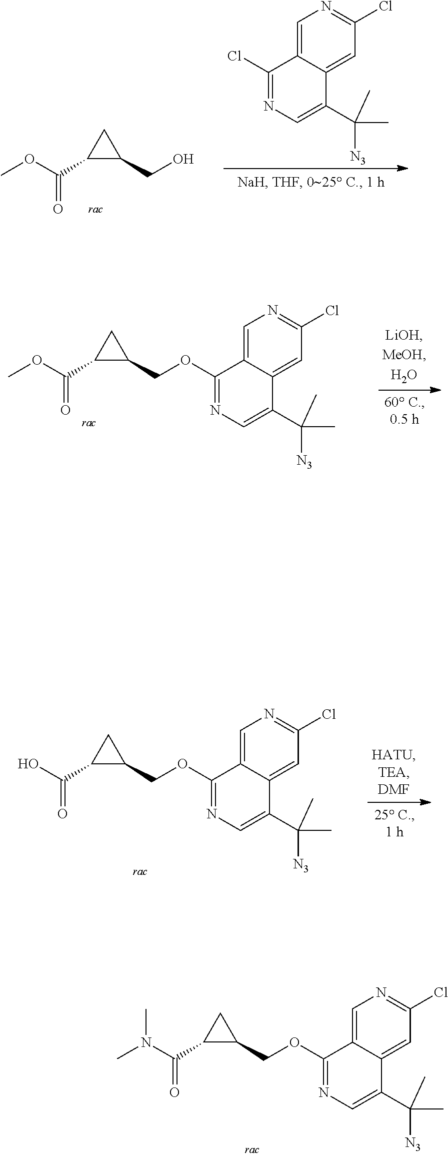

### Step 1: Methyl (1R,2R and 1S,2S)-2-(((4-(2-azidopropan-2-yl)-6-chloro-2,7-naphthyridin-1-yl)oxy)methyl)cyclopropane-1-carboxylate

The title compound was prepared from methyl (1R,2R and 1S,2S)-2-(hydroxymethyl)cyclopropane-1-carboxylate and 4-(2-azidopropan-2-yl)-1,6-dichloro-2,7-naphthyridine using a procedure similar to that described in Step 1 of Intermediate 9.

### Step 2: (1R,2R and 1S,2S)-2-(((4-(2-Azidopropan-2-yl)-6-chloro-2,7-naphthyridin-1-yl)oxy)methyl)cyclopropane-1-carboxylic acid

To a solution of methyl (1R,2R and 1S,2S)-2-(((4-(2-azidopropan-2-yl)-6-chloro-2,7-naphthyridin-1-yl)oxy)methyl)cyclopropane-1-carboxylate (510 mg, 1.36 mmol) in MeOH (10 mL) and water (2 mL) was added LiOH (97.5 mg, 4.07 mmol). The reaction mixture was stirred at 60° C. for 0.5 h, then was removed from heat and extracted EA (25 mL). The aqueous phase was acidified to pH<7 with aqueous HCl, then was extracted with EA (25 mL×3). The combined organic layers were dried over anhydrous sodium sulfate, filtered and concentrated to give the title compound (310 mg, 63% yield) as a colorless oil that was used in the next step without further purification.

### Step 3: (1R,2R and 1S,2S)-2-(((4-(2-Azidopropan-2-yl)-6-chloro-2,7-naphthyridin-1-yl)oxy)methyl)-N,N-dimethylcyclopropane-1-carboxamide

To a solution of (1R,2R and 1S,2S)-2-(((4-(2-azidopropan-2-yl)-6-chloro-2,7-naphthyridin-1-yl)oxy)methyl)cyclopropane-1-carboxylic acid (260 mg, 719 μmol) in DMF (10 mL) was added Et₃N (145 mg, 1.44 mmol, 200 μL) and HATU (547 mg, 1.44 mmol). The reaction mixture was stirred at 25° C. for 0.5 h, and then dimethylamine (162 mg, 3.59 mmol, 182 μL) was added. The mixture was stirred at 25° C. for 0.5 h, then was diluted with water (25 mL) and extracted with EA (25 mL×3). The combined organic layers were dried over anhydrous sodium sulfate, filtered and concentrated to give a residue. The residue was purified by prep-HPLC (column: 3_Phenomenex Luna C18 75*30 mm*3 um; mobile phase: [water (0.1% TFA)-ACN]; B %: 56%-76%, 7 min). The aqueous phase was extracted with ethyl acetate (15 mL×3). The combined organic layers were dried over anhydrous sodium sulfate, filtered and concentrated to give the title compound (150 mg, 54% yield) as a colorless oil. MS (ES+) C₁₈H₂₁ClN₆O₂ requires: 388, found: 389[M+H]⁺.

### Example 2ax

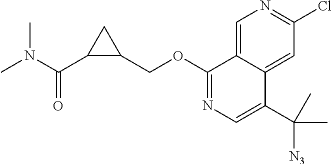 [SMILES: CN(C)C(=O)C1CC1COc1ncc(C(C)(C)N=[N+]=[N-])c2cc(Cl)ncc12]

### Intermediate 75: 2-(((4-(2-Azidopropan-2-yl)-6-chloro-2,7-naphthyridin-1-yl)oxy)methyl)-N,N-dimethylcyclopropane-1-carboxamide

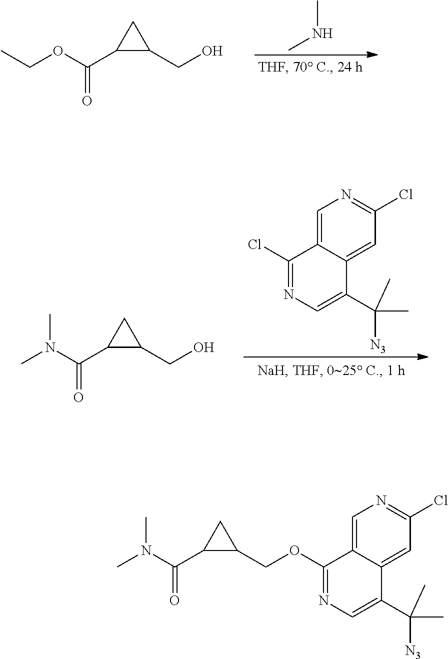

### Step 1: 2-(Hydroxymethyl)-N,N-dimethylcyclopropane-1-carboxamide

To a solution of ethyl 2-(hydroxymethyl)cyclopropane-1-carboxylate (500 mg, 3.47 mmol) in THF (2 mL) was added dimethylamine (2.0 M, 10 mL). The reaction mixture was stirred at 70° C. for 24 h, then was concentrated to give the title compound (550 mg, crude) as a yellow oil that was used without further purification.

### Step 2: 2-(((4-(2-Azidopropan-2-yl)-6-chloro-2,7-naphthyridin-1-yl)oxy)methyl)-N,N-dimethylcyclopropane-1-carboxamide

The title compound was prepared from 2-(hydroxymethyl)-N,N-dimethylcyclopropane-1-carboxamide and 4-(2-azidopropan-2-yl)-1,6-dichloro-2,7-naphthyridine using a procedure similar to that described in Step 1 of Intermediate 9. MS (ES+) C₁₈H₂₁ClN₆O₂ requires: 388, found: 389[M+H]⁺.

### Example 2ay

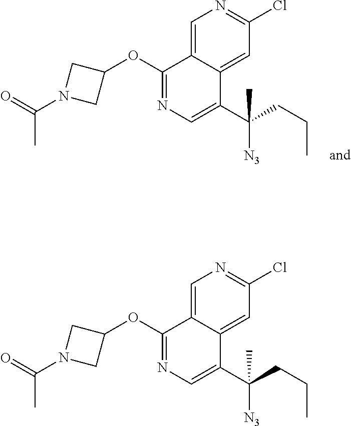 [SMILES: CCC[C@](C)(N=[N+]=[N-])c1cnc(OC2CN(C(C)=O)C2)c2cnc(Cl)cc12 ; CCC[C@@](C)(N=[N+]=[N-])c1cnc(OC2CN(C(C)=O)C2)c2cnc(Cl)cc12]

### Intermediates 76 and 77: (S)-1-(3-((4-(2-Azidopentan-2-yl)-6-chloro-2,7-naphthyridin-1-yl)oxy)azetidin-1-yl)ethan-1-one and (R)-1-(3-((4-(2-azidopentan-2-yl)-6-chloro-2,7-naphthyridin-1-yl)oxy)azetidin-1-yl)ethan-1-one

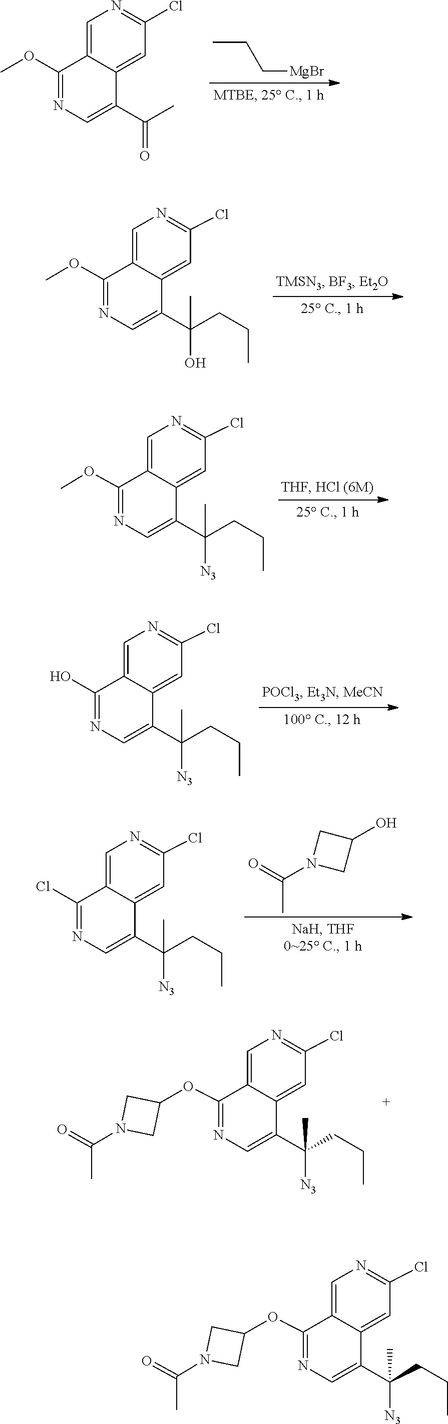

### Step 1: 2-(6-Chloro-1-methoxy-2,7-naphthyridin-4-yl)pentan-2-ol

To a solution of 1-(6-chloro-1-methoxy-2,7-naphthyridin-4-yl)ethan-1-one (3.00 g, 12.7 mmol) in MTBE (30 mL) was added propylmagnesium bromide (2.00 M, 12.7 mL). The reaction mixture was stirred at 25° C. for 1 h, then was diluted with saturated aqueous ammonium chloride solution (150 mL) and extracted with EA (150 mL×3). The combined organic layers were dried over anhydrous sodium sulfate, filtered and concentrated to give a residue. The residue was purified by column chromatography (SiO₂, PE/EA=1:0 to 3:2) to give the title compound (2.00 g, 4.99 mmol, 39% yield, 70% purity) as a yellow oil. MS (ES+) C₁₄H₁₇ClN₂O₂ requires: 280, found: 281[M+H]⁺.

### Step 2: 4-(2-Azidopentan-2-yl)-6-chloro-1-methoxy-2,7-naphthyridine

To a mixture of 2-(6-chloro-1-methoxy-2,7-naphthyridin-4-yl)pentan-2-ol (1.80 g, 4.49 mmol, 70% purity) in DCM (30 mL) was added BF₃OEt₂ (1.27 g, 8.98 mmol, 1.11 mL) and TMSN₃ (2.07 g, 18.0 mmol, 2.36 mL). The reaction mixture was stirred at 25° C. for 1 h, then was diluted with water (50 mL) and extracted with EA (50 mL×3). The combined organic layers were dried over anhydrous sodium sulfate, filtered and concentrated to give a residue. The residue was purified by column chromatography (SiO₂, PE/EA=1:0 to 9:1) to give the title compound (1.30 g, 4.25 mmol, 95% yield) as a yellow solid.

### Step 3: 4-(2-Azidopentan-2-yl)-6-chloro-2,7-naphthyridin-1(2H)-one

To a solution of 4-(2-azidopentan-2-yl)-6-chloro-1-methoxy-2,7-naphthyridine (1.30 g, 4.25 mmol) in THF (20 mL) was added aqueous HCl (6 M, 10 mL). The reaction mixture was stirred at 25° C. for 1 h, then was diluted with saturated aqueous sodium bicarbonate solution to pH>7 and extracted with EA (50 mL×3). The combined organic layers were dried over anhydrous sodium sulfate, filtered and concentrated to give a residue. The residue was purified by column chromatography (SiO₂, PE/EA=1:0 to 7:3) to give the title compound (1.00 g, 81% yield) as a white solid.

### Step 4: 4-(2-Azidopentan-2-yl)-1,6-dichloro-2,7-naphthyridine

To a solution of 4-(2-azidopentan-2-yl)-6-chloro-2,7-naphthyridin-1(2H)-one (800 mg, 2.74 mmol) in ACN (15 mL) was added POCl₃ (2.10 g, 13.7 mmol, 1.27 mL) and Et₃N (721 mg, 7.13 mmol, 992 μL). The reaction mixture was stirred at 100° C. for 12 h, then was diluted with saturated aqueous sodium bicarbonate solution (50 mL) and extracted with EA (25 mL×3). The combined organic layers were dried over anhydrous sodium sulfate, filtered and concentrated to give a residue. The residue was purified by column chromatography (SiO₂, PE/EA=1:0 to 9:1) to give the title compound (700 mg, 82% yield) as a yellow solid.

### Step 5: (S)-1-(3-((4-(2-Azidopentan-2-yl)-6-chloro-2,7-naphthyridin-1-yl)oxy)azetidin-1-yl)ethan-1-one or (R)-1-(3-((4-(2-azidopentan-2-yl)-6-chloro-2,7-naphthyridin-1-yl)oxy)azetidin-1-yl)ethan-1-one

To a solution of 1-(3-hydroxyazetidin-1-yl)ethan-1-one (204 mg, 1.77 mmol) in THF (10 mL) was added NaH (85.1 mg, 2.13 mmol, 60% purity) at 0° C. The mixture was stirred at 25° C. for 0.5 h, then 4-(2-azidopentan-2-yl)-1,6-dichloro-2,7-naphthyridine (550 mg, 1.77 mmol) was added and the reaction mixture was stirred at 25° C. for 0.5 h. The reaction mixture was then diluted with saturated aqueous sodium chloride solution (20 mL) and extracted with EA (20 mL×3). The combined organic layers were dried over anhydrous sodium sulfate, filtered and concentrated to give a residue. The residue was purified by column chromatography (SiO₂, PE/EA=1:0 to 0:1) to give a racemic mixture of title compounds (680 mg, 99% yield) as a colorless oil. The mixture was separated by chiral SFC (column: Daicel Chiralpak AD-H (250 mm*30 mm, 5 um); mobile phase: [0.1% NH₃H₂O MeOH]; B %: 30%-30%) to give the first of (S)-1-(3-((4-(2-azidopentan-2-yl)-6-chloro-2,7-naphthyridin-1-yl)oxy)azetidin-1-yl)ethan-1-one or (R)-1-(3-((4-(2-azidopentan-2-yl)-6-chloro-2,7-naphthyridin-1-yl)oxy)azetidin-1-yl)ethan-1-one (peak 1, Intermediate 76, 325 mg, 44% yield) as a colorless oil and the second of (S)-1-(3-((4-(2-azidopentan-2-yl)-6-chloro-2,7-naphthyridin-1-yl)oxy)azetidin-1-yl)ethan-1-one or (R)-1-(3-((4-(2-azidopentan-2-yl)-6-chloro-2,7-naphthyridin-1-yl)oxy)azetidin-1-yl)ethan-1-one (peak 2, Intermediate 77, 380 mg, 51% yield) as a colorless oil. MS (ES+) C₁₈H₂₁ClN₆O₂ requires: 388, found: 389[M+H]⁺.

### Example 2az

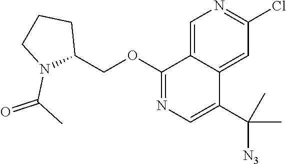 [SMILES: CC(=O)N1CCC[C@@H]1COc1ncc(C(C)(C)N=[N+]=[N-])c2cc(Cl)ncc12]

### Intermediate 78: (R)-1-(2-(((4-(2-Azidopropan-2-yl)-6-chloro-2,7-naphthyridin-1-yl)oxy)methyl)pyrrolidin-1-yl)ethan-1-one

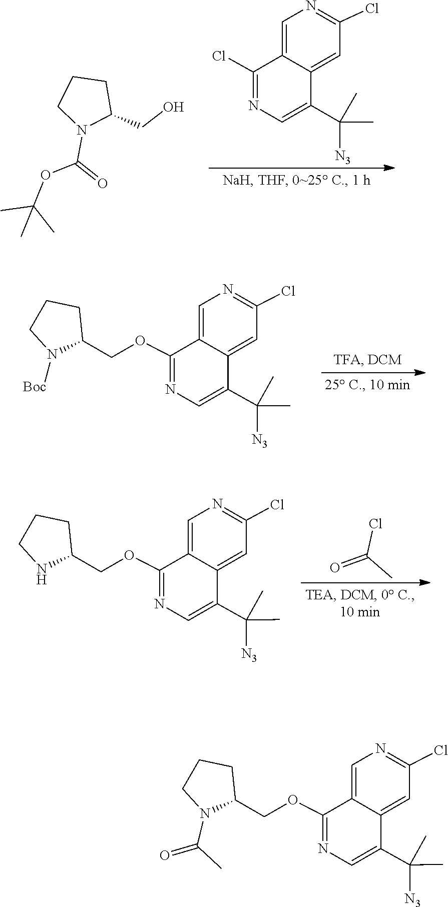

### Step 1: tert-Butyl (R)-2-(((4-(2-azidopropan-2-yl)-6-chloro-2,7-naphthyridin-1-yl)oxy)methyl)pyrrolidine-1-carboxylate

The title compound was prepared from tert-butyl (R)-2-(hydroxymethyl)pyrrolidine-1-carboxylate and 4-(2-azidopropan-2-yl)-1,6-dichloro-2,7-naphthyridine using a procedure similar to that described in Step 1 of Intermediate 9. MS (ES+) C₂₁H₂₇ClN₆O₃ requires: 446, found: 447[M+H]⁺.

### Step 2: (R)-4-(2-Azidopropan-2-yl)-6-chloro-1-(pyrrolidin-2-ylmethoxy)-2,7-naphthyridine

To a solution of tert-butyl (R)-2-(((4-(2-azidopropan-2-yl)-6-chloro-2,7-naphthyridin-1-yl)oxy)methyl)pyrrolidine-1-carboxylate (200 mg, 448 μmol) in DCM (3 mL) was added TFA (1.54 g, 13.5 mmol, 1 mL). The reaction mixture was stirred at 25° C. for 10 min, then was diluted with saturated aqueous sodium bicarbonate solution (10 mL) and extracted with EA (10 mL×3). The combined organic layers were dried over anhydrous sodium sulfate, filtered and concentrated to give the title compound (220 mg, crude) as a yellow oil that was used in the next step without further purification.

### Step 3: (R)-1-(2-(((4-(2-Azidopropan-2-yl)-6-chloro-2,7-naphthyridin-1-yl)oxy)methyl)pyrrolidin-1-yl)ethan-1-one

To a solution of (R)-4-(2-azidopropan-2-yl)-6-chloro-1-(pyrrolidin-2-ylmethoxy)-2,7-naphthyridine (171 mg, 492 μmol) in DCM (5.00 mL) was added Et₃N (149 mg, 1.48 mmol, 206 μL) and acetyl chloride (70.3 μL, 985 μmol). The reaction mixture was stirred at 0° C. for 10 min, then was diluted with water (10 mL) and extracted with EA (10 mL×3). The combined organic layers were dried over anhydrous sodium sulfate, filtered and concentrated to give a residue. The residue was purified by column chromatography (SiO₂, PE/EA=1:0 to 2:3) to give the title compound (95.0 mg, 50% yield) as a yellow oil. MS (ES+) C₁₈H₂₁ClN₆O₂ requires: 388, found: 389[M+H]⁺.

### Example 2ba

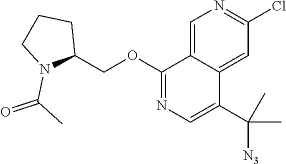 [SMILES: CC(=O)N1CCC[C@H]1COc1ncc(C(C)(C)N=[N+]=[N-])c2cc(Cl)ncc12]

### Intermediate 79: (S)-1-(2-(((4-(2-Azidopropan-2-yl)-6-chloro-2,7-naphthyridin-1-yl)oxy)methyl)pyrrolidin-1-yl)ethan-1-one

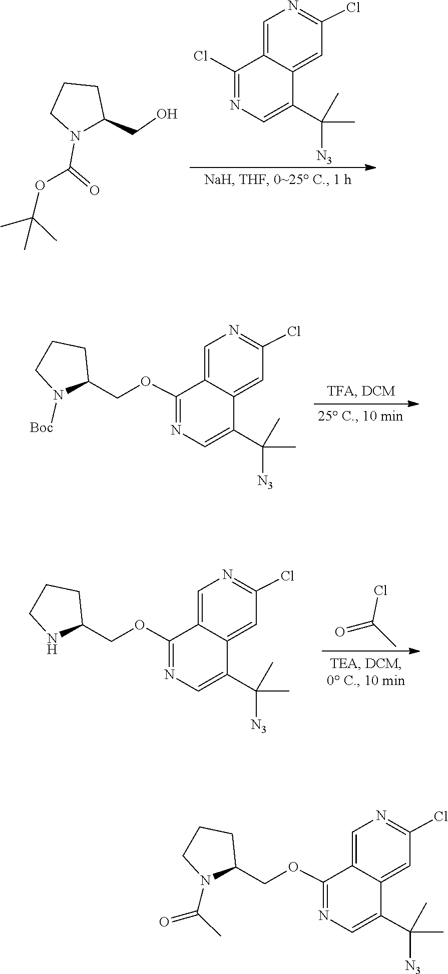

### Steps 1-3: (S)-1-(2-(((4-(2-Azidopropan-2-yl)-6-chloro-2,7-naphthyridin-1-yl)oxy)methyl)pyrrolidin-1-yl)ethan-1-one

The title compound was prepared from tert-butyl (S)-2-(hydroxymethyl)pyrrolidine-1-carboxylate and 4-(2-azidopropan-2-yl)-1,6-dichloro-2,7-naphthyridine using a procedure similar to that described in Steps 1-3 of Intermediate 78. MS (ES+) C₁₈H₂₁ClN₆O₂ requires: 388, found: 389[M+H]⁺.

### Example 2bb

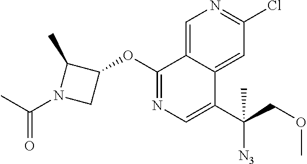 [SMILES: COC[C@@](C)(N=[N+]=[N-])c1cnc(O[C@@H]2CN(C(C)=O)[C@H]2C)c2cnc(Cl)cc12]

### Intermediate 80: 1-((2S,3R)-3-((4-((S)-2-Azido-1-methoxypropan-2-yl)-6-chloro-2,7-naphthyridin-1-yl)oxy)-2-methylazetidin-1-yl)ethan-1-one

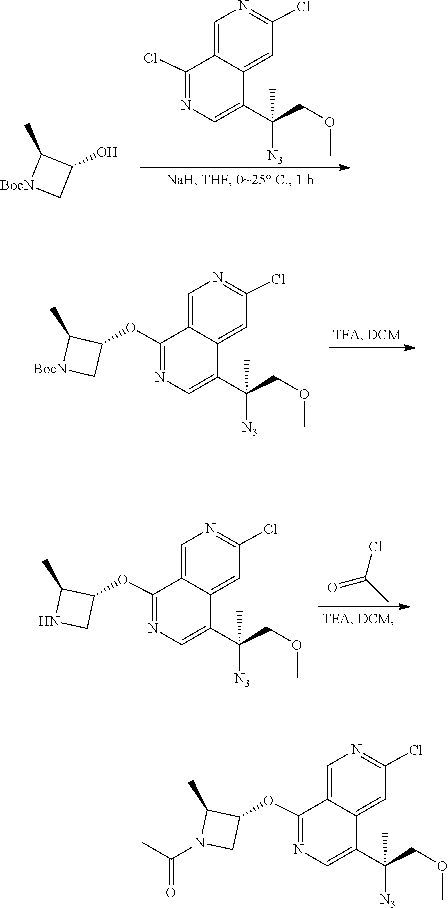

### Steps 1-3: (S)-1-(2-(((4-(2-Azidopropan-2-yl)-6-chloro-2,7-naphthyridin-1-yl)oxy)methyl)pyrrolidin-1-yl)ethan-1-one

The title compound was prepared from tert-butyl (2S,3R)-3-hydroxy-2-methylazetidine-1-carboxylate and (S)-4-(2-azido-1-methoxypropan-2-yl)-1,6-dichloro-2,7-naphthyridine (Intermediate 15) using a procedure similar to that described in Steps 1-3 of Intermediate 9. MS (ES+) C₁₈H₂₁ClN₆O₃ requires: 404, found: 405[M+H]⁺.

### Example 2bc

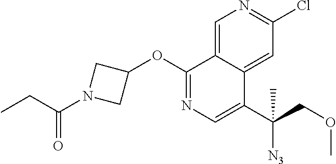 [SMILES: CCC(=O)N1CC(Oc2ncc([C@@](C)(COC)N=[N+]=[N-])c3cc(Cl)ncc23)C1]

### Intermediate 81: (S)-1-(3-((4-(2-Azido-1-methoxypropan-2-yl)-6-chloro-2,7-naphthyridin-1-yl)oxy)azetidin-1-yl)propan-1-one

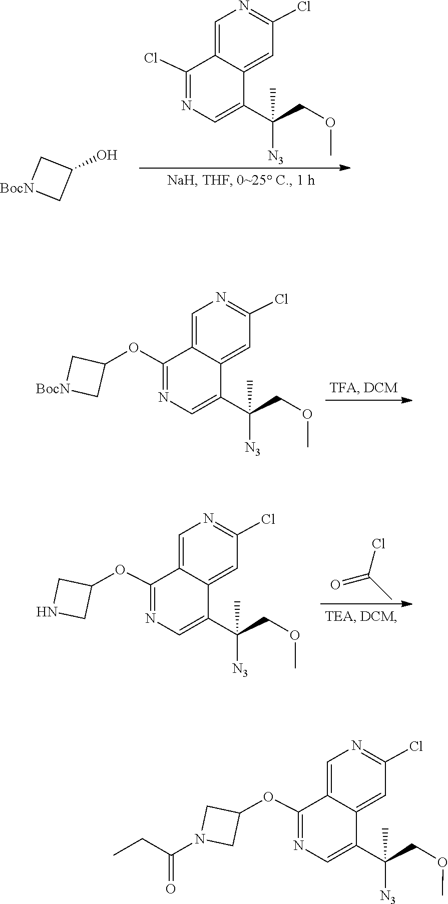

### Steps 1-3: (S)-1-(3-((4-(2-Azido-1-methoxypropan-2-yl)-6-chloro-2,7-naphthyridin-1-yl)oxy)azetidin-1-yl)propan-1-one

The title compound was prepared from tert-butyl 3-hydroxyazetidine-1-carboxylate and (S)-4-(2-azido-1-methoxypropan-2-yl)-1,6-dichloro-2,7-naphthyridine (Step 1, Intermediate 80) using a procedure similar to that described in Steps 1-3 of Intermediate 9. MS (ES+) C₁₈H₂₁ClN₆O₃ requires: 404, found: 405[M+H]⁺.

### Example 2bd

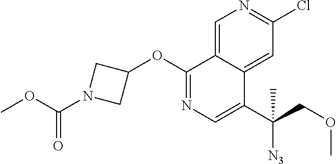

### Intermediate 82: Methyl (S)-3-((4-(2-azido-1-methoxypropan-2-yl)-6-chloro-2,7-naphthyridin-1-yl)oxy)azetidine-1-carboxylate

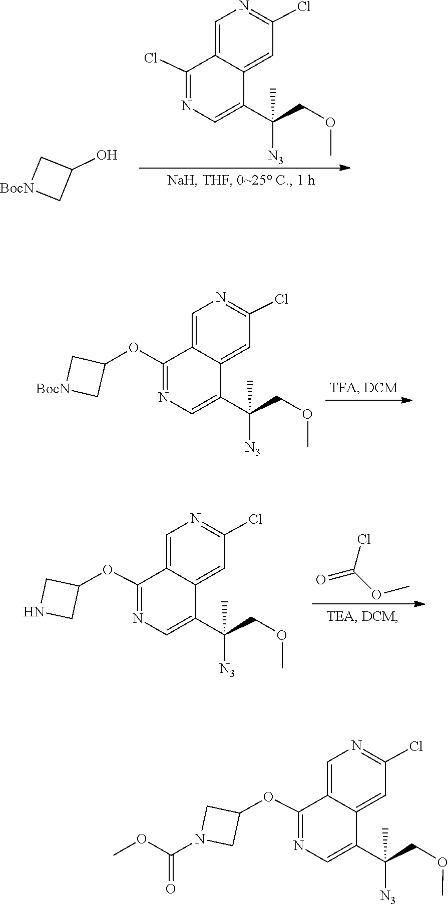

### Steps 1-3: Methyl (S)-3-((4-(2-azido-1-methoxypropan-2-yl)-6-chloro-2,7-naphthyridin-1-yl)oxy)azetidine-1-carboxylate

The title compound was prepared from tert-butyl 3-hydroxyazetidine-1-carboxylate and (S)-4-(2-azido-1-methoxypropan-2-yl)-1,6-dichloro-2,7-naphthyridine (Intermediate 15) using a procedure similar to that described in Steps 1-3 of Intermediate 9, except that methyl chloroformate was used in the last step. MS (ES+) C₁₇H₁₉ClN₆O₄ requires: 406, found: 407[M+H]⁺.

### Example 3: Representative Methods to Synthesize Compounds in Table 1

### Example 3a: Synthetic Method 1

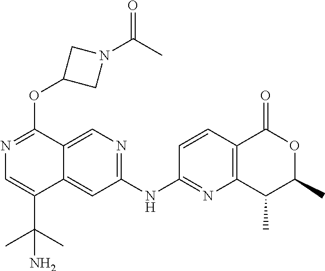 [SMILES: CC(=O)N1CC(Oc2ncc(C(C)(C)N)c3cc(Nc4ccc5c(n4)[C@@H](C)[C@H](C)OC5=O)ncc23)C1]

### (7S,8R)-2-((8-((1-acetylazetidin-3-yl)oxy)-5-(2-aminopropan-2-yl)-2,7-naphthyridin-3-yl)amino)-7,8-dimethyl-7,8-dihydro-5H-pyrano[4,3-b]pyridin-5-one (Compound 1)

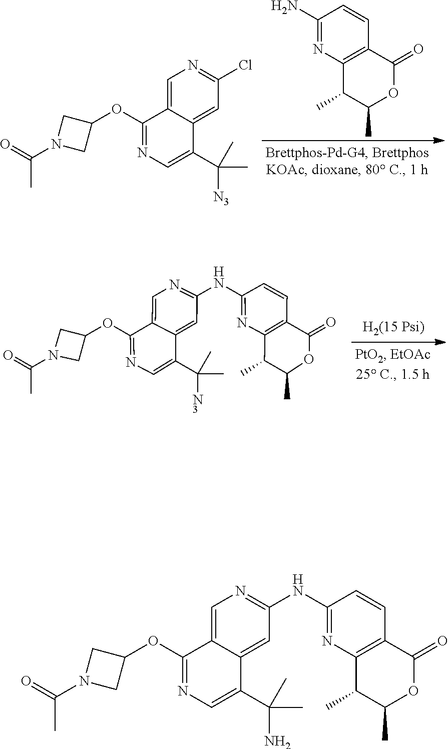 [SMILES: CC(=O)N1CC(Oc2ncc(C(C)(C)N=[N+]=[N-])c3cc(Cl)ncc23)C1 ; C[C@@H]1OC(=O)c2ccc(N)nc2[C@H]1C ; CC(=O)N1CC(Oc2ncc(C(C)(C)N=[N+]=[N-])c3cc(Nc4ccc5c(n4)[C@@H](C)[C@H](C)OC5=O)ncc23)C1 ; CC(=O)N1CC(Oc2ncc(C(C)(C)N)c3cc(Nc4ccc5c(n4)[C@@H](C)[C@H](C)OC5=O)ncc23)C1]

### Step 1: (7S,8R)-2-((8-((1-acetylazetidin-3-yl)oxy)-5-(2-azidopropan-2-yl)-2,7-naphthyridin-3-yl)amino)-7,8-dimethyl-7,8-dihydro-5H-pyrano[4,3-b]pyridin-5-one

BrettPhos Pd G4 (25.5 mg, 27.7 umol), BrettPhos (14.9 mg, 27.7 umol), and potassium acetate (81.60 mg, 831 umol) were added to a solution of 1-(3-((4-(2-azidopropan-2-yl)-6-chloro-2,7-naphthyridin-1-yl)oxy)azetidin-1-yl)ethan-1-one (Intermediate 7)(100 mg, 277 umol) and (7S,8R)-2-amino-7,8-dimethyl-7,8-dihydro-5H-pyrano[4,3-b]pyridin-5-one (Intermediate 1)(79.9 mg, 416 umol) in dioxane (4 mL). The reaction mixture was stirred at 80° C. for 1 h under nitrogen, then was concentrated to give a residue. The residue was purified by flash-column chromatography on silica gel (gradient elution, 50% to 100% EA-PE) to give the title compound (140 mg, 80% yield) as a yellow solid. MS (ES+) C₂₆H₂₈N₈O₄ requires: 516, found: 517[M+H]⁺.

### Step 2: (7S,8R)-2-((8-((1-acetylazetidin-3-yl)oxy)-5-(2-aminopropan-2-yl)-2,7-naphthyridin-3-yl)amino)-7,8-dimethyl-7,8-dihydro-5H-pyrano[4,3-b]pyridin-5-one

A solution of (7S,8R)-2-((8-((1-acetylazetidin-3-yl)oxy)-5-(2-azidopropan-2-yl)-2,7-naphthyridin-3-yl)amino)-7,8-dimethyl-7,8-dihydro-5H-pyrano[4,3-b]pyridin-5-one (120 mg, 2321 umol) in EA (2 mL) was degassed and backfilled with nitrogen. Platinum (IV) oxide hydrate (Adam's catalyst, 38.5 mg, 170 umol) was then added and the reaction mixture was evacuated and backfilled with hydrogen at ˜15 psi. The mixture was stirred at 25° C. for 1.5 h under ˜15 psi hydrogen. The reaction mixture was then filtered and concentrated to give a residue. The residue was purified by prep-HPLC (column: Phenomenex Synergi C18 150*25*10 um; mobile phase: [water (0.225% FA)-ACN] 10%-40% gradient over 10 min) and dried to give the title compound (8.1 mg, 6.0% yield, formic acid salt) as an off-white solid. MS (ES+) C₂₆H₃₀N₆O₄ requires: 490, found: 491[M+H]⁺. ¹H-NMR (400 MHz, CD₃OD): δ ppm 9.51 (s, 1H), 9.05 (s, 1H), 8.52 (s, 1H), 8.20 (d, J=8.7 Hz, 1H), 8.04 (s, 1H), 7.40 (d, J=8.7 Hz, 1H), 5.82-5.56 (m, 1H), 4.78-4.65 (m, 2H), 4.51 (dd, J=6.7, 11.2 Hz, 1H), 4.43 (dd, J=3.9, 10.0 Hz, 1H), 4.14 (dd, J=4.0, 11.4 Hz, 1H), 3.08-2.92 (m, 1H), 2.00 (d, J=6.6 Hz, 6H), 1.96 (s, 3H), 1.53 (d, J=7.2 Hz, 3H), 1.48 (d, J=6.5 Hz, 3H).

### Example 3b: Synthetic Method 1

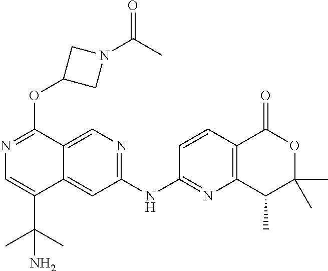 [SMILES: CC(=O)N1CC(Oc2ncc(C(C)(C)N)c3cc(Nc4ccc5c(n4)[C@@H](C)C(C)(C)OC5=O)ncc23)C1]

### (R)-2-((8-((1-Acetylazetidin-3-yl)oxy)-5-(2-aminopropan-2-yl)-2,7-naphthyridin-3-yl)amino)-7,7,8-trimethyl-7,8-dihydro-5H-pyrano[4,3-b]pyridin-5-one (Compound 4)

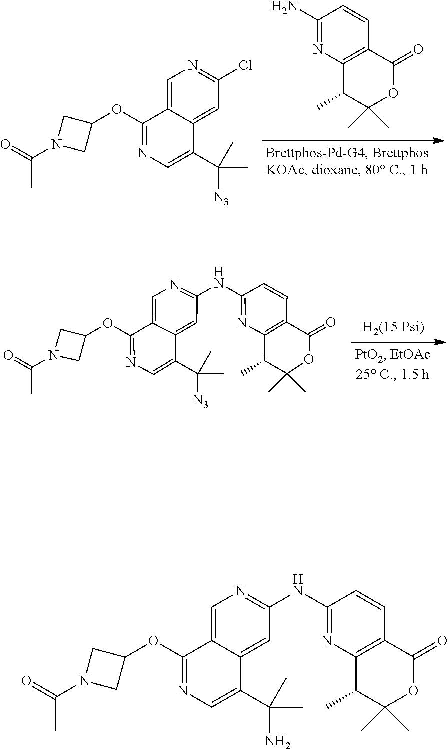 [SMILES: CC(=O)N1CC(Oc2ncc(C(C)(C)N=[N+]=[N-])c3cc(Cl)ncc23)C1 ; C[C@@H]1c2nc(N)ccc2C(=O)OC1(C)C ; CC(=O)N1CC(Oc2ncc(C(C)(C)N=[N+]=[N-])c3cc(Nc4ccc5c(n4)[C@@H](C)C(C)(C)OC5=O)ncc23)C1 ; CC(=O)N1CC(Oc2ncc(C(C)(C)N)c3cc(Nc4ccc5c(n4)[C@@H](C)C(C)(C)OC5=O)ncc23)C1]

### Step 1: (R)-2-((8-((1-Acetylazetidin-3-yl)oxy)-5-(2-azidopropan-2-yl)-2,7-naphthyridin-3-yl)amino)-7,7,8-trimethyl-7,8-dihydro-5H-pyrano[4,3-b]pyridin-5-one

BrettPhos Pd G4 (128 mg, 139 μmol), BrettPhos (74.6 mg, 139 μmol), and potassium acetate (341 mg, 3.47 mmol) were added to a solution of 1-(3-((4-(2-azidopropan-2-yl)-6-chloro-2,7-naphthyridin-1-yl)oxy)azetidin-1-yl)ethan-1-one (Intermediate 7)(500 mg, 1.39 mmol) and (R)-2-amino-7,7,8-trimethyl-7,8-dihydro-5H-pyrano[4,3-b]pyridin-5-one (Intermediate 3)(287 mg, 1.39 mmol) in dioxane (10 mL). The reaction mixture was stirred at 80° C. for 1 h under nitrogen, then was concentrated to give a residue. The residue was purified by flash-column chromatography on silica gel (gradient elution, 50% to 100% EA-PE) to give the title compound (600 mg, 81% yield) as a yellow solid. MS (ES+) C₂₇H₃₀N₈O₄ requires: 530, found: 531[M+H]⁺.

### Step 2: (R)-2-((8-((1-Acetylazetidin-3-yl)oxy)-5-(2-aminopropan-2-yl)-2,7-naphthyridin-3-yl)amino)-7,7,8-trimethyl-7,8-dihydro-5H-pyrano[4,3-b]pyridin-5-one

A solution of (R)-2-((8-((1-acetylazetidin-3-yl)oxy)-5-(2-azidopropan-2-yl)-2,7-naphthyridin-3-yl)amino)-7,7,8-trimethyl-7,8-dihydro-5H-pyrano[4,3-b]pyridin-5-one (600 mg, 1.13 mmol) in EA (150 mL) was degassed and backfilled with nitrogen. Platinium (IV) oxide hydrate (Adam's catalyst, 193 mg, 850 μmol) was then added and the reaction mixture was evacuated and backfilled with hydrogen at ˜15 psi. The mixture was stirred at 25° C. for 1.5 h under ˜15 psi hydrogen. The reaction mixture was then filtered and concentrated to give a residue. The residue was purified flash-column chromatography on silica gel (0-10% MeOH-EA) to give the title compound (346 mg, 60% yield) as a yellow solid formic acid salt) as an off-white solid. MS (ES+) C₂₇H₃₂N₆O₄ requires: 504, found: 505[M+H]⁺. ¹H-NMR (400 MHz, CD₃OD): δ ppm 9.43 (s, 1H), 9.39 (s, 1H), 8.17 (s, 1H), 8.14 (d, J=8.4 Hz, 1H), 7.23 (d, J=8.8 Hz, 1H), 5.65-5.57 (m, 1H), 4.77-4.68 (m, 1H), 4.55-4.45 (m, 1H), 4.44-4.35 (m, 1H), 4.16-4.09 (m, 1H), 3.09-3.00 (m, 1H), 1.93 (s, 3H), 1.81 (d, J=8.0 Hz, 6H), 1.54 (s, 3H), 1.45 (s, 3H), 1.40 (d, J=7.2 Hz, 3H).

### Example 3c: Synthetic Method 2

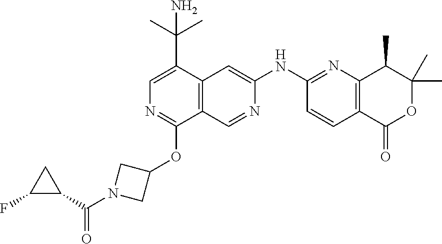 [SMILES: C[C@@H]1c2nc(Nc3cc4c(C(C)(C)N)cnc(OC5CN(C(=O)[C@H]6C[C@H]6F)C5)c4cn3)ccc2C(=O)OC1(C)C]

### (R)-2-((5-(2-aminopropan-2-yl)-8-((1-((1R,2R)-2-fluorocyclopropane-1-carbonyl)azetidin-3-yl)oxy)-2,7-naphthyridin-3-yl)amino)-7,7,8-trimethyl-7,8-dihydro-5H-pyrano[4,3-b]pyridin-5-one (Compound 13)

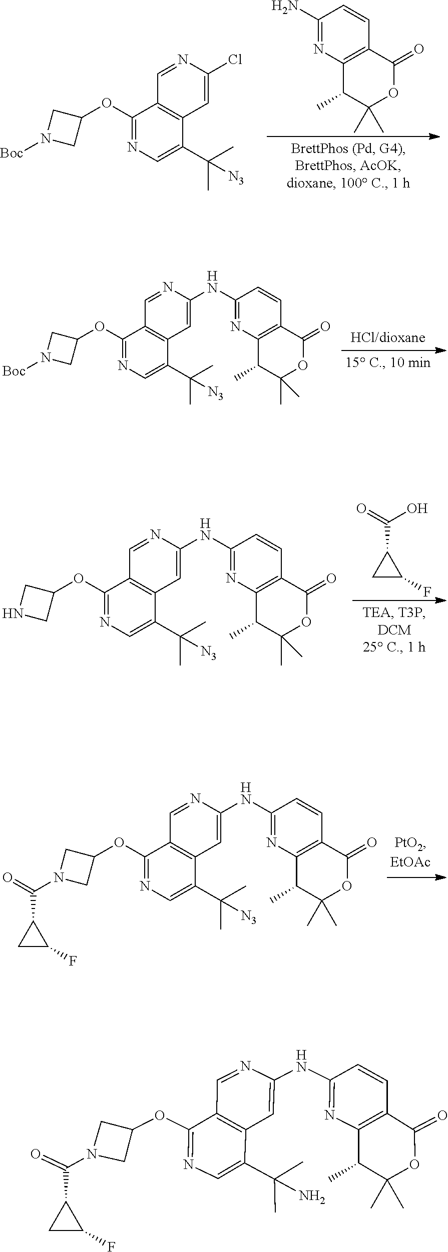

### Step 1: tert-butyl (R)-3-((4-(2-azidopropan-2-yl)-6-((7,7,8-trimethyl-5-oxo-7,8-dihydro-5H-pyrano[4,3-b]pyridin-2-yl)amino)-2,7-naphthyridin-1-yl)oxy)azetidine-1-carboxylate

The title compound was prepared from tert-butyl 3-((4-(2-azidopropan-2-yl)-6-chloro-2,7-naphthyridin-1-yl)oxy)azetidine-1-carboxylate (Intermediate 9, Step 1) and Intermediate 3 using a similar procedure as described above in Step 1 of Example 3a, except at 100° C.

### Step 2: (R)-2-((8-(azetidin-3-yloxy)-5-(2-azidopropan-2-yl)-2,7-naphthyridin-3-yl)amino)-7,7,8-trimethyl-7,8-dihydro-5H-pyrano[4,3-b]pyridin-5-one

HCl in dioxane (4 M, 6 mL) was added to a solution of tert-butyl (R)-3-((4-(2-azidopropan-2-yl)-6-((7,7,8-trimethyl-5-oxo-7,8-dihydro-5H-pyrano[4,3-b]pyridin-2-yl)amino)-2,7-naphthyridin-1-yl)oxy)azetidine-1-carboxylate (0.800 g, 1.36 mmol) in dioxane (6 mL). The reaction mixture was stirred at 25° C. for 10 min, then was concentrated to give the title compound (1.00 g, crude) as a yellow solid that was used in the next step without further purification.

### Step 3: (R)-2-((5-(2-azidopropan-2-yl)-8-((1-((1R,2R)-2-fluorocyclopropane-1-carbonyl)azetidin-3-yl)oxy)-2,7-naphthyridin-3-yl)amino)-7,7,8-trimethyl-7,8-dihydro-5H-pyrano[4,3-b]pyridin-5-one

(1R,2R)-2-fluorocyclopropane-1-carboxylic acid (Combi-Blocks #QM-4290)(63.9 mg, 614 μmol) was added to a solution of (R)-2-((8-(azetidin-3-yloxy)-5-(2-azidopropan-2-yl)-2,7-naphthyridin-3-yl)amino)-7,7,8-trimethyl-7,8-dihydro-5H-pyrano[4,3-b]pyridin-5-one (300 mg, 313 μmol, 50% purity) and TEA (155 mg, 1.54 mmol) in DCM (4 mL) and T3P (244 mg, 768 μmol). The reaction mixture was stirred at 25° C. for 1 h, then was diluted with water (10 mL) and extracted with DCM (10 mL×3). The combined organic layers were dried over sodium sulfate, filtered and concentrated to give a residue. The residue was purified by flash-column chromatography on silica gel (gradient elution, 0% to 80% EA-PE) to give the title compound (90 mg, 51% yield) as yellow solid.

### Step 4: (R)-2-((5-(2-aminopropan-2-yl)-8-((1-((1R,2R)-2-fluorocyclopropane-1-carbonyl)azetidin-3-yl)oxy)-2,7-naphthyridin-3-yl)amino)-7,7,8-trimethyl-7,8-dihydro-5H-pyrano[4,3-b]pyridin-5-one

The title compound was prepared from (R)-2-((5-(2-azidopropan-2-yl)-8-((1-((1R,2R)-2-fluorocyclopropane-1-carbonyl)azetidin-3-yl)oxy)-2,7-naphthyridin-3-yl)amino)-7,7,8-trimethyl-7,8-dihydro-5H-pyrano[4,3-b]pyridin-5-one using the same procedure described above in Step 2 of Example 3a. MS (ES+) C₂₉H₃₃FN₆O₄ requires: 548, found: 549[M+H]⁺. ¹H-NMR (400 MHz, CD₃OD): δ ppm 9.44 (d, J=3.6 Hz, 1H), 9.40 (s, 1H), 8.19 (s, 1H), 8.14 (d, J=8.8 Hz, 1H), 7.23 (d, J=8.8 Hz, 1H), 5.71-5.62 (m, 1H), 4.96-4.88 (m, 1H), 4.82-4.71 (m, 1H), 4.60-4.42 (m, 2H), 4.22-4.12 (m, 1H), 3.09-3.01 (m, 1H), 1.92-1.84 (m, 1H), 1.81 (d, J=7.2 Hz, 6H), 1.75-1.63 (m, 1H), 1.54 (s, 3H), 1.45 (s, 3H), 1.40 (d, J=7.2 Hz, 3H), 1.18-1.06 (m, 1H).

### Example 3ca: Synthetic Method 2

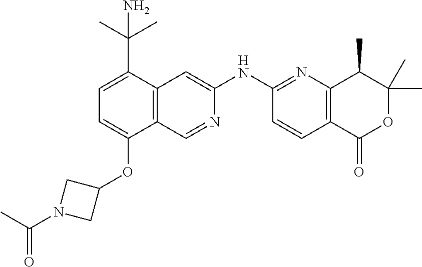 [SMILES: CC(=O)N1CC(Oc2ccc(C(C)(C)N)c3cc(Nc4ccc5c(n4)[C@@H](C)C(C)(C)OC5=O)ncc23)C1]

### (R)-2-((8-((1-Acetylazetidin-3-yl)oxy)-5-(2-aminopropan-2-yl)isoquinolin-3-yl)amino)-7,7,8-trimethyl-7,8-dihydro-5H-pyrano[4,3-b]pyridin-5-one (Compound 21)

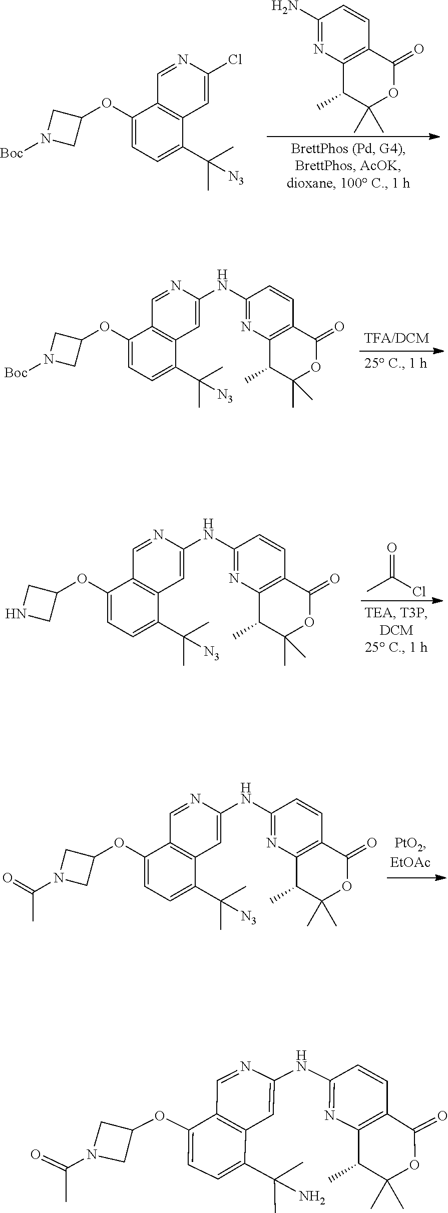

### Step 1: tert-Butyl (R)-3-((5-(2-azidopropan-2-yl)-3-((7,7,8-trimethyl-5-oxo-7,8-dihydro-5H-pyrano[4,3-b]pyridin-2-yl)amino)isoquinolin-8-yl)oxy)azetidine-1-carboxylate

The title compound was prepared from tert-butyl 3-((5-(2-azidopropan-2-yl)-3-chloroisoquinolin-8-yl)oxy)azetidine-1-carboxylate (Intermediate 38) and Intermediate 3 using a similar procedure as described above in Step 1 of Example 3a, except at 100° C.

### Step 2: (R)-2-((8-(Azetidin-3-yloxy)-5-(2-azidopropan-2-yl)isoquinolin-3-yl)amino)-7,7,8-trimethyl-7,8-dihydro-5H-pyrano[4,3-b]pyridin-5-one

A mixture of tert-butyl (R)-3-((5-(2-azidopropan-2-yl)-3-((7,7,8-trimethyl-5-oxo-7,8-dihydro-5H-pyrano[4,3-b]pyridin-2-yl)amino)isoquinolin-8-yl)oxy)azetidine-1-carboxylate (50.0 mg, 85.1 μmol) in DCM (1 ml) and TFA (0.3 ml) was stirred at 20° C. for 1 h. The reaction mixture was quenched by addition of saturated aqueous sodium bicarbonate solution (5 mL), then extracted with EA (5 ml×3). The combined organic layers were washed with brine, dried over anhydrous sodium sulfate, filtered and concentrated give the title compound (35.0 mg, crude) as a yellow oil.

### Step 3: (R)-2-((8-((1-Acetylazetidin-3-yl)oxy)-5-(2-azidopropan-2-yl)isoquinolin-3-yl)amino)-7,7,8-trimethyl-7,8-dihydro-5H-pyrano[4,3-b]pyridin-5-one

To a solution of (R)-2-((8-(Azetidin-3-yloxy)-5-(2-azidopropan-2-yl)isoquinolin-3-yl)amino)-7,7,8-trimethyl-7,8-dihydro-5H-pyrano[4,3-b]pyridin-5-one (35.0 mg, 71.8 mol) and NEt₃ (36.3 mg, 359 μmol) in DCM (1 ml) was added acetyl chloride (5.07 mg, 64.6 μmol). The reaction mixture was stirred at 0° C. for 30 min, then was concentrated and purified by prep-TLC (SiO₂, PE:EA=1:2) to give the title compound (20.0 mg, 47% yield) as a yellow oil.

### Step 4: (R)-2-((8-((1-Acetylazetidin-3-yl)oxy)-5-(2-aminopropan-2-yl)isoquinolin-3-yl)amino)-7,7,8-trimethyl-7,8-dihydro-5H-pyrano[4,3-b]pyridin-5-one

The title compound was prepared from (R)-2-((8-((1-Acetylazetidin-3-yl)oxy)-5-(2-azidopropan-2-yl)isoquinolin-3-yl)amino)-7,7,8-trimethyl-7,8-dihydro-5H-pyrano[4,3-b]pyridin-5-one using a similar procedure described above in Step 2 of Example 3a. MS (ES+) C₂₈H₃₃N₅O₄ requires: 503, found: 504[M+H]⁺. ¹H-NMR (400 MHz, CD₃OD): δ ppm 9.50 (s, 1H), 9.25 (s, 1H), 8.51 (s, 1H), 8.11 (d, J=8.8 Hz, 1H), 7.49 (d, J=8.4 Hz, 1H), 7.21 (d, J=8.8 Hz, 1H), 6.64 (d, J=8.4 Hz, 1H), 5.30 (s, 1H), 4.78-4.74 (m, 1H), 4.53-4.51 (m, 1H), 4.44-4.41 (m, 1H), 4.15-4.10 (m, 1H), 3.00-2.90 (m, 1H), 2.05 (s, 3H), 2.02 (s, 3H), 1.95 (s, 3H), 1.54 (s, 3H), 1.45 (s, 3H), 1.41 (d, J=7.6 Hz, 3H).

### Example 3d: Synthetic Method 3

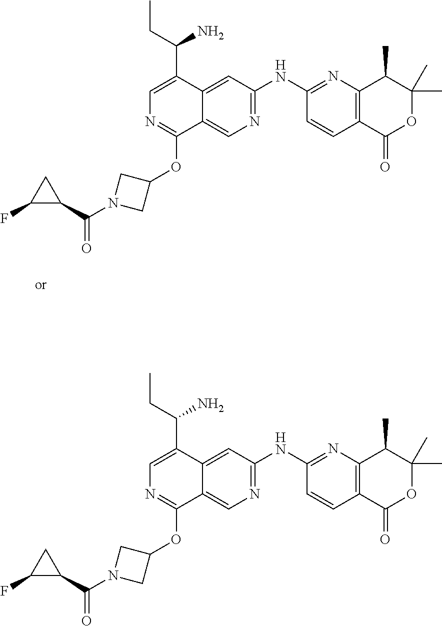 [SMILES: CC[C@@H](N)c1cnc(OC2CN(C(=O)[C@@H]3C[C@@H]3F)C2)c2cnc(Nc3ccc4c(n3)[C@@H](C)C(C)(C)OC4=O)cc12 ; CC[C@H](N)c1cnc(OC2CN(C(=O)[C@@H]3C[C@@H]3F)C2)c2cnc(Nc3ccc4c(n3)[C@@H](C)C(C)(C)OC4=O)cc12]

### (R)-2-((5-((R)-1-aminopropyl)-8-((1-((1S,2S)-2-fluorocyclopropane-1-carbonyl)azetidin-3-yl)oxy)-2,7-naphthyridin-3-yl)amino)-7,7,8-trimethyl-7,8-dihydro-5H-pyrano[4,3-b]pyridin-5-one or (R)-2-((5-((S)-1-aminopropyl)-8-((1-((1S,2S)-2-fluorocyclopropane-1-carbonyl)azetidin-3-yl)oxy)-2,7-naphthyridin-3-yl)amino)-7,7,8-trimethyl-7,8-dihydro-5H-pyrano[4,3-b]pyridin-5-one (Compound 11)

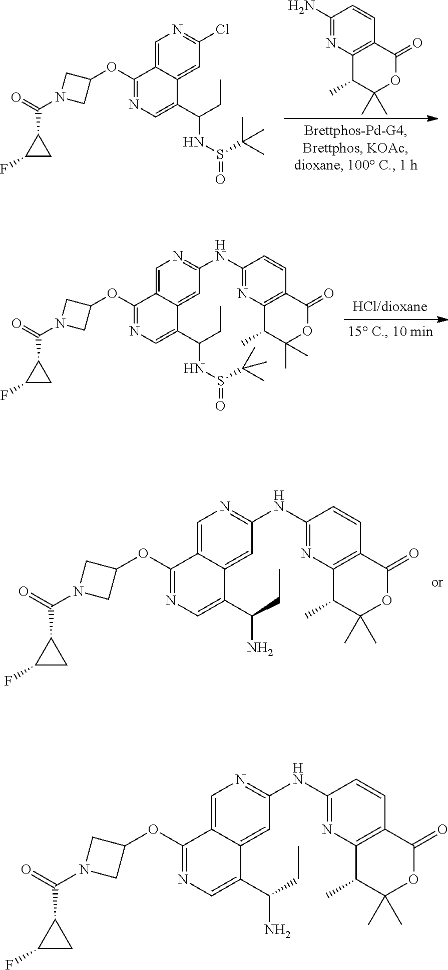 [SMILES: CCC(N[S@@](=O)C(C)(C)C)c1cnc(OC2CN(C(=O)[C@@H]3C[C@@H]3F)C2)c2cnc(Cl)cc12 ; C[C@@H]1c2nc(N)ccc2C(=O)OC1(C)C ; CCC(N[S@@](=O)C(C)(C)C)c1cnc(OC2CN(C(=O)[C@@H]3C[C@@H]3F)C2)c2cnc(Nc3ccc4c(n3)[C@@H](C)C(C)(C)OC4=O)cc12 ; CC[C@@H](N)c1cnc(OC2CN(C(=O)[C@@H]3C[C@@H]3F)C2)c2cnc(Nc3ccc4c(n3)[C@@H](C)C(C)(C)OC4=O)cc12 ; CC[C@H](N)c1cnc(OC2CN(C(=O)[C@@H]3C[C@@H]3F)C2)c2cnc(Nc3ccc4c(n3)[C@@H](C)C(C)(C)OC4=O)cc12]

### Step 1: (S)—N—((R)-1-(1-((1-((1S,2S)-2-fluorocyclopropane-1-carbonyl)azetidin-3-yl)oxy)-6-(((R)-7,7,8-trimethyl-5-oxo-7,8-dihydro-5H-pyrano[4,3-b]pyridin-2-yl)amino)-2,7-naphthyridin-4-yl)propyl)-2-methylpropane-2-sulfinamide or (S)—N—((S)-1-(1-((1-((1S,2S)-2-fluorocyclopropane-1-carbonyl)azetidin-3-yl)oxy)-6-(((R)-7,7,8-trimethyl-5-oxo-7,8-dihydro-5H-pyrano[4,3-b]pyridin-2-yl)amino)-2,7-naphthyridin-4-yl)propyl)-2-methylpropane-2-sulfinamide

BrettPhos Pd G4 (3.81 mg, 4.14 μmol), BrettPhos (2.22 mg, 4.14 μmol), and potassium acetate (20.3 mg, 207 μmol) were added to a solution of Intermediate 13 (20 mg, 41.4 μmol), and Intermediate 3 (9.39 mg, 45.5 μmol in dioxane (1 mL). The reaction mixture was stirred at 100° C. for 1 h, then was filtered and concentrated to give a residue. The residue was purified by prep-TLC (SiO2, 10% MeOH-EA) to give the title compound (15 mg, 55% yield) as a yellow solid.

### Step 2: (R)-2-((5-((R)-1-aminopropyl)-8-((1-((1S,2S)-2-fluorocyclopropane-1-carbonyl)azetidin-3-yl)oxy)-2,7-naphthyridin-3-yl)amino)-7,7,8-trimethyl-7,8-dihydro-5H-pyrano[4,3-b]pyridin-5-one or (R)-2-((5-((S)-1-aminopropyl)-8-((1-((1S,2S)-2-fluorocyclo propane-1-carbonyl)azetidin-3-yl)oxy)-2,7-naphthyridin-3-yl)amino)-7,7,8-trimethyl-7,8-dihydro-5H-pyrano[4,3-b]pyridin-5-one

HCl in dioxane (4 M, 1 mL) was added to (S)—N—((R)-1-(1-((1-((1S,2S)-2-fluorocyclopropane-1-carbonyl)azetidin-3-yl)oxy)-6-(((R)-7,7,8-trimethyl-5-oxo-7,8-dihydro-5H-pyrano[4,3-b]pyridin-2-yl)amino)-2,7-naphthyridin-4-yl)propyl)-2-methylpropane-2-sulfinamide or (S)—N—((S)-1-(1-((1-((1S,2S)-2-fluorocyclopropane-1-carbonyl)azetidin-3-yl)oxy)-6-(((R)-7,7,8-trimethyl-5-oxo-7,8-dihydro-5H-pyrano[4,3-b]pyridin-2-yl)amino)-2,7-naphthyridin-4-yl)propyl)-2-methylpropane-2-sulfinamide (15 mg, 22.9 μmol) at 15° C. The reaction mixture was stirred for 10 min, then was concentrated to give a residue. The residue was purified by prep-HPLC (column: Phenomenex Gemini-NX C18 75*30 mm*3 um; mobile phase: [water (0.225% FA)-ACN]; B %: 15%-45%, over 7 min) to give the title compound (5.7 mg, 38% yield) as yellow solid. MS (ES+) C₂₉H₃₃FN₆O₄ requires: 548, found: 549[M+H]⁺. ¹H-NMR (400 MHz, CD₃OD): δ ppm 9.45 (s, 1H), 8.86 (s, 1H), 8.55 (s, 1H), 8.18-8.14 (m, 2H), 7.31 (d, J=8.8 Hz, 1H), 5.70 (s, 1H), 4.81-4.76 (m, 1H), 4.61-4.52 (m, 4H), 4.21-4.18 (m, 1H), 3.23-3.17 (m, 1H), 2.12-2.03 (m, 2H), 1.90 (m, 1H), 1.55-1.46 (m, 1H), 1.46 (s, 3H), 1.12-1.10 (m, 1H), 1.07-1.02 (m, 3H).

### Example 3e: Synthetic Method 4

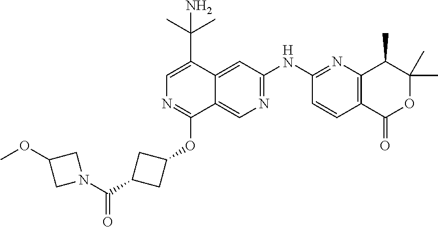

### (R)-2-((5-(2-aminopropan-2-yl)-8-((cis)-3-(3-methoxyazetidine-1-carbonyl)cyclobutoxy)-2,7-naphthyridin-3-yl)amino)-7,7,8-trimethyl-7,8-dihydro-5H-pyrano[4,3-b]pyridin-5-one (Compound 15)

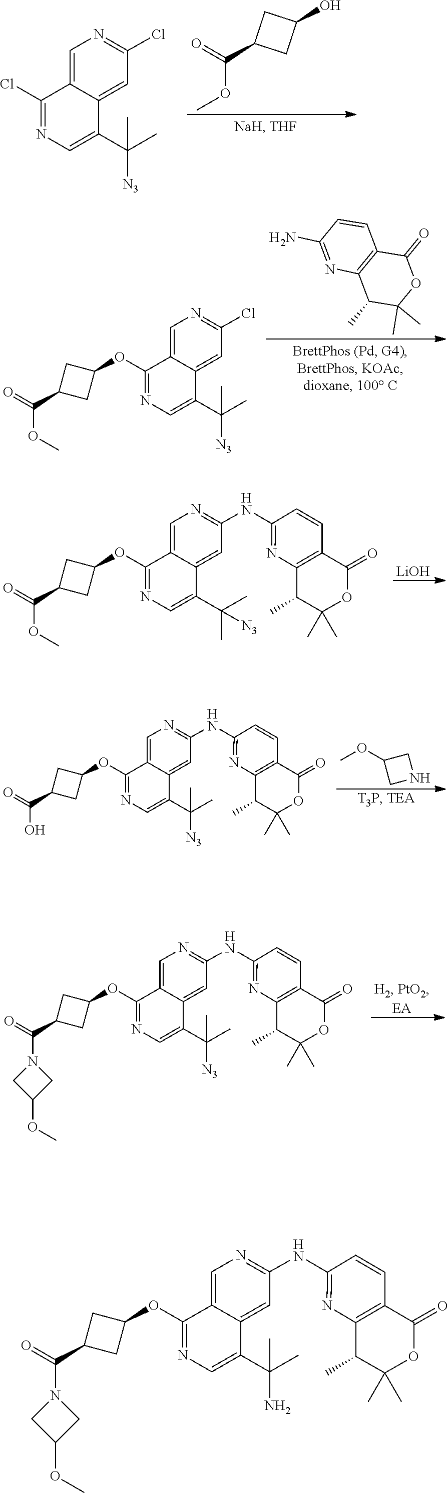

### Step 1: Methyl (cis)-3-((4-(2-azidopropan-2-yl)-6-chloro-2,7-naphthyridin-1-yl)oxy)cyclobutane-1-carboxylate

The title compound was prepared from 4-(2-azidopropan-2-yl)-1,6-dichloro-2,7-naphthyridine (Intermediate 6) and methyl (cis)-3-hydroxycyclobutane-1-carboxylate (Combi-Blocks #OT-2003) using the same procedure as described in Step 1 of Intermediate 7.

### Step 2: Methyl (cis)-3-((4-(2-azidopropan-2-yl)-6-(((R)-7,7,8-trimethyl-5-oxo-7,8-dihydro-5H-pyrano[4,3-b]pyridin-2-yl)amino)-2,7-naphthyridin-1-yl)oxy)cyclobutane-1-carboxylate

The title compound was prepared from methyl (cis)-3-((4-(2-azidopropan-2-yl)-6-chloro-2,7-naphthyridin-1-yl)oxy)cyclobutane-1-carboxylate and Intermediate 3 using a similar procedure described in Step 1 of Example 3a, except at 100° C.

### Step 3: (cis)-3-((4-(2-Azidopropan-2-yl)-6-(((R)-7,7,8-trimethyl-5-oxo-7,8-dihydro-5H-pyrano[4,3-b]pyridin-2-yl)amino)-2,7-naphthyridin-1-yl)oxy)cyclobutane-1-carboxylic acid

Lithium hydroxide hydrate (13.2 mg, 550 umol) was added to a solution of (cis)-3-((4-(2-azidopropan-2-yl)-6-(((R)-7,7,8-trimethyl-5-oxo-7,8-dihydro-5H-pyrano[4,3-b]pyridin-2-yl)amino)-2,7-naphthyridin-1-yl)oxy)cyclobutane-1-carboxylic acid (100 mg, 183 umol) in THF (1 mL) and water (1 mL). The mixture was stirred at 25° C. for 1 h, then was extracted with EA (20 mL×3), the aqueous phase adjusted to pH=3 with 1N HCl, then extracted with EA (20 mL×3), the combined organic phases were dried over sodium sulfate, filtered and concentrated to give the title compound (90 mg, 92% yield) as a yellow solid.

### Step 4: (R)-2-((5-(2-Azidopropan-2-yl)-8-((cis)-3-(3-methoxyazetidine-1-carbonyl)cyclobutoxy)-2,7-naphthyridin-3-yl)amino)-7,7,8-trimethyl-7,8-dihydro-5H-pyrano[4,3-b]pyridin-5-one

T₃P (53.9 mg, 169 umol) and TEA (17.1 mg, 169 umol) were added to a solution of cis)-3-((4-(2-azidopropan-2-yl)-6-(((R)-7,7,8-trimethyl-5-oxo-7,8-dihydro-5H-pyrano[4,3-b]pyridin-2-yl)amino)-2,7-naphthyridin-1-yl)oxy)cyclobutane-1-carboxylic acid and 3-methoxyazetidine (Combi-Blocks #ST-1092)(13.9 mg, 113 umol) in DCM (1 mL) was added. The mixture was stirred at 25° C. for 1 h, then was poured into water 20 mL and extracted with EA (20 mL×3). The combined organic layers were washed with water (20 mL×3), dried over sodium sulfate, filtered and concentrated to give the title compound (30.0 mg, 88% yield) as a yellow solid.

### Step 5: (R)-2-((5-(2-Aminopropan-2-yl)-8-((cis)-3-(3-methoxyazetidine-1-carbonyl)cyclobutoxy)-2,7-naphthyridin-3-yl)amino)-7,7,8-trimethyl-7,8-dihydro-5H-pyrano[4,3-b]pyridin-5-one

The title compound was prepared from (R)-2-((5-(2-Azidopropan-2-yl)-8-((cis)-3-(3-methoxyazetidine-1-carbonyl)cyclobutoxy)-2,7-naphthyridin-3-yl)amino)-7,7,8-trimethyl-7,8-dihydro-5H-pyrano[4,3-b]pyridin-5-one using the same procedure as described in Step 2 of Example 3a. MS (ES+) C₃₁H₃₈N₆O₅ requires: 574, found: 575[M+H]⁺. ¹H-NMR (400 MHz, CD₃OD): δ ppm 9.41 (s, 1H), 9.18 (s, 1H), 8.52 (s, 1H), 8.16 (d, J=8.8 Hz, 1H), 8.02 (s, 1H), 7.27 (d, J=8.8 Hz, 1H), 5.40-5.30 (m, 1H), 4.45-4.35 (m, 1H), 4.40-4.32 (m, 1H), 4.20-4.17 (m, 1H), 4.15-4.05 (m, 1H), 3.85-3.78 (m, 1H), 3.05-2.90 (m, 2H), 2.85-2.75 (m, 2H), 2.51-2.41 (m, 2H), 1.96 (d, J=11.4 Hz, 6H), 1.54 (s, 3H), 1.45 (s, 3H), 1.41 (d, J=7.2 Hz, 3H).

### Example 3f: Synthetic Method 5

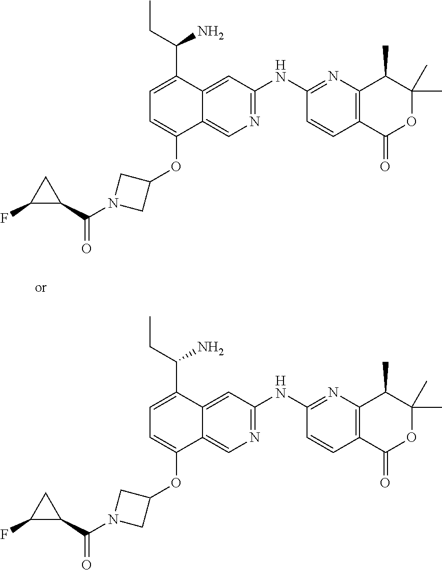 [SMILES: CC[C@@H](N)c1ccc(OC2CN(C(=O)[C@@H]3C[C@@H]3F)C2)c2cnc(Nc3ccc4c(n3)[C@@H](C)C(C)(C)OC4=O)cc12 ; CC[C@H](N)c1ccc(OC2CN(C(=O)[C@@H]3C[C@@H]3F)C2)c2cnc(Nc3ccc4c(n3)[C@@H](C)C(C)(C)OC4=O)cc12]

(R)-2-((5-((R)-1-Aminopropyl)-8-((1-((1S,2S)-2-fluorocyclopropane-1-carbonyl)azetidin-3-yl)oxy)isoquinolin-3-yl)amino)-7,7,8-trimethyl-7,8-dihydro-5H-pyrano[4,3-b]pyridin-5-one or (R)-2-((5-((S)-1-aminopropyl)-8-((1-((1S,2S)-2-fluorocyclopropane-1-carbonyl)azetidin-3-yl)oxy)isoquinolin-3-yl)amino)-7,7,8-trimethyl-7,8-dihydro-5H-pyrano[4,3-b]pyridin-5-one (Compound 83)

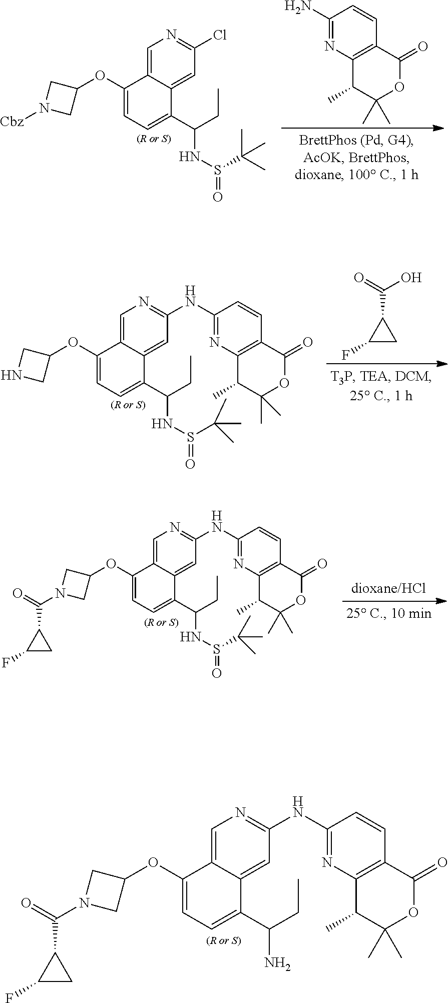 [SMILES: CCC(N[S@@](=O)C(C)(C)C)c1ccc(OC2CN(C(=O)OCc3ccccc3)C2)c2cnc(Cl)cc12 ; C[C@@H]1c2nc(N)ccc2C(=O)OC1(C)C ; CCC(NS(=O)C(C)(C)C)c1ccc(OC2CNC2)c2cnc(Nc3ccc4c(n3)[C@@H](C)C(C)(C)OC4=O)cc12 ; O=C(O)[C@@H]1C[C@@H]1F ; CCC(N[S@@](=O)C(C)(C)C)c1ccc(OC2CN(C(=O)[C@@H]3C[C@@H]3F)C2)c2cnc(Nc3ccc4c(n3)[C@@H](C)C(C)(C)OC4=O)cc12 ; CCC(N)c1ccc(OC2CN(C(=O)[C@@H]3C[C@@H]3F)C2)c2cnc(Nc3ccc4c(n3)[C@@H](C)C(C)(C)OC4=O)cc12]

### Step 1: (S)—N—((R)-1-(8-(Azetidin-3-yloxy)-3-(((R)-7,7,8-trimethyl-5-oxo-7,8-dihydro-5H-pyrano[4,3-b]pyridin-2-yl)amino)isoquinolin-5-yl)propyl)-2-methylpropane-2-sulfinamide or (S)—N—((S)-1-(8-(azetidin-3-yloxy)-3-(((R)-7,7,8-trimethyl-5-oxo-7,8-dihydro-5H-pyrano[4,3-b]pyridin-2-yl)amino)isoquinolin-5-yl)propyl)-2-methylpropane-2-sulfinamide

A mixture of benzyl 3-((5-((R)-1-(((S)-tert-butylsulfinyl)amino)propyl)-3-chloroisoquinolin-8-yl)oxy)azetidine-1-carboxylate or benzyl 3-((5-((S)-1-(((S)-tert-butylsulfinyl)amino)propyl)-3-chloroisoquinolin-8-yl)oxy)azetidine-1-carboxylate (Intermediate 39, 50.0 mg, 94.3 mol), (R)-2-amino-7,7,8-trimethyl-7,8-dihydro-5H-pyrano[4,3-b]pyridin-5-one (19.5 mg, 94.3 mol), BrettPhos (Pd, G4) (8.68 mg, 9.43 μmol), BrettPhos (5.06 mg, 9.43 μmol) and KOAc (23.1 mg, 236 mmol) in dioxane (2 mL) was degassed and purged with nitrogen 3 times, then the reaction mixture was stirred at 100° C. for 1 h. The reaction mixture was then concentrated, and the residue was purified by silica gel chromatography (PE:EA=1:0 to 0:1) to give the title compound as a yellow solid. Note that under the conditions of this coupling reaction, the Cbz group was also removed.

### Step 2: (S)—N—((R)-1-(8-((1-((1S,2S)-2-Fluorocyclopropane-1-carbonyl)azetidin-3-yl)oxy)-3-(((R)-7,7,8-trimethyl-5-oxo-7,8-dihydro-5H-pyrano[4,3-b]pyridin-2-yl)amino)isoquinolin-5-yl)propyl)-2-methylpropane-2-sulfinamide or (S)—N—((S)-1-(8-((1-((1S,2S)-2-fluorocyclopropane-1-carbonyl)azetidin-3-yl)oxy)-3-(((R)-7,7,8-trimethyl-5-oxo-7,8-dihydro-5H-pyrano[4,3-b]pyridin-2-yl)amino)isoquinolin-5-yl)propyl)-2-methylpropane-2-sulfinamide

To a solution of (S)—N—((R)-1-(8-(azetidin-3-yloxy)-3-(((R)-7,7,8-trimethyl-5-oxo-7,8-dihydro-5H-pyrano[4,3-b]pyridin-2-yl)amino)isoquinolin-5-yl)propyl)-2-methylpropane-2-sulfinamide or (S)—N—((S)-1-(8-(azetidin-3-yloxy)-3-(((R)-7,7,8-trimethyl-5-oxo-7,8-dihydro-5H-pyrano[4,3-b]pyridin-2-yl)amino)isoquinolin-5-yl)propyl)-2-methylpropane-2-sulfinamide (15.0 mg, 26.5 μmol) and Et₃N (13.4 mg, 133 mol) in DCM (2 mL) was added (1S,2S)-2-fluorocyclopropane-1-carboxylic acid (63.9 mg, 614 μmol) and T₃P (21.1 mg, 66.3 mol). The reaction mixture was stirred at 25° C. for 1 h, then was diluted with water (10 mL) and extracted with DCM (10 mL×3). The combined organic layers were dried over anhydrous sodium sulfate, filtered and concentrated to give a residue. The residue was purified by prep-TLC (PE:EA=0:1) to give the title compound (15.0 mg, 87% yield) as a yellow solid.

### Step 3: (R)-2-((5-((R)-1-Aminopropyl)-8-((1-((1S,2S)-2-fluorocyclopropane-1-carbonyl)azetidin-3-yl)oxy)isoquinolin-3-yl)amino)-7,7,8-trimethyl-7,8-dihydro-5H-pyrano[4,3-b]pyridin-5-one or (R)-2-((5-((S)-1-aminopropyl)-8-((1-((1S,2S)-2-fluorocyclopropane-1-carbonyl)azetidin-3-yl)oxy)isoquinolin-3-yl)amino)-7,7,8-trimethyl-7,8-dihydro-5H-pyrano[4,3-b]pyridin-5-one (Compound 83)

A solution of (S)—N—((R)-1-(8-((1-((1S,2S)-2-fluorocyclopropane-1-carbonyl)azetidin-3-yl)oxy)-3-(((R)-7,7,8-trimethyl-5-oxo-7,8-dihydro-5H-pyrano[4,3-b]pyridin-2-yl)amino)isoquinolin-5-yl)propyl)-2-methylpropane-2-sulfinamide or (S)—N—((S)-1-(8-((1-((1S,2S)-2-fluorocyclopropane-1-carbonyl)azetidin-3-yl)oxy)-3-(((R)-7,7,8-trimethyl-5-oxo-7,8-dihydro-5H-pyrano[4,3-b]pyridin-2-yl)amino)isoquinolin-5-yl)propyl)-2-methylpropane-2-sulfinamide (10.0 mg, 15.3 μmol) in HCl/dioxane (4 M, 452 uL) was stirred at 25° C. for 10 min. The reaction mixture was then concentrated to give a residue. The residue was purified by prep-HPLC (column: Waters Xbridge 150*25 mm*5 um; mobile phase: [water (10 mM NH₄HCO₃)-ACN]; B %: 22%-52%, 10 min) to give the title compound (2.2 mg, 26% yield) as a white solid. MS (ES+) C₃₀H₃₄FN₅O₄ requires: 547, found: 548[M+H]⁺. ¹H NMR (400 M Hz, CD₃OD): δ ppm 9.43 (d, J=4.4 Hz, 1H), 8.92 (s, 1H), 8.09 (d, J=8.4 Hz, 1H), 7.72-7.62 (m, 1H), 7.20 (d, J=8.8 Hz, 1H), 6.66 (d, J=8.0 Hz, 1H), 5.38-5.29 (m, 1H), 4.97-4.90 (m, 1H), 4.81-4.70 (m, 1H), 4.65-4.42 (m, 3H), 4.22-4.12 (m, 1H), 3.19-3.10 (m, 1H), 2.04-1.83 (m, 3H), 1.78-1.63 (m, 1H), 1.54-1.47 (m, 6H), 1.45 (s, 3H), 1.18-1.07 (m, 1H), 0.97 (t, J=7.4 Hz, 3H).

### Example 3g: Synthetic Method 5

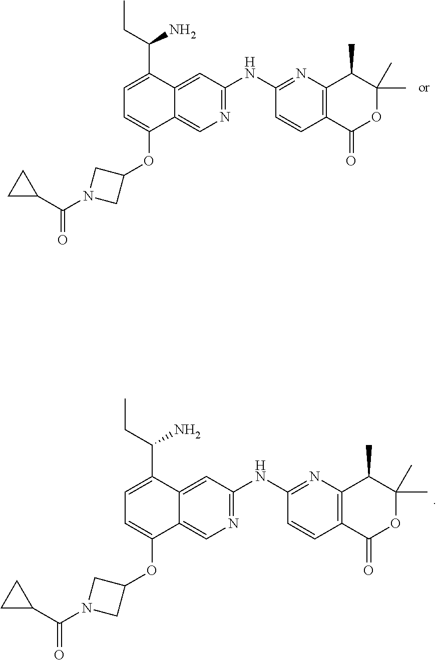 [SMILES: CC[C@@H](N)c1ccc(OC2CN(C(=O)C3CC3)C2)c2cnc(Nc3ccc4c(n3)[C@@H](C)C(C)(C)OC4=O)cc12 ; CC[C@H](N)c1ccc(OC2CN(C(=O)C3CC3)C2)c2cnc(Nc3ccc4c(n3)[C@@H](C)C(C)(C)OC4=O)cc12]

(R)-2-((5-((R)-1-Aminopropyl)-8-((1-(cyclopropanecarbonyl)azetidin-3-yl)oxy)isoquinolin-3-yl)amino)-7,7,8-trimethyl-7,8-dihydro-5H-pyrano[4,3-b]pyridin-5-one or (R)-2-((5-((S)-1-aminopropyl)-8-((1-(cyclopropanecarbonyl)azetidin-3-yl)oxy)isoquinolin-3-yl)amino)-7,7,8-trimethyl-7,8-dihydro-5H-pyrano[4,3-b]pyridin-5-one (Compound 40)

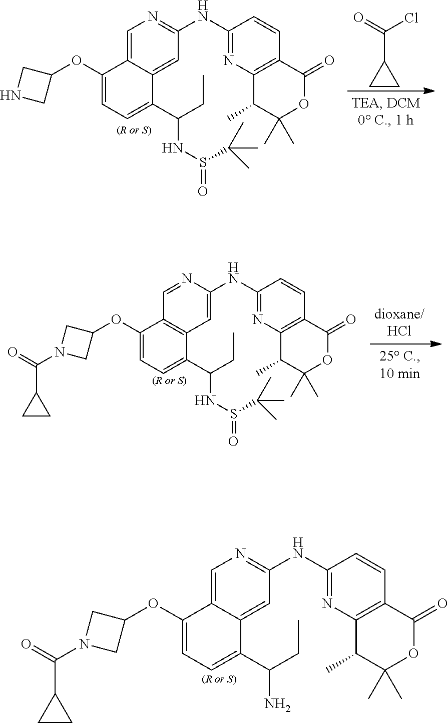 [SMILES: CCC(N[S@@](=O)C(C)(C)C)c1ccc(OC2CNC2)c2cnc(Nc3ccc4c(n3)[C@@H](C)C(C)(C)OC4=O)cc12 ; O=C(Cl)C1CC1 ; CCC(N[S@@](=O)C(C)(C)C)c1ccc(OC2CN(C(=O)C3CC3)C2)c2cnc(Nc3ccc4c(n3)[C@@H](C)C(C)(C)OC4=O)cc12 ; CCC(N)c1ccc(OC2CN(C(=O)C3CC3)C2)c2cnc(Nc3ccc4c(n3)[C@@H](C)C(C)(C)OC4=O)cc12]

### Step 1: (S)—N—((R)-1-(8-((1-(Cyclopropanecarbonyl)azetidin-3-yl)oxy)-3-(((R)-7,7,8-trimethyl-5-oxo-7,8-dihydro-5H-pyrano[4,3-b]pyridin-2-yl)amino)isoquinolin-5-yl)propyl)-2-methylpropane-2-sulfinamide or (S)—N—((S)-1-(8-((1-(cyclopropanecarbonyl)azetidin-3-yl)oxy)-3-(((R)-7,7,8-trimethyl-5-oxo-7,8-dihydro-5H-pyrano[4,3-b]pyridin-2-yl)amino)isoquinolin-5-yl)propyl)-2-methylpropane-2-sulfinamide

A solution of (S)—N—((R)-1-(8-(azetidin-3-yloxy)-3-(((R)-7,7,8-trimethyl-5-oxo-7,8-dihydro-5H-pyrano[4,3-b]pyridin-2-yl)amino)isoquinolin-5-yl)propyl)-2-methylpropane-2-sulfinamide or (S)—N—((S)-1-(8-(azetidin-3-yloxy)-3-(((R)-7,7,8-trimethyl-5-oxo-7,8-dihydro-5H-pyrano[4,3-b]pyridin-2-yl)amino)isoquinolin-5-yl)propyl)-2-methylpropane-2-sulfinamide (40.0 mg, 70.7 μmol) and Et₃N (35.8 mg, 354 mol) in DCM (1 mL) was added cyclopropanecarbonyl chloride (8.13 mg, 77.8 μmol) at 0° C. The reaction mixture was stirred at 0° C. for 1 h, then was diluted with water (10 mL) and extracted with DCM (10 mL×3). The combined organic layers were dried over anhydrous sodium sulfate, filtered and concentrated to give a residue. The residue was purified by prep-TLC (PE:EA=0:1) to give the title compound (20.0 mg, 45% yield) as yellow solid.

### Step 2: (R)-2-((5-((R)-1-Aminopropyl)-8-((1-(cyclopropanecarbonyl)azetidin-3-yl)oxy)isoquinolin-3-yl)amino)-7,7,8-trimethyl-7,8-dihydro-5H-pyrano[4,3-b]pyridin-5-one or (R)-2-((5-((S)-1-aminopropyl)-8-((1-(cyclopropanecarbonyl)azetidin-3-yl)oxy)isoquinolin-3-yl)amino)-7,7,8-trimethyl-7,8-dihydro-5H-pyrano[4,3-b]pyridin-5-one (Compound 40)

The title compound was prepared from (S)—N—((R)-1-(8-((1-(cyclopropanecarbonyl)azetidin-3-yl)oxy)-3-(((R)-7,7,8-trimethyl-5-oxo-7,8-dihydro-5H-pyrano[4,3-b]pyridin-2-yl)amino)isoquinolin-5-yl)propyl)-2-methylpropane-2-sulfinamide or (S)—N—((S)-1-(8-((1-(cyclopropanecarbonyl)azetidin-3-yl)oxy)-3-(((R)-7,7,8-trimethyl-5-oxo-7,8-dihydro-5H-pyrano[4,3-b]pyridin-2-yl)amino)isoquinolin-5-yl)propyl)-2-methylpropane-2-sulfinamide using a procedure similar to the one described in Step 3 of Example 3f. MS (ES+) C₃₀H₃₅N₅O₄ requires: 529, found: 530[M+H]⁺. ¹H NMR (400 M Hz, CD₃OD): δ ppm 9.43 (s, 1H), 8.91 (s, 1H), 8.09 (d, J=8.8 Hz, 1H), 7.67 (d, J=8.0 Hz, 1H), 7.20 (d, J=8.8 Hz, 1H), 6.65 (d, J=8.0 Hz, 1H), 5.39-5.27 (m, 1H), 4.90 (s, 1H), 4.60-4.46 (m, 3H), 4.16-4.07 (m, 1H), 3.20-3.09 (m, 1H), 2.03-1.82 (m, 2H), 1.71-1.60 (m, 1H), 1.54-1.48 (m, 6H), 1.45 (s, 3H), 0.97 (t, J=7.4 Hz, 3H), 0.94-0.82 (m, 4H).

### Example 4: Inhibition of MAP4K1 Biochemical Enzymatic Activity

MAP4K1 (HPK1) and relevant off-target enzymatic activity was monitored using the Perkin Elmer electrophoretic mobility shift technology platform—the EZReader 2. Fluorescent labeled substrate peptide was incubated in the presence of kinase and ATP, and in the presence of dosed compound, such that each dose of compound resulted in a reflective proportion of the peptide to be phosphorylated. Within the linear, steady-state phase of the kinase enzymatic reaction, the mixed pool of phosphorylated (product) and non-phosphorylated (substrate) peptides was passed through the microfluidic system of the PerkinElmer EZ Reader 2, under an applied electric potential difference. The presence of the phosphate group on the product peptide provided a difference in mass and charge between that of the substrate peptide, resulting in a separation of the substrate and product pools in the sample (Perrin et al. 2010). As the product and substrate peptide mixture passes the lasers within the instrument, these pools are detected (λₑₓ=488 nm, λₑₘ=568 nm) and resolved as separate peaks. The ratio between these peaks reflects the activity of the compound at that concentration, in that well, under those conditions.

### Enzyme Activity Inhibition Assay Protocol:

Inhibitors were dissolved in 100% DMSO at a stock concentration of 10 mM. A 100×, 10-point, 4-fold serial dilution of each inhibitor was created in 100% DMSO either manually or on a Hamilton STAR liquid handler, starting at a relevant concentration, usually 1 mM. A volume of 0.130 μL of each concentration was transferred to the relevant wells of a 384-well plate (Greiner 781 201) in diplicate using a TTPLabtech Mosquito nano-litre dispenser. Using a Multidrop Combi, the remaining constituents of the kinase reaction were added to the 130 nL of dosed compound as follows (see table below for final reaction details):

Enzyme activity assays at the ^{APP}K_{M} for ATP or 1 mM ATP: In each well of a 384-well plate, 0.1-15 nM of untreated enzyme was incubated in a total of 13 μL of buffer (100 mM HEPES pH 7.5, 0.015% Brij 35, 10 mM MgCl₂, 1 mM DTT) with 1.5 μM fluorescent peptid and 20-1000 μM ATP, at 25° C., for 60-180 minutes in the presence or absence of a dosed concentration series of compound (1% DMSO final concentration). The kinase reactions were stopped by the addition of 70 μl of Stop buffer (100 mM HEPES pH 7.5, 0.015% Brij 35, 35 mM EDTA and 0.2% of Coating Reagent 3 (Caliper Lifesciences)). The plates were read on a Caliper EZReader 2 as described above.

TABLE 2
Kinase Reaction Conditions

| Enzyme | Enzyme | | ATP | | Substrate Peptide & | Kinase Reaction | |
|---|---|---|---|---|---|---|---|
| (Source) | Concentration | | Concentration | | Concentration | Time | |
| HPK1 | 0.5 | nM | 26 | μM | S6K2tide, 1.5 μM | 120 | min. |
| (Invitrogen) | | | | | | | |
| HPK1 | 0.25 | nM | 1000 | μM | S6K2tide, 1.5 μM | 120 | min. |
| (Invitrogen) | | | | | | | |
| LCK | 12 | nM | 26 | μM | FL4tide, 1.5 μM | 60 | min. |
| (Invitrogen) | | | | | | | |
| HGK | 0.1 | nM | 50 | μM | FL25tide, 1.5 μM | 60 | min. |
| (Invitrogen) | | | | | | | |
| GLK | 15 | nM | 20 | μM | PKAtide, 2 μM | 180 | min. |
| (SignalChem) | | | | | | | |

S6K2tide;
Carna Biosciences (5-FAM-Proprietary Sequence-CONH2)
FL4tide;
Perkin Elmer (5-FAM-EGIYGVLFKKK (SEQ ID NO: 1)-CONH₂)
FL25tide; Perkin Elmer
(5-FAM-VDGKEIYNTIRRK (SEQ ID NO: 2)-CONH₂)
PKAtide;
Anaspec Peptide Co.(5-FAM-GRTGRRNSI (SEQ ID NO: 3)-CONH₂)

Perrin D, Frémaux C, Shutes A. Capillary microfluidic electrophoretic mobility shift assays: application to enzymatic assays in drug discovery. Expert Opin Drug Discov. 2010, 5(1):51-63.

The results obtained in these experiments for compounds prepared according to the examples are summarized in Table 3 below.

TABLE 3

| Compound | MAP4K1 | IL-2 Primary T |
|---|---|---|
| No. | IC50 (nM) | Cell EC50 (nM) |
| 1 | 0.18 | 34.7 |
| 2 | 0.38 | |
| 3 | 0.33 | 33.5 |
| 4 | 0.15 | 19.9 |
| 5 | 0.17 | 17.7 |
| 6 | 0.20 | 22.5 |
| 7 | 0.12 | 22.3 |
| 8 | 0.16 | 32.7 |
| 9 | 0.16 | 16.8 |
| 10 | 0.13 | 8.1 |
| 11 | 0.63 | 38.6 |
| 12 | 0.16 | 10.5 |
| 13 | 0.17 | 23.2 |
| 14 | 0.19 | 15.3 |
| 15 | 0.13 | 42.0 |
| 16 | 0.47 | |
| 17 | 0.49 | |
| 18 | 3.06 | |
| 19 | 0.20 | 41.1 |
| 20 | 0.26 | 99.3 |
| 21 | 0.14 | 16.8 |
| 22 | 0.54 | 479.3 |
| 23 | 0.32 | |
| 24 | 0.89 | |
| 25 | 1.38 | |
| 26 | 0.31 | |
| 27 | 0.30 | |
| 28 | 0.29 | |
| 29 | 0.12 | 11.0 |
| 30 | 0.20 | |
| 31 | 0.21 | 42.8 |
| 32 | 0.17 | |
| 33 | 0.15 | 9.2 |
| 34 | 0.19 | 43.3 |
| 35 | 0.30 | |
| 36 | 0.65 | |
| 37 | 0.15 | |
| 38 | 0.23 | |
| 39 | 0.14 | 14.0 |
| 40 | 0.19 | 160.4 |
| 41 | 0.10 | |
| 42 | 0.33 | |
| 43 | 0.22 | |
| 44 | 0.12 | |
| 45 | 0.16 | |
| 46 | 0.23 | |
| 47 | 0.18 | |
| 48 | 0.12 | 28.7 |
| 49 | 0.24 | 25.7 |
| 50 | 0.32 | |
| 51 | 0.36 | |
| 52 | 0.18 | |
| 53 | 0.54 | 57.4 |
| 54 | 0.21 | 29.3 |
| 55 | 0.16 | |
| 56 | 0.17 | 23.8 |
| 57 | 0.13 | 21.1 |
| 58 | 0.75 | |
| 59 | 0.24 | |
| 60 | 0.16 | |
| 61 | 0.21 | |
| 62 | 0.29 | 106.6 |
| 63 | 0.19 | 23.2 |
| 64 | 0.16 | |
| 65 | 0.20 | 17.5 |
| 66 | 0.22 | |
| 67 | 0.20 | |
| 68 | 0.16 | |
| 69 | 0.14 | |
| 70 | 0.18 | 12.5 |
| 71 | 0.27 | |
| 72 | 0.21 | 30.5 |
| 73 | 0.32 | |
| 74 | 0.36 | |
| 75 | 0.38 | |
| 76 | 0.17 | 47.3 |
| 77 | 0.36 | 47.8 |
| 78 | 0.43 | |
| 79 | 0.31 | |
| 80 | 0.17 | 62.9 |
| 81 | 0.16 | 71.2 |
| 82 | 0.24 | 576.6 |
| 83 | 0.11 | 23.8 |
| 84 | 0.24 | |
| 85 | 0.10 | 17.8 |
| 86 | 0.27 | |
| 87 | 0.22 | 50 |
| 88 | 0.21 | |
| 89 | 0.35 | |
| 90 | 0.18 | |
| 91 | 0.31 | |
| 92 | 0.17 | 34.0 |
| 93 | 0.17 | |
| 94 | 0.14 | |
| 95 | 0.22 | 19.8 |
| 96 | 0.09 | 10.3 |
| 97 | 0.36 | 50.1 |
| 98 | 0.19 | |
| 99 | 0.14 | 24.7 |
| 100 | 0.11 | 22.1 |
| 101 | 0.13 | 17.8 |
| 102 | 0.12 | 24.0 |
| 103 | 0.24 | |
| 104 | 0.20 | |
| 105 | 0.20 | |
| 106 | 0.39 | |
| 107 | 0.31 | |
| 108 | 2.86 | |
| 109 | 4.00 | |
| 110 | 0.62 | |
| 111 | 0.43 | 9.6 |
| 112 | 0.24 | 10.2 |
| 113 | 0.11 | 7.4 |
| 114 | 0.07 | |
| 115 | 0.10 | 5.4 |
| 116 | 1.13 | |
| 117 | 2.02 | |
| 118 | 0.17 | 17.7 |
| 119 | 0.20 | 90 |
| 120 | 0.25 | 29.4 |
| 121 | 0.20 | 41.5 |
| 122 | 0.12 | 53.7 |
| 123 | 0.10 | 60.8 |
| 124 | 0.26 | |
| 125 | 0.17 | 130 |
| 126 | 1.81 | |
| 127 | 0.09 | 74.2 |

### Example 5: T Cell Enhancement of Cytokines

Isolation and Expansion of T Cells from Whole Blood

T cells are isolated from whole blood of healthy donors by immunomagnetic negative selection following manufacture's protocol (StemCell Technologies, human T cell isolation kit). Purity of isolated cells is assessed by flow cytometry and yields 95-98% CD3⁺ T cells. For expansion of T cells, 1×10⁶ cells/well are plated in serum free cell expansion media (ThermoFisher) containing 30U of recombinant human IL2 (R&D) and stimulated with 25 ul of CD3/CD28 beads (Invitrogen) in 24 well plates for 3-4 days. T cells are then expanded in 175 cm flasks and maintained at a cell density of 1 to 2.5×10⁶ cells/ml days by addition of ⅔ of fresh media every 2-3 days. After 10-14 days, cells are frozen in BamBanker freezing media (Thermo) and stored in liquid nitrogen. Phenotypic analysis of expanded T cells by flow cytometry, routinely shows 60% cells are CD8+ T cells upon freezing.

Cytokine Measurement

For IL2 measurement, expanded CD3+ T cells are dispensed at 100K cells/well (cultured in X-VIVO 10 Serum-free media) and are stimulated with plate-bound anti-CD3 and soluble anti-CD28 in the presence of vehicle or compound of the disclosure at various concentrations for 24 h. As outlined in the manufacturer's protocol (Cisbio), 16 μL of conditioned media is transferred to a white 384-well low volume plate. Following a 24 h incubation with the anti-IL2 antibodies, the homogenous time resolved fluorescence (HTRF) is measured.

### Example 6: Inhibition of Anti-Tumor Activity in a Syngeneic Mouse Model

Generation of the MCA205 Syngeneic Xenograft Anti-Tumor Efficacy Study

Six to eight-week-old female, C57BL/6 mice (Jackson Labs, Bar Harbor, ME) are implanted subcutaneously on the left flank with 1×10⁶ MCA205 cells/mouse. After tumors reach an average volume of 50 mm³, mice are randomized into treatment groups, 10 mice per group, with tumors in the size range of 30-70 mm³. Compounds of the disclosure 10-30 mg/kg, anti-mouse PD-L1 mAb (B7 H1, clone #10F.9G2 Bio-X-cell, Lebanon, NH) and vehicle either alone or in different combinations are administered to tumor bearing mice. Reduction in tumor volume is measured [mm³] over time.

### EQUIVALENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those skilled in the art will recognize or be able to ascertain using no more than routine experimentation, many equivalents to the specific embodiments of the disclosure described and claimed herein. Such equivalents are intended to be encompassed by the following claims.

1. A compound of Formula I 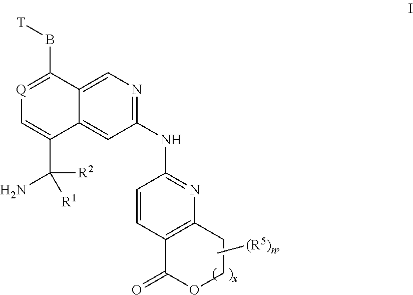
or a pharmaceutically acceptable salt thereof,
wherein:
T is selected from 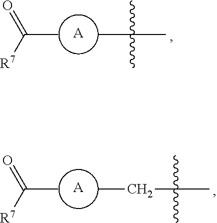
-L¹-C(O)—NR¹¹R¹², -L¹-O—C(O)—NR¹¹R¹² and -L²-NR¹³—C(O)—R¹⁴;
Ring A is C₄₋₆ cycloalkyl or 4-6 membered heterocycle containing nitrogen, wherein said cycloalkyl or heterocycle is optionally substituted with 1-2 R⁶; each R⁶ is independently selected from CH₃, methoxy, CF₃, CH₂F, and CHF₂;
L¹ and L² are each independently selected from C₁-C₃ alkylene optionally substituted with 1-2 R¹⁵;
each s is independently selected from 1, 2, and 3;
B is O or NH;
Q is N or CH;
x is 0, 1, or 2;
R¹ and R² are each independently selected from hydrogen, C₁₋₆ alkyl, C₃₋₆ cycloalkyl, and 4 to 6-membered heterocycle, wherein said alkyl is optionally substituted with 1-2 R³;
each R³ is independently selected from halogen, hydroxyl and OR⁴; or
each R⁴ is independently selected from C₁₋₃ alkyl, CF₃, CH₂F, and CHF₂;
each R⁵ is independently selected from C₁₋₂ alkyl, CF₃, CH₂F, and CHF₂, or
two R⁵ attached to the same carbon atom taken together with the carbon atom to which they attach form C₃₋₅ cycloalkyl; or two R⁵ attached to two adjacent carbon atoms taken together with the two adjacent carbon atoms to which they attach form C₄₋₆ cycloalkyl;
n is 0, 1, 2, 3, or 4;
R⁷ is selected from C₁₋₃ alkyl, C₃₋₆ cycloalkyl, OC₁₋₄ alkyl, NR⁹R¹⁰, and 3-5 membered heterocycle containing nitrogen or oxygen, wherein said alkyl, cycloalkyl, or heterocycle is optionally substituted with 1-3 R⁸;
each R⁸ is independently selected from halogen, C₁₋₃ alkyl, CF₃, CH₂F, CHF₂, hydroxyl, OC₁₋₃ alkyl, OCF₃, OCH₂F, and OCHF₂;
R⁹ is selected from C₁₋₂ alkyl;
R¹⁰ is selected from C₁₋₂ alkyl
R¹¹ is selected from C₁₋₃alkyl;
R¹² is selected from C₁₋₃ alkyl; or R¹¹ and R¹² taken together with the nitrogen atom to which they attach form 4 to 6-membered heterocycle containing nitrogen;
R¹³ is H or C₁₋₂alkyl;
R¹⁴ is C₁₋₃alkyl, or R¹³ and R¹⁴ taken together with the atoms to which attach form 4 to 6-membered heterocycle containing nitrogen; and
each R¹⁵ is independently selected from halogen, methoxy, and C₁₋₂alkyl, or two R¹⁵ taken together with the two adjacent carbon atoms to which they attach form cyclopropyl.

2. The compound of claim 1, or a pharmaceutically acceptable salt thereof, wherein:
T is selected from 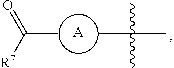
-L¹-C(O)NR¹¹R¹², and -L²-NR¹³C(O)R¹⁴.

3. The compound of claim 1, or a pharmaceutically acceptable salt thereof, wherein:
L¹ is selected from —(CH₂)₃— and: 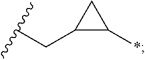
L² is —(CH₂)₂—;
R¹ and R² are each independently selected from hydrogen, C₁₋₄ alkyl, C₃₋₄ cycloalkyl, and
4 to 5-membered heterocycle, wherein said alkyl is optionally substituted with one R³; each R³ is independently selected from F, Cl, hydroxyl and OR⁴;
each R⁴ is independently selected from CH₃, CH₂CH₃, and CF₃;
each R⁵ is independently selected from CH₃ and CF₃, or
two R⁵ attached to the same carbon atom taken together with the carbon atom to which they attach form C₃₋₄ cycloalkyl; or two R⁵ attached to two adjacent carbon atoms taken together with the two adjacent carbon atoms to which they attach form C₄₋₅ cycloalkyl;
n is 1, 2, 3, or 4;
each R⁶ is independently selected from CH₃, CF₃, CH₂F and CHF₂;
R⁷ is selected from C₁₋₂ alkyl, C₃₋₅ cycloalkyl, OC₁₋₃ alkyl, NR⁹R¹⁰, and 3-5 membered heterocycle containing nitrogen or oxygen, wherein said alkyl, cycloalkyl, or heterocycle is optionally substituted with 1-3 R⁸;
each R⁸ is independently selected from halogen, C₁₋₂ alkyl, CF₃, CHF₂, hydroxyl, OC₁₋₂ alkyl, OCF₃, and OCHF₂;
R⁹ is CH₃;
R¹⁰ is CH₃;
R¹¹ is CH₃;
R¹² is CH₃;
R¹³ is CH₃; and
R¹⁴ is CH₃; or R¹³ and R¹⁴ taken together with the atoms to which they attach form 5-membered heterocycle containing nitrogen,
wherein: 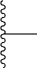
represents the point of attachment to B; and [symbol]
represents the point of attachment to —C(O)NR¹¹R¹².

4. The compound of claim 1, wherein the compound is represented by Formula II: 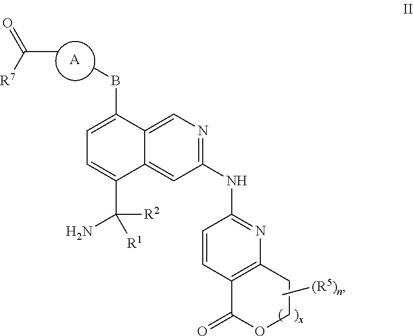
or a pharmaceutically acceptable salt thereof.

5. The compound of claim 1, wherein the compound is represented by Formula III: 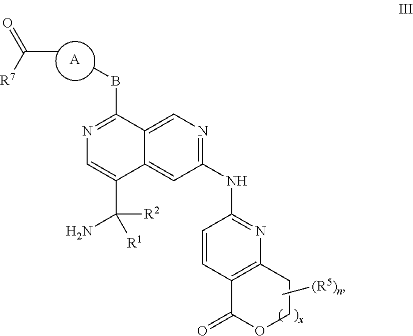
or a pharmaceutically acceptable salt thereof.

6.-8. (canceled)

9. The compound of claim 5, or a pharmaceutically acceptable salt thereof, wherein:
Ring A is selected from cyclobutylene, azetidinylene, and pyrrolidinylene, wherein said cyclobutylene, azetidinylene, or pyrrolidinylene is optionally substituted with one R⁶.

10. The compound of claim 5, or a pharmaceutically acceptable salt thereof, wherein:
Ring A is C₄₋₆ cycloalkyl optionally substituted with 1-2 R⁶, and R⁷ is NR⁹R¹⁰ or 3-5 membered heterocycle containing nitrogen, wherein a ring nitrogen of the 3-5 membered heterocycle is bonded to R⁷—C(O [symbol] , and the heterocycle is optionally substituted with 1-3 R⁸;
Ring A is 4-6 membered heterocycle containing nitrogen, wherein a ring nitrogen of the 4-6 membered heterocycle is bonded to R⁷—C(O [symbol] , and the heterocycle is optionally substituted with 1-2 R⁶.

11. The compound of claim 5, wherein the compound is represented by Formula VII: 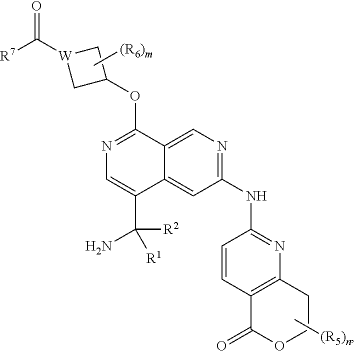
or a pharmaceutically acceptable salt thereof,
wherein:
W is N or CH; and
m is 0 or 1.

12.-13. (canceled)

14. The compound of claim 5, wherein the compound is represented by Formula VIII(A), VIII(B), or VIII(C): 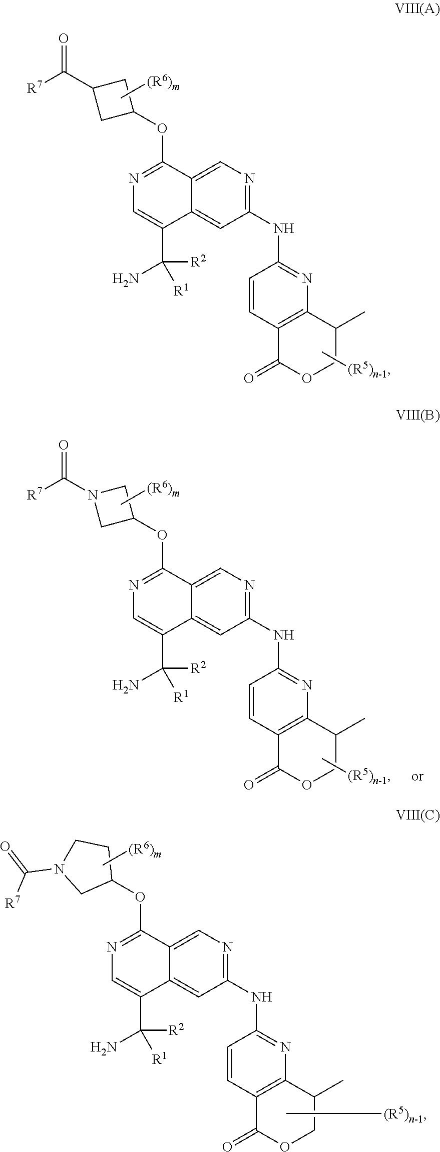
or a pharmaceutically acceptable salt thereof, wherein:
m is 0 or 1.

15.-16. (canceled)

17. The compound of claim 14, or a pharmaceutically acceptable salt thereof, wherein:
R⁶ is CH₃;
R¹ and R² are each independently selected from hydrogen, CH₃, CH₂CH₃, and CH₂OCH₃;
each R⁵ is CH₃ or two R⁵ attached to the same carbon atom taken together with the carbon atom to which they attach form a cyclopropyl:
n is 1, 2, 3, and 4; and
R⁷ is selected from CH₃, CH₂CH₃, N(CH₃)₂, OC₁₋₂alkyl, cyclopropyl, azetidinyl, oxetanyl, tetrahydrofuranyl, wherein R⁷ is optionally substituted with 1-2 R⁸; and
each R⁸ is independently selected from F, CH₃, CH₂CH₃, OH, OCH₃, and CF₃.

18.-20. (canceled)

21. The compound of claim 17, or a pharmaceutically acceptable salt thereof, wherein:
when R⁷—C(O [symbol] is bonded to a ring carbon, R⁷ is N(CH₃)₂ or azetidinyl, wherein said azetidinyl is optionally substituted with 1-2 R⁸;
when R⁷—C(O [symbol] is bonded to a ring nitrogen, R⁷ is selected from CH₃, CH₂CH₃, N(CH₃)₂, OC₁₋₂alkyl, cyclopropyl, azetidinyl, oxetanyl, and tetrahydrofuranyl, wherein said CH₃, CH₂CH₃, OC₁₋₂alkyl, cyclopropyl, azetidinyl, oxetanyl or tetrahydrofuranyl is optionally substituted with 1-2 R⁸; and
each R⁸ is independently selected from F, CH₃, CH₂CH₃, OH, OCH₃, and CF₃.

22. The compound of claim 17, or a pharmaceutically acceptable salt thereof, wherein:
R⁷ is independently selected from CH₃, CH₂CH₃, N(CH₃)₂, OCH₃, OCH₂CH₃, CH₂OCH₃, 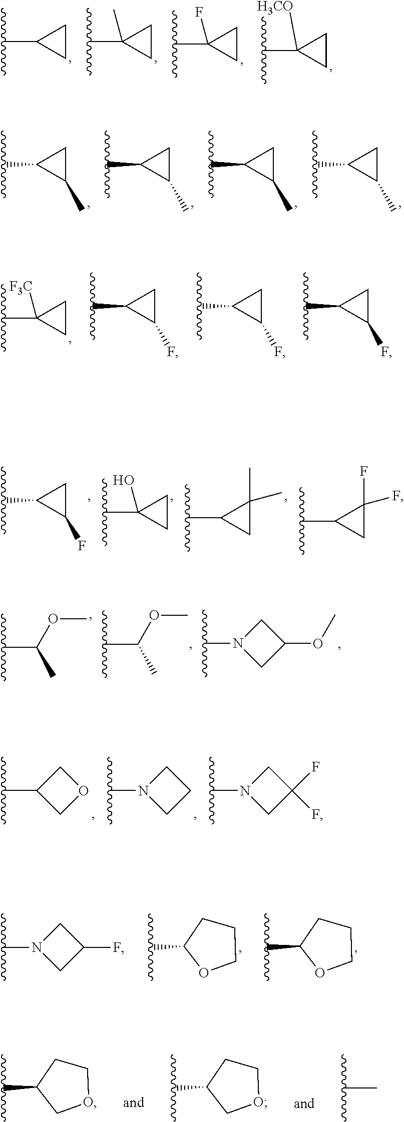
represents the point of attachment to —C(O).

23. The compound of claim 22, or a pharmaceutically acceptable salt thereof, wherein:
when R⁷—C(O [symbol] is bonded to a ring carbon, R⁷ is selected from N(CH₃)₂, 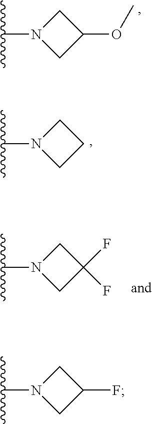
when R⁷—C(O [symbol] is bonded to a ring nitrogen, R⁷ is selected from CH₃, CH₂CH₃, N(CH₃)₂, OCH₃, OCH₂CH₃, CH₂OCH₃, 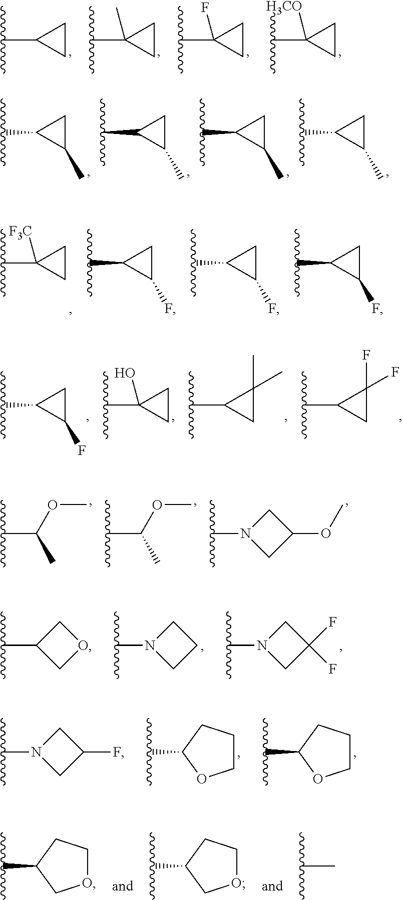
represents the point of attachment to —C(O).

24. The compound of claim 1, wherein the compound is represented by Formula XI: 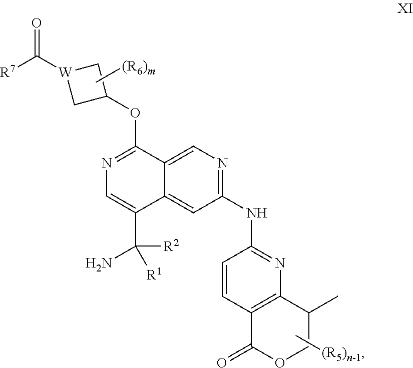
or a pharmaceutically acceptable salt thereof,
wherein:
W is N or CH;
R¹ and R² are each independently selected from hydrogen, CH₃, and CH₂CH₃;
each R⁵ is CH₃;
n is 2 or 3;
R⁶ is CH₃;
m is 0 or 1;
R⁷ is selected from CH₃, CH₂CH₃, cyclopropyl, 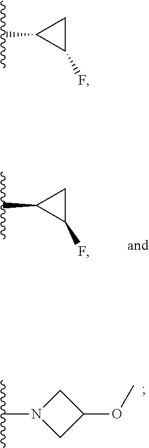
and
wherein 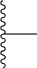
represents the point of attachment —C(O).

25. The compound of claim 24, or a pharmaceutically acceptable salt thereof, wherein:
when R⁷—C(O [symbol] is bonded to a ring carbon, R⁷ is 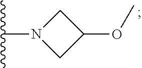 [SMILES: *N1CC(OC)C1]
when R⁷—C(O [symbol] is bonded to a ring nitrogen, R⁷ is selected from CH₃, CH₂CH₃, cyclopropyl 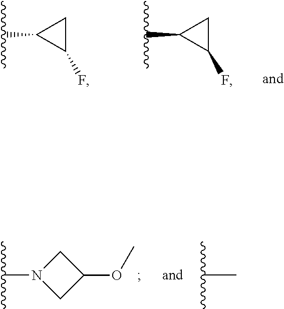
represents the point of attachment —C(O).

26.-28. (canceled)

29. A compound of Table 1 or a pharmaceutically acceptable salt thereof.

30. A pharmaceutical composition comprising the compound of claim 1, or a pharmaceutically acceptable salt thereof, and a pharmaceutically acceptable carrier or excipient.

31.-32. (canceled)

33. A method for treating a MAP4K1-dependent disorder or disease in a subject in need thereof, comprising administering to said subject an effective amount of the compound of claim 1 or a pharmaceutically acceptable salt thereof; or the pharmaceutical composition of claim 30.

34. The method of claim 33, wherein said MAP4K1-dependent disease or disorder is a cancer.

35. The method of claim 34, wherein the cancer comprises at least one cancer selected from the group consisting of colon cancer, pancreatic cancer, breast cancer, prostate cancer, lung cancer, ovarian cancer, cervical cancer, renal cancer, bladder cancer, stomach cancer, liver cancer, cancer of the head and neck, lymphoma, leukemia, and melanoma.

36.-37. (canceled)
